(19) United States
(12) Patent Application Publication
(10) Pub. No.: US 2021/0369710 A1
(43) Pub. Date: Dec. 2, 2021

(54) MODULATORS OF MYC FAMILY PROTO-ONCOGENE PROTEIN

(71) Applicant: Nalo Therapeutics, Belmont, CA (US)

(72) Inventors: Keith Wilson, Belmont, CA (US); William Greenlee, Teaneck, NJ (US); Ken Brameld, Menlo Park, CA (US); Anand Kumar Raichurkar, Karnataka (IN); Prashant Latthe, Karnataka (IN); Ganesh Babu Karunakaran, Hosur (IN)

(73) Assignee: Nalo Therapeutics, Belmont, CA (US)

(21) Appl. No.: 17/404,529

(22) Filed: Aug. 17, 2021

Related U.S. Application Data
(63) Continuation of application No. PCT/US2020/018804, filed on Feb. 19, 2020.
(60) Provisional application No. 62/807,452, filed on Feb. 19, 2019.

(51) Int. Cl. A61K 31/506; A61K 45/06; C07D 401/14; C07D 403/12; C07D 405/14; C07D 413/14; C07D 409/14; C07D 493/08; C07D 403/14; C07D 451/04; C07D 453/02

(52) U.S. Cl. CPC A61K 31/506; A61K 45/06; C07D 401/14; C07D 403/12; C07D 405/14; C07D 453/02; C07D 409/14; C07D 493/08; C07D 403/14; C07D 451/04; C07D 413/14

(57) ABSTRACT

Disclosed herein are compounds and compositions having potency in the modulation of Myc family proteins. Such compounds and compositions can be used in the treatment of proliferative diseases, such as cancer, or in the treatment of disease where modulation of Myc family proteins is desired. Also disclosed herein are methods of using said compounds and compositions.

### CROSS REFERENCE TO RELATED APPLICATIONS

This application is a continuation of an International Patent Application No. PCT/US2020/018804, filed Feb. 19, 2020, which claims the benefit of and priority to U.S. Provisional Patent Application No. 62/807,452, filed Feb. 19, 2019, wherein the content of each is hereby incorporated by reference in its entirety.

### BACKGROUND

The MYC proto-oncogene family comprises three members: C-MYC, MYCN, and MYCL. These oncogenes encode c-Myc, N-Myc, and L-Myc oncoproteins, respectively, which belong to a family of “super-transcription factors” that regulate the transcription of more than 15% of the entire genome. Recent studies in mouse models have suggested that the regulation of oncogenic Myc proteins could potentially lead to the development of cancer therapeutics, as it has been demonstrated that even transient inactivation of Myc causes tumor regression. However, the development of drugs and therapeutics that directly targets Myc proteins has met with two major challenges. First, Myc proteins lack a well-defined active site for the binding of small molecules, thus providing challenges for the functional modulation or inhibition of their activities. Second, Myc proteins are predominantly located in cell nuclei, and targeting nuclear Myc proteins with antibodies can be technically challenging. These challenges have spawned strategies for indirect regulation of Myc proteins.

For example, amplification and overexpression of N-Myc can lead to tumorigenesis. Excess N-Myc is associated with a variety of tumors, e.g., neuroblastomas. MYCN can also be activated in tumors through somatic mutation.

C-Myc can also be constitutively expressed in various cancers such as cervix, colon, breast, lung and stomach cancers. Such constitutive expression can lead to increased expression of other genes that are involved in cell proliferation.

Amplification of the, e.g., N-Myc gene in patients frequently results in poor health outcomes. However, strategies for direct modulation of Myc proteins remain elusive, as the Myc proteins are not easily targeted.

Therefore, an ongoing need exists for small-molecule therapeutic modulators of Myc proteins for the treatment of various ailments, diseases and disorders, e.g., cancer.

### SUMMARY

The present disclosure provides compounds and compositions that are useful as Myc protein modulators, and methods of using the same. Furthermore, the present disclosure contemplates using disclosed compounds and compositions as direct modulators of Myc proteins in the treatment of proliferative disease, such as cancer, or in the treatment of diseases where modulation of Myc family proteins is desired.

For example, the present disclosure provides a compound of Formula (I):

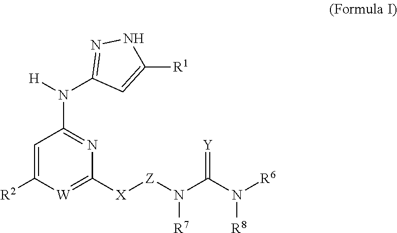

or a pharmaceutically acceptable salt, stereoisomer and/or N-oxide thereof, wherein:
   W is selected from the group consisting of N, C—H, and C—F;
   X is selected from the group consisting of N—R^{A}, O, S, CH₂, C(CH₃)₂, CF₂ and C(CH₂)₂;
   Y is selected from the group consisting of O and N—R^{B};
   Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, and —C(O)—O—C₁₋₄alkyl;
   R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
   R² is selected from the group consisting of H, F, —O-methyl, methyl, C₃-C₇ cycloalkyl and heterocyclyl;
   R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-heteroaryl, —C(O)-heteroaryl, and phenoxy; wherein R⁶ may be optionally substituted by one, two or three substituents each independently selected from the group consisting of R^{P};
   R⁷ is selected from the group consisting of H and C₁-C₆ alkyl; wherein C₁-C₆ alkyl may be optionally substituted by one, two or three substituents each independently selected from the group consisting of halogen, hydroxyl, cyano, oxo and C₁₋₆alkoxy (optionally substituted by one, two or three substituents each selected from halo, cyano, hydroxyl, and C₁₋₃alkoxy);
   R⁸ is selected from the group consisting of H and C₁-C₆-alkyl; wherein C₁-C₆ alkyl may be optionally substituted by one, two or three substituents each independently selected from the group consisting of halogen, hydroxyl, cyano, oxo and C₁₋₆alkoxy (optionally substituted by one, two or three substituents each selected from halo, cyano, hydroxyl, and C₁₋₃alkoxy);
      wherein at least one of R⁷ or R⁸ must be H;
   R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
   R^{B} is selected from the group consisting of H, C₁-C₄ alkyl, —C(O)—C₁₋₄ alkyl, S(O)_{w}—C₁₋₄alkyl, (wherein w is 0, 1 or 2) and cyano; wherein C₁-C₄ alkyl may be optionally substituted by one, two or three flouro substituents;
   R^{P} is selected from the group consisting of halo, cyano, C₁₋₆alkyl, C₂₋₆alkenyl, C₂₋₆ alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆ alkenyl, C₂₋₆alkynyl and C₃₋₆cycloalkyl may each be optionally substituted by one, two or three substituents each selected from halo, cyano, hydroxyl, heteroaryl, and NR′R′; and
   R′ for each occurrence is independently selected from the group consisting of H, methyl, ethyl, heterocyclyl (optionally substituted by C₁₋₃alkyl or halo), phenyl, and C₃₋₆ cycloalkyl, or two R's together with the nitrogen to which they are attached form a heterocyclyl which may optionally be substituted by methyl, halo, cyano, oxo, or hydroxyl.

A compound represented by Formula (III) is also provided:

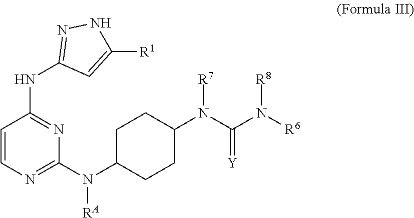

or a pharmaceutically acceptable salt, stereoisomer and/or N-oxide thereof, wherein:
   R¹ is selected from the group consisting of C₃-C₆cycloalkyl, heterocyclyl, and methyl, wherein R¹ is optionally substituted by halogen;
   R⁶ is selected from the group consisting of a saturated C₃-C₆ monocyclic carbocyclic ring, a saturated or partially unsaturated 8-10 membered bicyclic carbocylic ring, a monocyclic or bicyclic saturated or partially unsaturated heterocyclic ring having at least one heteroatom moiety selected from O, S(O)_{w} (wherein w is 0, 1, or 2), and NR^{C}, phenyl, phenoxy, naphthyl, a monocylic or bicyclic heteroaryl, benzyl, and —CR⁷R⁸— heteroaryl; wherein:
   R⁶ is optionally substituted on an available carbon by one, two or three substituents each independently selected from the group consisting of halogen, cyano, hydroxyl, oxo, C₁-C₆-alkyl (optionally substituted by one, two or three halogens or hydroxyl), C₃-C₆-cycloalkyl (optionally substituted by one, two or three halogens or hydroxyl), C₁-C₆-alkoxy (optionally substituted by one, two or three substituents each selected from the group consisting of halogen, methyoxy, and ethyoxy), heterocyclyl (optionally substituted by one or more substituents each selected from methyl, ethyl, hydroxyl, halogen and oxo), heterocyclyloxy (optionally substituted by one or more substituents each selected from methyl, ethyl, halogen, hydroxyl and oxo), heteroaryl (optionally substituted by one or more substituents each selected from methyl, ethyl, hydroxyl, halogen and oxo), heteroaryloxy (optionally substituted by one or more substituents each selected from methyl, ethyl, halogen, hydroxyl and oxo), —NR^{a}R^{b}; —C(O)O(R^{a}), —C(O)—N(R^{b})(R^{c}), —S(O)_{w}—R^{a}, —NR^{b}—S(O)_{w}—R^{a}, and —S(O)_{w}—N(R^{b})(R^{c}) (wherein w is 0, 1, or 2); and wherein
   R^{C}, if present, is selected from the group consisting of hydrogen, C₁-C₆alkyl (optionally substituted by phenyl or heteroaryl; wherein phenyl or heteroaryl is optionally substituted by halogen, hydroxyl, or methyl), cyclopropyl, C(O)O(R^{a}), C(O)R^{a}, and —S(O)_{w}—R^{a} (wherein w is 0, 1, or 2);
   R^{A} is selected from the group consisting of H and methyl;
   R⁷ is selected from the group consisting of H and methyl;
   R⁷ is selected from the group consisting of H and methyl;
      wherein at least one of R⁷ and R⁸ must be hydrogen;
   R^{a} is independently selected for each occurrence from the group consisting of hydrogen, C₁-C₆-alkyl, C₃-C₆-cycloalkyl, heterocyclyl, phenyl and heteroaryl; wherein C₁-C₆alkyl, C₃-C₆cycloalkyl, heterocyclyl, phenyl or heteroaryl may optionally be substituted by one or more substituents each independently selected from the group consisting of halogen, cyano, oxo, and hydroxyl;
   R^{b} and R^{c} are each independently selected for each occurrence from the group consisting of hydrogen, C₁-C₆alkyl, C₃-C₆cycloalkyl, phenyl, benzyl, and heteroaryl; or
   R^{b} and R^{c} may form, together with the nitrogen to which they are attached, a 4-6 membered heterocyclyl which may have an additional heteroatom and may be optionally substituted with oxo, C₁-C₃alkyl, or cyclopropyl;
   R⁷ and R⁸ are each independently selected from the group consisting of hydrogen, halogen, and C₁-C₃alkyl (optionally substituted by one, two or three halogens), or R⁷ and R⁸ taken together form an oxo;
   Y is selected from O and N—R^{B}; and
   R^{B} is selected from the group consisting of H, C₁-C₄alkyl, —S(O)_{w}—C₁-C₄alkyl (where w is 0, 1, or 2), —C(O)C₁-C₄alkyl, and CN; wherein C₁-C₄alkyl is optionally substituted by one, two or three halogens.

Pharmaceutical compositions comprising a disclosed compound or a pharmaceutically acceptable salt, stereoisomer and/or N-oxide thereof, as described herein, for example a disclosed pharmaceutical composition may include least one or more pharmaceutically acceptable carriers, diluents, stabilizers, excipients, dispersing agents, suspending agents, and/or thickening agents. The present disclosure also provides a method of manufacturing of the compounds described herein, or a pharmaceutically acceptable salt, stereoisomer and/or N-oxide thereof.

A method of modulating the amount and activity of a Myc family protein (i.e., C-Myc, N-Myc, L-Myc, or human Myc) is also provided, for example, an activity of a Myc family protein may be modulated in a cell by contacting a cell with an effective amount of a compound as described herein, or a pharmaceutically acceptable salt, stereoisomer and/or N-oxide thereof.

The present disclosure also provides a method of treating a Myc family protein associated disease in a subject in need thereof, the method comprising administering a therapeutically effective amount of a compound described herein, or a pharmaceutically acceptable salt, stereoisomer and/or N-oxide thereof, including embodiments in any examples, tables, or figures. In some embodiments, the subject is a human subject and the disease is a proliferative disease, such as cancer.

### DETAILED DESCRIPTION

### Definitions

Unless defined otherwise, all technical and scientific terms used herein have the same meaning as commonly understood by one of ordinary skill in the art to which this disclosure pertains.

It is understood that the definitions provided herein are not intended to be mutually exclusive. Accordingly, some chemical moieties may fall within the definition of more than one term.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, n-butoxy, tert-butoxy, sec-butoxy, n-pentoxy, n-hexoxy, 1,2-dimethylbutoxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

As used herein, the term “alkylene” refers to a di-radical alkyl group. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

As used herein, the terms “alkenylene,” “alkynylene,” “arylene,” “arylalkylene,” and “alkylarylene” refer to di-radical alkenyl, alkynyl, aryl, aryl alkyl, and alkylaryl groups, respectively.

As used herein, the term “azido” refers to group —N₃.

As used herein, the term “carboxyl,” “carboxy” or “carboxylate” refers to —CO₂H or salts thereof.

As used herein, the term “carbamoyl” refers to the group NH₂CO—.

The terms “cycloalkyl” or a “carbocyclic group” as used herein refers to a saturated or partially unsaturated hydrocarbon group of, for example, 3-10, 3-6, or 4-6 carbons, referred to herein as C₃₋₁₀cycloalkyl, or C₄₋₆cycloalkyl, respectively, and which may be monocyclic or bicyclic ring structures, e.g. 4-9 or 4-6 membered saturated ring structures, including bridged, fused or spirocyclic rings. Exemplary cycloalkyl groups include, but are not limited to, adamantanyl, cyclohexyl, cyclopentyl, cyclopentenyl, cyclobutyl, cyclopropyl, and indanyl.

As used herein, the groups

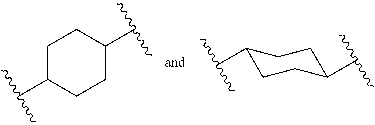 [SMILES: *C1CCC(*)CC1 ; *C1CCC(*)CC1]

are used interchangeably and refer to a cyclohexyl group.

As used herein, the term “cyano” and “carbonitrile” refer to the group —CN.

As used herein, the term “formyl” refers to the group —C(O)H.

As used herein, the term “guanidino” refers to the group —NHC(═NH)NH₂.

As used herein, the terms “halo” and “halogen” are used in the conventional sense to refer to a chloro, bromo, fluoro or iodo substituent.

As used herein, the terms “hydroxy” and “hydroxyl” refer to the group —OH.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl” or “heterocyclic group” are art-recognized and refer to e.g. saturated or partially unsaturated, 4-10 membered monocyclic or bicyclic ring structures, or e.g. 4-9 or 4-6 membered saturated ring structures, including bridged, fused or spirocyclic rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

As used herein, the term “nitro” refers to the group —NO₂.

As used herein, the term “oxo” refers to the group (═O) or (O).

As used herein, the term “isomers” refers to compounds comprising the same numbers and types of atoms or components, but with different structural arrangement and connectivity of the atoms.

As used herein, the term “tautomer” refers to one of two or more structural isomers which readily convert from one isomeric form to another and which exist in equilibrium.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “A” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or configuration wherein the terms “Z” and are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. Substituents around a carbocyclic or heterocyclic ring may be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the present disclosure embrace both solvated and unsolvated forms. In one embodiment, a disclosed compound is amorphous. In one embodiment, a disclosed compound is a single polymorph. In another embodiment, a disclosed compound is a mixture of polymorphs. In another embodiment, a disclosed compound is in a crystalline form.

The present disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent

As used herein, singular articles such as “a,” “an” and “the” and similar referents in the context of describing the elements are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (i.e., “such as”) provided herein, is intended merely to better illuminate the embodiments and does not pose a limitation on the scope of the claims unless otherwise stated.

In some embodiments, where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remains operable. Moreover, two or more steps or actions can be conducted simultaneously.

As used herein, a dash (“-”) that is not between two letters or symbols refers to a point of bonding or attachment for a substituent. For example, —NH₂ is attached through the nitrogen atom.

As used herein, the terms “active agent,” “drug,” “pharmacologically active agent” and “active pharmaceutical ingredient” are used interchangeably to refer to a compound or composition which, when administered to a subject, induces a desired pharmacologic or physiologic effect by local or systemic action or both.

As used herein, the term “prodrug” refers to compounds that are transformed in vivo to provide a compound or pharmaceutically acceptable salt, hydrate or solvate of the compound described herein. The transformation can occur by various mechanisms (i.e., esterase, amidase, phosphatase, oxidative and/or reductive metabolism) in various locations (i.e., in the intestinal lumen or upon transit into the intestine, blood, or liver).

As used herein, the term “modulator” refers to a compound or composition that increases or decreases the level of a target or the function of a target, which may be, but is not limited to, a Myc family protein, such as c-Myc, N-Myc, L-Myc and human Myc.

As used herein, the term “degrader” refers to a compound or composition that decreases the amount of a target or the activity of a target. In some embodiments, the target may be, but is not limited to, a Myc family protein comprising c-Myc, N-Myc, L-Myc and human Myc.

As used herein, the term “degrading” refers to a method or process that decreases the amount of a target or the activity of a target. In some embodiments, the target may be, but is not limited to, a Myc family protein comprising c-Myc, N-Myc, L-Myc and human Myc.

As used herein, the term “Myc family protein” refers to any one of the proteins c-Myc, N-Myc, or L-Myc as described herein. In some embodiments, a Myc protein is a c-Myc protein. In some embodiments, a Myc protein is a N-Myc protein. In some embodiments, a Myc protein is a L-Myc protein. In some embodiments, a Myc protein is a human c-Myc protein. In some embodiments, a Myc protein is a human N-Myc protein. In some embodiments, a Myc protein is a human L-Myc protein. In some embodiments, a Myc family protein is a human Myc family protein.

As used herein, the terms “N-Myc” and “MycN” can be used interchangeably and refer to the protein “V-Myc myelocytomatosis viral related oncogene, neuroblastoma derived” and include the wildtype and mutant forms of the protein. In some embodiments, MycN refers to the protein associated with one or more of database entries of Entrez Gene 4613, OMIM 164840, UniProt P04198, and RegSeq NP_005369.

As used herein, the term “c-Myc” refers to the protein “V-Myc myelocytomatosis viral oncogene” and include the wildtype and mutant forms of the protein. In some embodiments, c-Myc refers to the protein associated with one or more of database entries of Entrez Gene 4609, OMIM 190080, UniProt P01106, and RegSeq NP_002458.

As used herein, the term “L-Myc” refers to the protein “V-Myc myelocytomatosis viral oncogene homolog, lung carcinoma derived” and include the wildtype and mutant forms of the protein. In some embodiments, L-Myc refers to the protein associated with one or more of database entries of Entrez Gene 4610, OMIM 164850, UniProt PI 2524, and RegSeq NP_001028253.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines, felines, equines, bovines, ovines, rodentia, etc. and primates, i.e., non-human primates, and humans. Non-human animal models, i.e., mammals, non-human primates, murines, lagomorpha, etc. may be used for experimental investigations.

As used herein, the terms “treating,” “treatment,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human and includes: (a) preventing the disease or a symptom of a disease from occurring in a subject which may be predisposed to the disease but has not yet been diagnosed as having it (i.e., including diseases that may be associated with or caused by a primary disease); (b) inhibiting the disease, i.e., arresting its development; and (c) relieving the disease, i.e., causing regression of the disease (I.e., reduction in of tumor burden). In some embodiments, certain methods described herein treat cancer associated with the signaling pathway of a Myc family protein, such as c-Myc, N-Myc, L-Myc or human Myc.

As used herein, the term “pharmaceutically acceptable salt” refers to a salt which is acceptable for administration to a subject. It is understood that such salts, with counter ions, will have acceptable mammalian safety for a given dosage regime. Such salts can also be derived from pharmaceutically acceptable inorganic or organic bases and from pharmaceutically acceptable inorganic or organic acids, and may comprise organic and inorganic counter ions. The neutral forms of the compounds described herein may be converted to the corresponding salt forms by contacting the compound with a base or acid and isolating the resulting salts.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like.

Other examples of salts include anions of the compounds of the present disclosure compounded with a suitable cation such as N⁺, NH₄⁺, and NW₄⁺ (where W can be a C₁-C₈ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

As used herein, the terms “determining,” “measuring,” “assessing,” and “assaying” are used interchangeably and include both quantitative and qualitative determinations.

As used herein, the phrase “signaling pathway” refers to a series of interactions between cellular components, both intracellular and extracellular, that conveys a change to one or more other components in a living organism, which may cause a subsequent change to additional component. Optionally, the changes conveyed by one signaling pathway may propagate to other signaling pathway components. Examples of cellular components include, but are not limited to, proteins, nucleic acids, peptides, lipids and small molecules.

As used herein, the terms “effective amount” and “therapeutically effective amount” are used interchangeably and refer to the amount of a compound that, when administered to a mammal or other subject for treating a disease, condition, or disorder, is sufficient to affect such treatment for the disease, condition, or disorder. The “effective amount” or “therapeutically effective amount” will vary depending on the compound, the disease and its severity and the age, weight, etc., of the subject to be treated.

As used herein, the terms “pharmaceutically acceptable excipient,” “pharmaceutically acceptable diluent,” “pharmaceutically acceptable carrier,” and “pharmaceutically acceptable adjuvant” refer to an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “a pharmaceutically acceptable excipient, diluent, carrier and adjuvant” as used in the specification and claims includes both one and more than one such excipient, diluent, carrier, and adjuvant.

As used herein, the term “pharmaceutical composition” is meant to encompass a composition suitable for administration to a subject, such as a mammal, especially a human. In general a “pharmaceutical composition” is sterile, and free of contaminants that are capable of eliciting an undesirable response within the subject (i.e., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of (R)-enantiomer and (S)-enantiomer, and enantio-enriched stereomeric mixtures comprising of (R)- and (S)-enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### Compounds

The disclosure is generally directed to compounds that modulate (e.g., degrade) MycN and/or MycC, and may therefore have significant antineoplastic properties. The disclosed compounds and pharmaceutical compositions thereof find use in a variety of applications in which the modulation of the amount and activity of a Myc protein is desired, including use as potent antineoplastic agents.

For example, provided herein is a compound of Formula (I):

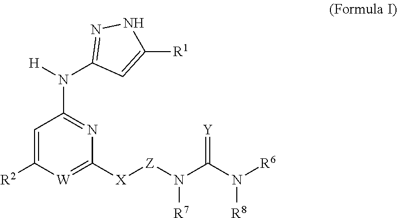

or a pharmaceutically acceptable salt, stereoisomer and/or N-oxide thereof, wherein:
   W is selected from the group consisting of N, C—H, and C—F;
   X is selected from the group consisting of N—R^{A}, O, S, CH₂, C(CH₃)₂, CF₂ and C(CH₂)₂;
   Y is selected from the group consisting of O and N—R^{B};
   Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, and —C(O)—O—C₁₋₄alkyl;
   R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
   R² is selected from the group consisting of H, F, —O-methyl, methyl, C₃-C₇ cycloalkyl and heterocyclyl;
   R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-heteroaryl, —C(O)-heteroaryl, and phenoxy; wherein R⁶ may be optionally substituted by one, two or three substituents each independently selected from the group consisting of R^{P};
   R⁷ is selected from the group consisting of H and C₁-C₆ alkyl; wherein C₁-C₆ alkyl may be optionally substituted by one, two or three substituents each independently selected from the group consisting of halogen, hydroxyl, cyano, oxo and C₁₋₆alkoxy (optionally substituted by one, two or three substituents each selected from halo, cyano, hydroxyl, and C₁₋₃alkoxy);
   R⁸ is selected from the group consisting of H and C₁-C₆-alkyl; wherein C₁-C₆ alkyl may be optionally substituted by one, two or three substituents each independently selected from the group consisting of halogen, hydroxyl, cyano, oxo and C₁₋₆alkoxy (optionally substituted by one, two or three substituents each selected from halo, cyano, hydroxyl, and C₁₋₃alkoxy);
      wherein at least one of R⁷ or R⁸ must be H;
   R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
   R^{B} is selected from the group consisting of H, C₁-C₄ alkyl, —C(O)—C₁₋₄ alkyl, S(O)_{w}—C₁₋₄alkyl, (wherein w is 0, 1 or 2) and cyano; wherein C₁-C₄ alkyl may be optionally substituted by one, two or three flouro substituents;
   R^{P} is selected from the group consisting of halo, cyano, C₁₋₆alkyl, C₂₋₆alkenyl, C₂₋₆ alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆ alkenyl, C₂₋₆alkynyl and C₃₋₆cycloalkyl may each be optionally substituted by one, two or three substituents each selected from halo, cyano, hydroxyl, heteroaryl, and NR′R′; and
   R′ for each occurrence is independently selected from the group consisting of H, methyl, ethyl, heterocyclyl (optionally substituted by C₁₋₃alkyl or halo), phenyl, and C₃₋₆ cycloalkyl, or two R's together with the nitrogen to which they are attached form a heterocyclyl which may optionally be substituted by methyl, halo, cyano, oxo, or hydroxyl.

In some embodiments, for example, W is N, and a compound of the disclosure has the Formula Ia:

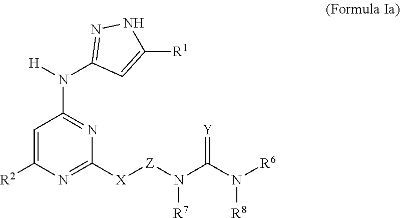

or a pharmaceutically acceptable salt, stereoisomer and/or N-oxide thereof.

In another embodiment, Y is O, and a compound of the disclosure has the Formula Ib:

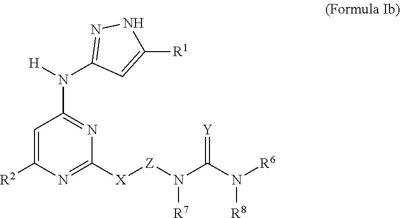

or a pharmaceutically acceptable salt, stereoisomer and/or N-oxide thereof.

In some embodiments, Y is N—R^{B}, and a compound of the disclosure has the Formula Ic:

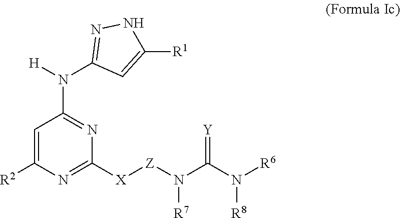

or a pharmaceutically acceptable salt, stereoisomer and/or N-oxide thereof, wherein:
   R^{B} is selected from the group consisting of H, C₁-C₄ alkyl, —SO₂—C₁-C₄-alkyl, C(O)C₁-C₄-alkyl, CN, and CH₂CF₃.

In some embodiments, R¹ is a 5-6 membered heterocyclyl or C₃₋₆cycloalkyl. For example, R¹ is selected from the group consisting of: 2-tetrahydrofuranyl, 3-tetrahydrofuranyl, 2-oxetanyl, cyclohexyl, cyclopropyl, cyclobutyl and cyclopentyl.

In other embodiments, R¹ is selected from the group consisting of, e.g., methyl and ethyl.

In an exemplary embodiment, Z is selected from the group consisting of cyclohexyl, cyclopentyl and cyclobutyl.

In some embodiments, Z is a C₅-C₉ bridged cycloalkyl.

In some embodiments, Z is a spiro C₅-C₁₀ bicycloalkyl.

In some embodiments, Z is a fused bicycloalkyl.

In some embodiments, Z is selected from the group consisting of:

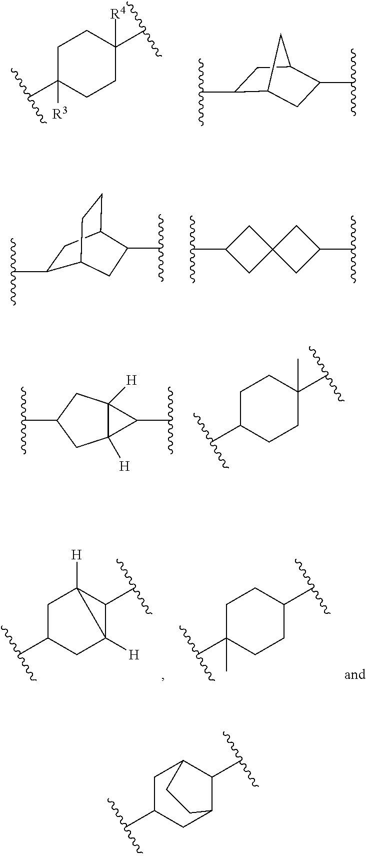

or a pharmaceutically acceptable salt, stereoisomer and/or N-oxide thereof, wherein:
R³ is selected from the group consisting of H, C₁-C₄-alkyl, CO₂H and —C(O)—O—C₁₋₄alkyl;
R⁴ is H or C₁-C₄-alkyl; or
R³ and R⁴ together form —CH₂— or —CH₂CH₂—.

For example, Z is selected from the group consisting of:

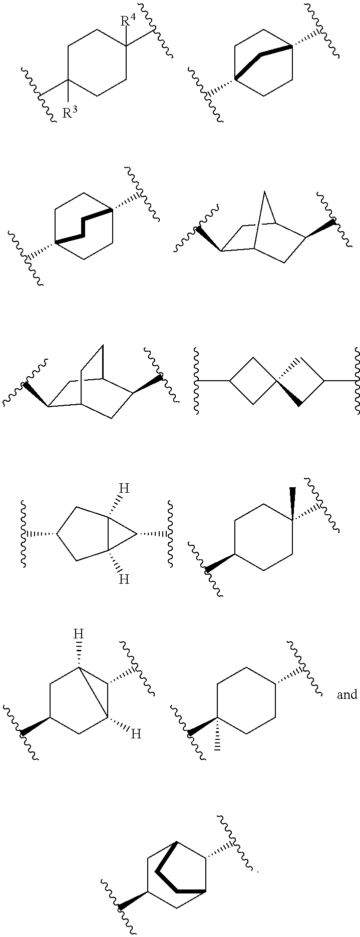

Also disclosed herein are compounds represented by Formula IF

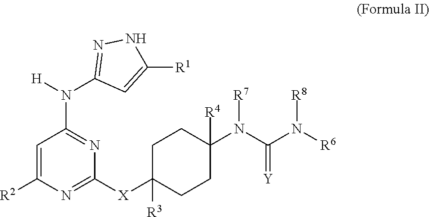

or a pharmaceutically acceptable salt, stereoisomer and/or N-oxide thereof, wherein:
   R³ is selected from the group consisting of H, C₁-C₄alkyl, CO₂H and —C(O)—O—C₁₋₄ alkyl; and
   R⁴ is selected from H or C₁-C₄alkyl.

Exemplary disclosed compounds may be represented by Formula IIa:

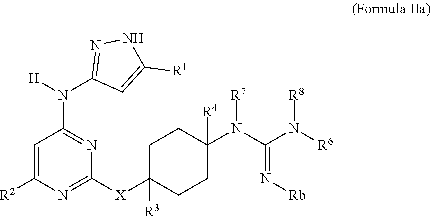

or a pharmaceutically acceptable salt, stereoisomer and/or N-oxide thereof, wherein:
   R³ is selected from the group consisting of H, C₁-C₄-alkyl, CO₂H and —C(O)—O—C₁₋₄ alkyl; and
   R⁴ is selected from the group consisting of H and C₁-C₄-alkyl;
   R^{B} is selected from the group consisting of H, C₁-C₄ alkyl, —SO₂—C₁-C₄-alkyl, C(O)C₁-C₄-alkyl, CN, and CH₂CF₃.

In some embodiments, R⁶ is selected from the group consisting of a 8-10 membered bicyclic cycloalkyl and a 8-10 membered bicyclic heterocyclyl.

In some embodiments, R⁶ is selected from the group consisting of a monocyclic or bridged C₃₋₆cycloalkyl, a monocyclic or bridged heterocyclyl, a bicyclic or fused heterocyclyl, and a heteroaryl.

In some embodiments, R⁶ is selected from the group consisting of: indanyl, cyclohexyl, cyclobutyl, and cyclopentyl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms), C₁₋₄alkoxy (optionally substituted by one, two or three fluorine atoms), S(O)₂—CH₃; cyclopropyl, cyclobutyl, —O-heterocyclyl, heterocyclyl and heteroaryl. For example, R⁶ is indanyl.

In some embodiments, R⁶ is selected from the group consisting of heterocyclyl, phenyl, and heteroaryl.

In some embodiments, R⁶ is represented by:

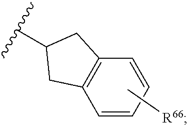

wherein R⁶⁶ is selected from the group consisting of hydrogen, cyano, heterocyclyl, heteroaryl, —C(═N)—R′R′; and S(O)₂—CH₃.

For example, R⁶ is selected from the group consisting of:

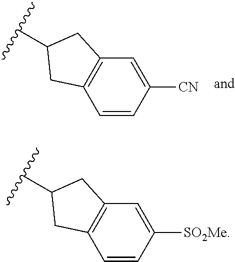 [SMILES: *C1Cc2ccc(C#N)cc2C1 ; *C1Cc2ccc(S(C)(=O)=O)cc2C1]

In other embodiments, R⁶ is represented by:

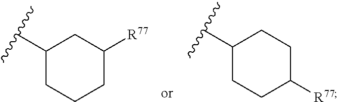

wherein R⁷⁷ is selected from the group of hydrogen, C₁₋₄alkyl (optionally substituted by one, two or three fluorine atoms), C₁₋₄alkoxy (optionally substituted by methoxy or by one, two or three fluorine atoms), heterocyclyl, and S(O)₂—C₁₋₄alkyl. For example, R⁷⁷ is selected from the group consisting of —CF₃, —OCH₃, —OCHF₂, —SO₂CH₃, and —OCH₂CH₂OCH₃. For example, R⁶ is selected from the group consisting of:

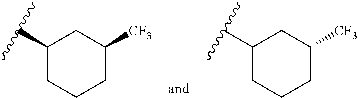 [SMILES: *[C@@H]1CCC[C@H](C(F)(F)F)C1 ; *C1CCC[C@@H](C(F)(F)F)C1]

In further embodiments, R⁶ is selected from the group consisting of:

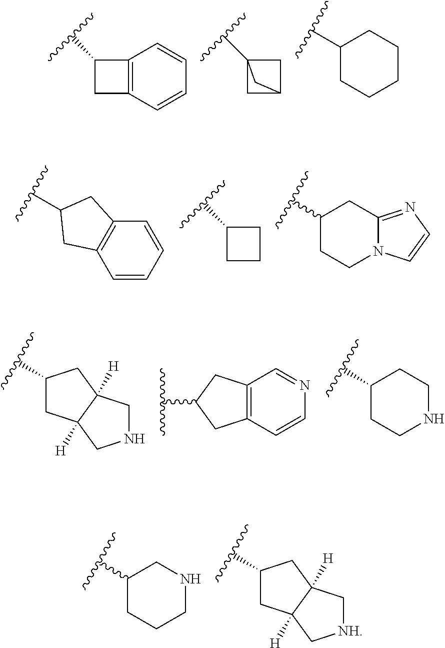

In other embodiments, R⁶ is selected from the group consisting of:

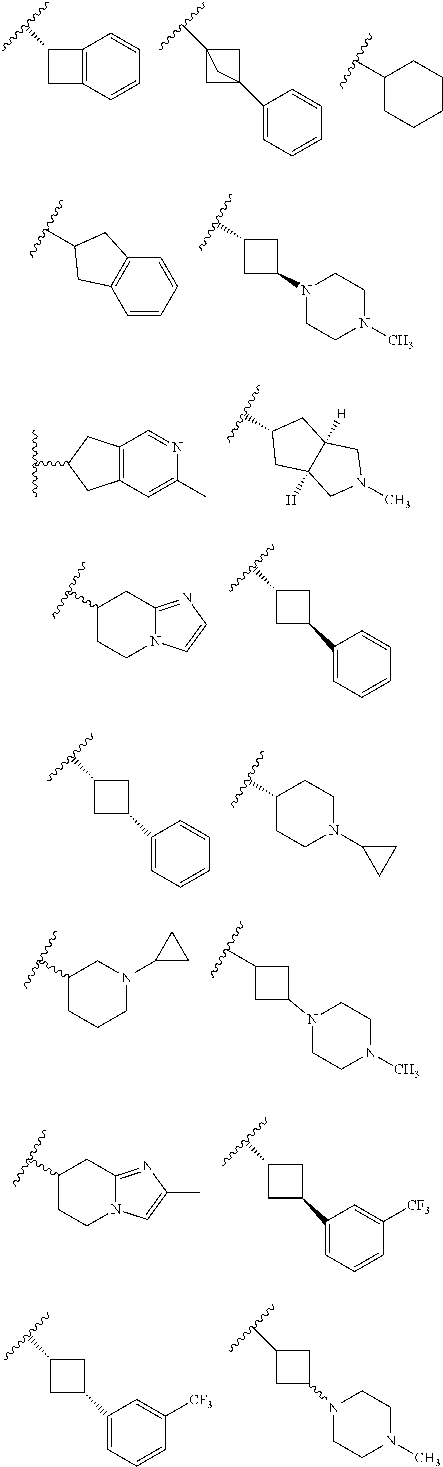

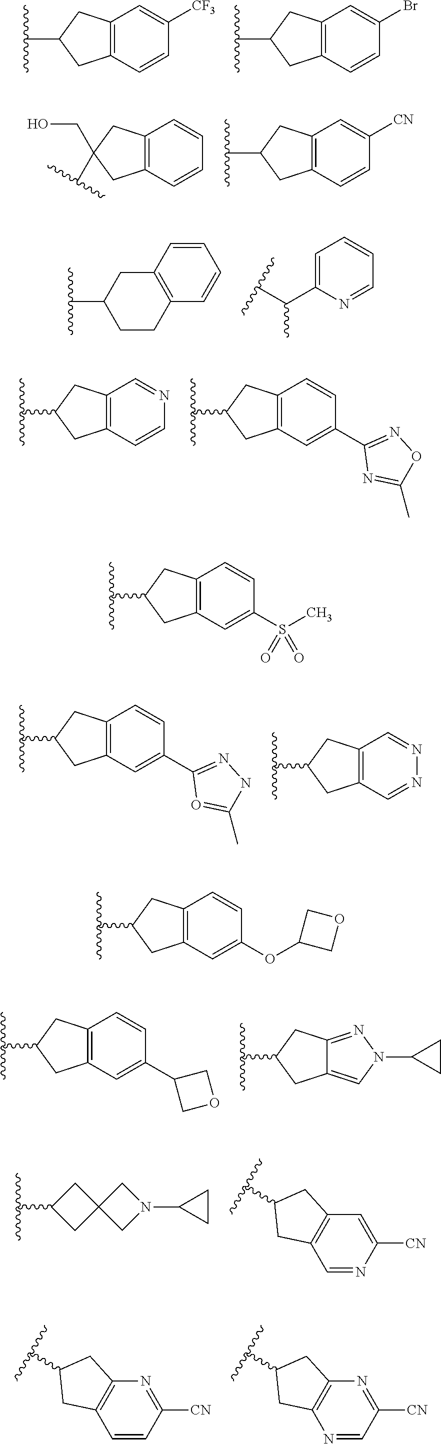

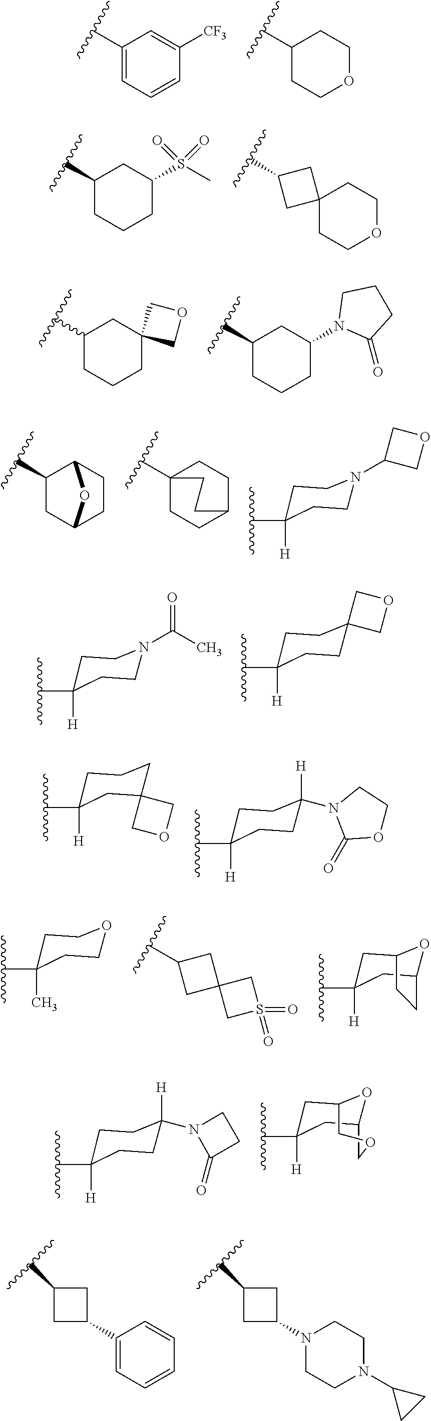

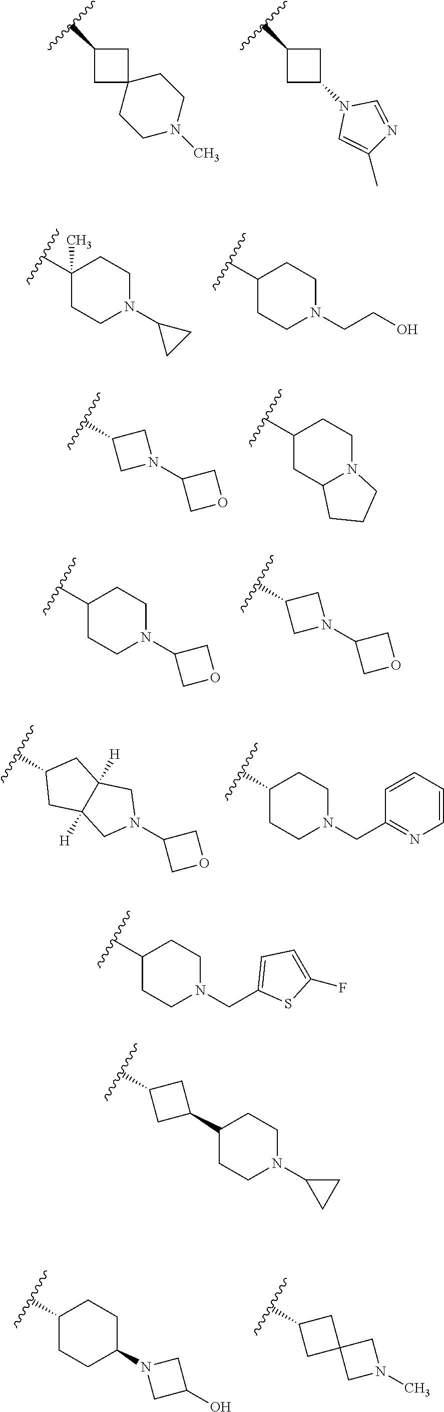

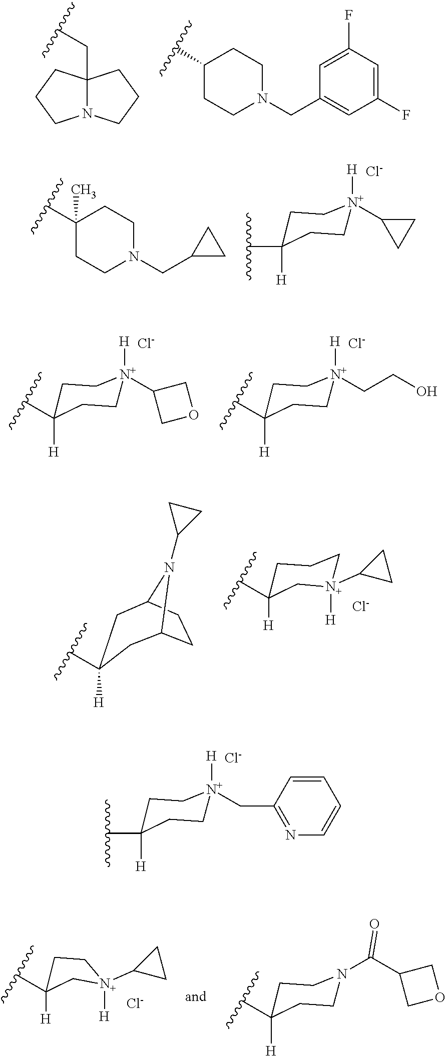

or a pharmaceutically acceptable salt, stereoisomer and/or N-oxide thereof.

In some embodiments, X is N—H. In other embodiments, X is O.

In some embodiments, R⁷ is H and R⁸ is methyl. In other embodiments, R⁷ is methyl and R⁸ is H. In further embodiments, R⁷ and R⁸ are each H.

Further disclosed herein is a compound represented by Formula (III):

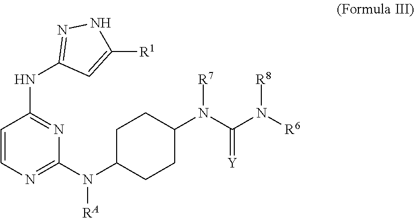

or a pharmaceutically acceptable salt, stereoisomer and/or N-oxide thereof, wherein:
   R¹ is selected from the group consisting of C₃-C₆cycloalkyl, heterocyclyl, and methyl, wherein R¹ is optionally substituted by halogen;
   R⁶ is selected from the group consisting of a saturated C₃-C₆ monocyclic carbocyclic ring, a saturated or partially unsaturated 8-10 membered bicyclic carbocylic ring, a monocyclic or bicyclic saturated or partially unsaturated heterocyclic ring having at least one heteroatom moiety selected from O, S(O)_{w} (wherein w is 0, 1, or 2), and NR^{C}, phenyl, phenoxy, naphthyl, a monocylic or bicyclic heteroaryl, benzyl, and —CR⁷R⁸— heteroaryl; wherein:
   R⁶ is optionally substituted on an available carbon by one, two or three substituents each independently selected from the group consisting of halogen, cyano, hydroxyl, oxo, C₁-C₆-alkyl (optionally substituted by one, two or three halogens or hydroxyl), C₃-C₆-cycloalkyl (optionally substituted by one, two or three halogens or hydroxyl), C₁-C₆-alkoxy (optionally substituted by one, two or three substituents each selected from the group consisting of halogen, methyoxy, and ethyoxy), heterocyclyl (optionally substituted by one or more substituents each selected from methyl, ethyl, hydroxyl, halogen and oxo), heterocyclyloxy (optionally substituted by one or more substituents each selected from methyl, ethyl, halogen, hydroxyl and oxo), heteroaryl (optionally substituted by one or more substituents each selected from methyl, ethyl, hydroxyl, halogen and oxo), heteroaryloxy (optionally substituted by one or more substituents each selected from methyl, ethyl, halogen, hydroxyl and oxo), —NR^{a}R^{b}; —C(O)O(R^{a}), —C(O)—N(R^{b})(R^{c}), —S(O)_{w}—R^{a}, —NR^{b}—S(O)_{w}—R^{a}, and —S(O)_{w}—N(R^{b})(R^{c}) (wherein w is 0, 1, or 2); and wherein
   R^{C}, if present, is selected from the group consisting of hydrogen, C₁-C₆alkyl (optionally substituted by phenyl or heteroaryl; wherein phenyl or heteroaryl is optionally substituted by halogen, hydroxyl, or methyl), cyclopropyl, C(O)O(R^{a}), C(O)R^{a}, and —S(O)_{w}—R^{a} (wherein w is 0, 1, or 2);
   R^{A} is selected from the group consisting of H and methyl;
   R⁷ is selected from the group consisting of H and methyl;
   R⁷ is selected from the group consisting of H and methyl;
      wherein at least one of R⁷ and R⁸ must be hydrogen;
   R^{a} is independently selected for each occurrence from the group consisting of hydrogen, C₁-C₆-alkyl, C₃-C₆-cycloalkyl, heterocyclyl, phenyl and heteroaryl; wherein C₁-C₆alkyl, C₃-C₆cycloalkyl, heterocyclyl, phenyl or heteroaryl may optionally be substituted by one or more substituents each independently selected from the group consisting of halogen, cyano, oxo, and hydroxyl;
   R^{b} and R^{c} are each independently selected for each occurrence from the group consisting of hydrogen, C₁-C₆alkyl, C₃-C₆cycloalkyl, phenyl, benzyl, and heteroaryl; or
   R^{b} and R^{c} may form, together with the nitrogen to which they are attached, a 4-6 membered heterocyclyl which may have an additional heteroatom and may be optionally substituted with oxo, C₁-C₃alkyl, or cyclopropyl;
   R⁷ and R⁸ are each independently selected from the group consisting of hydrogen, halogen, and C₁-C₃alkyl (optionally substituted by one, two or three halogens), or R⁷ and R⁸ taken together form an oxo;
   Y is selected from O and N—R^{B}; and
   R^{B} is selected from the group consisting of H, C₁-C₄alkyl, —S(O)_{w}—C₁-C₄alkyl (where w is 0, 1, or 2), —C(O)C₁-C₄alkyl, and CN; wherein C₁-C₄alkyl is optionally substituted by one, two or three halogens.

In some embodiments, R⁶ is a partially unsaturated bicyclic carbocycle. For example, R⁶ is represented by:

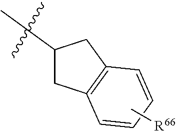

wherein R⁶⁶ is selected from the group consisting of cyano, heterocyclyl, heterocyclyloxy, C₁-C₃-alkyl (optionally substituted by halogen), —C(═N)—R^{b}R^{c}; and —S(O)₂—C₁-C₃-alkyl.

In other embodiments, R⁶ is a heterocyclic ring having a heteroatom moiety NR^{C}. For example, R⁶ is represented by

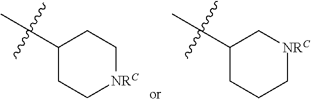

wherein R^{C} is selected from the group consisting of hydrogen, C₁-C₃-alkyl (optionally substituted by phenyl or heteroaryl; wherein phenyl or heteroaryl is optionally substituted by halogen, hydroxyl, or methyl), C₃-C₆-cycloalkyl, C(O)O(R^{a}), C(O)R^{a}, and —S(O)_{w}—R^{a}.

In some embodiments, R^{C} is C₁-C₆ alkyl, C₃-C₈-cycloalkyl, C₃-C₈-heterocycloalkyl, —CH₂-heteroaryl, —C(O)—C₁-C₆-alkyl, —C(O)-heteroaryl, —C(O)—C₃-C₈-cycloalkyl, and —C(O)—C₃-C₈-heterocycloalkyl.

In some embodiments, R⁶ is selected from the group consisting of: indanyl, cyclohexyl, cyclobutyl, and cyclopentyl, wherein R⁶ is optionally substituted by one or two substituents each selected from the group consisting of: cyano, halo, phenyl, —C(═N)—NR′R′, C₁₋₄alkyl (optionally substituted by one, two or three fluoros), C₁₋₄alkoxy (optionally substituted by methoxy or one, two or three fluoros), S(O)₂—CH₃; cyclopropyl, cyclobutyl, —O— heterocyclyl, and heterocyclyl.

For example, R⁶ is represented by:

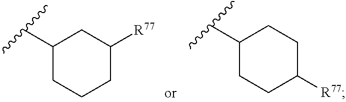

wherein R⁷⁷ is selected from the group of hydrogen, C₁₋₄alkyl (optionally substituted by one, two or three fluorine atoms), C₁₋₄alkoxy (optionally substituted by methoxy or by one, two or three fluorine atoms), heterocyclyl, and S(O)₂—C₁₋₄alkyl. For example, R⁷⁷ is selected from the group consisting of —CF₃, —OCH₃. OCHF₂, —SO₂CH_{3J}—OCH₂CH₂OCH₃. For example, R⁶ is selected from the group consisting of:

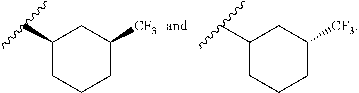 [SMILES: *[C@@H]1CCC[C@H](C(F)(F)F)C1 ; *C1CCC[C@@H](C(F)(F)F)C1]

A contemplated compound, for example, may selected from the group consisting of:

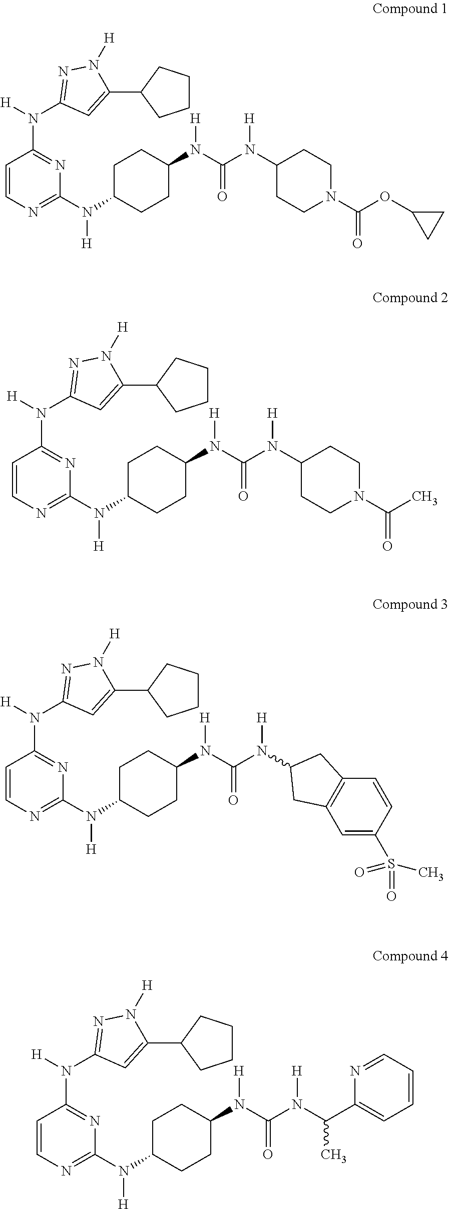 [SMILES: O=C(NC1CCN(C(=O)OC2CC2)CC1)N[C@H]1CC[C@H](Nc2nccc(Nc3cc(C4CCCC4)[nH]n3)n2)CC1 ; CC(=O)N1CCC(NC(=O)N[C@H]2CC[C@H](Nc3nccc(Nc4cc(C5CCCC5)[nH]n4)n3)CC2)CC1 ; CS(=O)(=O)c1ccc2c(c1)CC(NC(=O)N[C@H]1CC[C@H](Nc3nccc(Nc4cc(C5CCCC5)[nH]n4)n3)CC1)C2 ; CC(NC(=O)N[C@H]1CC[C@H](Nc2nccc(Nc3cc(C4CCCC4)[nH]n3)n2)CC1)c1ccccn1]

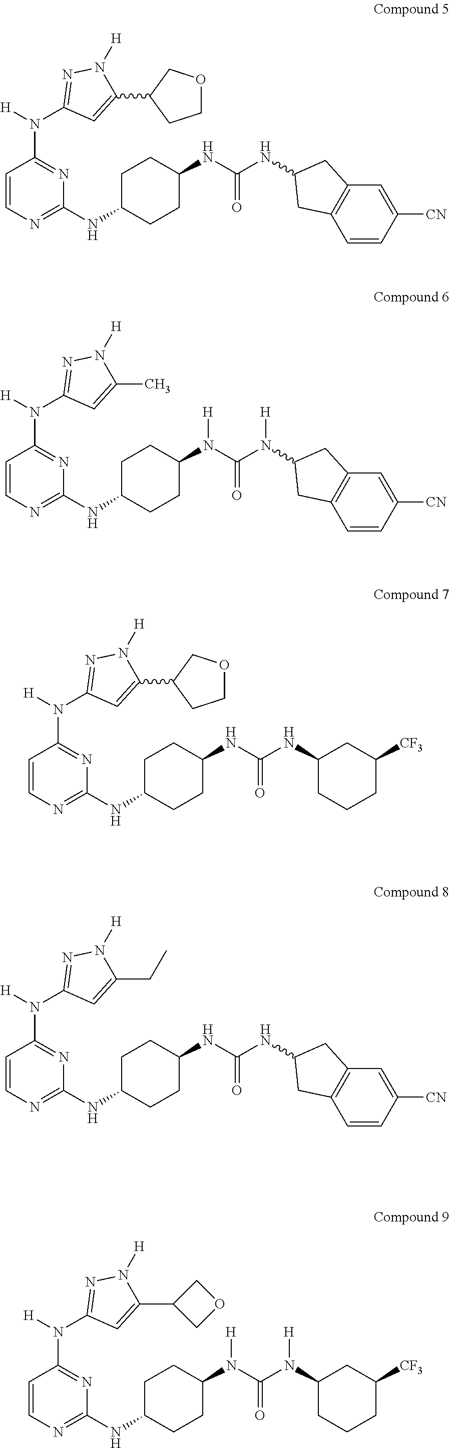 [SMILES: N#Cc1ccc2c(c1)CC(NC(=O)N[C@H]1CC[C@H](Nc3nccc(Nc4cc(C5CCOC5)[nH]n4)n3)CC1)C2 ; Cc1cc(Nc2ccnc(N[C@H]3CC[C@H](NC(=O)NC4Cc5ccc(C#N)cc5C4)CC3)n2)n[nH]1 ; O=C(N[C@@H]1CCC[C@H](C(F)(F)F)C1)N[C@H]1CC[C@H](Nc2nccc(Nc3cc(C4CCOC4)[nH]n3)n2)CC1 ; CCc1cc(Nc2ccnc(N[C@H]3CC[C@H](NC(=O)NC4Cc5ccc(C#N)cc5C4)CC3)n2)n[nH]1 ; O=C(N[C@@H]1CCC[C@H](C(F)(F)F)C1)N[C@H]1CC[C@H](Nc2nccc(Nc3cc(C4COC4)[nH]n3)n2)CC1]

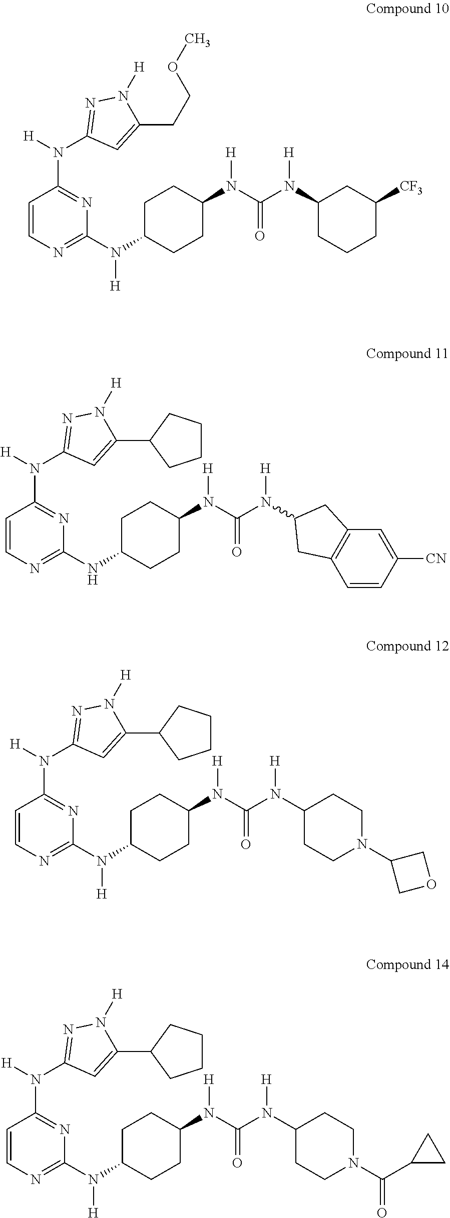 [SMILES: COCCc1cc(Nc2ccnc(N[C@H]3CC[C@H](NC(=O)N[C@@H]4CCC[C@H](C(F)(F)F)C4)CC3)n2)n[nH]1 ; N#Cc1ccc2c(c1)CC(NC(=O)N[C@H]1CC[C@H](Nc3nccc(Nc4cc(C5CCCC5)[nH]n4)n3)CC1)C2 ; O=C(NC1CCN(C2COC2)CC1)N[C@H]1CC[C@H](Nc2nccc(Nc3cc(C4CCCC4)[nH]n3)n2)CC1 ; O=C(NC1CCN(C(=O)C2CC2)CC1)N[C@H]1CC[C@H](Nc2nccc(Nc3cc(C4CCCC4)[nH]n3)n2)CC1]

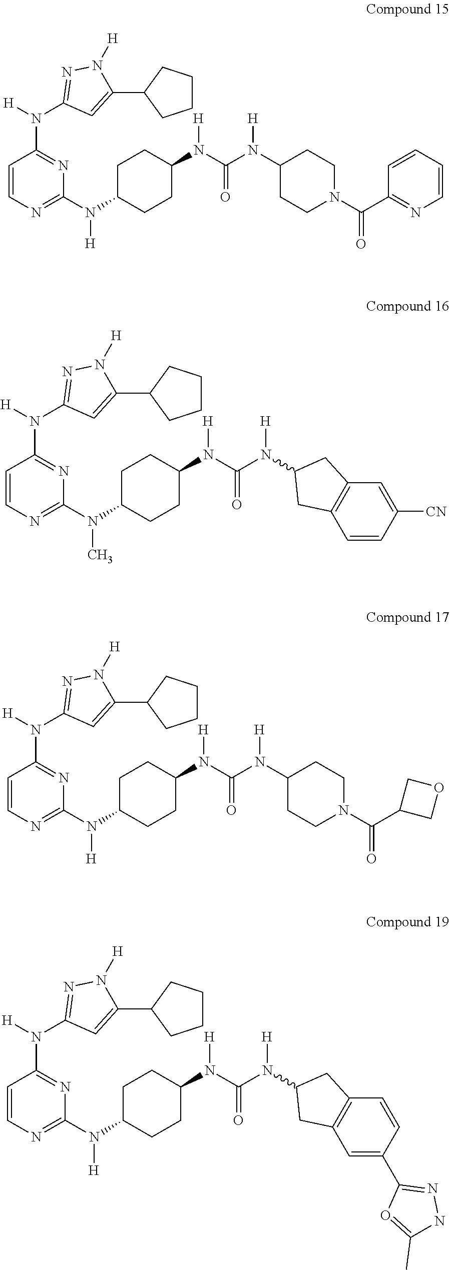

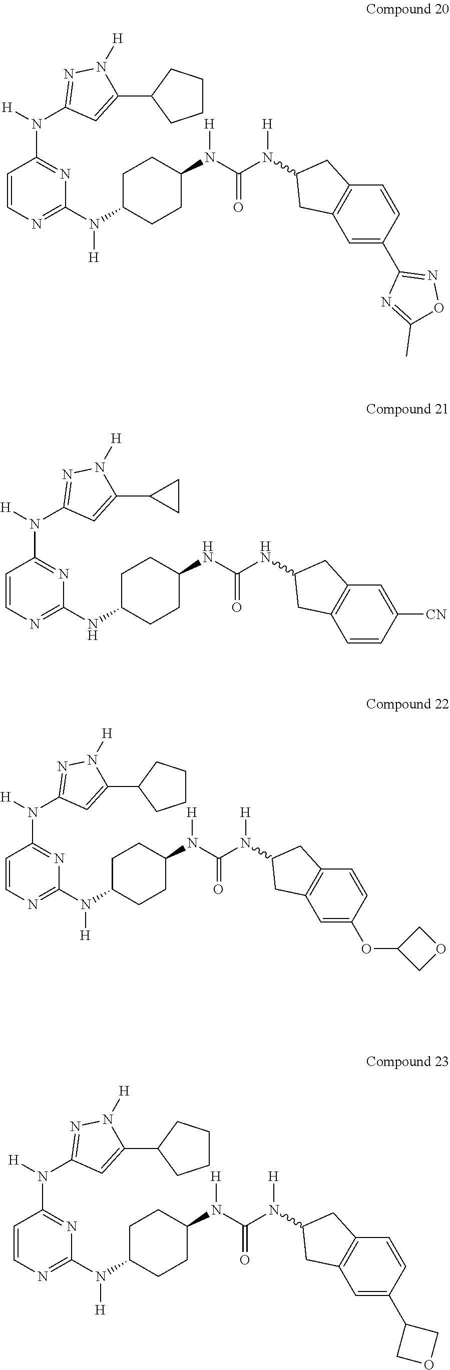 [SMILES: Cc1nc(-c2ccc3c(c2)CC(NC(=O)N[C@H]2CC[C@H](Nc4nccc(Nc5cc(C6CCCC6)[nH]n5)n4)CC2)C3)no1 ; N#Cc1ccc2c(c1)CC(NC(=O)N[C@H]1CC[C@H](Nc3nccc(Nc4cc(C5CC5)[nH]n4)n3)CC1)C2 ; O=C(NC1Cc2ccc(OC3COC3)cc2C1)N[C@H]1CC[C@H](Nc2nccc(Nc3cc(C4CCCC4)[nH]n3)n2)CC1 ; O=C(NC1Cc2ccc(C3COC3)cc2C1)N[C@H]1CC[C@H](Nc2nccc(Nc3cc(C4CCCC4)[nH]n3)n2)CC1]

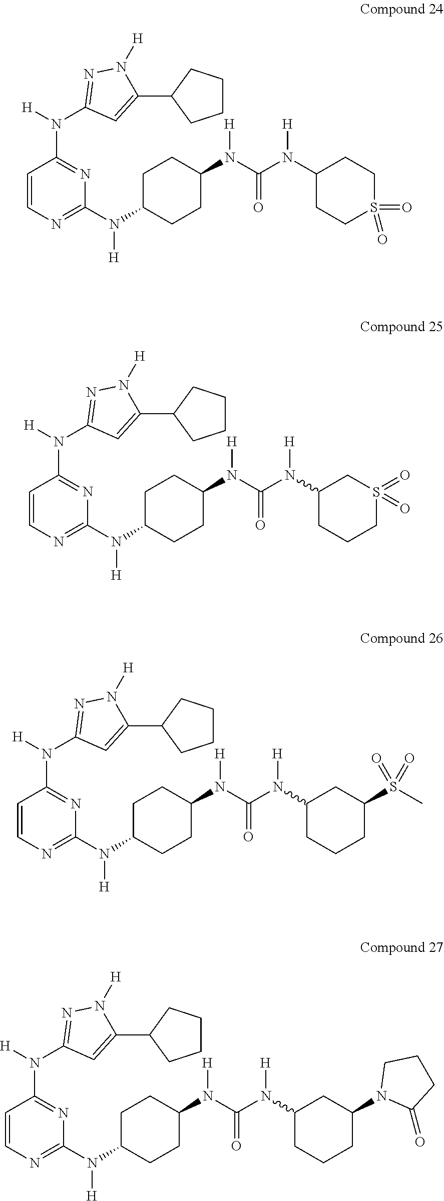 [SMILES: O=C(NC1CCS(=O)(=O)CC1)N[C@H]1CC[C@H](Nc2nccc(Nc3cc(C4CCCC4)[nH]n3)n2)CC1 ; O=C(NC1CCCS(=O)(=O)C1)N[C@H]1CC[C@H](Nc2nccc(Nc3cc(C4CCCC4)[nH]n3)n2)CC1 ; CS(=O)(=O)[C@H]1CCCC(NC(=O)N[C@H]2CC[C@H](Nc3nccc(Nc4cc(C5CCCC5)[nH]n4)n3)CC2)C1 ; O=C(NC1CCC[C@H](N2CCCC2=O)C1)N[C@H]1CC[C@H](Nc2nccc(Nc3cc(C4CCCC4)[nH]n3)n2)CC1]

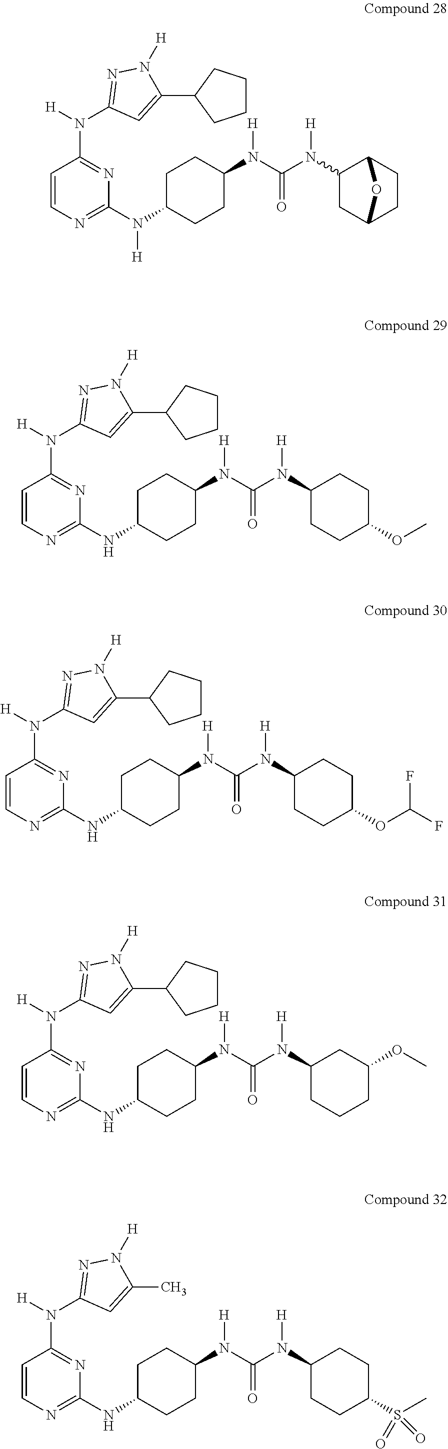

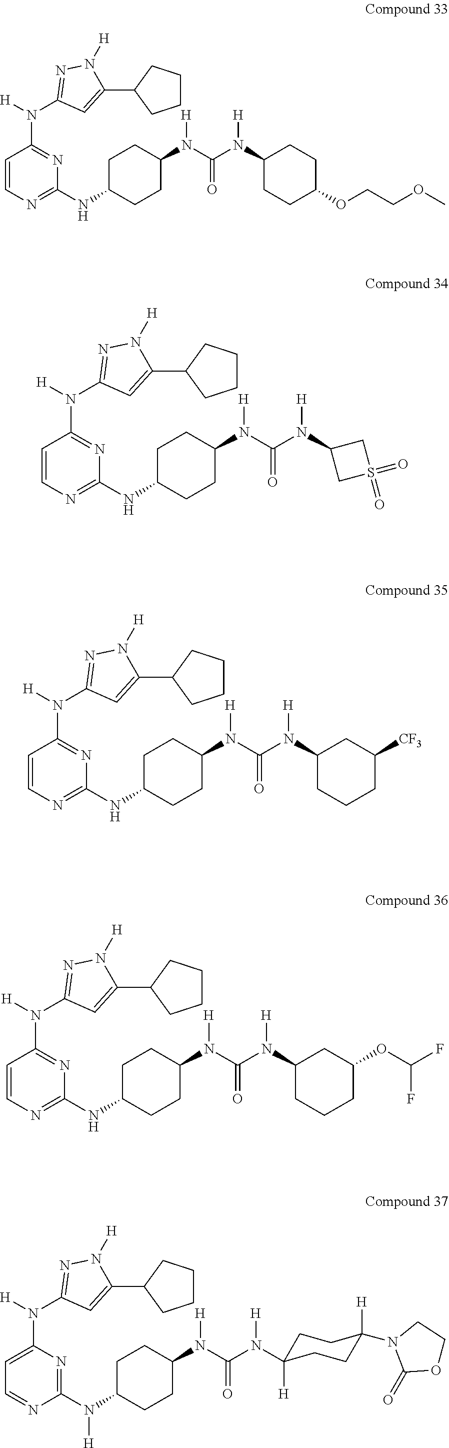

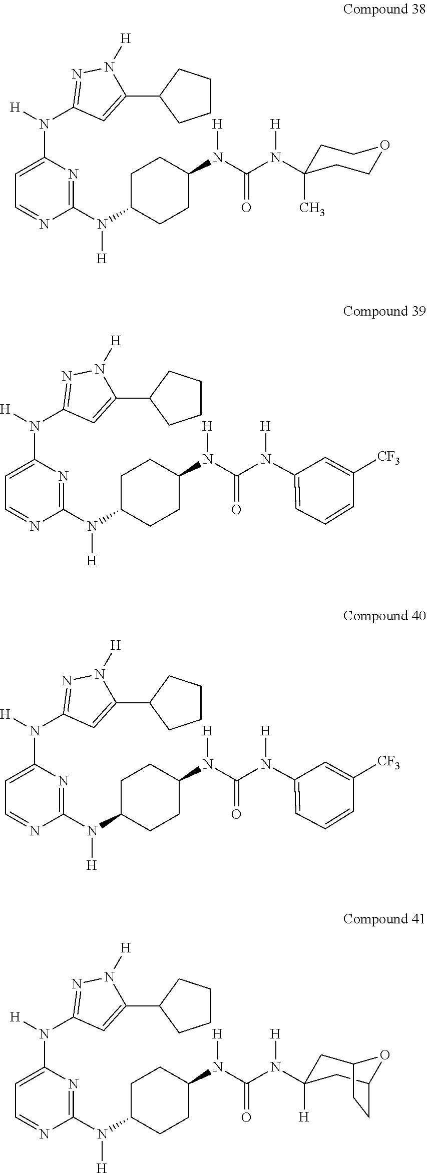 [SMILES: CC1(NC(=O)N[C@H]2CC[C@H](Nc3nccc(Nc4cc(C5CCCC5)[nH]n4)n3)CC2)CCOCC1 ; O=C(Nc1cccc(C(F)(F)F)c1)N[C@H]1CC[C@H](Nc2nccc(Nc3cc(C4CCCC4)[nH]n3)n2)CC1 ; O=C(Nc1cccc(C(F)(F)F)c1)N[C@H]1CC[C@@H](Nc2nccc(Nc3cc(C4CCCC4)[nH]n3)n2)CC1 ; O=C(NC1CC2CCC(C1)O2)N[C@H]1CC[C@H](Nc2nccc(Nc3cc(C4CCCC4)[nH]n3)n2)CC1]

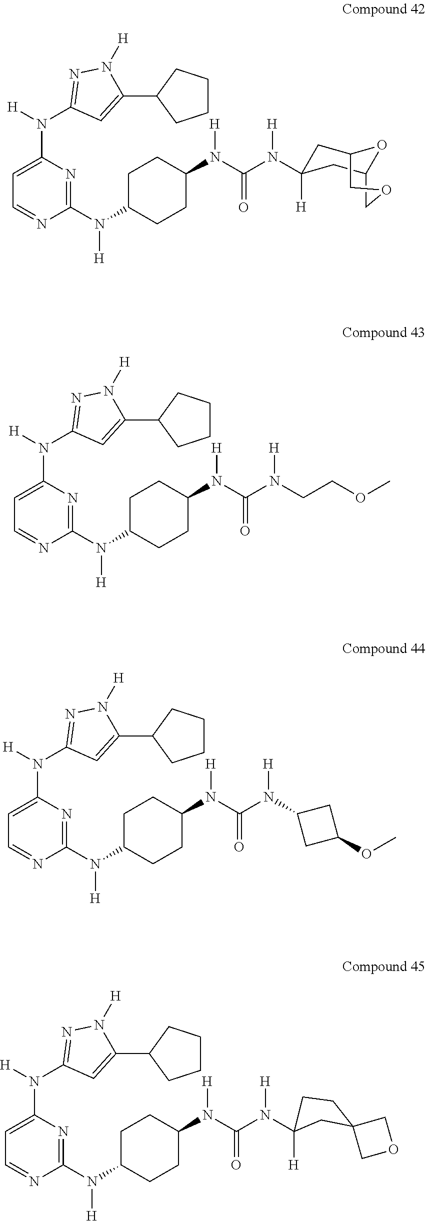

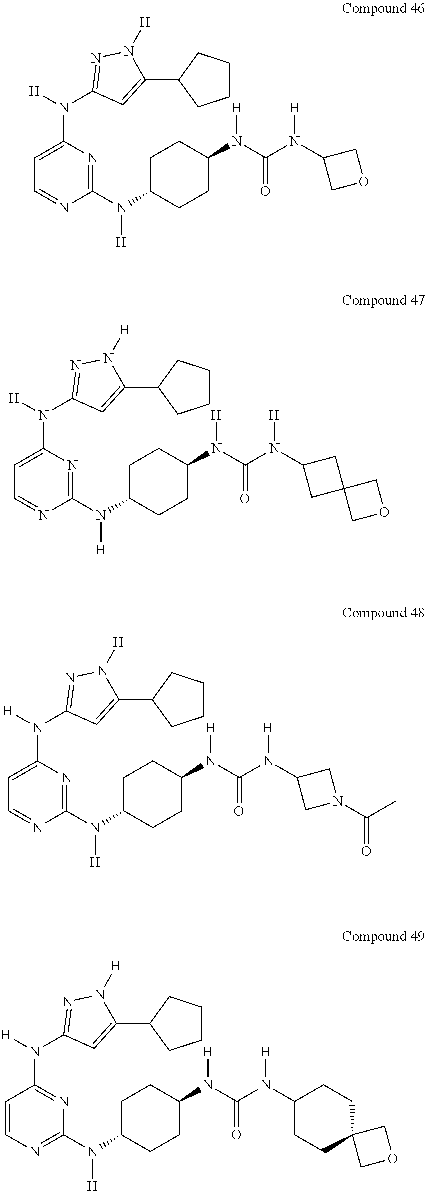

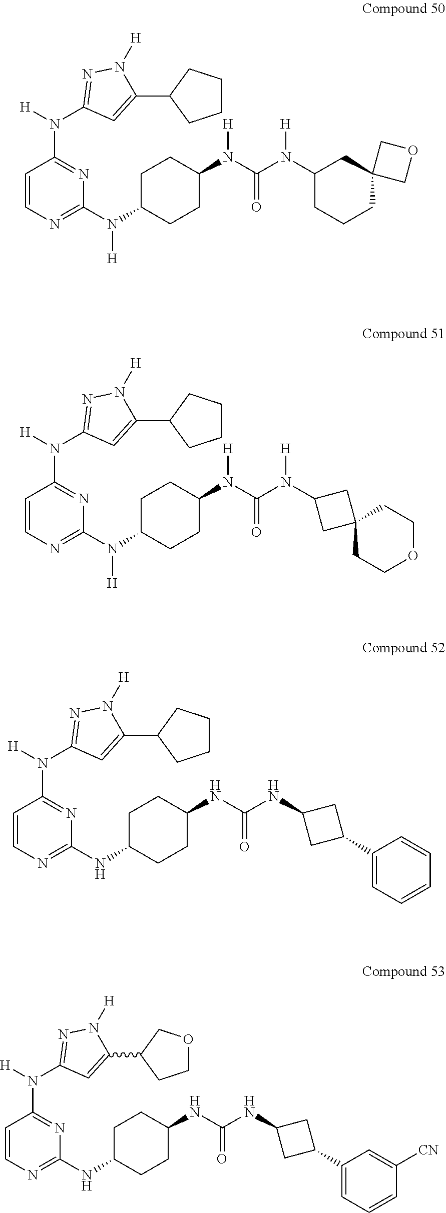

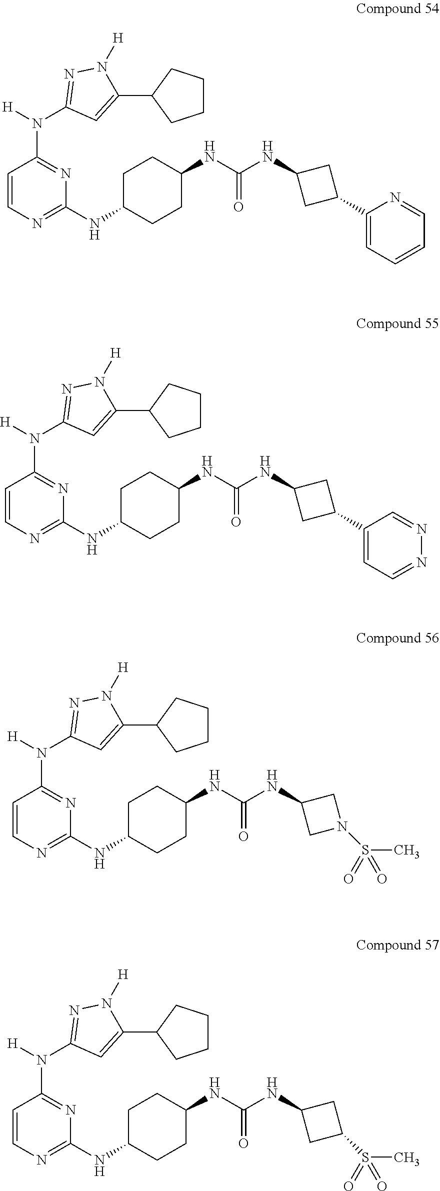

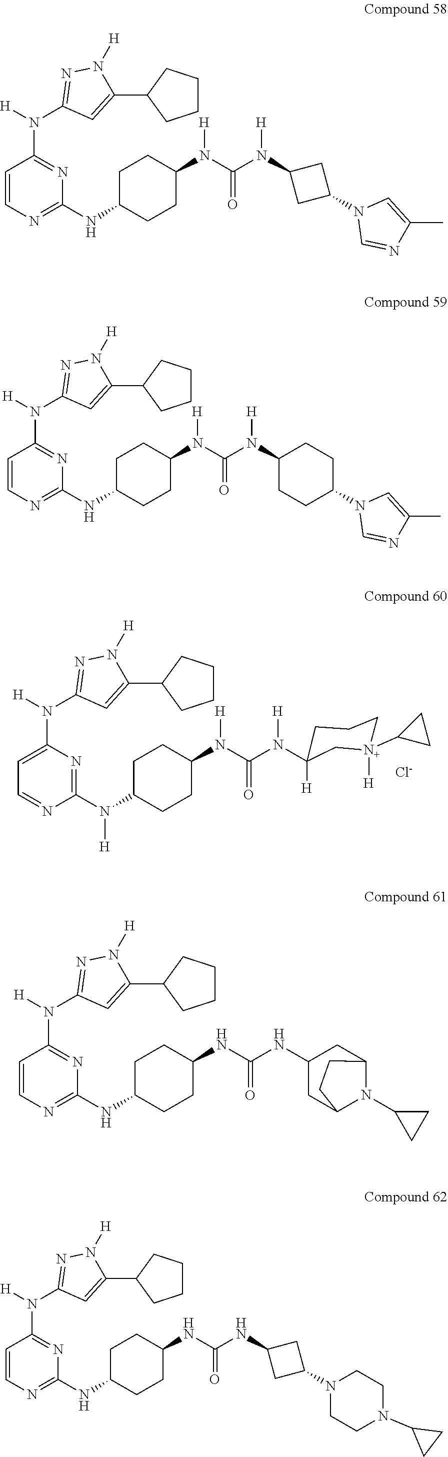

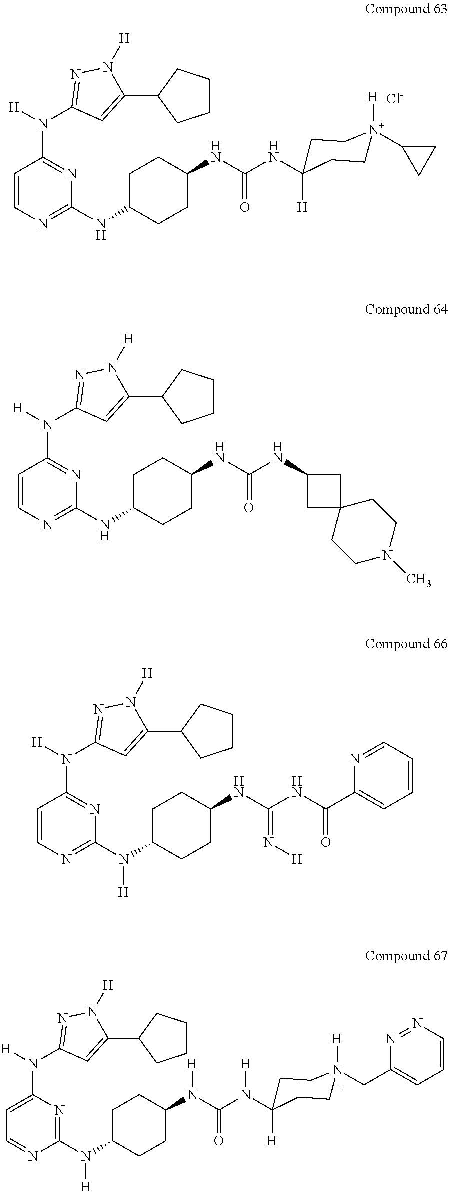

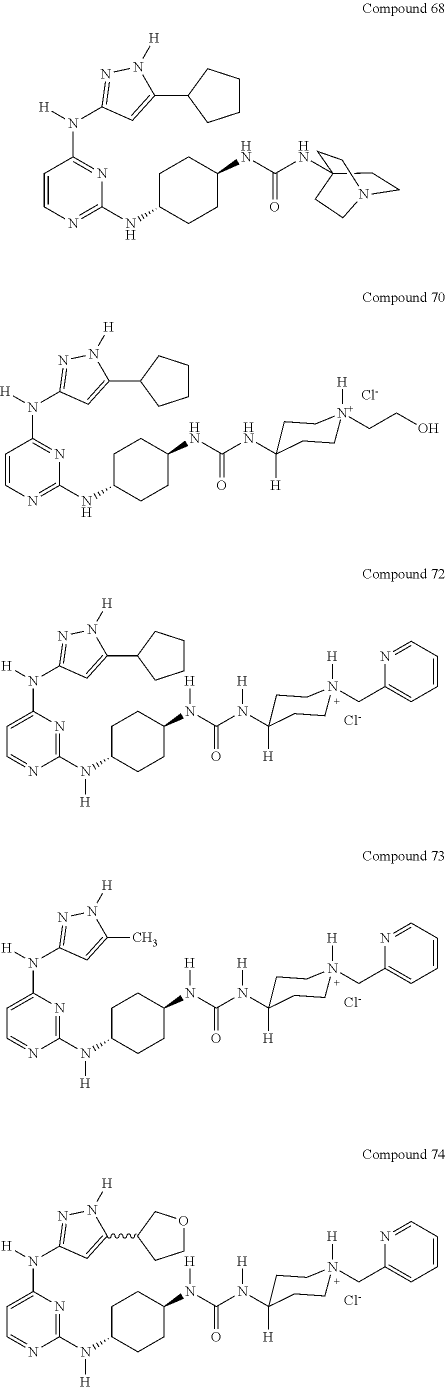

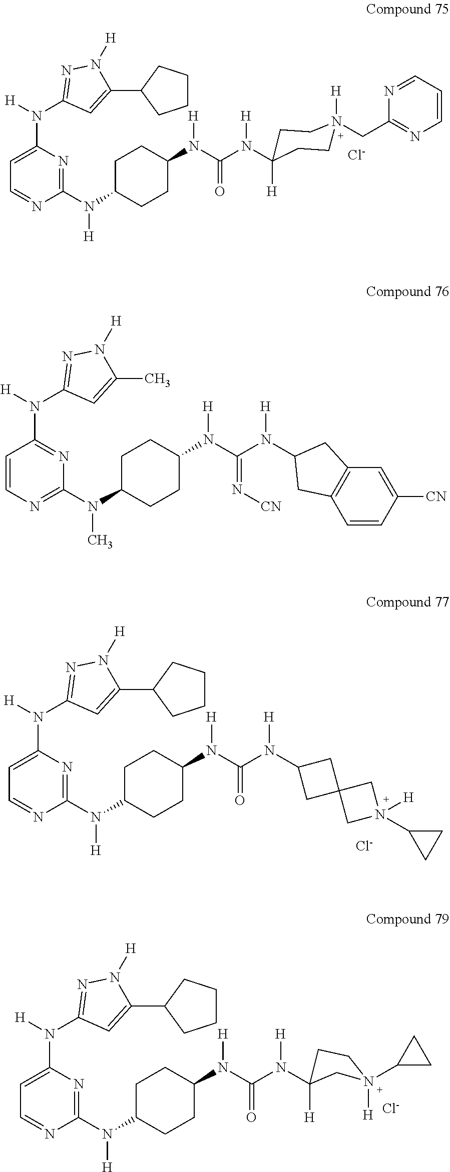

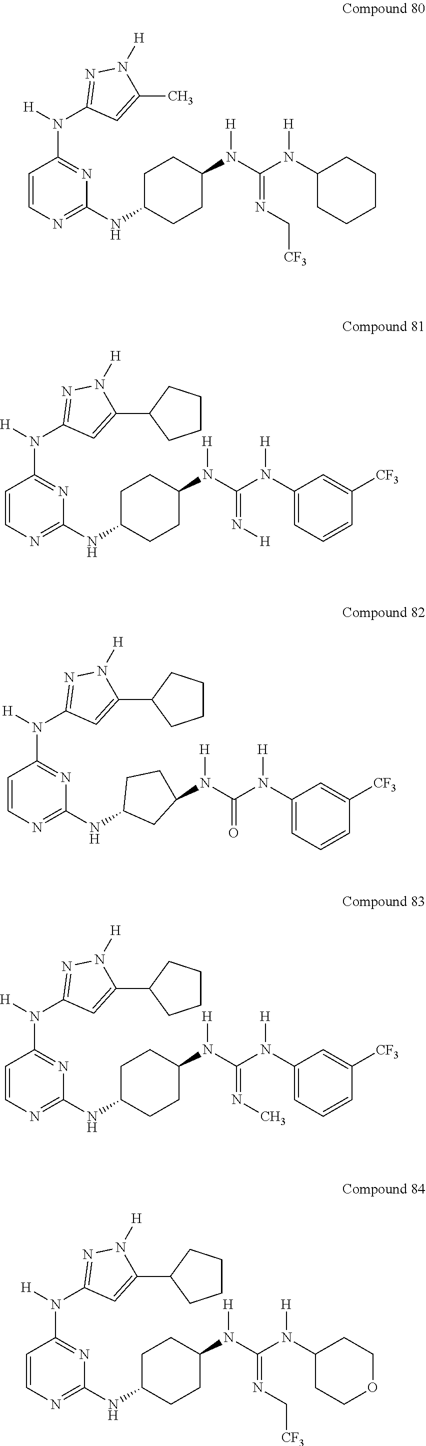 [SMILES: Cc1cc(Nc2ccnc(N[C@H]3CC[C@H](N/C(=N/CC(F)(F)F)NC4CCCCC4)CC3)n2)n[nH]1 ; [H]/N=C(\Nc1cccc(C(F)(F)F)c1)N[C@H]1CC[C@H](Nc2nccc(Nc3cc(C4CCCC4)[nH]n3)n2)CC1 ; O=C(Nc1cccc(C(F)(F)F)c1)N[C@@H]1CC[C@@H](Nc2nccc(Nc3cc(C4CCCC4)[nH]n3)n2)C1 ; C/N=C(\Nc1cccc(C(F)(F)F)c1)N[C@H]1CC[C@H](Nc2nccc(Nc3cc(C4CCCC4)[nH]n3)n2)CC1 ; FC(F)(F)C/N=C(\NC1CCOCC1)N[C@H]1CC[C@H](Nc2nccc(Nc3cc(C4CCCC4)[nH]n3)n2)CC1]

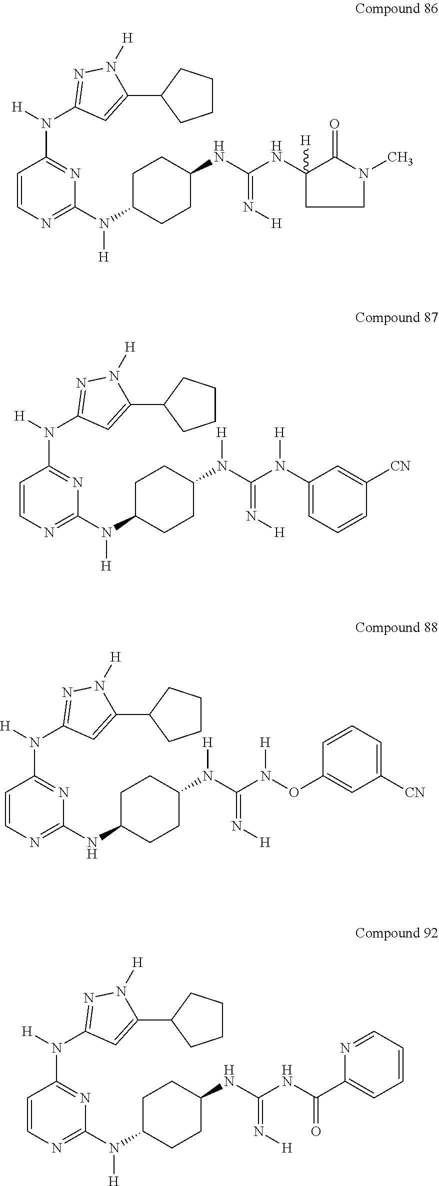 [SMILES: [H]/N=C(\NC1CCN(C)C1=O)N[C@H]1CC[C@H](Nc2nccc(Nc3cc(C4CCCC4)[nH]n3)n2)CC1 ; [H]/N=C(\Nc1cccc(C#N)c1)N[C@H]1CC[C@H](Nc2nccc(Nc3cc(C4CCCC4)[nH]n3)n2)CC1 ; [H]/N=C(\NOc1cccc(C#N)c1)N[C@H]1CC[C@H](Nc2nccc(Nc3cc(C4CCCC4)[nH]n3)n2)CC1 ; [H]/N=C(\NC(=O)c1ccccn1)N[C@H]1CC[C@H](Nc2nccc(Nc3cc(C4CCCC4)[nH]n3)n2)CC1]

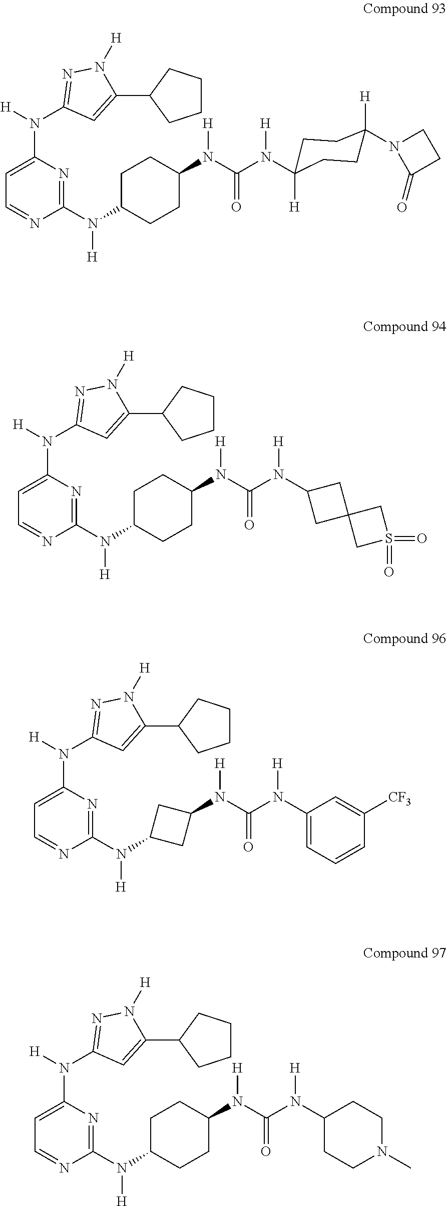

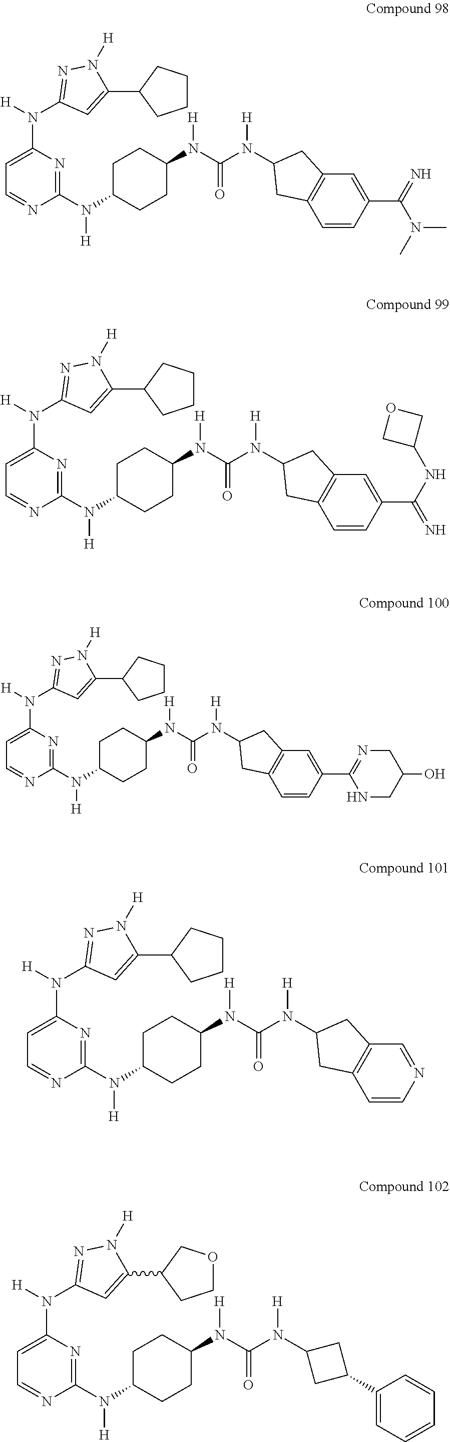

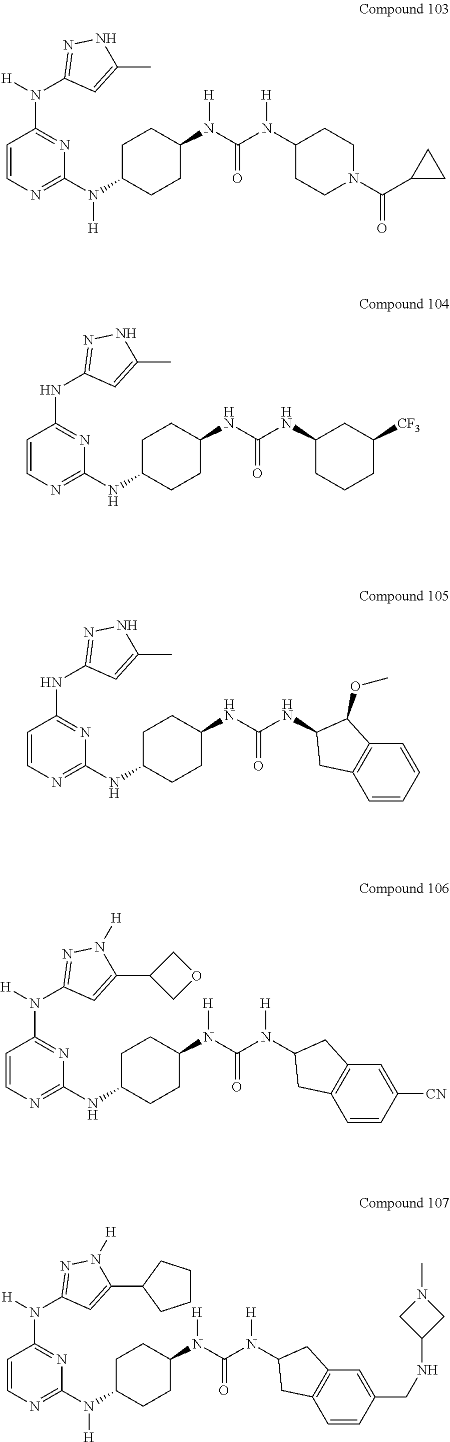 [SMILES: Cc1cc(Nc2ccnc(N[C@H]3CC[C@H](NC(=O)NC4CCN(C(=O)C5CC5)CC4)CC3)n2)n[nH]1 ; Cc1cc(Nc2ccnc(N[C@H]3CC[C@H](NC(=O)N[C@@H]4CCC[C@H](C(F)(F)F)C4)CC3)n2)n[nH]1 ; CO[C@H]1c2ccccc2C[C@H]1NC(=O)N[C@H]1CC[C@H](Nc2nccc(Nc3cc(C)[nH]n3)n2)CC1 ; N#Cc1ccc2c(c1)CC(NC(=O)N[C@H]1CC[C@H](Nc3nccc(Nc4cc(C5COC5)[nH]n4)n3)CC1)C2 ; CN1CC(NCc2ccc3c(c2)CC(NC(=O)N[C@H]2CC[C@H](Nc4nccc(Nc5cc(C6CCCC6)[nH]n5)n4)CC2)C3)C1]

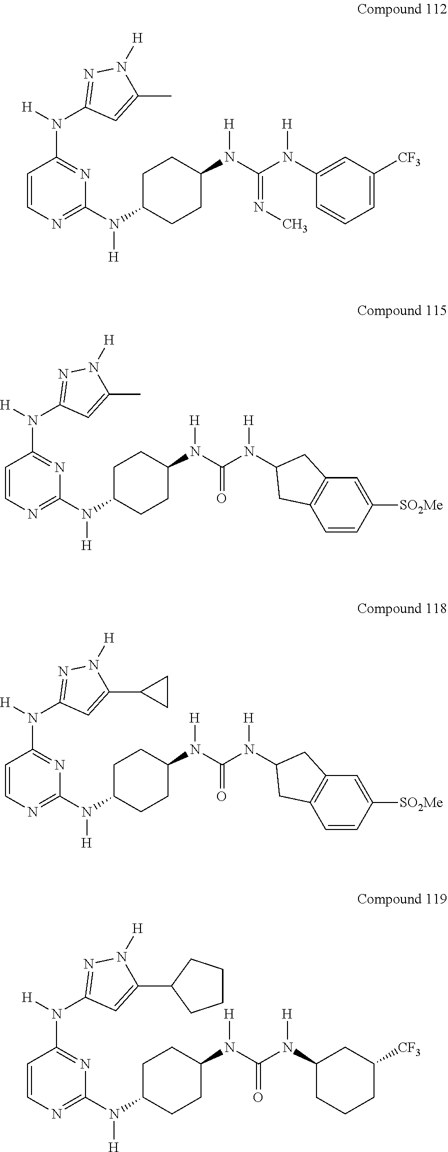 [SMILES: C/N=C(\Nc1cccc(C(F)(F)F)c1)N[C@H]1CC[C@H](Nc2nccc(Nc3cc(C)[nH]n3)n2)CC1 ; Cc1cc(Nc2ccnc(N[C@H]3CC[C@H](NC(=O)NC4Cc5ccc(S(C)(=O)=O)cc5C4)CC3)n2)n[nH]1 ; CS(=O)(=O)c1ccc2c(c1)CC(NC(=O)N[C@H]1CC[C@H](Nc3nccc(Nc4cc(C5CC5)[nH]n4)n3)CC1)C2 ; O=C(N[C@@H]1CCC[C@@H](C(F)(F)F)C1)N[C@H]1CC[C@H](Nc2nccc(Nc3cc(C4CCCC4)[nH]n3)n2)CC1]

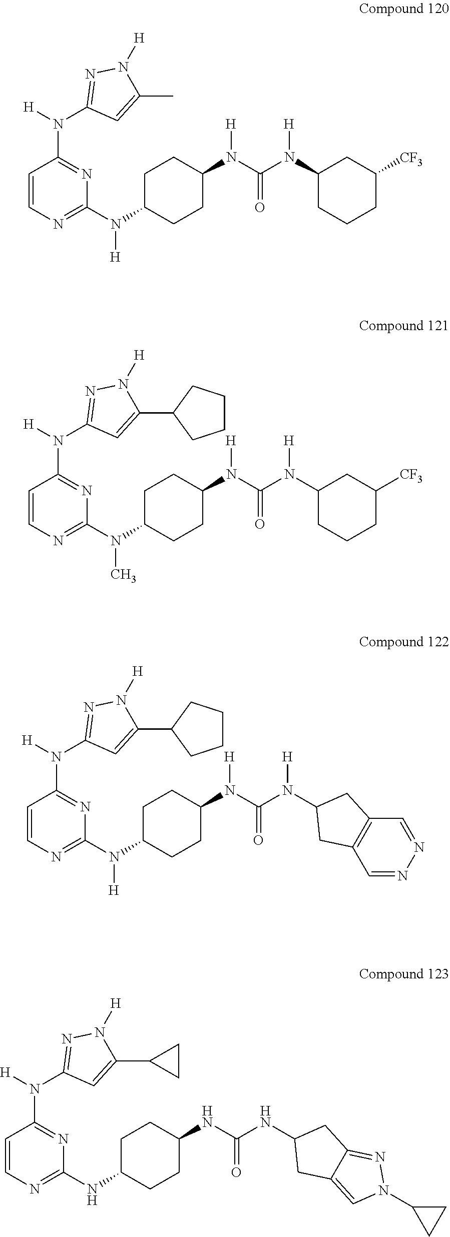 [SMILES: Cc1cc(Nc2ccnc(N[C@H]3CC[C@H](NC(=O)N[C@@H]4CCC[C@@H](C(F)(F)F)C4)CC3)n2)n[nH]1 ; CN(c1nccc(Nc2cc(C3CCCC3)[nH]n2)n1)[C@H]1CC[C@H](NC(=O)NC2CCCC(C(F)(F)F)C2)CC1 ; O=C(NC1Cc2cnncc2C1)N[C@H]1CC[C@H](Nc2nccc(Nc3cc(C4CCCC4)[nH]n3)n2)CC1 ; O=C(NC1Cc2cn(C3CC3)nc2C1)N[C@H]1CC[C@H](Nc2nccc(Nc3cc(C4CC4)[nH]n3)n2)CC1]

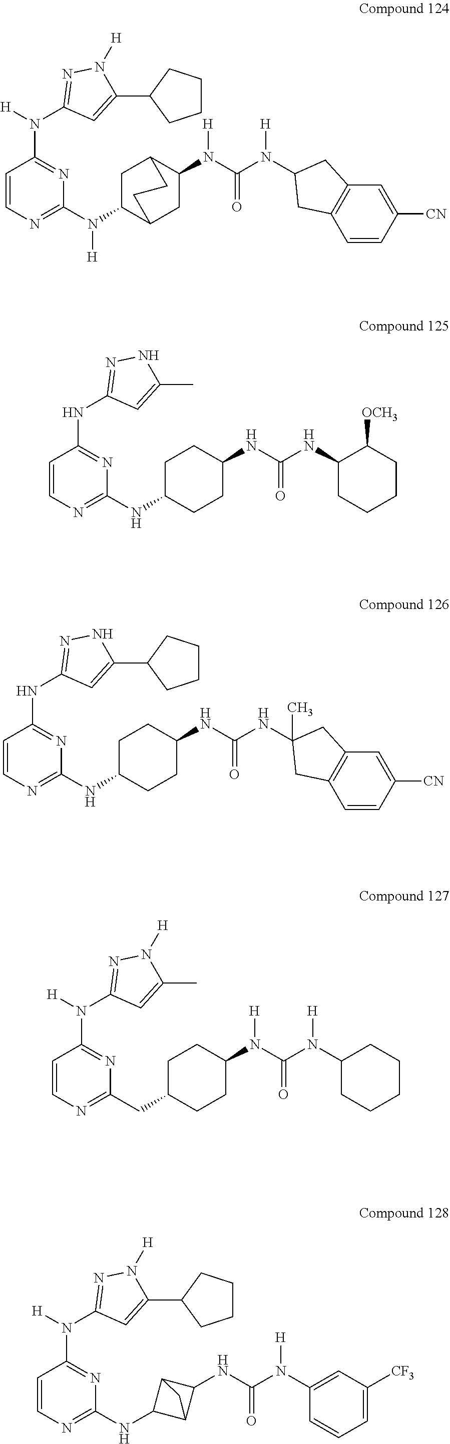 [SMILES: N#Cc1ccc2c(c1)CC(NC(=O)N[C@H]1CC3CCC1C[C@H]3Nc1nccc(Nc3cc(C4CCCC4)[nH]n3)n1)C2 ; CO[C@H]1CCCC[C@H]1NC(=O)N[C@H]1CC[C@H](Nc2nccc(Nc3cc(C)[nH]n3)n2)CC1 ; CC1(NC(=O)N[C@H]2CC[C@H](Nc3nccc(Nc4cc(C5CCCC5)[nH]n4)n3)CC2)Cc2ccc(C#N)cc2C1 ; Cc1cc(Nc2ccnc(C[C@H]3CC[C@H](NC(=O)NC4CCCCC4)CC3)n2)n[nH]1 ; O=C(Nc1cccc(C(F)(F)F)c1)NC1C2CC1C2Nc1nccc(Nc2cc(C3CCCC3)[nH]n2)n1]

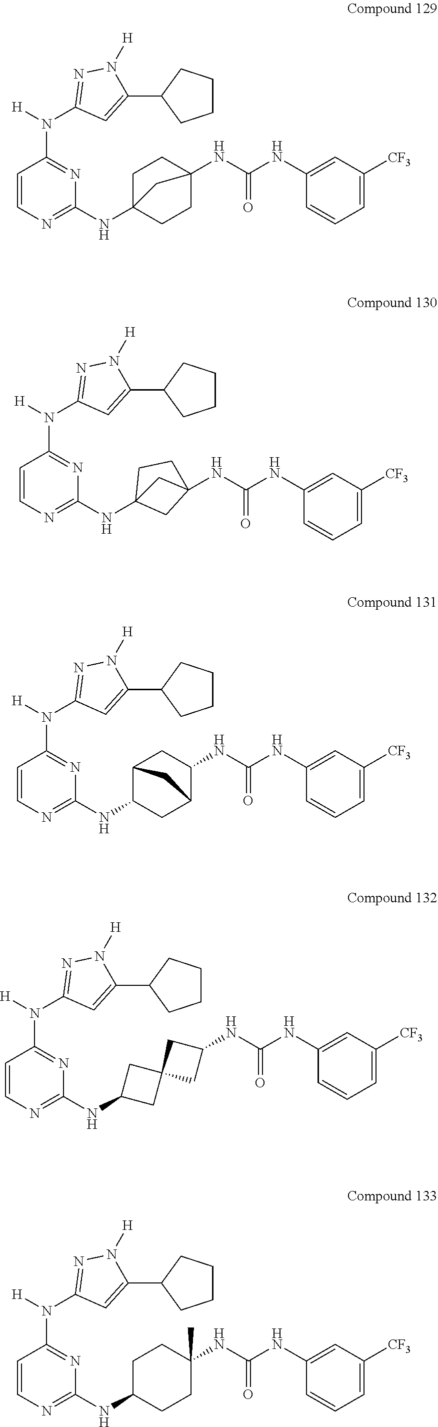

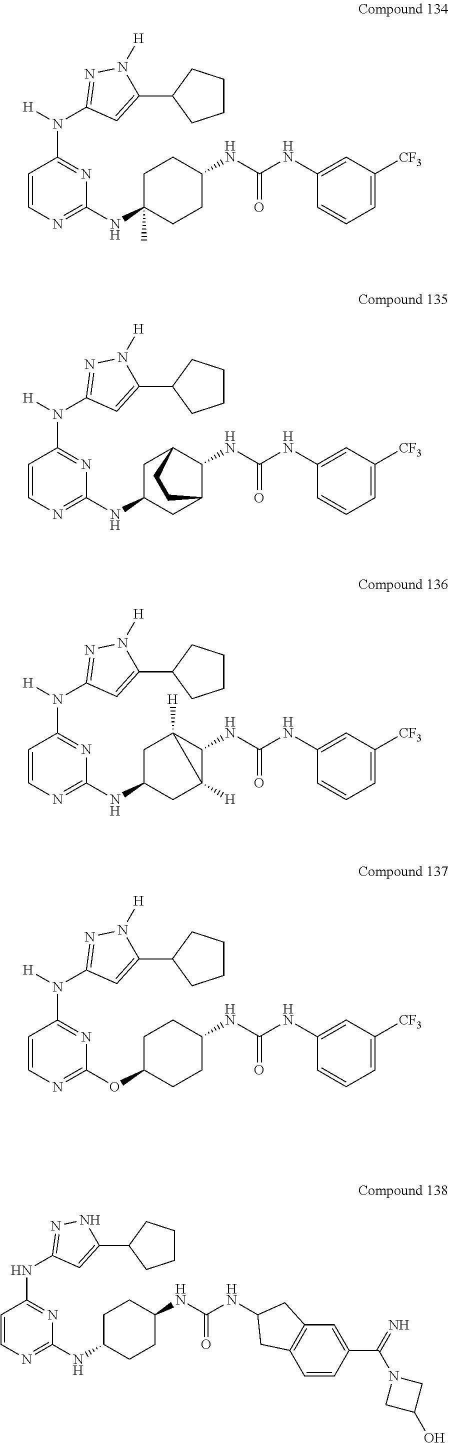

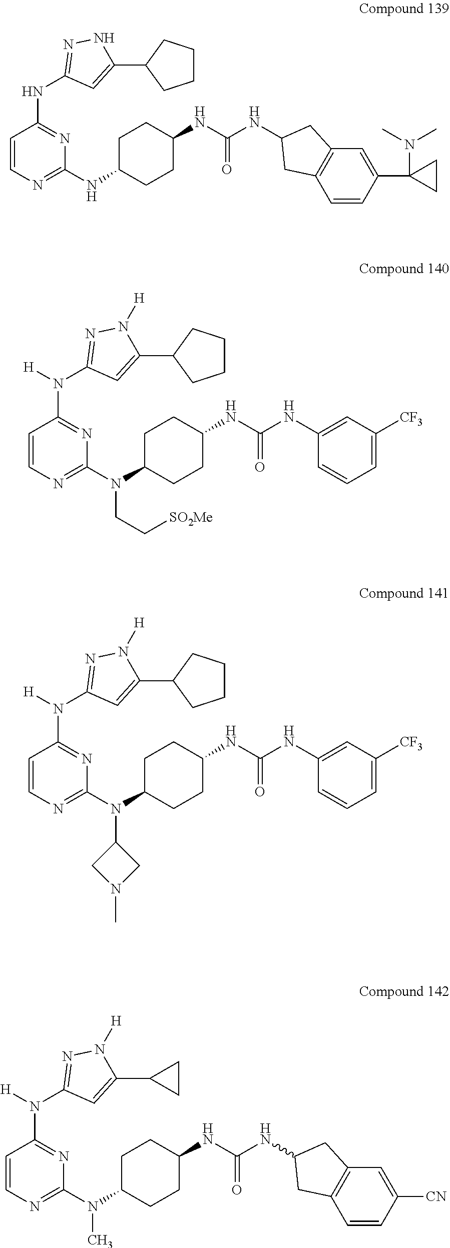 [SMILES: CN(C)C1(c2ccc3c(c2)CC(NC(=O)N[C@H]2CC[C@H](Nc4nccc(Nc5cc(C6CCCC6)[nH]n5)n4)CC2)C3)CC1 ; CS(=O)(=O)CCN(c1nccc(Nc2cc(C3CCCC3)[nH]n2)n1)[C@H]1CC[C@H](NC(=O)Nc2cccc(C(F)(F)F)c2)CC1 ; CN1CC(N(c2nccc(Nc3cc(C4CCCC4)[nH]n3)n2)[C@H]2CC[C@H](NC(=O)Nc3cccc(C(F)(F)F)c3)CC2)C1 ; CN(c1nccc(Nc2cc(C3CC3)[nH]n2)n1)[C@H]1CC[C@H](NC(=O)NC2Cc3ccc(C#N)cc3C2)CC1]

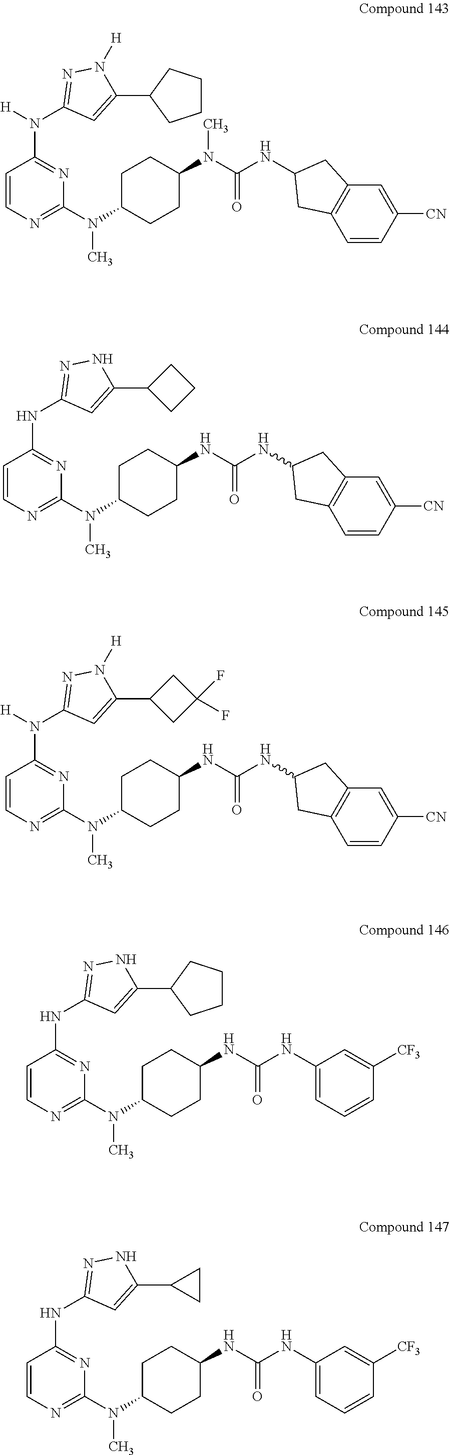 [SMILES: CN(C(=O)NC1Cc2ccc(C#N)cc2C1)[C@H]1CC[C@H](N(C)c2nccc(Nc3cc(C4CCCC4)[nH]n3)n2)CC1 ; CN(c1nccc(Nc2cc(C3CCC3)[nH]n2)n1)[C@H]1CC[C@H](NC(=O)NC2Cc3ccc(C#N)cc3C2)CC1 ; CN(c1nccc(Nc2cc(C3CC(F)(F)C3)[nH]n2)n1)[C@H]1CC[C@H](NC(=O)NC2Cc3ccc(C#N)cc3C2)CC1 ; CN(c1nccc(Nc2cc(C3CCCC3)[nH]n2)n1)[C@H]1CC[C@H](NC(=O)Nc2cccc(C(F)(F)F)c2)CC1 ; CN(c1nccc(Nc2cc(C3CC3)[nH]n2)n1)[C@H]1CC[C@H](NC(=O)Nc2cccc(C(F)(F)F)c2)CC1]

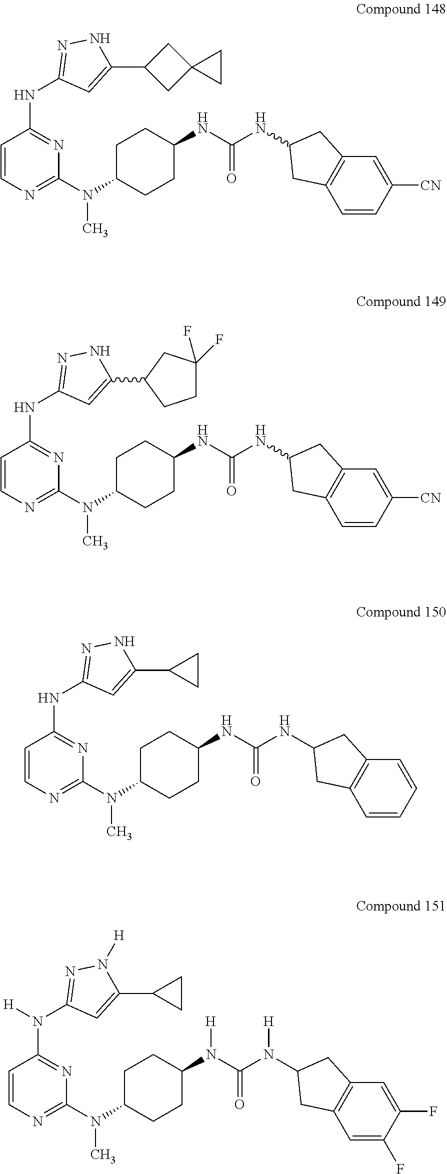 [SMILES: CN(c1nccc(Nc2cc(C3CC4(CC4)C3)[nH]n2)n1)[C@H]1CC[C@H](NC(=O)NC2Cc3ccc(C#N)cc3C2)CC1 ; CN(c1nccc(Nc2cc(C3CCC(F)(F)C3)[nH]n2)n1)[C@H]1CC[C@H](NC(=O)NC2Cc3ccc(C#N)cc3C2)CC1 ; CN(c1nccc(Nc2cc(C3CC3)[nH]n2)n1)[C@H]1CC[C@H](NC(=O)NC2Cc3ccccc3C2)CC1 ; CN(c1nccc(Nc2cc(C3CC3)[nH]n2)n1)[C@H]1CC[C@H](NC(=O)NC2Cc3cc(F)c(F)cc3C2)CC1]

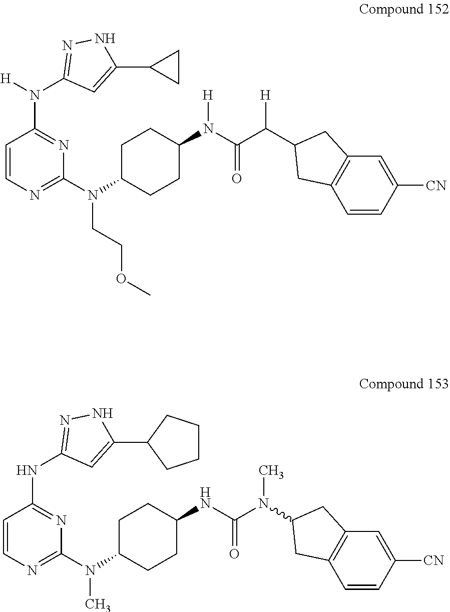 [SMILES: COCCN(c1nccc(Nc2cc(C3CC3)[nH]n2)n1)[C@H]1CC[C@H](NC(=O)CC2Cc3ccc(C#N)cc3C2)CC1 ; CN(C(=O)N[C@H]1CC[C@H](N(C)c2nccc(Nc3cc(C4CCCC4)[nH]n3)n2)CC1)C1Cc2ccc(C#N)cc2C1]

and a pharmaceutically acceptable salt, stereoisomer and/or N-oxide thereof.

Disclosed compounds described herein may be present in a salt form, and the salt form of the compound is a pharmaceutically acceptable salt, and/or compounds described herein may be present in a prodrug form. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)). Compounds described herein may be present in a solvate form.

In some embodiments, the compounds, or a prodrug form thereof, are provided in the form of pharmaceutically acceptable salts. Compounds containing an amine functional group or a nitrogen-containing heteroaryl group may be basic in nature and may react with any number of inorganic and organic acids to from the corresponding pharmaceutically acceptable salts. Inorganic acids commonly employed to form such salts include hydrochloric, hydrobromic, hydroiodic, sulfuric, and phosphoric acids, and related inorganic acids. Organic acids commonly employed to form such salts include para-toluenesulfonic, methanesulfonic, oxalic, para-bromophenylsulfonic, fumaric, maleic, carbonic, succinic, citric, benzoic and acetic acid, and related organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, O-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the related salts.

It is understood that all variations of salts, solvates, hydrates, prodrugs and stereoisomers are meant to be encompassed by the present disclosure.

### Pharmaceutical Compositions and Formulations

The compounds, prodrugs, and compositions described herein can be useful as pharmaceutical compositions for administration to a subject in need thereof.

Accordingly, pharmaceutical compositions are presented that can comprise at least a compound described herein, a pharmaceutically acceptable salt thereof, or a prodrug thereof, and at least one pharmaceutically acceptable carriers, diluent, stabilizers, excipients, dispersing agents, suspending agents, or thickening agents. For example, a disclosed pharmaceutical compositions may include one or more of the disclosed compounds, pharmaceutically acceptable salts, or prodrugs described herein. Contemplated compositions may include a compound, a pharmaceutically acceptable salt thereof, or a prodrug thereof in a therapeutically effective amount, for example, a disclosed pharmaceutical composition may be formulated for parenteral administration to a subject in need thereof, formulated for intravenous administration to a subject in need thereof, or formulated for subcutaneous administration to a subject in need thereof.

### Methods of Treatment

As described above, embodiments of the present disclosure include the use of compounds, prodrugs, and pharmaceutical compositions described herein to treat a Myc protein associated proliferative disease in a subject in need thereof. Such proliferative diseases include cancer, for example, a cancer selected from a group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

In some embodiments, a contemplated method of treating includes treating a cancer that is a Myc protein associated cancer, e.g., wherein the Myc protein is selected from the group consisting of a N-Myc protein, a c-MYc protein, a L-Myc protein, a human N-Myc protein, a human c-Myc protein, and a human L-Myc protein.

For example, provided herein is a method of treating a cancer selected from the group consisting of neuroblastoma, small cell lung carcinoma, breast cancer or a hematopoietic cancer.

In some embodiments, a disclosed method to treat cancer further comprises a second therapy, wherein the secondary therapy is an antineoplastic therapy, e.g., a contemplated method may further comprise administering an antineoplastic therapy such as one or more agents selected from a DNA topoisomerase I or II inhibitor, a DNA damaging agent, an immunotherapeutic agent (e.g., an antibody, cytokine, immune checkpoint inhibitor or cancer vaccine), an antimetabolite or a thymidylate synthase (TS) inhibitor, a microtubule targeted agent, ionizing radiation, an inhibitor of a mitosis regulator or a mitotic checkpoint regulator, an inhibitor of a DNA damage signal transducer, and an inhibitor of a DNA damage repair enzyme. For example, additional antineoplastic therapy may be selected from the group consisting of immunotherapy (e.g., immuno-oncologic therapy), radiation therapy, photodynamic therapy, gene-directed enzyme prodrug therapy (GDEPT), antibody-directed enzyme prodrug therapy (ADEPT), gene therapy, and controlled diets.

The present disclosure also contemplates the use of compounds, prodrugs, and pharmaceutical compositions described herein to modulate the amount and activity of a Myc protein (in vitro or in a patient), where the Myc protein may be for example a N-Myc protein, a c-MYc protein, a L-Myc protein, a human N-Myc protein, a human c-Myc protein, and/or a human L-Myc protein.

For example, the disclosure provides a method of modulating the amount (e.g., the concentration) and/or activity of a Myc protein such as (e.g. degrading a Myc protein, or modulating the rate of degradation of a Myc protein) that comprises contacting a Myc protein with an effective amount of a compound described herein, or a pharmaceutically acceptable salt, stereoisomer and/or N-oxide thereof, including embodiments or from any examples, tables or figures.

Contemplated methods include methods of modulating the protein-protein interactions of the Myc family protein, or a method of decreasing the amount and decreasing the level of activity of a Myc protein.

A disclosed method of modulating the amount and activity of a Myc protein may include co-administering a compound described herein, or a pharmaceutically acceptable salt thereof, and a therapeutically effective amount of a second agent, e.g., therapeutic agent.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (i.e., amounts, temperatures, etc.), but some experimental error and deviation should, of course, be allowed for.

### General Experimental

Final compounds were confirmed by HPLC/MS analysis and determined to be >90% pure by weight. ¹H and ¹³C NMR spectra were recorded in CDCl₃ (residual internal standard CHCl₃=δ 7.26), DMSO-d₆ (residual internal standard CD₃SOCD₂H=δ 2.50), methanol-d₄ (residual internal standard CD₂HOD=δ 3.20), or acetone-d₆ (residual internal standard CD₃COCD₂H=δ 2.05). The chemical shifts (6) reported are given in parts per million (ppm) and the coupling constants (J) are in Hertz (Hz). The spin multiplicities are reported as s=singlet, bs=broad singlet, bm=broad multiplet, d=doublet, t=triplet, q=quartet, p=pentuplet, dd=doublet of doublet, ddd=doublet of doublet of doublet, dt=doublet of triplet, td=triplet of doublet, tt=triplet of triplet, and m=multiplet.

HPLC-MS analysis was carried out with gradient elution. Medium pressure liquid chromatography (MPLC) was performed with silica gel columns in both the normal phase and reverse phase. It will be appreciated that compounds reported as a salt form (e.g., a TFA salt) may or may not have a 1:1 stoichiometry, and/or for example, reported potency concentrations or other assay results may be, e.g., slightly higher or lower.

### Example 1: Synthesis of Intermediate E

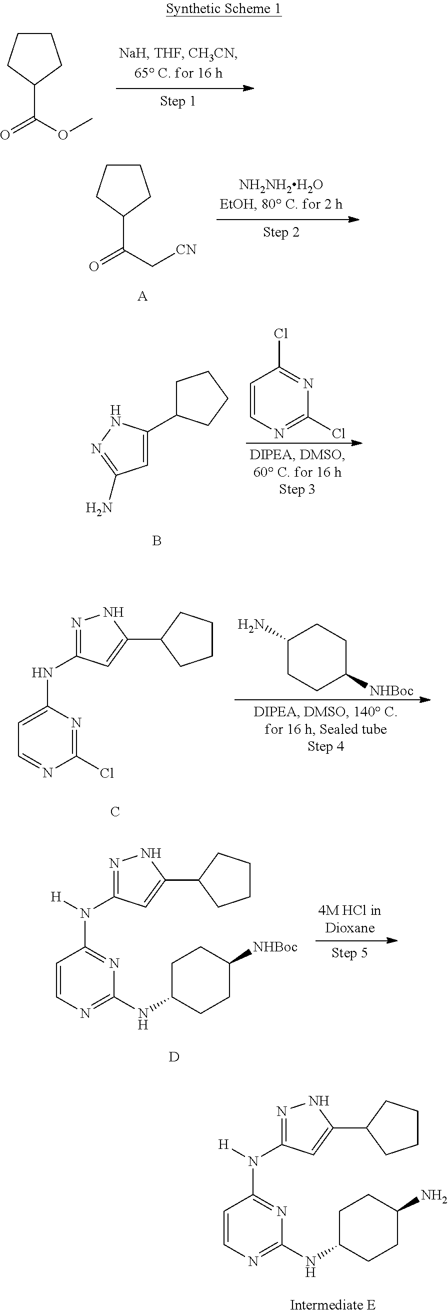

### Step-1: Synthesis of Compound A—3-cyclopentyl-3-oxopropanenitrile

To a slurry of sodium hydride (37 g, 937 mmol) in THF (600 mL) at 65° C. was added a mixture of methyl cyclopentanecarboxylate (60 g, 468 mmol) and acetonitrile (38 g, 937 mmol) dropwise. The reaction was stirred at 65° C. for 16 h. The progress of the reaction was monitored by TLC after complete consumption of starting material, the reaction mixture was poured into ice and extracted with diethyl ether. The aqueous layer was acidified to a pH=4-5 then extracted with diethyl ether. The organic layer was dried over anhydrous Na₂SO₄, filtered and the solvent was concentrated to give compound A—3-cyclopentyl-3-oxopropanenitrile (59 g, 92.18% yield). ¹H NMR (400 MHz, DMSO-d₆): δ 4.11 (s, 2H), 3.00-2.92 (m, 1H), 1.81-1.77 (m, 2H), 1.76-1.73 (m, 2H), 1.70-1.65 (m, 4H).

### Step-2: Synthesis of Compound B—5-cyclopentyl-1H-pyrazol-3-amine

To a stirred solution of 3-cyclopentyl-3-oxopropanenitrile (59 g, 430 mmol) in ethanol (600 mL) was added hydrazine hydrate (42 mL, 861 mmol). The reaction mixture was heated to 80° C. for 2 h. The progress of the reaction was monitored by TLC after complete consumption of starting material, the reaction mixture was concentrated under reduced pressure and the crude was washed with diethyl ether to give compound B—5-cyclopentyl-1H-pyrazol-3-amine (60 g, 92.30% yield). ¹H NMR (400 MHz, DMSO-d₆): δ 5.17 (s, 1H), 2.87-2.83 (m, 1H), 1.92-1.90 (m, 2H), 1.89-1.47 (m, 9H). LC purity: 98.45%; m/z: 152.2 [M+H]⁺ (Mol. formula C₈H₁₃N₃, calcd. mol. wt. 151.21).

### Step-3: Synthesis of Compound C—2-chloro-N-(5-cyclopentyl-1H-pyrazol-3-yl)pyrimidin-4-amine

To a stirred solution of 5-cyclopentyl-1H-pyrazol-3-amine (60 g, 397 mmol) in DMSO (600 mL) was added DIPEA (101 mL, 595 mmol) and 2, 4-dichloropyrimidine (71 g, 476 mmol). The reaction was heated to 60° C. for 16 h. The progress of the reaction was monitored by TLC after complete consumption of starting material, the reaction mixture was quenched with ice water and the solid was filtered, washed with dichloromethane and dried under vacuum to give compound C—2-chloro-N-(5-cyclopentyl-1H-pyrazol-3-yl)pyrimidin-4-amine (60 g, 57.69% yield). LC purity: 93.55%; m/z: 264.1 [M+H]⁺ (Mol. formula C₁₂H₁₄ClN₅, calcd. mol. wt. 263.73).

### Step-4: Synthesis of Compound D—tert-butyl((1R,4R)-4-((4-((5-cyclopentyl-1H-pyrazol-yl)amino)pyrimidin-2-yl)amino)cyclohexyl)carbamate

To a stirred solution of 2-chloro-N-(5-cyclopentyl-1H-pyrazol-3-yl)pyrimidin-4-amine (7.0 g, 26.315 mmol) in dry DMSO (75 mL) in a sealed tube was added DIPEA (9.3 mL, 52.63 mmol). The reaction mixture was cooled to 0° C. and added tert-butyl((1R,4R)-4-aminocyclohexyl)carbamate (8.6 g, 39.572 mmol). The reaction mixture was heated to 140° C. for 16 h. The reaction was monitored by TLC, after complete consumption of starting material, the reaction mixture was quenched with ice cold water and extracted with ethyl acetate. The resulting organic layer was washed with brine solution then dried over anhydrous Na₂SO₄ and concentrated to obtain crude compound. The crude was purified by Biotage-isolera using silica gel (230-400 mesh) with a gradient elution of 0-16% dichloromethane/methanol to give compound D—tert-butyl((1R,4R)-4-((4-((5-cyclopentyl-1H-pyrazol-3-yl)amino)pyrimidin-2-yl)amino)cyclohexyl)carbamate (6.0 g, 51% yield). LC purity: 90.25%; m/z: 442.3 [M+H]⁺ (Mol. formula C₂₇H₃₅N₇O₂, calcd. mol. wt. 441.0).

### Step-5: Synthesis of Intermediate E—N²-((1R,4R)-4-(aminocyclohexyl)-N4-((5-cyclopentyl-1H-pyrazol-3-yl)) pyrimidine-2,4-diamine

To a stirred solution of tert-butyl((1R,4R)-4-((4-((5-cyclopentyl-1H-pyrazol-3-yl)amino)pyrimidin-2-yl)amino)cyclohexyl)carbamate (6.0 g, 13.605 mmol) in dry DCM (60 mL). The reaction mixture was cooled to 0° C. and added HCl in Dioxane (60 mL, 4M solution). The reaction was allowed to stir at room temperature for 3 h. The progress of the reaction was monitored by TLC after complete consumption of starting material, the resulting mixture was concentrated and triturated with pet ether and concentrated under high vacuum to yield N²-((1R,4R)-4-(aminocyclohexyl)-N4-((5-cyclopentyl-1H-pyrazol-3-yl)) pyrimidine-2,4-diamine as a HCl salt (5.0 g, quantitative yield). LC purity: 99.78%; m/z: 342.3 [M+H]⁺ (Mol. formula C₂₈H₂₇N₇, calcd. mol. wt. 341.0).

### Example 2: Synthesis of Compound 39

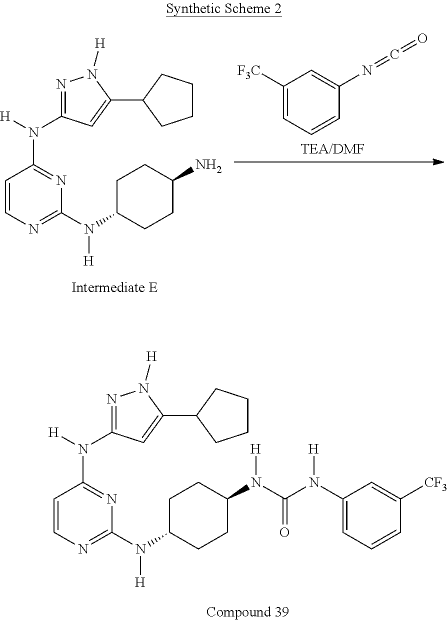

To a stirred solution of Intermediate E—N²-((1R,4R)-4-aminocyclohexyl)-N⁴-(5-cyclopentyl-1H-pyrazol-3-yl)pyrimidine-2,4-diamine (0.25 g, 0.73 mmol) in dry DMF (1 mL) was cooled to 0° C. was added triethylamine (0.37 mL, 3.6 mmol) and 1-isocyanato-3-(trifluoromethyl)benzene (0.137 mL, 0.73 mmol) dropwise. The reaction was stirred at room temperature for 4 h (the reaction mixture was monitored by TLC). After completion of the reaction, reaction mixture was diluted with dichloromethane, washed with water, brine, dried over anhydrous Na₂SO₄ and concentrated to yield the crude product. The crude compound was purified by reverse phase prep HPLC to yield 1-((1R,4R)-4-((4-((5-cyclopentyl-1H-pyrazol-3-yl)amino)pyrimidin-2-yl)amino)cyclohexyl)-3-(3-(trifluoromethyl)phenyl)urea (25 mg, 8.9% yield) as a TFA salt. LC purity: 99.19%; m/z: 529.3 [M+H]⁺ (Mol. formula C₂₆H₃₁F₃N₈O, calcd. mol. wt. 528.61). ¹H NMR (400 MHz, CD₃OD): δ 7.86 (s, 1H), 7.69 (d, J=7.16 Hz, 1H), 7.50 (d, J=7.96 Hz, 1H), 7.42 (t, J=7.8 Hz, 1H), 7.25 (d, J=7.64 Hz, 1H), 6.51 (bs, 1H), 6.45 (bs, 1H), 3.93-3.91 (m, 1H), 3.61 (t, J=11.56 Hz, 1H), 3.14-3.12 (m, 1H), 2.16-2.15 (m, 6H), 1.84-1.73 (m, 6H), 1.59-1.54 (m, 2H), 1.44-1.38 (m, 3H).

### Example 3: Synthesis of Compound 40

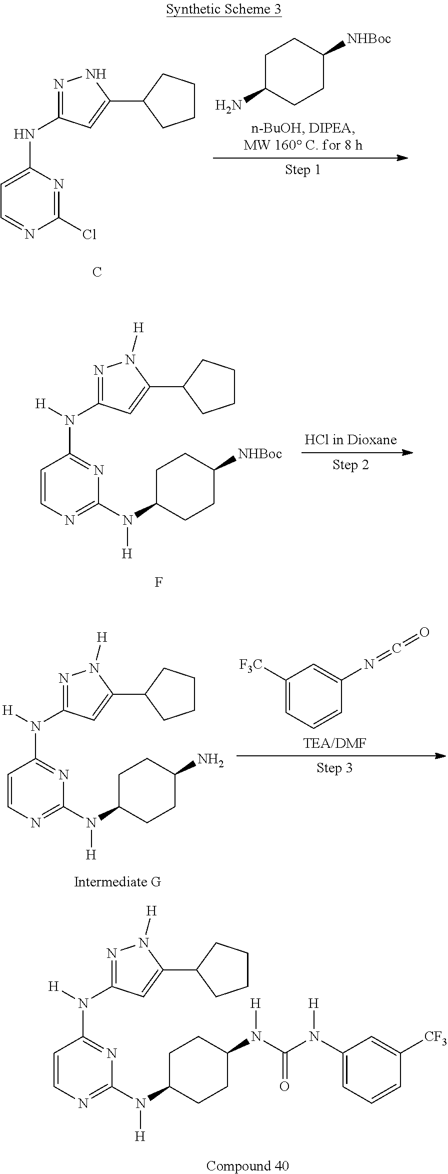

### Step-1: Synthesis of Compound F—tert-butyl((1S,4S)-4-((4-((5-cyclopentyl-1H-pyrazol-3-yl)amino)pyrimidin-2-yl)amino)cyclohexyl)carbamate

To a mixture of compound C—2-chloro-N-(5-cyclopentyl-1H-pyrazol-3-yl)pyrimidin-4-amine (0.2 g, 0.76 mmol) and tert-butyl ((1S,4S)-4-aminocyclohexyl)carbamate (0.152 g, 0.74 mmol) in n-butanol (2 mL) in a 20 mL microwave vial was added DIPEA (0.7 mL, 3.8 mmol). The reaction mixture was subjected to microwave at 160° C. for 8 h. The progress of the reaction was monitored by TLC, and after complete consumption of starting material, the reaction mixture was cooled to room temperature and concentrated to remove n-butanol. The residue obtained was diluted with water and extracted with dichloromethane. The combined organic layer was dried over anhydrous Na₂SO₄ and concentrated under reduced pressure to give compound F—tert-butyl ((1S,4S)-4-((4-((5-cyclopentyl-1H-pyrazol-3-yl)amino)pyrimidin-2-yl)amino)cyclohexyl)carbamate (0.2 g, crude). LC purity: 45.76%; m/z: 442.2 [M+H]⁺ (Mol. formula C₂₃H₃₅N₇O₂, calcd. mol. wt. 441.58).

### Step-2: Synthesis of Intermediate G—N²-((1S,4S)-4-aminocyclohexyl)-N⁴-(5-cyclopentyl-1H-pyrazol-3-yl)pyrimidine-2,4-diamine

To a cooled 0° C. solution of compound F—tert-butyl((1S,4S)-4-((4-((5-cyclopentyl-1H-pyrazol-3-yl)amino)pyrimidin-2-yl)amino)cyclohexyl)carbamate (0.2 g, 0.45 mmol) in dichloromethane (2 mL) was added HCl in dioxane (2 mL, 4M solution). The reaction was allowed to stir at room temperature for 3 h. The reaction mixture was monitored by TLC, and starting material was consumed. The resulting mixture was concentrated to obtain intermediate G—N²-((1S,4S)-4-(aminocyclohexyl)-N⁴-((5-cyclopentyl-1H-pyrazol-3-yl))pyrimidine-2,4-diamine (180 mg, quantitative yield) as the HCl salt. LC purity: 51.85%; m/z: 342.2 [M+H]⁺ (Mol. formula C₁₈H₂₇N₇, calcd. mol. wt. 341.46).

### Step-3: Synthesis of Compound 40—1-((1S,4S)-4-((4-((5-cyclopentyl-1H-pyrazol-3-yl)amino)pyrimidin-2-yl)amino)cyclohexyl)-3-(3-(trifluoromethyl)phenyl)urea

To a stirred solution of intermediate G—N²-((1s,4s)-4-aminocyclohexyl)-N⁴-(5-cyclopentyl-1H-pyrazol-3-yl)pyrimidine-2,4-diamine (0.18 g, 0.527 mmol) in dry DMF (2 mL) was cooled to 0° C. and was added triethylamine (0.268 mL, 2.63 mmol) and 1-isocyanato-3-(trifluoromethyl)benzene (0.098 mL, 0.527 mmol) drop wise. The reaction mixture was stirred at room temperature for 4 h (the reaction mixture was monitored by TLC). After completion of the reaction, reaction mixture was diluted with dichloromethane washed with water, brine, dried over anhydrous Na₂SO₄ and concentrated to yield the crude product. The crude compound was purified by reverse phase prep HPLC to yield compound 40—1-((1S,4S)-4-((4-((5-cyclopentyl-1H-pyrazol-3-yl)amino)pyrimidin-2-yl)amino)cyclohexyl)-3-(3-(trifluoromethyl)phenyl)urea (25 mg, 8.9% yield) as a TFA salt. LC purity: 95.26%; m/z: 529.3 [M+H]⁺ (Mol. formula C₂₆H₃₁F₃N₈O, calcd. mol. wt. 528.26). ¹H NMR (400 MHz, CD₃OD): δ 7.82 (s, 1H), 7.70 (s, 1H), 7.51 (d, J=8.04 Hz, 1H), 7.41 (t, J=7.6 Hz, 1H), 7.24 (d, J=7.04 Hz 1H), 6.50 (bs, 1H), 6.31 (bs, 1H), 4.04-4.02 (m, 1H), 3.84 (s, 1H), 3.15 (d, J=7.76 Hz, 1H), 2.12-2.10 (m, 2H), 1.93-1.71 (s, 14H).

### Example 4: Synthesis of Intermediate H

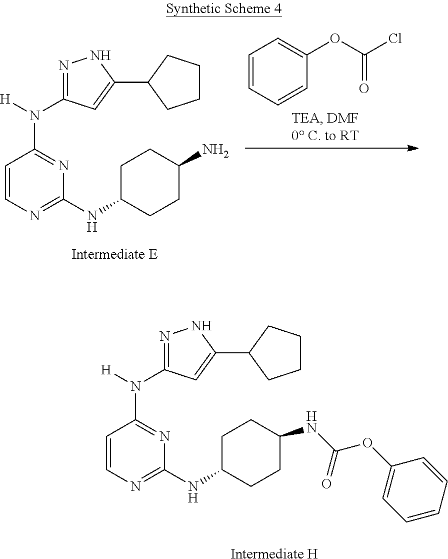

To a stirred solution of intermediate E, N²-((1R,4R)-4-aminocyclohexyl)-N⁴-(5-cyclopentyl-1H-pyrazol-3-yl)pyrimidine-2,4-diamine (0.5 g, 1.4 mmol) in anhydrous DMF (5 mL) was added triethylamine (0.62 mL, 0.439 mmol). The reaction mixture was stirred at 0° C. for 15 min. Then added phenyl chloroformate (0.22 mL, 0.146 mmol) dropwise. The reaction was stirred at 0° C.-10° C. for 1 h. The progress of the reaction was monitored by TLC after complete consumption of starting material, the reaction mixture was diluted with water and extracted with ethyl acetate. The resulting organic layer was washed with brine solution then dried over anhydrous Na₂SO₄ and concentrated to obtain crude which was triturated with pet ether to get intermediate H—phenyl((1R,4R)-4-((4-((5-cyclopentyl-1H-pyrazol-3-yl)amino)pyrimidin-2-yl)amino)cyclohexyl)carbamate (0.3 g, 67.59% yield). LC purity: 19.61%; m/z: 462.3 [M+H]⁺ (Mol. formula C₂₅H₃₁N₇O₂, calcd. mol. wt. 461.57)

### Example 5: Synthesis of Compound 72

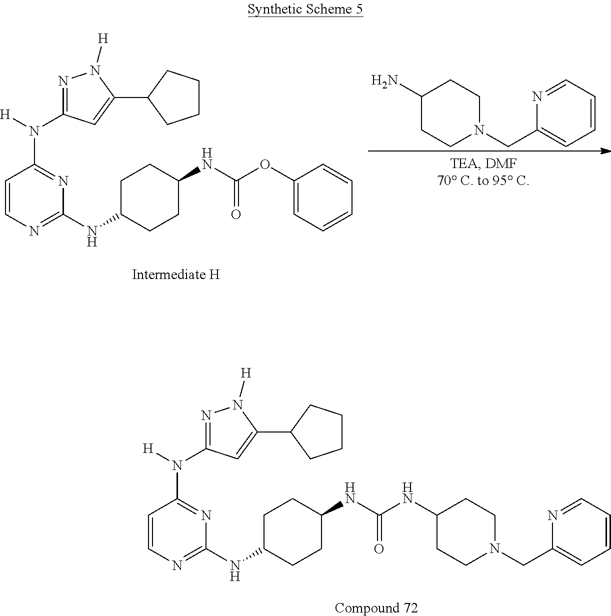

To a stirred solution of intermediate H—phenyl((1R,4R)-4-((4-((5-cyclopentyl-1H-pyrazol-3-yl)amino)pyrimidin-2-yl)amino)cyclohexyl)carbamate (300 mg, 0.651 mmol) in dry DMF (6.0 mL) was added triethylamine (0.27 mL, 1.952 mmol). The reaction mixture was stirred at 70° C., after 2 h stirring reaction mixture was cooled to room temperature and 1-(pyridin-2-ylmethyl)piperidin-4-amine (124 mg, 0.651 mmol) was added and heated to 95° C. for 16 h. After completion of the reaction, reaction mixture was diluted with water extracted with 5% methanol in dichloromethane. The combined organic layers were dried over anhydrous Na₂SO₄ concentrated under reduced pressure to get crude material. The crude material was purified by prep HPLC to obtain compound 67 1-((1R,4R)-4-((4-((5-cyclopentyl-1H-pyrazol-3-yl)amino)pyrimidin-2-yl)amino)cyclohexyl)-3-(1-(pyridin-2-ylmethyl)piperidin-4-yl)urea (80 mg, 22% yield) as a TFA salt. LC purity: 96.17%; m/z: 557.3 [M−H]⁺ (Mol. formula C₃₀H₄₂N₁₀O, calcd. mol. wt. 558.740). ¹H NMR (400 MHz, CD₃OD): δ 8.70 (s, 1H), 7.94-7.90 (m, 1H), 7.769 (d, J=7.2 Hz, 1H), 7.51-7.46 (m, 2H), 6.51-6.29 (m, 2H), 4.49 (s, 2H), 3.84-3.82 (m, 2H), 3.58-3.36 (m, 3H), 3.32-3.25 (m, 2H), 3.18-3.14 (m, 1H), 2.18-2.05 (m, 8H), 1.86-1.73 (m, 8H), 1.82-1.76 (m, 2H), 1.53-1.47 (m, 2H), 1.37-1.30 (m, 2H).

### Example 6: Synthesis of Compound 61

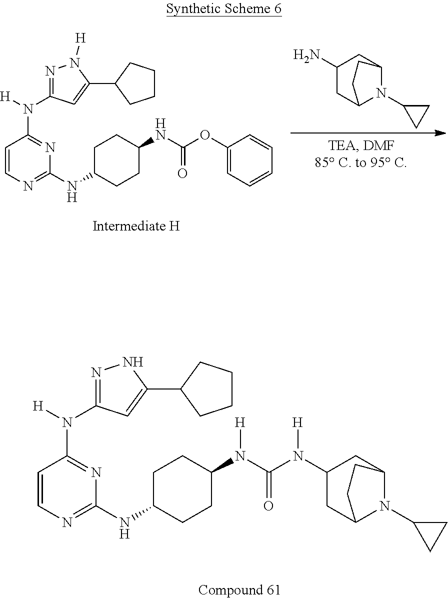

To a stirred solution of phenyl ((1R,4R)-4-((4-((5-cyclopentyl-1H-pyrazol-3-yl)amino)pyrimidin-2-yl)amino)cyclohexyl)carbamate (200 mg, 0.433 mmol) in dry DMF (4 mL) was added triethylamine (0.18 mL, 1.30 mmol). The reaction mixture was heated to 85° C. for 1 h. The reaction mixture was cooled to room temperature and 8-cyclopropyl-8-azabicyclo[3.2.1]octan-3-amine (72 mg, 0.433 mmol) was added. The reaction mixture was heated to 95° C. for 16 h. The progress of the reaction was monitored by TLC, and after complete consumption of starting material, the reaction mixture was diluted with water and extracted with dichloromethane. The resulting organic layer was washed with brine solution then dried over anhydrous Na₂SO₄ and concentrated to obtain crude compound. The crude compound was purified by reverse phase prep HPLC to yield compound 61—1-((1R,4R)-4-((4-((5-cyclopentyl-1H-pyrazol-3-yl)amino)pyrimidin-2-yl)amino)cyclohexyl)-3-(8-cyclopropyl-8-azabicyclo[3.2.1]octan-3-yl)urea (25 mg, 10.8% yield) as a TFA salt. LC purity: 99.94%; m/z: 532.4 [M−H]⁺ (Mol. formula C₂₉H₄₃N₉O, calcd. mol. wt.: 533.73). ¹H NMR (400 MHz, CD₃OD): δ 7.74 (s, 1H), 6.24 (bs, 2H), 4.05-4.01 (m, 1H), 3.99-3.76 (m, 3H), 3.51-3.47 (m, 1H), 3.11-3.07 (t, J=7.2 Hz, 1H), 2.70-2.62 (m, 1H), 2.33-2.25 (m, 3H), 2.09-1.98 (m, 9H), 1.82-1.70 (m, 7H), 1.52-1.32 (m, 4H), 1.06-1.00 (m, 4H).

### Example 7: Synthesis of Compound 11

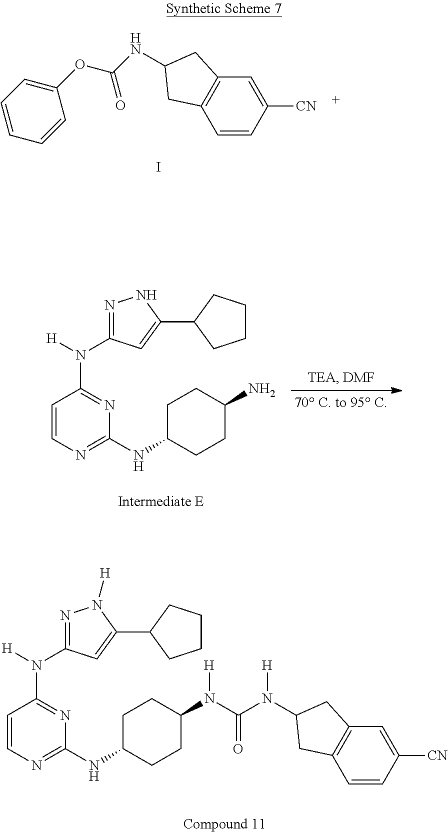

To a stirred solution of compound I—phenyl(5-cyano-2,3-dihydro-1H-inden-2-yl)carbamate (500 mg, 1.466 mmol) in dry DMF (5.0 mL), was added triethylamine (1.01 mL, 7.331 mmol) and the resulting reaction mixture was stirred at 70° C. After 2 h of stirring, the reaction mixture was cooled to room temperature and intermediate E—N²-((1R,4R)-4-aminocyclohexyl)-N⁴-(5-cyclopentyl-1H-pyrazol-3-yl)pyrimidine-2,4-diamine (448 mg, 1.466 mmol) was added and the resulting mixture was heated to 95° C. After 16 h of stirring, the reaction mixture was diluted with water (20 mL) and extracted with 5% methanol in dichloromethane (2×30 mL). The combined organic layers were dried over anhydrous Na₂SO₄, and concentrated under reduced pressure to afford crude material. The crude material was purified by preparative HPLC to afford compound 11—1-(5-cyano-2,3-dihydro-1H-inden-2-yl)-3-((1R,4R)-4-((4-((5-cyclopentyl-1H-pyrazol-3-yl)amino)pyrimidin-2-yl)amino)cyclohexyl)urea (200 mg, 26%) as a TFA salt. LC Purity: 99.8%; m/z: 526.4 [M+H]⁺ (Mol. formula C₂₉H₃₅N₉O, calcd. mol. wt. 525.66). ¹H NMR (400 MHz, DMSO-d6): δ 12.13 (s, 1H), 10.80 (s, 1H), 8.02 (s, 1H), 7.79 (d, J=7.2 Hz, 1H), 7.63 (s, 1H), 7.56 (d, J=7.6 Hz, 1H), 7.41 (d, J=8.8 Hz, 1H), 6.42-6.36 (m, 2H), 6.01 (s, 1H), 5.53 (s, 1H), 4.38-4.37 (m, 1H), 3.80-3.72 (m, 1H), 3.49-3.40 (m, 1H), 3.25-3.17 (m, 2H), 3.09-3.06 (m, 1H), 2.83-2.75 (m, 2H), 2.04-1.93 (m, 6H), 1.73-1.59 (m, 6H), 1.44-1.38 (m, 2H), 1.38-1.29 (m, 2H).

### Example 8: Synthesis of Compound 6

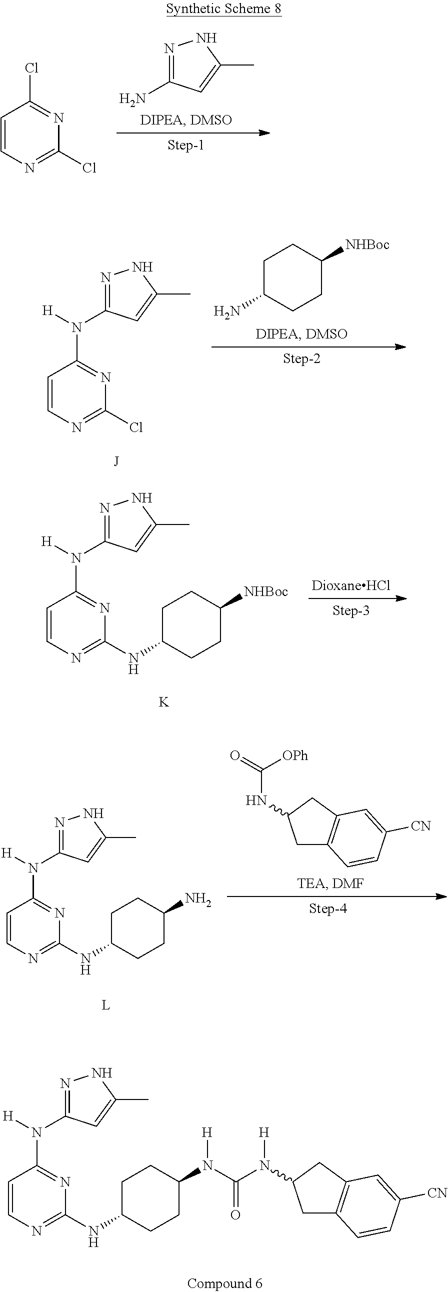

### Step-1: Synthesis of Compound J—2-chloro-N-(5-methyl-1H-pyrazol-3-yl)pyrimidin-4-amine

A mixture of 2,4-dichloropyrimidine (0.5 g, 3.3 mmol), 3-chloro-5-methyl-1H-pyrazole (0.355 g, 3.6 mmol) and DIPEA (0.6 mL, 3.3 mmol) in DMSO (5 mL) were stirred at 60° C. for 16 h. The reaction was monitored by TLC, starting material was consumed. The reaction mixture was cooled to room temperature, added water and solid was precipitated. The solid was filtered, washed with pet ether and dried under vacuum to yield compound J—2-chloro-N-(5-methyl-1H-pyrazol-3-yl)pyrimidin-4-amine (0.596 g, 90% yield). LC purity: 96.69%; m/z: 210.2 [M+H]⁺ (Mol. formula C₈H₈ClN5, calcd. mol. wt. 209.64).

### Step-2: Synthesis of Compound K—tert-butyl ((1R,4R)-4-((4-((5-methyl-1H-pyrazol-3-yl)amino)pyrimidin-2-yl)amino)cyclohexyl)carbamate

A mixture of compound J—2-chloro-N-(5-methyl-1H-pyrazol-3-yl)pyrimidin-4-amine (0.5 g, 2.38 mmol), tert-butyl((1R,4R)-4-aminocyclohexyl)carbamate (0.511 g, 2.39 mmol) and DIPEA (2.14 mL, 11.9 mmol) in DMSO (5 mL) was stirred at 120° C. for 16 h. The reaction was monitored by LCMS, and starting material was consumed. The reaction mixture was cooled to room temperature was added water and extracted with dichloromethane. The organic layer was dried over anhydrous Na₂SO₄ and concentrated to obtain the crude compound. The crude compound was purified by silica gel column chromatography with gradient elution of 50-100% ethyl acetate in pet ether to yield compound K—tert-butyl ((1R,4R)-4-((4-((5-methyl-1H-pyrazol-3-yl)amino)pyrimidin-2-yl)amino)cyclohexyl)carbamate (0.45 g, 48.6% yield). LC purity: 58.18%; m/z: 388.2 [M+H]⁺ (Mol. formula C₁₉H₂₉N₇O₂, calcd. mol. wt. 387.49).

### Step-3: Synthesis of Intermediate L—N²-((1R,4R)-4-aminocyclohexyl)-N⁴-(5-methyl-1H-pyrazol-3-yl) pyrimidine-2,4-diamine

To the mixture of compound K—tert-butyl((1R, 4R)-4-((4-((5-methyl-1H-pyrazol-3-yl)amino)pyrimidin-2-yl)amino)cyclohexyl)carbamate (0.45 g, 1.16 mmol) in dry dichloromethane (2 mL) was added HCl in dioxane (2 mL, 4M solution). The reaction mixture was stirred at room temperature for 3 h. The progress of the reaction was monitored by TLC analysis. The reaction mixture was concentrated to obtain pure intermediate L—N²-((1R,4R)-4-aminocyclohexyl)-N⁴-(5-methyl-1H-pyrazol-3-yl)pyrimidine-2,4-diamine (0.3 g, quantitative yield). LC purity: 70.67%; m/z: 288.2 [M+H]⁺ (Mol. formula C₁₄H₂₁N₇, calcd. mol. wt. 287.37).

### Step-4: Synthesis of Compound 6—N²-((1R,4R)-4-aminocyclohexyl)-N⁴-(5-methyl-1H-pyrazol-3-yl) pyrimidine-2,4-diamine

To the mixture of compound K tert-butyl((1R, 4R)-4-((4-((5-methyl-1H-pyrazol-3-yl)amino)pyrimidin-2-yl)amino)cyclohexyl)carbamate (0.45 g, 1.16 mmol) in dry dichloromethane (2 mL) was added HCl in dioxane (2 mL, 4M solution). The reaction mixture was stirred at room temperature for 3 h. The progress of the reaction was monitored by TLC analysis. The reaction mixture was concentrated to obtain pure compound 6—N²-((1R,4R)-4-aminocyclohexyl)-N⁴-(5-methyl-1H-pyrazol-3-yl)pyrimidine-2,4-diamine (0.3 g, quantitative yield). LC purity: 70.67%; m/z: 288.2 [M+H]⁺ (Mol. formula C₁₄H₂₁N₇, calcd. mol. wt. 287.37).

### Example 9: Synthesis of Compound 20

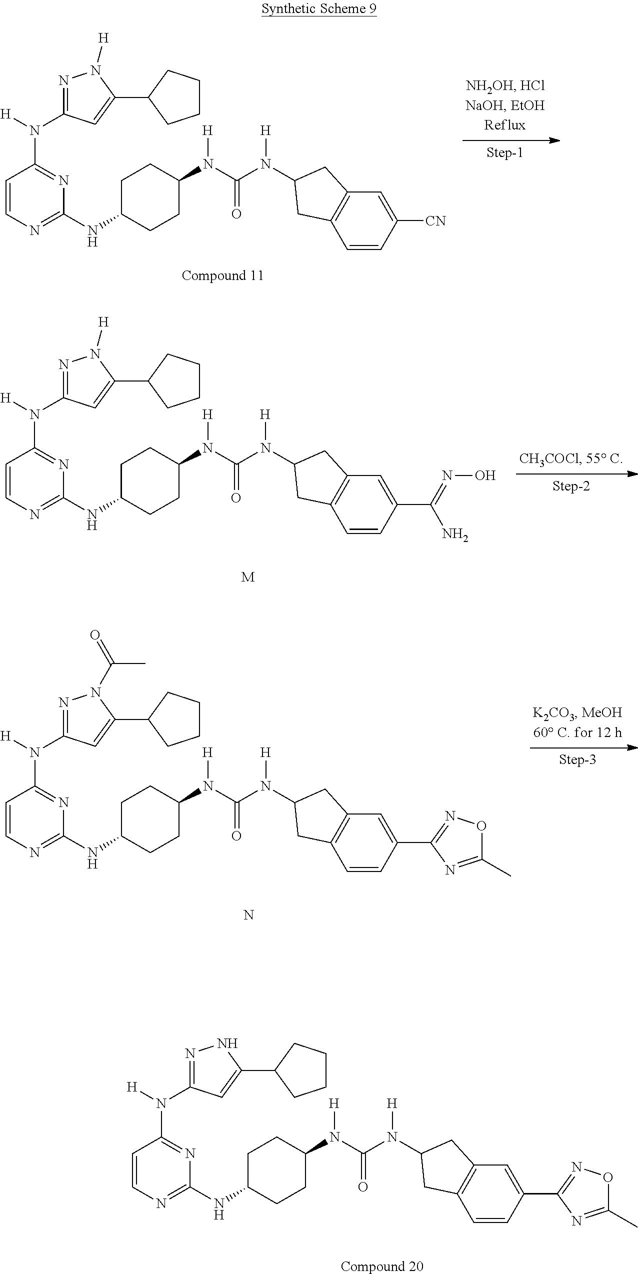

### Step-1: Synthesis of Compound L—(Z)-2-(3-((1R,4R)-4-((4-((5-cyclopentyl-1H-pyrazol-3-yl)amino)pyrimidin-2-yl)amino)cyclohexyl)ureido)-N′-hydroxy-2,3-dihydro-1H-indene-5-carboximidamide

To a stirred solution of compound 11 1-(5-cyano-2,3-dihydro-1H-inden-2-yl)-3-((1R,4R)-4-((4-((5-cyclopentyl-1H-pyrazol-3-yl)amino)pyrimidin-2-yl)amino)cyclohexyl)urea (150 mg, 0.285 mmol) in ethanol (3 mL) was added hydroxyl amine hydrochloride (0.15 mL, 2.198 mmol) at 0° C. The reaction was refluxed for 16 h. The progress of the reaction was monitored by TLC after complete consumption of starting material, the solvent was evaporated under reduced pressure and water was added to the residue. The mixture was extracted with dichloromethane and the organic extract was evaporated under reduced pressure. The residue was purified by column chromatography Biotage isolera 230-400 silica gel with gradient elution of 0-85% ethyl acetate in pet ether to yield compound L—(Z)-2-(3-((1R,4R)-4-((4-((5-cyclopentyl-1H-pyrazol-3-yl)amino)pyrimidin-2-yl)amino)cyclohexyl)ureido)-N′-hydroxy-2,3-dihydro-1H-indene-5-carboximidamide (130 mg, 81%). LC purity: 65.18%; m/z: 559.3 [M+H]⁺ (Mol. formula C₂₉H₃₈N₁₀O₂, calcd. mol. wt. 558.69).

### Step-2: Synthesis of Compound K— 1-((1R,4R)-4-((4-((1-acetyl-5-cyclopentyl-1H-pyrazol-3-yl)amino)pyrimidin-2-yl)amino)cyclohexyl)-3-(5-(5-methyl-1,2,4-oxadiazol-3-yl)-2,3-dihydro-1H-inden-2-yl)urea

A mixture of compound L—(Z)-2-(3-((1R,4R)-4-((4-((5-cyclopentyl-1H-pyrazol-3-yl)amino)pyrimidin-2-yl)amino) cyclohexyl)ureido)-N′-hydroxy-2,3-dihydro-1H-indene-5-carboximidamide (130 mg, 1.465 mmol) and acetyl chloride (1.3 mL, 10 vol) was heated at 55° C. for 16 h. The progress of the reaction was monitored by TLC after complete consumption of starting material, the reaction mixture was diluted with water, extracted with dichloromethane. The resulting organic layer was washed with brine solution then dried over anhydrous Na₂SO₄ and concentrated to obtain crude compound M—1-((1R,4R)-4-((4-((1-acetyl-5-cyclopentyl-1H-pyrazol-3-yl)amino)pyrimidin-2-yl)amino) cyclohexyl)-3-(5-(5-methyl-1,2,4-oxadiazol-3-yl)-2,3-dihydro-1H-inden-2-yl)urea (120 mg, crude). LC purity: 15.2%; m/z: 526.3 [M-100]⁺ (Mol. formula C₃₃H₄₀N₁₀O₃, calcd. mol. wt. 624.75).

### Step-3: Synthesis of Compound 20—1-((1S,4S)-4-((4-((5-cyclopentyl-1H-pyrazol-3-yl)amino)pyrimidin-2-yl)amino)cyclohexyl)-3-(3-(trifluoromethyl)phenyl)urea

To a solution of compound M—1-((1R,4R)-4-((4-((1-acetyl-5-cyclopentyl-1H-pyrazol-3-yl)amino)pyrimidin-2-yl)amino)cyclohexyl)-3-(5-(5-methyl-1,2,4-oxadiazol-3-yl)-2,3-dihydro-1H-inden-2-yl)urea (120 mg, 0.192 mmol) in methanol (5 mL) was added potassium carbonate (80 mg, 0.576 mmol) at room temperature. The reaction was heated to 60° C. for 12 h. The progress of the reaction was monitored by TLC after complete consumption of starting material, the reaction mixture was concentrated, added water and extracted with dichloromethane. The combined organic layers were washed with brine, dried over anhydrous Na₂SO₄ and concentrated. The crude compound was purified by reverse phase prep HPLC to yield compound 20—1-((1R,4R)-4-((4-((5-cyclopentyl-1H-pyrazol-3-yl)amino)pyrimidin-2-yl)amino)cyclohexyl)-3-(5-(5-methyl-1,2,4-oxadiazol-3-yl)-2,3-dihydro-1H-inden-2-yl)urea (18 mg, 16%) as a TFA salt. LC purity: 98.95%; m/z: 583.4 [M+H]⁺ (Mol. formula C₃₁H₃₈N₁₀O₂, calcd. mol. wt. 582.71). ¹H NMR (400 MHz, CD₃OD): δ 7.87 (s, 1H), 7.84 (d, J=8 Hz, 1H), 7.68 (d, J=6.4 Hz, 1H), 7.35 (d, J=7.6 Hz, 1H), 6.51 (s, 1H), 6.28 (s, 1H), 4.54-4.51 (m, 1H), 3.88-3.82 (m, 1H), 3.58-3.53 (m, 1H), 3.14-3.10 (m, 1H), 2.87-2.82 (m, 2H), 2.63 (s, 3H), 2.27-2.05 (m, 6H), 1.81-1.80 (m, 2H), 1.75-1.71 (m, 4H), 1.55-1.46 (m, 2H), 1.34-1.25 (m, 5H).

### Example 10: Synthesis of Intermediate R

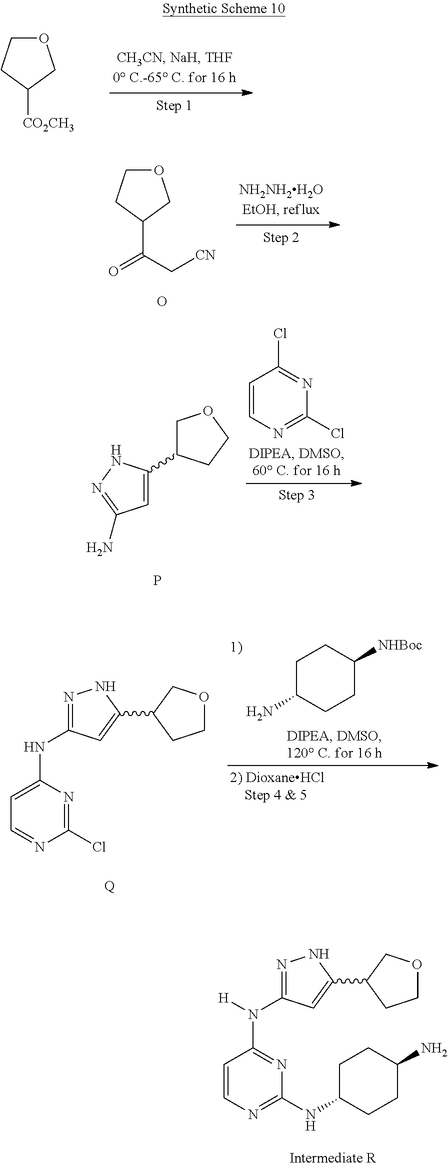

### Step-1: Synthesis of Compound O—3-oxo-3-(tetrahydrofuran-3-yl)propanenitrile

To a stirred suspension of sodium hydride (3.07 g, 76.9 mmol) in dry THE was heated to 65° C. To this added a mixture of methyl tetrahydrofuran-3-carboxylate (5 g, 38.4 mmol) and acetonitrile (4.1 mL, 76.92 mmol) dropwise over the period of 45 min. The resulting pale-yellow suspension was heated at 65° C. for further 15 h (monitored by TLC). After complete consumption of the starting material, reaction mixture was cooled to room temperature. The reaction mixture was poured into ice cold water and the resulting solution was extracted with petroleum ether. The aqueous layer was separated and acidified to pH-2 with 6N HCl solution and extracted with ethyl acetate. The organic layer was dried over anhydrous Na₂SO₄ and concentrated under reduced pressure to get compound O—3-oxo-3-(tetrahydrofuran-3-yl)propanenitrile (4.3 g, 82.07% N yield), which was taken to the next step without further purification.

### Step-2: Synthesis of Compound P—5-(tetrahydrofuran-3-yl)-1H-pyrazol-3-amine

To a stirred solution of compound O—3-oxo-3-(tetrahydrofuran-3-yl)propanenitrile (4.3 g, 31.4 mmol) in 2-propanol (40 mL) was added hydrazine monohydrate (4.6 mL, 94.3 mmol). The reaction was heated at 80° C. for 10 h. After completion of the reaction as monitored by TLC., the reaction mixture was cooled to ambient temperature and solvent was removed in vacuum. The residue was purified by Biotage isolara using silica gel (230-400) with gradient elution of 0-10% methanol in dichloromethane to obtain compound P—5-(tetrahydrofuran-3-yl)-1H-pyrazol-3amine (3.1 g, 65.9% yield). LC purity: 90.67%; m/z: 154.2 [M+H]⁺ (Mol. formula C₇H₁₁N₃O calcd. mol. wt. 153.09).

### Step-3: Synthesis of Compound Q—2-chloro-N-(5-(tetrahydrofuran-3-yl)-1H-pyrazol-3-yl)pyrimidine-4-amine

To a solution of compound P—5-(tetrahydrofuran-3-yl)-1H-pyrazol-3-amine (3.1 g, 20.2 mmol) in DMSO (25 mL) was added 2,4-dichloropyrimidine (3.01 g, 20.2 mmol) and DIPEA (5.2 mL, 30.3 mmol). The reaction was heated at 60° C. for 16 h. Completion of the reaction was monitored by TLC. The reaction mixture was diluted with cold water and extracted with ethyl acetate. The organic layer was dried over anhydrous Na₂SO₄, filtered and concentrated under reduced pressure. The crude was purified by Biotage isolara using silica gel (230-400) with gradient elution of 0-10% methanol in dichloromethane to get compound Q—2-chloro-N-(5-(tetrahydrofuran-3-yl)-1H-pyrazol-3-yl)pyrimidine-4-amine (2.9 g, 54.7% yield). LC purity: 94.8%; m/z: 266.2 [M+H]⁺ (Mol. Formula C₁₁H₁₂ClN₅O calcd. mol. wt. 265.70).

### Steps 4 and 5: Synthesis of Intermediate R—N²-((1R,4R)-4-aminocyclohexyl)-N⁴-(5-(tetrahydrofuran-3-yl)-1H-pyrazol-3-yl)pyrimidine-2,4-diamine

To a solution of compound Q—2-chloro-N-(5-(tetrahydrofuran-3-yl)-1H-pyrazol-3-yl)pyrimidine-4-amine (2.78 g, 10 mmol) in DMSO (25 mL) was added tert-butyl((1R,4R)-4-aminocyclohexyl) carbamate (2.91 g, 13.6 mmol) and DIPEA (5.48 mL, 31.47 mmol). The reaction was heated at 140° C. for 16 h. Completion of the reaction was monitored by TLC. The reaction mixture was diluted with water and extracted with ethyl acetate. The organic layer was dried over anhydrous Na₂SO₄, filtered and concentrated under reduced pressure. The crude was purified by Biotage isolara using silica gel (230-400) with gradient elution of 0-20% methanol in dichloromethane to obtain tert-butyl((1R,4R)-4-((4-((5-tetrahydrofuran-3-yl)-1H-pyrazol-3-yl)amino)cyclohexyl)carbamate (3.5 g, 76.5% yield). LC purity: 84.86%; m/z: 444.3[M+H]⁺ (Mol. Formula C₂₂H₃₃N₇O₃ calcd, mol. wt. 443.58).

To a solution of tert-butyl((1R,4R)-4-((4-((5-(tetrahydrofuran-3-yl)-1H-pyrazol-3-yl)amino)pyrimidine-2-yl)amino)cyclohexyl)carbamate (3.5 g, 7.9 mmol) in dichloromethane (30 mL) was added 30 mL of 4M HCl in dioxane at 0° C. The reaction was stirred at room temperature for 4 h. After complete consumption of the starting material (monitored by TLC), the reaction mixture was concentrated under reduced pressure to get intermediate R—N²-((1R,4R)-4-aminocyclohexyl)-N⁴-(5-(tetrahydrofuran-3-yl)-1H-pyrazol-3-yl)pyrimidine-2,4-diamine (2.6 g, 96% yield). This was taken to the next step without further purification. LC purity: 81%; m/z: 344.3 [M+H]⁺ (Mol. C₁₇H₂₅N₇O calcd, mol. wt. 343.44).

### Example 11: Synthesis of Compound 102

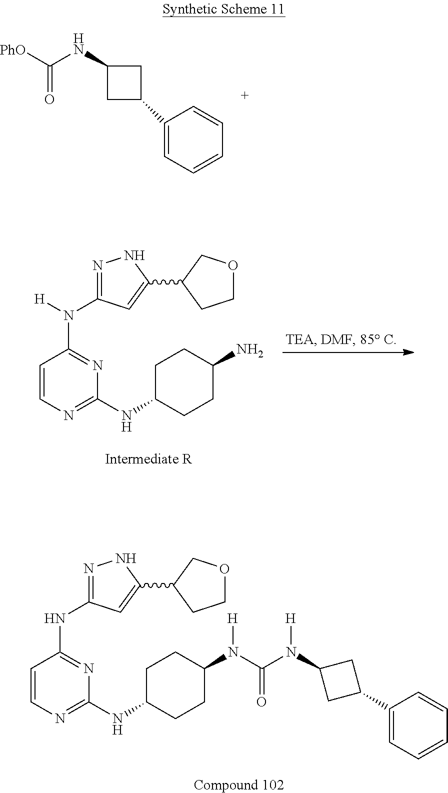 [SMILES: O=C(N[C@H]1C[C@H](c2ccccc2)C1)Oc1ccccc1 ; N[C@H]1CC[C@H](Nc2nccc(Nc3cc(C4CCOC4)[nH]n3)n2)CC1 ; "Intermediate R" ; O=C(N[C@H]1CC[C@H](Nc2nccc(Nc3cc(C4CCOC4)[nH]n3)n2)CC1)N[C@H]1C[C@H](c2ccccc2)C1 ; "Compound 102"]

### Synthesis of Compound 102—1-((1R,3R)-3-phenylcyclobutyl)-3-((1,4R)-4-((4-((5-(tetrahydrofuran-3-yl)1H-pyrazol-3-yl)amino)pyrimidine-2-yl)amino)cyclohexyl)urea

To a solution of phenyl((1R,3R)-3-phenyl)cyclobutylcarbamate (0.155 g, 0.58 mmol) in N,N-dimethylformamide (5 mL) was added triethylamine (0.24 mL, 1.74 mmol) and heated at 65° C. for 1 h. After 1 h, the reaction mixture was cooled to room temperature followed by addition of N²-((1R,4R)-4-aminocyclohexyl)-N⁴-(5-(tetrahydrofuran-3-yl)-1H-pyrazol-3-yl)pyrimidine-2,4-diamine (0.200 g, 0.58 mmol) and the reaction was heated at 85° C. for 16 h. The reaction mixture was cooled to room temperature was added water and extracted with dichloromethane. The organic layer was separated and dried over anhydrous Na₂SO₄, filtered and concentrated under reduced pressure. The residue was purified by reverse phase prep HPLC to yield 1-((1R,3R)-3-phenylcyclobutyl)-3-((1,4R)-4-((4-((5-(tetrahydrofuran-3-yl)1H-pyrazol-3-yl)amino)pyrimidine-2-yl)amino)cyclohexyl)urea (60 mg, 20% yield) as a TFA salt. LC purity: 98.91%; m/z: 517.3 [M+H]⁺ (Mol. C₂₈H₃₆N₈O₂ calcd. mol. wt. 516.65). ¹H NMR (400 MHz, CD₃OD): δ 7.69 (d, J=7.2 Hz, 1H), 7.31-7.30 (m, 3H), 7.19-7.16 (m, 1H), 6.65 (s, 1H), 6.29 (d, J=6.8 Hz, 1H), 4.30 (t, J=6.8 Hz, 1H), 4.06-3.90 (m, 5H), 3.57-3.52 (m, 3H), 2.51-2.33 (m, 4H), 2.11-2.04 (m, 5H), 1.52-1.30 (m, 4H).

### Example 12: Chiral Separation of (R)- and (S)-enantiomers of Compound 11

The individual enantiomers of compound 11 were separated by chiral SFC using
Column: Chiral Pak OX-H
Flowrate: 5 ml/min
Co-Solvent: 50%
Co-Solvent: Methanol
Injected Volume: 15 μL
Outlet Pressure: 100 bar
Temperature: 35° C.
   The first eluted (RT=3.03) fractions and the second eluted (RT=4.6) fractions were both collected and concentrated under reduced pressure.

First eluting isomer: LC Purity: 98.97%; m/z: 526.3 [M+H]⁺ (Mol. formula C₂₉H₃₅N₉O, calcd. mol. wt. 525.66). ¹H NMR (400 MHz, CD₃OD): δ 7.69 (d, J=7.2 Hz, 1H), 7.59 (s, 1H), 7.54 (d, J=8 Hz, 1H), 7.41 (d, J=8 Hz, 1H), 6.55-6.29 (m, 2H), 4.54-4.51 (m, 1H), 3.95-3.80 (m, 1H), 3.56-3.49 (m, 1H), 3.36-3.33 (m, 2H), 3.15-3.11 (m, 1H), 2.90-2.83 (m, 2H), 2.13-2.06 (m, 6H), 1.83-1.71 (m, 6H), 1.56-1.48 (m, 2H), 1.35-1.26 (m, 2H).

Second eluting isomer: LC Purity: 98.35%; m/z: 526.3 [M+H]⁺ (Mol. formula C₂₉H₃₅N₉O, calcd. mol. wt. 525.66). ¹H NMR (400 MHz, CD₃OD): δ 7.69 (d, J=7.2 Hz, 1H), 7.59 (s, 1H), 7.54 (d, J=8 Hz, 1H), 7.41 (d, J=7.6 Hz, 1H), 6.55-6.29 (m, 2H), 4.55-4.49 (m, 1H), 3.90-3.87 (m, 1H), 3.56-3.50 (m, 1H), 3.32-3.30 (m, 2H), 3.15-3.11 (m, 1H), 2.90-2.83 (m, 2H), 2.13-2.06 (m, 6H), 1.83-1.71 (m, 6H), 1.53-1.48 (m, 2H), 1.35-1.26 (m, 2H).

### Example 13: Synthesis of Compound 12

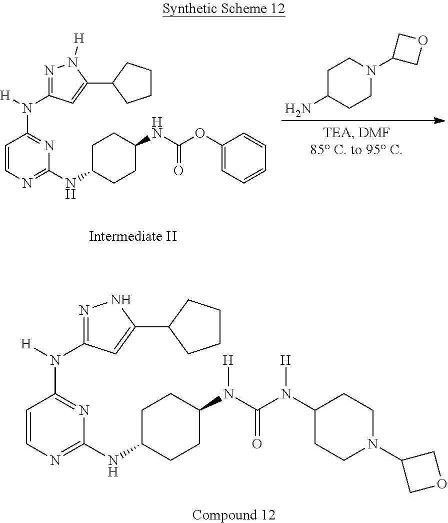 [SMILES: O=C(N[C@H]1CC[C@H](Nc2nccc(Nc3cc(C4CCCC4)[nH]n3)n2)CC1)Oc1ccccc1 ; NC1CCN(C2COC2)CC1 ; "Intermediate H" ; O=C(NC1CCN(C2COC2)CC1)N[C@H]1CC[C@H](Nc2nccc(Nc3cc(C4CCCC4)[nH]n3)n2)CC1 ; "Compound 12"]

To a stirred solution of intermediate H—phenyl ((1R,4R)-4-((4-((5-cyclopentyl-1H-pyrazol-3-yl)amino)pyrimidin-2-yl)amino)cyclohexyl)carbamate (200 mg, 0.433 mmol) in anhydrous DMF (4 mL) was added triethylamine (0.18 mL, 1.30 mmol). The reaction mixture was heated to 85° C. for 1 h. The reaction mixture was cooled to room temperature and 1-(oxetan-3-yl)piperidin-4-amine (67 mg, 0.433 mmol) was added. The reaction was heated to 95° C. for 16 h. The reaction was monitored by TLC, and after complete consumption of starting material, the reaction mixture was diluted with water and extracted with dichloromethane. The resulting organic layer was washed with brine solution then dried over anhydrous Na₂SO₄ and concentrated to obtain the crude compound. The crude compound was purified by reverse phase prep HPLC to yield compound 12 1-((1R,4R)-4-((4-((5-cyclopentyl-1H-pyrazol-3-yl)amino)pyrimidin-2-yl)amino) cyclohexyl)-3-(1-(oxetan-3-yl)piperidin-4-yl)urea (25 mg, 11.06% yield) as a TFA salt. LC purity: 99.85%; m/z: 524.3 [M+H]⁺ (Mol. formula C₂₇H₄₁N₉O₂, calcd. mol. wt. 523.69). ¹H NMR (400 MHz, CD₃OD): δ 7.69 (d, J=7.2 Hz, 1H), 6.52 (bs, 1H), 6.31 (bs, 1H), 4.89-4.80 (m, 4H), 4.39-4.36 (m, 1H), 3.90-3.78 (m, 2H), 3.52-3.49 (m, 3H), 3.14-3.12 (m, 1H), 2.97-2.94 (m, 2H), 2.13-2.04 (m, 8H), 1.83-1.72 (m, 8H), 1.52-1.49 (m, 2H), 1.34-1.31 (m, 2H).

### Example 14: Synthesis of Compound 70

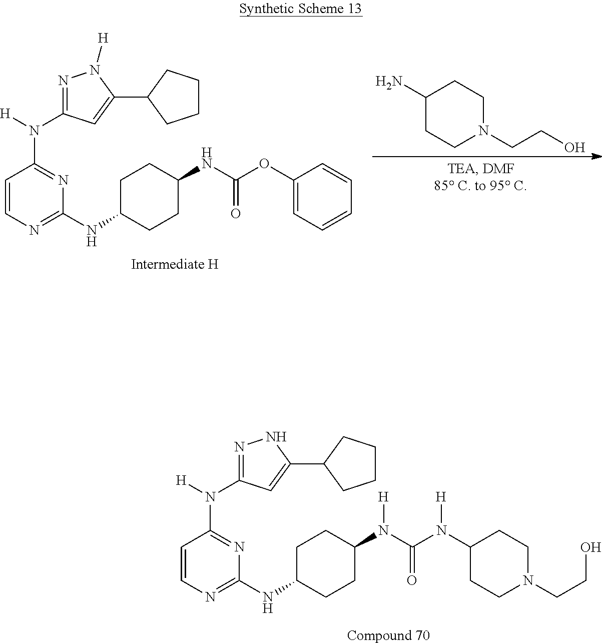

To a stirred solution of Intermediate H—phenyl ((1R,4R)-4-((4-((5-cyclopentyl-1H-pyrazol-3-yl)amino)pyrimidin-2-yl)amino)cyclohexyl)carbamate (200 mg, 0.433 mmol) in anhydrous DMF (4 mL) was added triethylamine (0.18 mL, 1.30 mmol). The reaction mixture was heated to 85° C. for 1 h. The reaction mixture was cooled to room temperature, and 2-(4-aminopiperidin-1-yl)ethan-1-ol (62 mg, 0.433 mmol) was added. The reaction was heated to 95° C. for 16 h. The reaction mixture was diluted with water and extracted with dichloromethane. The resulting organic layer was washed with brine solution then dried over anhydrous Na₂SO₄ and concentrated to obtain crude compound. The crude compound was purified by reverse phase prep HPLC to yield compound 70 1-((1R,4R)-4-((4-((5-cyclopentyl-1H-pyrazol-3-yl)amino)pyrimidin-2-yl)amino)cyclohexyl)-3-(1-(2-hydroxyethyl)piperidin-4-yl)urea (25 mg, 11.3% yield) as a TFA salt. LC purity: 97.93%; m/z: 512.4 [M+H]⁺ (Mol. formula C₂₆H₄₁N₉O₂, calcd. mol. wt. 511.68). ¹H NMR (400 MHz, CD₃OD): δ 7.71 (d, J=6.0 Hz, 1H), 6.55 (bs, 1H), 6.30 (bs, 1H), 3.89 (t, J=5.2 Hz, 2H), 3.70-3.68 (m, 1H), 3.68-3.65 (m, 3H), 3.65-3.58 (m, 2H), 3.50-3.56 (m, 1H), 3.28-3.26 (m, 2H), 3.15-3.13 (m, 2H), 2.17-2.04 (m, 7H), 1.84-1.72 (m, 7H), 1.53-1.50 (d, J=12 Hz, 2H), 1.36-1.28 (m, 3H).

### Example 15: Synthesis of Compound 35

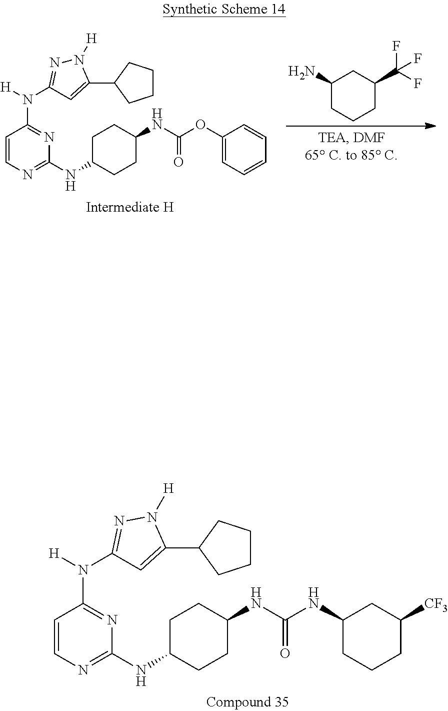

To a stirred solution of intermediate H—phenyl ((1R,4R)-4-((4-((5-cyclopentyl-1H-pyrazol-3-yl)amino)pyrimidin-2-yl)amino)cyclohexyl)carbamate (200 mg, 0.433 mmol) in dry DMF (2 mL) was added triethylamine (0.2 mL, 1.301 mmol). The reaction mixture was heated to 65° C. for 1 h. The reaction mixture was cooled to room temperature and trans-3-(trifluoromethyl)cyclohexan-1-amine (90 mg, 0.433 mmol) was added. The reaction was heated to 85° C. for 16 h. The progress of the reaction was monitored by TLC, and after complete consumption of starting material, the reaction mixture was diluted with water and extracted with dichloromethane. The resulting organic layer was washed with brine solution then dried over anhydrous Na₂SO₄ and concentrated to obtain crude compound. The crude compound was purified by reverse phase prep HPLC to yield compound 35 1-((1R,4R)-4-((4-((5-cyclopentyl-1H-pyrazol-3-yl)amino)pyrimidin-2-yl)amino)cyclohexyl)-3-((1R,3S/1S,3R)-3-(trifluoromethyl)cyclohexyl)urea (70 mg, 35% yield) as a TFA salt. LC purity: 99.54%; m/z: 535.4 [M+H]⁺ (Mol. formula C₂₆H₃₇F₃N₈O, calcd. mol. wt. 534.63). ¹H NMR (400 MHz, CD₃OD): δ 7.65-7.64 (m, 1H), 6.52 (s, 1H), 6.26 (s, 1H), 3.87 (s, 1H), 3.49 (s, 2H), 3.11 (s, 1H), 2.23 (d, J=8.40 Hz, 8H), 1.80-1.69 (m, 12H), 1.19-1.16 (m, 5H).

### Example 16: Synthesis of Compound 101

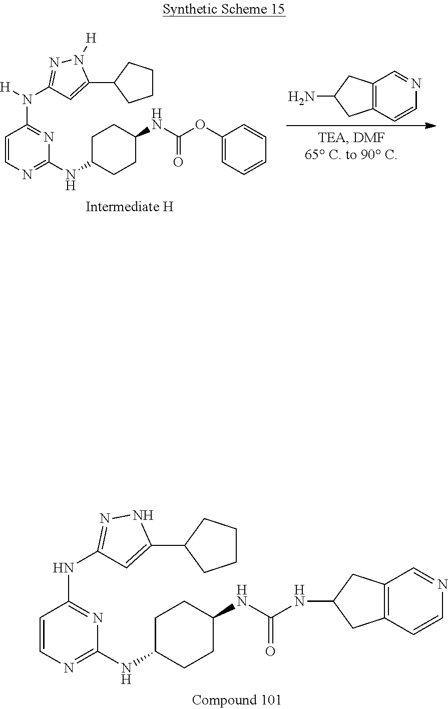

To a stirred solution of intermediate H—phenyl ((1R,4R)-4-((4-((5-cyclopentyl-1H-pyrazol-3-yl)amino)pyrimidin-2-yl)amino)cyclohexyl)carbamate (120 mg, 0.260 mmol) in dry DMF (2 mL) was added triethylamine (0.1 mL, 0.780 mmol). The reaction was heated to 65° C. for 1 h. The reaction mixture was cooled to room temperature and 6,7-dihydro-5H-cyclopenta[c]pyridin-6-amine (70 mg, 0.520 mmol) was added. The reaction mixture was heated to 90° C. for 18 h. The reaction mixture was diluted with water and extracted with dichloromethane. The resulting organic layer was washed with brine solution then dried over anhydrous Na₂SO₄ and concentrated to obtain crude compound. The crude compound was purified by reverse phase prep HPLC to yield Compound 101 1-((1R,4R)-4-((4-((5-cyclopentyl-1H-pyrazol-3-yl)amino)pyrimidin-2-yl)amino)cyclohexyl)-3-(6,7-dihydro-5H-cyclopenta[c]pyridin-6-yl)urea (25 mg, 20% yield) as a TFA salt. LC purity: 97.75%; m/z: 502.3 [M+H]⁺ (Mol. formula C₂₇H₃₅N₉O, calcd. mol. wt. 501.64). ¹H NMR (400 MHz, CD₃OD): δ 8.69 (s, 1H), 8.63 (d, J=6.0 Hz, 1H), 7.97 (d, J=6.0 Hz, 1H), 7.69 (d, J=7.2 Hz, 1H), 6.55 (bs, 1H), 6.29 (d, J=6.8 Hz, 1H), 4.71-4.64 (m, 1H), 3.91-3.87 (m, 1H), 3.62-3.49 (m, 3H), 3.22-3.08 (m, 3H), 2.13-2.05 (m, 6H), 1.83-1.72 (m, 6H), 1.53-1.48 (m, 2H), 1.37-1.29 (m, 2H).

### Example 17: Synthesis of Compound 27

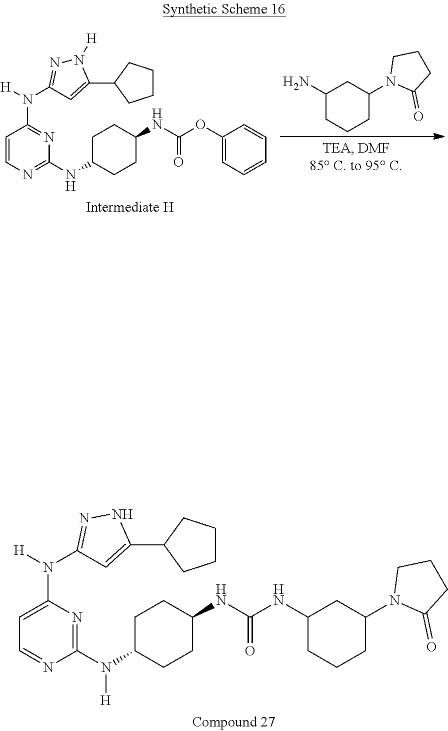

To a stirred solution of Intermediate H—phenyl ((1R,4R)-4-((4-((5-cyclopentyl-1H-pyrazol-3-yl)amino)pyrimidin-2-yl)amino)cyclohexyl)carbamate (100 mg, 0.217 mmol) in dry DMF (2 mL) was added triethylamine (0.1 mL, 0.651 mmol). The reaction was heated to 85° C. for 1 h. The reaction mixture was cooled to room temperature and added 1-(3-aminocyclohexyl)pyrrolidin-2-one (43 mg, 0.239 mmol). The reaction mixture was heated to 95° C. for 16 h. The progress of the reaction was monitored by TLC after complete consumption of starting material, the reaction mixture was diluted with water and extracted with dichloromethane. The resulting organic layer was washed with brine solution then dried over anhydrous Na₂SO₄ and concentrated to obtain the crude compound. The crude compound was purified by reverse phase prep HPLC to yield Compound 27 1-((1R,4R)-4-((4-((5-cyclopentyl-1H-pyrazol-3-yl)amino)pyrimidin-2-yl)amino)cyclohexyl)-3-(3-(2-oxopyrrolidin-1-yl)cyclohexyl)urea (15 mg, 5% yield) as a TFA salt. LC purity: 95.14%; m/z: 550.5 [M+H]⁺ (Mol. formula C₂₉H₄₃N₉O₂, calcd. mol. wt. 549.72). ¹H NMR (400 MHz, CD₃OD): δ 7.82 (s, 1H), 6.52 (bs, 2H), 3.93-3.91 (m, 1H), 3.95-3.90 (m, 1H), 3.58-3.42 (m, 4H), 3.13 (t, J=7.4 Hz, 1H), 2.36 (t, J=7.6 Hz, 2H), 2.13-1.98 (m, 8H), 1.95-1.93 (m, 2H), 1.82 (t, J=5.6 Hz, 3H), 1.71-1.68 (m, 6H), 1.54-1.26 (m, 7H).

### Example 18: Synthesis of Compound 38

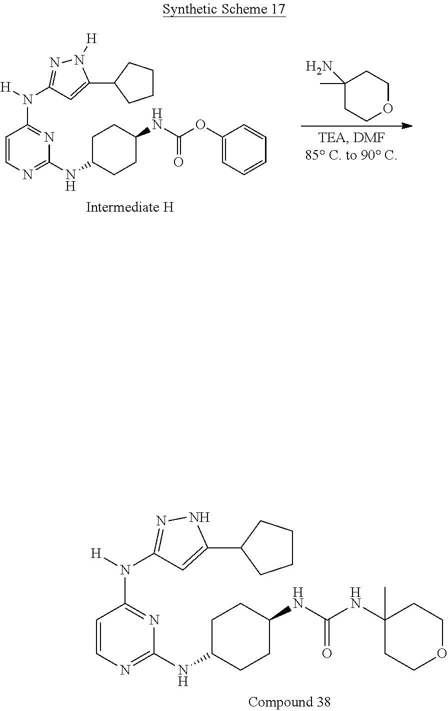

To a stirred solution of intermediate H—phenyl ((1R,4R)-4-((4-((5-cyclopentyl-1H-pyrazol-3-yl)amino)pyrimidin-2-yl)amino)cyclohexyl)carbamate (200 mg, 0.432 mmol) in dry DMF (2 mL) was added triethylamine (0.2 mL, 1.29 mmol). The reaction mixture was heated to 85° C. for 1 h. The reaction mixture was cooled to room temperature and 4-methyltetrahydro-2H-pyran-4-amine (55 mg, 0.432 mmol) was added. The reaction mixture was heated to 90° C. for 16 h. The reaction mixture was monitored by LCMS. The reaction mixture was diluted with water and extracted with dichloromethane. The resulting organic layer was washed with brine solution then dried over anhydrous Na₂SO₄ and concentrated to obtain crude compound. The crude compound was purified by reverse phase prep HPLC to yield Compound 38 1-((1R,4R)-4-((4-((5-cyclopentyl-1H-pyrazol-3-yl)amino)pyrimidin-2-yl)amino)cyclohexyl)-3-(4-methyltetrahydro-2H-pyran-4-yl)urea (40 mg, 19.2% yield) as a TFA salt. LC purity: 99.13%; m/z: 483.4 [M+H]⁺ (Mol. formula C₂₅H₃₈N₈O₂, calcd. mol. wt. 482.63). ¹H NMR (400 MHz, CD₃OD): δ 7.69 (d, J=8.3 Hz, 1H), 6.57 (s, 1H), 6.29 (d, J=7.2 Hz, 1H), 3.91-2.89 (m, 1H), 3.72-3.63 (m, 5H), 3.57 (t, J=8.21 Hz, 2H), 3.47 (t, J=8.03 Hz, 1H), 3.15-3.12 (m, 1H), 2.09-2.00 (m, 7H), 1.86-1.59 (m, 6H), 1.56-1.50 (m, 2H), 1.36 (s, 3H), 1.36-1.32 (s, 2H).

### Example 19: Synthesis of Compound 57

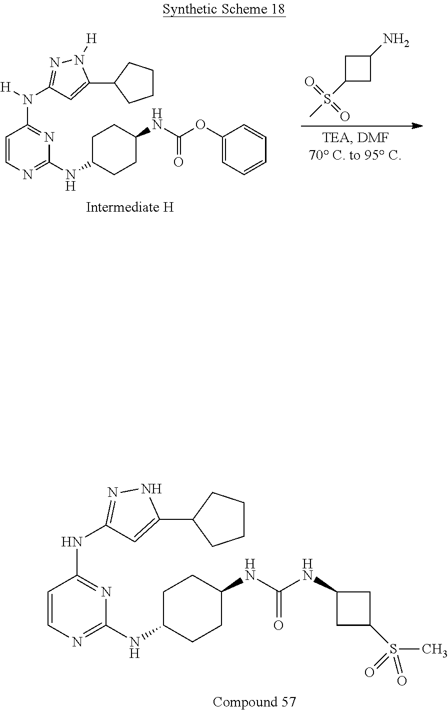

To a stirred solution of intermediate H—phenyl ((1R,4R)-4-((4-((5-cyclopentyl-1H-pyrazol-3-yl)amino)pyrimidin-2-yl)amino)cyclohexyl)carbamate (200 mg, 0.433 mmol) in anhydrous DMF (4.0 mL), triethylamine (0.18 mL, 1.30 mmol) was added, and the reaction mixture was stirred at 70° C., for 120 min. The reaction mixture was allowed to cool to room temperature, and then 3-(methylsulfonyl)cyclobutan-1-amine (64 mg, 0.433 mmol) was added, and the reaction mixture was heated to 95° C. After completion of 16 h stirring, the reaction mixture was diluted with water and extracted with 5% methanol in dichloromethane. The combined organic layers were dried over anhydrous Na₂SO₄ concentrated under reduced pressure to afford crude material. The crude material was purified by prep HPLC to afford Compound 57 1-((1R,4R)-4-((4-((S5-cyclopentyl-1H-pyrazol-3-yl)amino)pyrimidin-2-yl)amino)cyclohexyl)-3-(1-(pyridin-2-ylmethyl)piperidin-4-yl)urea (15 mg, 22% yield) as a TFA salt. LC purity: 97.95%; m/z: 517.3 [M+H]⁺ (Mol. formula C₂₄H₃₆N₈O₃S, calcd. mol. wt. 516.26); ¹H NMR (400 MHz, CD₃OD): δ 7.70 (d, J=6.8 Hz, 1H), 6.47-6.31 (m, 2H), 4.45-4.41 (m, 1H), 3.91-3.81 (m, 1H), 3.80-3.76 (m, 1H), 3.53-3.47 (m, 1H), 3.32-3.14 (m, 1H), 2.92 (s, 3H), 2.83-2.77 (m, 2H), 2.45-2.37 (m, 2H), 2.14-2.04 (m, 6H), 1.84-1.67 (m, 6H), 1.56-1.46 (m, 2H), 1.37-1.30 (m, 2

### Example 20: Synthesis of Compound 43

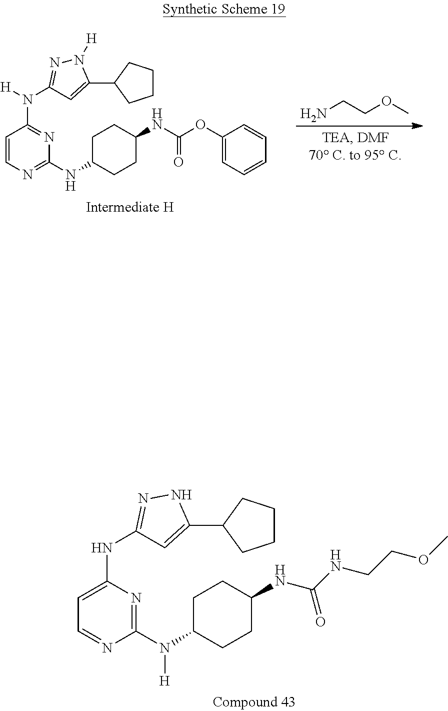

To a stirred solution of intermediate H—phenyl ((1R,4R)-4-((4-((5-cyclopentyl-1H-pyrazol-3-yl)amino)pyrimidin-2-yl)amino)cyclohexyl)carbamate (150 mg, 0.325 mmol) in dry DMF (4.0 mL), was added triethylamine (0.14 mL, 0.976 mmol) and the reaction mixture was stirred at 70° C., for 120 min. Then, the reaction mixture was cooled to room temperature, and 2-methoxyethan-1-amine was added, and the reaction mixture was heated to 95° C. After completion of 16 h stirring, the reaction mixture was diluted with water (10 mL) extracted with 5% methanol in dichloromethane (2×10 mL), and the combined organic layers were dried over anhydrous Na₂SO₄ and concentrated under reduced pressure to afford the crude material. The crude material was purified by prep HPLC to afford compound 43 1-((1R,4R)-4-((4-((5-cyclopentyl-1H-pyrazol-3-yl)amino)pyrimidin-2-yl)amino)cyclohexyl)-3-(2-methoxyethyl)urea (20 mg, 14% yield) as a TFA salt. LC purity: 96.6%; m/z: 443.3 [M+H]⁺ (Mol. formula C₂₂H₃₄N₈O₂, calcd. mol. wt. 442.57). ¹H NMR (400 MHz, CD₃OD): δ 7.69 (d, J=6.96 Hz, 1H), 6.56 (s, 1H), 6.29 (d, J=6.92 Hz, 1H), 3.97-3.91 (m, 1H), 3.51-3.45 (m, 2H), 3.44-3.43 (m, 2H), 3.37 (s, 3H), 3.14-3.13 (m, 1H), 2.13-2.06 (m, 6H), 1.89-1.85 (m, 2H), 1.83-1.73 (m, 4H), 1.53-1.50 (m, 2H), 1.36-1.30 (m, 2H).

### Example 21: Synthesis of Compound 97

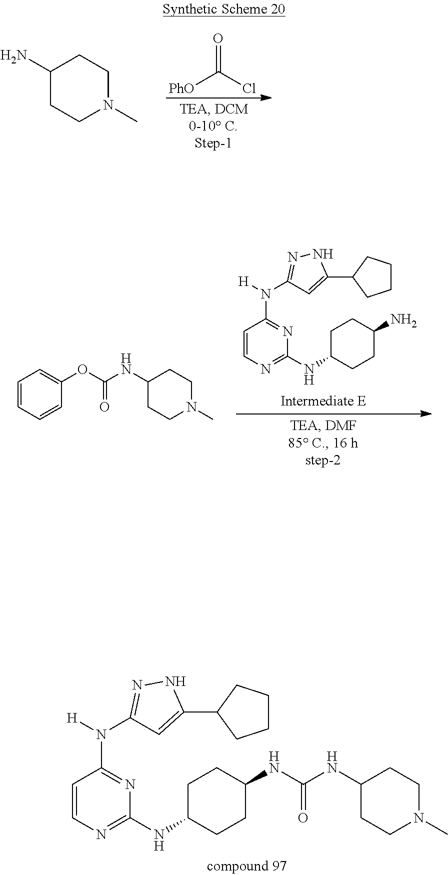

### Step-1: Synthesis of phenyl (1-methylpiperdine-4-yl)carbamate

To a solution of 1-methylpiperdine-4-amine (0.200 g, 1.33 mmol) in dichloromethane (5 mL) was added triethylamine (0.46 mL, 3.32 mmol) dropwise at 0° C., followed by addition of phenylchloroformate (0.17 mL, 1.33 mmol). The reaction mixture was stirred at 0° C. to 10° C. for 2 h. The reaction was monitored by TLC and after complete consumption of starting material, the reaction mixture was diluted with dichloromethane and washed with water. The organic layer separated was dried over anhydrous Na₂SO₄, filtered and concentrated under reduced pressure. The solid obtained was triturated with pet ether to obtain phenyl (1-methylpiperdine-4-yl)carbamate (220 mg, crude) as a white solid, which was taken to next step without further purification. LC purity: 99.5%; m/z: 235.2 [M+H]⁺ (Mol. C₂₅H₃₉N₉O calcd. mol. wt. 234.32).

### Step-2: Synthesis of 1-((1R, 4R)-4-((4-((5-cyclopentyl-1H-pyrazol-3-yl)amino)pyrimidin-2-yl) amino)cyclohexyl)-3-(1-methylpiperidin-4-yl)urea

To a solution of phenyl (1-methylpiperidin-4-yl)carbamate (0.05 g, 0.16 mmol) in N,N-dimethylformamide (3 mL) was added triethylamine (0.07 mL, 0.48 mmol) and the reaction mixture was heated at 65° C. for 1 h. After 1 h, the reaction mixture was cooled to room temperature, followed by addition of Intermediate E—N²-((1R,4R)-4-aminocyclohexyl)-N⁴-(5-cyclopentyl-1H-pyrazol-3-yl)pyrimidine-2,4-diamine (0.075 g, 0.16 mmol). The reaction mixture was then heated at 85° C. for 16 h. The reaction mixture was cooled to room temperature, diluted with water and extracted with dichloromethane. The organic layer was dried over anhydrous Na₂SO₄, filtered and concentrated under reduced pressure. The residue was purified by reverse phase preparative HPLC to yield compound 97 1-((1R,4R)-4-((4-((5-cyclopentyl-1H-pyrazol-3-yl)amino)pyrimidin-2-yl)amino) cyclohexyl)-3-(1-methylpiperidin-4-yl)urea (20 mg, 24.6% yield) as a TFA salt. LC purity: 99.09%; m/z: 482.4 [M+H]⁺ (Mol. C₂₅H₃₉N₉O calcd. mol. wt. 481.65). ¹H NMR (400 MHz, CD₃OD): δ 7.69 (d, J=6.8 Hz, 1H), 6.51-6.31 (m, 2H), 3.84-3.73 (m, 2H), 3.56-3.49 (m, 3H), 3.20-3.08 (m, 3H), 2.88 (s, 3H), 2.19-2.07 (m, 8H), 1.85-1.72 (m, 8H), 1.56-1.42 (m, 2H), 1.38-1.25 (m, 2H).

### Example 22: Synthesis of Compound 60

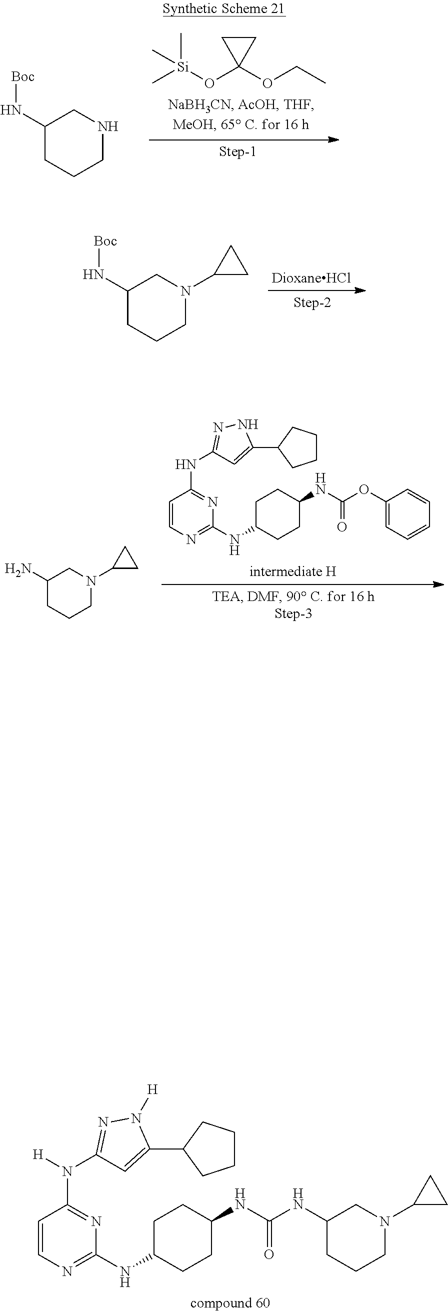

### Step-1: Synthesis of tert-butyl(1-cyclopropypiperdin-3-yl)carbamate

To a solution of tert-butyl piperdin-3-yl carbamate (0.150 g, 0.75 mmol) in 9:1 THF:MeOH (5 mL) was added 4 Å molecular sieves (0.150 g), (1-ethoxycyclopropoxy) trimethylsilane (0.390 g, 2.25 mmol), acetic acid (0.51 mL, 9 mmol) and sodium cyanoborohydride (0.141 g, 2.25 mmol) at room temperature. The reaction was heated at 65° C. for 16 h. The completion of reaction was monitored by TLC. The suspension was filtered and concentrated. The crude was diluted with sodium bicarbonate solution and extracted with dichloromethane. The organic layer was dried over anhydrous Na₂SO₄, filtered and concentrated under reduced pressure. The crude material was purified by Biotage isolara using silica gel (230-400) with gradient elution of 0-20% methanol in dichloromethane to obtain colorless oil tert-butyl(1-cyclopropypiperdine-3-yl)carbamate (120 mg, 66.6% yield). LC purity: 93.9%; m/z: 241.4 [M+H]⁺ (Mol. C₁₃H₂₄N₂O₂ calcd. mol. wt. 240.35).

### Step-2: Synthesis of 1-cyclopiperdine-3-amine

To a tert-butyl(1-cyclopropylpyrrolidin-3-yl)carbamate (0.103 g, 0.42 mmol) was added 2 mL of 4M HCl in dioxane at 0° C. The reaction was stirred at room temperature for 4 h. After complete consumption of the starting material (monitored by TLC) the reaction mixture was concentrated under reduced pressure to give 1-cyclopropylpiperidine-3-amine (50 mg, 83%) as a colorless gummy solid. This was taken to the next step without further purification. LC purity: 99.9%; m/z: 141.2 [M+H]⁺ (Mol. C₈H₁₆N₂ calcd. mol. wt. 140.13).

### Step-3: Synthesis of 1-((1R,4R)-4-((4-((5-cyclopentyl-1H-pyrazol-3-yl)amino)pyrimidin-2-yl) amino)cyclohexyl)-3-(1-cyclopropylpiperidine-3yl)urea

To a stirred solution of Intermediate H—phenyl ((1R,4R)-4-((4-((5-cyclopentyl-1H-pyrazol-3-yl)amino)pyrimidin-2-yl)amino)cyclohexyl)carbamate (0.150 g, 0.325 mmol) in dry DMF (2 mL) was added triethylamine (0.06 mL, 0.45 mmol). The reaction was heated to 65° C. for 1 h. The reaction mixture was cooled to room temperature and added 1-cyclopiperidine-3-amine (0.045 g, 0.325 mmol) and was heated to 85° C. for 16 h. The completion of reaction mixture was monitored by TLC, starting material was consumed. The reaction mixture was diluted with water and extracted with dichloromethane. The resulting organic layer was washed with brine solution then dried over anhydrous Na₂SO₄, filtered and concentrated under reduced pressure to obtain crude compound. The crude compound was purified by reverse phase prep HPLC to deliver Compound 60 1-((1R,4R)-4-((4-((5-cyclopentyl-1H-pyrazol-3-yl)amino)pyrimidin-2-yl)amino)cyclohexyl)-3-(1-cyclopropylpiperidine-3-yl)urea (16 mg, 9.7% yield). LC purity: 99.43%; m/z: 506.3 [M−H]⁺ (Mol. C₂₇H₄₁N₉O calcd. mol. wt. 507.69). ¹H NMR (400 MHz, CD₃OD): δ 7.78 (s, 1H), 6.14-(bs, 2H), 3.73-3.70 (m, 2H), 3.69-3.50 (m, 1H), 3.09-3.01 (m, 1H), 2.91-2.90 (m, 1H), 2.74-2.68 (m, 1H), 2.48-2.42 (m, 1H), 2.24-2.23 (m, 1H), 2.11-2.00 (m, 6H), 1.82-1.56 (m, 10H), 1.41-1.29 (m, 5H), 0.47-0.39 (m, 4H).

### Example 23: Synthesis of Compound 79

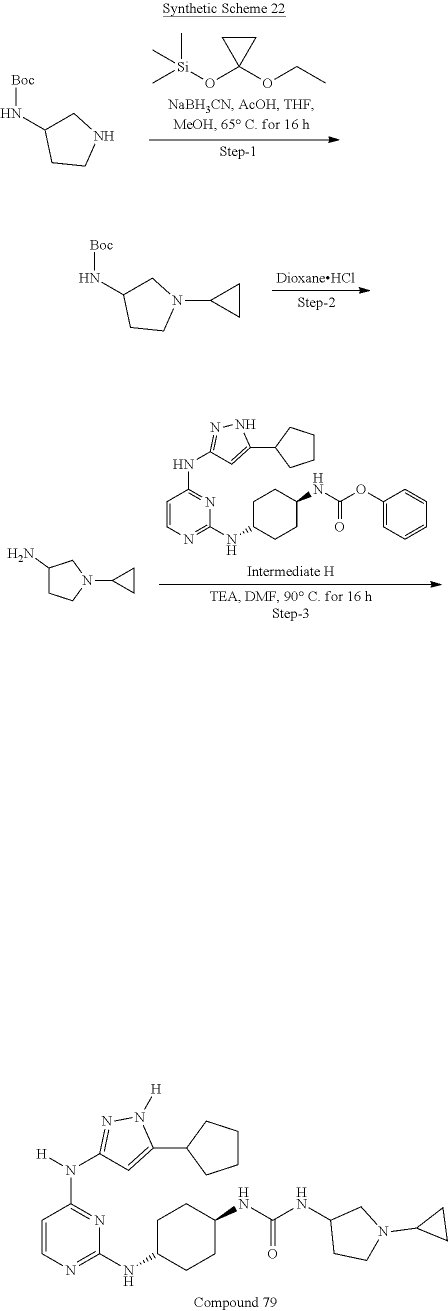

### Step-1: Synthesis of tert-butyl(1-cyclopropylpyrrolidin-3-yl)carbamate

To a solution of tert-butyl pyrrolidine-3-yl carbamate (0.150 g, 0.80 mmol) in 9:1 THF:MeOH (5 mL) was added 4 Å molecular sieves (0.150 g), (1-ethoxycyclopropoxy) trimethylsilane (0.421 g, 2.41 mmol), acetic acid (0.55 mL, 9.6 mmol) and sodium cyanoborohydride (0.152 g, 2.41 mmol) at room temperature. The reaction was heated at 65° C. for 16 h. The completion of the reaction was monitored by TLC. The suspension was filtered and concentrated. The crude material was diluted with sodium bicarbonate solution and extracted with dichloromethane. The organic layer was dried over anhydrous Na₂SO₄, filtered and concentrated under reduced pressure. The crude material was purified by Biotage isolara using silica gel (230-400) with gradient elution of 0-20% methanol in dichloromethane to obtain colorless oil tert-butyl(1-cyclopropylpyrrolidine-3-yl)carbamate (80 mg, 44.4% yield). LC purity: 99.7%; m/z: 227.3 [M+H]⁺ (Mol. C₁₂H₂₂N₂O₂ calcd. mol. wt. 226.32).

### Step-2: Synthesis of 1-cyclopropylpyrrolidine-3-amine

To a tert-butyl(1-cyclopropylpyrrolidin-3-yl)carbamate (0.080 g, 0.35 mmol) was added 2 mL of 4M HCl in dioxane at 0° C. The reaction mixture was stirred at room temperature for 4 h. After complete consumption of the starting material (monitored by TLC) the reaction mixture was concentrated under reduced pressure to obtain 1-cyclopropylpyrrolidine-3-amine (40 mg, 90%) as a colorless gummy solid. This was taken to the next step without further purification. LC purity: 99.6%; m/z: 127.2 [M+H]⁺ (Mol. C₇H₁₄N₂ calcd. mol. wt. 126.20).

### Step-3: Synthesis of 1-((1R,4R)-4-((4-((5-cyclopentyl-1H-pyrazol-3-yl)amino)pyrimidin-2-yl)amino)cyclohexyl)-3-(1-cyclopropylpiperdin-3-yl)urea

To a stirred solution of Intermediate H—phenyl ((1R,4R)-4-((4-((5-cyclopentyl-1H-pyrazol-3-yl)amino) pyrimidin-2-yl)amino)cyclohexyl)carbamate (0.07 g, 0.15 mmol) in anhydrous DMF (2 mL) was added triethylamine (0.06 mL, 0.45 mmol). The reaction mixture was heated to 65° C. for 1 h. The reaction mixture was cooled to room temperature and 1-cyclopropylpyrrolidine-3-amine (0.019 g, 0.15 mmol) was added. The reaction mixture was heated to 85° C. for 16 h. The reaction mixture was monitored by TLC, and after complete consumption of starting material, the reaction mixture was diluted with water and extracted with dichloromethane. The resulting organic layer was washed with brine solution then dried over anhydrous Na₂SO₄, filtered and concentrated under reduced pressure to obtain the crude compound. The crude compound was purified by reverse phase prep HPLC to obtain Compound 79 1-((1R,4R)-4-((4-((5-cyclopentyl-1H-pyrazol-3-yl)amino)pyrimidin-2-yl)amino)cyclohexyl)-3-(1-cyclopropylpyrrolidin-3-yl)urea (10 mg, 20% yield). LC purity: 99.06%; m/z: 494.3 [M+H]⁺ (Mol. C₂₆H₃₉N₉O calcd. mol. wt. 493.33). ¹H NMR (400 MHz, CD₃OD): δ 7.78 (d, J=6 Hz, 1H), 6.31-6.15 (m, 2H), 4.28-4.21 (m, 1H), 3.82-3.78 (m, 1H), 3.50-3.48 (m, 1H), 3.09-3.01 (m, 1H), 3.00-2.97 (m, 1H), 2.91-2.90 (m, 1H), 2.71-2.67 (m, 1H), 2.61-2.57 (m, 1H), 2.26-2.24 (m, 1H), 2.11-2.09 (m, 4H), 2.02-1.99 (m, 2H), 1.83-1.59 (m, 8H), 1.43-1.30 (m, 5H), 0.52-0.44 (m, 4H).

### Example 24: Synthesis of Compound 2

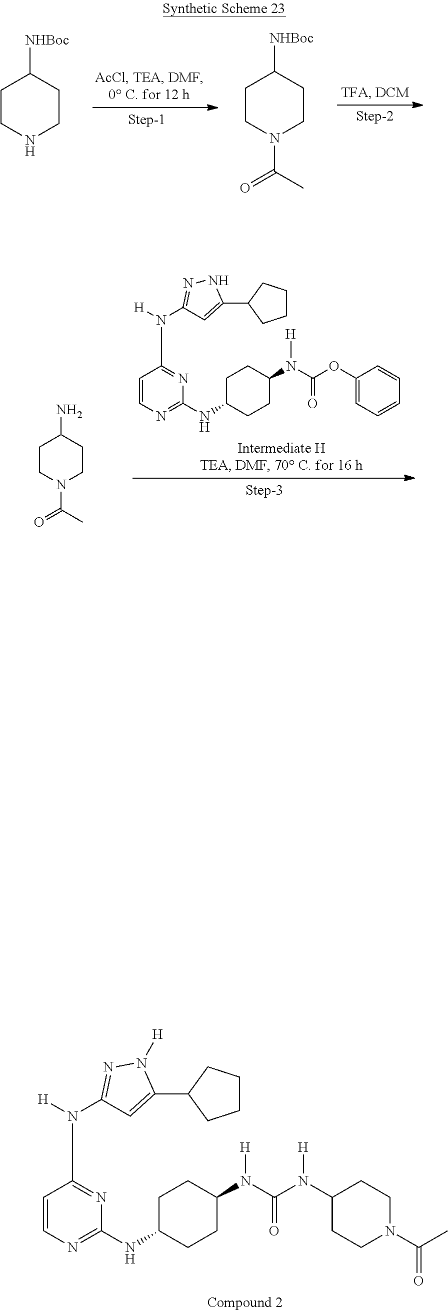

### Step-1: Synthesis of tert-butyl (1-acetylpiperidin-4-yl)carbamate

To a stirred solution of tert-butyl piperidin-4-yl carbamate (200 mg, 1.00 mmol) in dry DMF (5 ml) was added triethylamine (0.41 mL, 3.00 mmol) and acetyl chloride (0.11 mL, 1.50 mmol) at 0° C. The reaction mixture was stirred at ambient temperature for 12 h. The reaction mixture was monitored by TLC, starting material was consumed. The reaction mixture was diluted with water, extracted with dichloromethane. The resulting organic layer was washed with brine solution then dried over anhydrous Na₂SO₄ and concentrated to obtain the crude compound. The crude material thus obtained was purified by Biotage isolera (230-400 silica gel) with gradient elution of 0-80% ethyl acetate in petroleum ether to yield tert-butyl (1-acetylpiperidin-4-yl) carbamate (180 mg, 75% yield). LC purity: 58.69%; m/z: 187.2 [M+H−tBu]⁺ (Mol. formula C₁₂H₂₂F₃N₂O₃, calcd. mol. wt. 242.32).

### Step-2: Synthesis of 1-(4-aminopiperidin-1-yl)ethan-1-one

To a stirred solution of tert-butyl (1-acetylpiperidin-4-yl)carbamate (180 mg, 0.7428 mmol) in dichloromethane (5 mL) was added TFA (2 mL) at 0° C. The reaction mixture was slowly warmed to room temperature and stirred for 5 h. The solvents were removed under reduced pressure and co-distilled with toluene to obtain crude 1-(4-aminopiperidin-1-yl)ethan-1-one (120 mg, quantitative yield). LC purity: 93.36%; m/z: 143.2 [M+H]⁺ (Mol. formula C₇H₁₄N₂O, calcd. mol. wt. 142.2).

### Step-3: Synthesis of 1-(1-acetylpiperidin-4-yl)-3-((1R,4R)-4-((4-((5-cyclopentyl-1H-pyrazol-3-yl)amino)pyrimidin-2-yl)amino)cyclohexyl)urea

To a stirred solution of Intermediate H—phenyl ((1R,4R)-4-((4-((5-cyclopentyl-1H-pyrazol-3-yl)amino)pyrimidin-2-yl)amino)cyclohexyl)carbamate (120 mg, 0.260 mmol) in dry DMF (2 mL) was added tri ethylamine (0.1 mL, 0.780 mmol). The reaction mixture was heated to 65° C. for 1 h. The reaction was cooled to room temperature and added 1-(4-aminopiperidin-1-yl)ethan-1-one (40 mg, 0.260 mmol). The reaction mixture was heated to 70° C. for 16 h. The reaction mixture was monitored by TLC, starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 2 1-(1-acetylpiperidin-4-yl)-3-((1R,4R)-4-((4-((5-cyclopentyl-1H-pyrazol-3-yl)amino)pyrimidin-2-yl)amino)cyclohexyl)urea (30 mg, 23% yield) as a TFA salt. LC purity: 99.53%; m/z: 510.4 [M+H]⁺ (Mol. formula C₂₆H₃₉N₉O₂, calcd. mol. wt. 509.6). ¹H NMR (400 MHz, CD₃OD): δ 7.67 (s, 1H), 6.51 (bs, 1H), 6.29 (bs, 1H), 4.34-4.30 (m, 1H), 4.86-4.72 (m, 3H), 3.53-3.49 (m, 1H), 3.24-3.11 (m, 2H), 2.90-2.85 (m, 1H), 2.13-2.05 (m, 9H), 1.87-1.71 (m, 7H), 1.52-1.46 (m, 2H), 1.41-1.25 (m, 5H).

### Example 25: Synthesis of Compound 49

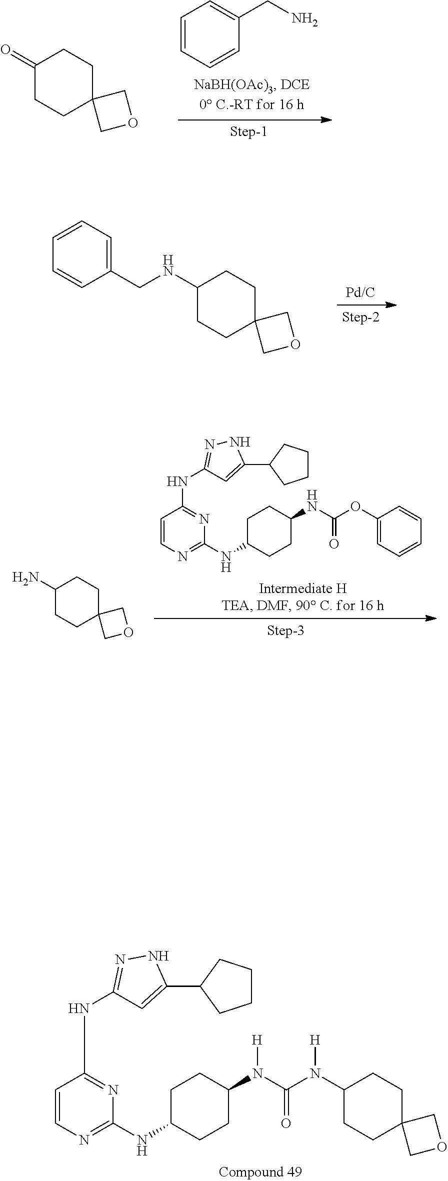

### Step-1: Synthesis of N-benzyl-2-oxaspiro[3.5]nonan-7-amine

To a stirred solution of 2-oxaspiro[3.5]nonan-7-one (200 mg, 1.428 mmol) in 1,2-dichloro ethane (5 mL) was added phenylmethanamine (0.2 mL, 1.714 mmol) and sodium triacetoxy borohydride (450 mg, 2.142 mmol) at 0° C. The reaction mixture was warmed to room temperature and stirred for 16 h. The reaction mixture was monitored by TLC and after complete consumption of starting material the reaction mixture was diluted with water and extracted with dichloromethane. The combined organic layers were washed with water, and brine, dried over anhydrous Na₂SO₄ and concentrated to yield the crude product, which was then purified by Biotage isolera using 230-400 silica gel with gradient elution of 0-80% ethyl acetate in pet ether to yield N-benzyl-2-oxaspiro[3.5]nonan-7-amine (220 mg, 66.6% yield). LC purity: 81.90%; m/z: 232.2 [M+H]⁺ (Mol. formula C₁₅H₂₁NO, calcd. mol. wt. 231.34).

### Step-2: Synthesis of 2-oxaspiro[3.5]nonan-7-amine

To a solution of N-benzyl-2-oxaspiro[3.5]nonan-7-amine (180 mg, 0.7792 mmol) in ethanol (5 mL) was added 100 mg of Pd(OH)₂ and catalytic amount of glacial acetic acid. The atmosphere in the vessel was replaced with H₂ under balloon pressure and the reaction was stirred at room temperature for 16 h. The reaction mixture was filtered through celite and washed with methanol. The solvent removed under vacuum to yield 2-oxaspiro[3.5]nonan-7-amine (140 mg, crude) which was directly used for next step without further purification.

### Step-3: Synthesis of 1-((1R,4R)-4-((4-((5-cyclopentyl-1H-pyrazol-3-yl)amino)pyrimidin-2-yl)amino)cyclohexyl)-3-(2-oxaspiro[3.5]nonan-7-yl)urea

To a stirred solution of Intermediate H—phenyl ((1R,4R)-4-((4-((5-cyclopentyl-1H-pyrazol-3-yl)amino)pyrimidin-2-yl)amino)cyclohexyl)carbamate (100 mg, 0.2169 mmol) in dry DMF (3 mL) was added triethylamine (0.1 mL, 0.6507 mmol). The reaction mixture was heated to 65° C. for 1 h. The reaction mixture was cooled to room temperature and 2-oxaspiro[3.5]nonan-7-amine (80 mg, 0.2169 mmol) was added. The reaction mixture was heated to 90° C. for 16 h. The reaction mixture was monitored by TLC, and starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 49 1-((1R,4R)-4-((4-((5-cyclopentyl-1H-pyrazol-3-yl)amino)pyrimidin-2-yl)amino)cyclohexyl)-3-(2-oxaspiro[3.5]nonan-7-yl)urea (15 mg, 14% yield) as a TFA salt. LC purity: 97.13%; m/z: 509.3 [M+H]⁺ (Mol. formula C₂₇H₄₀N₈O₂, calcd. mol. wt. 508.67). ¹H NMR (400 MHz, CD₃OD): δ 7.66 (bs, 1H), 6.52 (bs, 1H), 6.26 (bs, 1H), 4.42 (s, 2H), 4.34 (s, 2H), 3.86 (m, 1H), 3.46-3.43 (m, 2H), 3.18-3.12 (m, 1H), 2.09-2.01 (m, 8H), 1.81-1.58 (m, 10H), 1.55-1.46 (m, 3H), 1.28-1.25 (m, 2H), 1.19-1.13 (m, 2H).

### Example 26: Synthesis of Compound 44

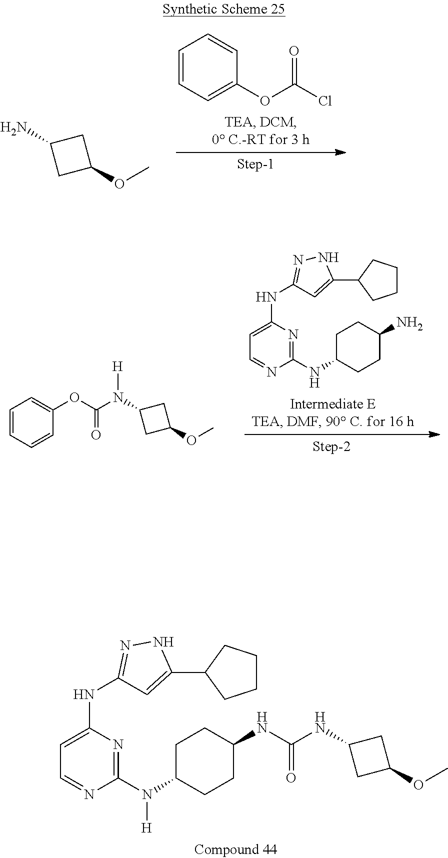

### Step-1: Synthesis of phenyl ((1R,3R)-3-methoxycyclobutyl)carbamate

To a stirred solution of (1R,3R)-3-methoxycyclobutan-1-amine (200 mg, 1.977 mmol) in DMF (5 ml) was added triethylamine (0.82 mL, 5.931 mmol) and phenyl chloroformate (0.37 mL, 2.965 mmol) at 0° C. The reaction was stirred at room temperature for 3 h. The reaction mixture was monitored by TLC and after complete consumption of starting material the reaction mixture was diluted with water, extracted with dichloromethane. The resulting organic layer was washed with brine solution then dried over anhydrous Na₂SO₄ and concentrated to obtain crude compound. The crude material thus obtained was triturated with pet ether and concentrated to yield phenyl ((1R,3R)-3-methoxycyclobutyl)carbamate (250 mg, 57.2% yield). LC purity: 99.21%; m/z: 222.2 [M+H]⁺ (Mol. formula C₁₂H₁₅NO₃, calcd. mol. wt. 221.26).

### Step-2: Synthesis of 1-((1R,4R)-4-((4-((5-cyclopentyl-1H-pyrazol-3-yl)amino)pyrimidin-2-yl)amino)cyclohexyl)-3-((1R,3R)-3-methoxycyclobutyl)urea

To a stirred solution of phenyl ((1R,3R)-3-methoxycyclobutyl)carbamate (250 mg, 1.129 mmol) in anhydrous DMF (5 mL) was added triethylamine (0.46 mL, 3.387 mmol). The reaction mixture was heated to 65° C. for 1 h. The reaction mixture was cooled to room temperature and added Intermediate E—N²-((1R,4R)-4-aminocyclohexyl)-N⁴-(5-cyclopentyl-1H-pyrazol-3-yl)pyrimidine-2,4-diamine (385 mg, 1.129 mmol). The reaction was heated to 90° C. for 16 h. The reaction mixture was monitored by TLC, starting material was consumed. The reaction mixture was diluted with water and extracted with dichloromethane. The resulting organic layer was washed with brine solution then dried over anhydrous Na₂SO₄ and concentrated to obtain the crude compound. The crude compound was purified by reverse phase preparative HPLC to yield Compound 44 1-((1R,4R)-4-((4-((5-cyclopentyl-1H-pyrazol-3-yl)amino)pyrimidin-2-yl)amino) cyclohexyl)-3-((1R,3R)-3-methoxycyclobutyl)urea (30 mg, 6% yield) as a TFA salt. LC purity: 99.64%; m/z: 469.4 [M+H]⁺ (Mol. formula C₂₄H₃₆N₈O₂, calcd. mol. wt. 468.61). ¹H NMR (400 MHz, CD₃OD): δ 7.67 (s, 1H), 6.45 (bs, 2H), 4.22-4.18 (m, 1H), 4.00-3.97 (m, 1H), 3.80-3.75 (m, 1H), 3.51-3.49 (m, 1H), 3.23 (s, 3H), 3.15-3.11 (m, 1H), 2.35-2.29 (m, 2H), 2.13-2.03 (m, 8H), 1.83-1.71 (m, 6H), 1.68-1.48 (m, 2H), 1.45-1.29 (m, 2H).

### Example 27: Synthesis of Compound 48

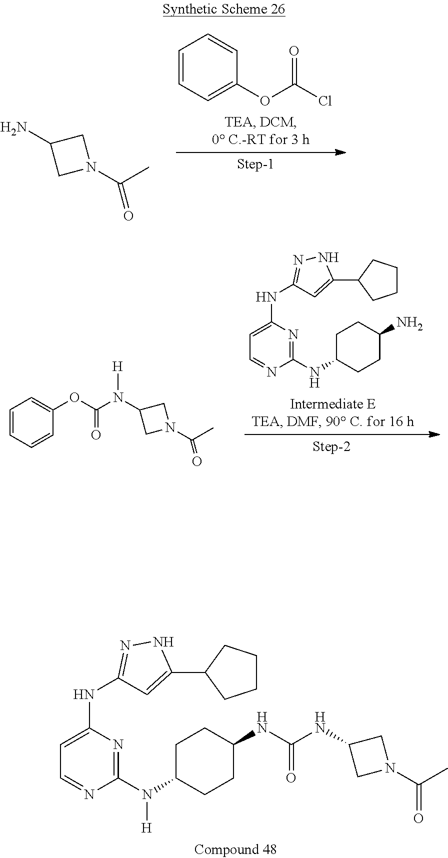

### Step-1: Synthesis of phenyl (1-acetylazetidin-3-yl)carbamate

To a stirred solution of 1-(3-aminoazetidin-1-yl)ethan-1-one (200 mg, 1.752 mmol) in dry DMF (5 ml) was added triethylamine (0.72 mL, 5.256 mmol) and phenyl chloroformate (0.33 mL, 2.628 mmol) at 0° C. The reaction was stirred at room temperature for 3 h. The reaction mixture was monitored by TLC after complete conversion of starting material the reaction mixture was diluted with water, extracted with dichloromethane. The resulting organic layer was washed with brine solution then dried over anhydrous Na₂SO₄ and concentrated to obtain the crude compound. The crude material thus obtained was washed with pet ether and concentrated to yield phenyl (1-acetylazetidin-3-yl)carbamate (190 mg, 46.3% yield). LC purity: 98.40%; m/z: 235.1 [M+H]⁺ (Mol. formula C₁₂H₁₄N₂O₃, calcd. mol. wt. 234.26).

### Step-2: Synthesis of 1-(1-acetylazetidin-3-yl)-3-((1R,4R)-4-((4-((5-cyclopentyl-1H-pyrazol-3-yl)amino)pyrimidin-2-yl)amino)cyclohexyl)urea

To a stirred solution of phenyl (1-acetylazetidin-3-yl)carbamate (190 mg, 0.811 mmol) in dry DMF (5 mL) was added triethylamine (0.33 mL, 2.433 mmol). The reaction mixture was heated to 65° C. for 1 h. The reaction mixture was cooled to room temperature and added Intermediate E—N²-((1R,4R)-4-aminocyclohexyl)-N⁴-(5-cyclopentyl-1H-pyrazol-3-yl)pyrimidine-2,4-diamine (277 mg, 0.811 mmol). The reaction mixture was heated to 90° C. for 16 h. The reaction mixture was monitored by TLC, starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase prep HPLC to yield Compound 48 1-(1-acetylazetidin-3-yl)-3-((1R,4R)-4-((4-((5-cyclopentyl-1H-pyrazol-3-yl)amino)pyrimidin-2-yl)amino)cyclohexyl)urea (20 mg, 5% yield) as a formic acid salt. LC purity: 97.87%; m/z: 482.8 [M+H]⁺ (Mol. formula C₂₄H₃₅N₉O₂, calcd. mol. wt. 481.61). ¹H NMR (400 MHz, CD₃OD): δ 7.72 (s, 1H), 6.25 (bs, 2H), 4.49-4.43 (m, 2H), 4.25-4.21 (m, 1H), 4.02-4.00 (m, 1H), 3.79-3.76 (m, 2H), 3.52-3.48 (m, 1H), 3.12-3.08 (m, 1H), 2.12-2.01 (m, 6H), 1.86 (s, 3H), 1.82-1.80 (m, 2H), 1.76-1.64 (m, 4H), 1.49-1.39 (m, 2H), 1.37-1.29 (m, 2H).

### Example 28: Synthesis of Compound 37

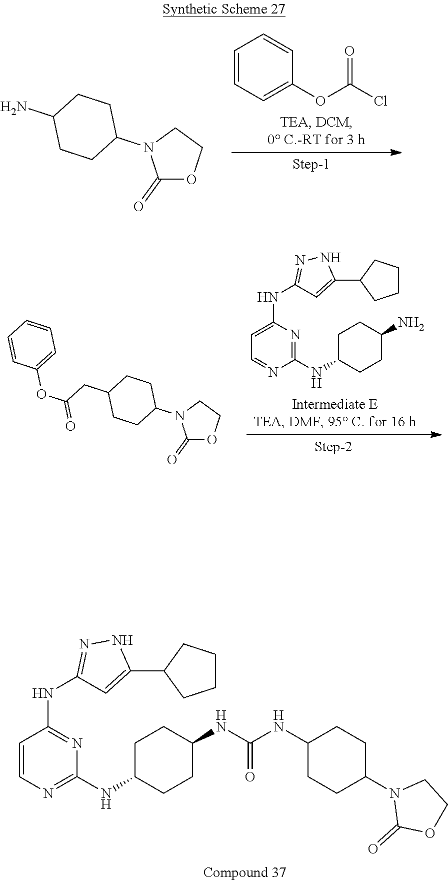 [SMILES: NC1CCC(N2CCOC2=O)CC1 ; O=C(Cl)Oc1ccccc1 ; O=C(CC1CCC(N2CCOC2=O)CC1)Oc1ccccc1 ; N[C@H]1CC[C@H](Nc2nccc(Nc3cc(C4CCCC4)[nH]n3)n2)CC1 ; O=C(NC1CCC(N2CCOC2=O)CC1)N[C@H]1CC[C@H](Nc2nccc(Nc3cc(C4CCCC4)[nH]n3)n2)CC1]

### Step-1: Synthesis of phenyl (4-(2-oxooxazolidin-3-yl)cyclohexyl)carbamate

To a stirred solution of 3-(4-aminocyclohexyl)oxazolidin-2-one (200 mg, 1.09 mmol) in dry dichloromethane (2 mL) was added triethylamine (0.46 mL, 3.2 mmol). The reaction mixture was stirred at 0° C. for 15 min. Then added phenyl chloroformate (0.16 mL, 1.3 mmol). The reaction was stirred at room temperature for 3 h. The reaction was monitored by TLC, starting material was consumed. The reaction mixture was diluted with water and extracted with ethyl acetate. The resulting organic layer was washed with brine solution then dried over anhydrous Na₂SO₄ and concentrated to obtain phenyl (4-(2-oxooxazolidin-3-yl)cyclohexyl)carbamate (160 mg, 48% yield). LC purity: 99.56%; m/z: 305.2 [M+H]⁺ (Mol. formula C₁₆H₂₀N₂O₄, calcd. mol. wt. 304.14).

### Step-2: Synthesis of 1-((1R,4R)-4-((4-((5-cyclopentyl-1H-pyrazol-3-yl)amino)pyrimidin-2-yl)amino)cyclohexyl)-3-(4-(2-oxooxazolidin-3-yl)cyclohexyl)urea

To a stirred solution of phenyl (4-(2-oxooxazolidin-3-yl)cyclohexyl)carbamate (160 mg, 0.526 mmol) in dry DMF (2 mL) was added triethylamine (0.22 mL, 1.57 mmol). The reaction mixture was heated to 85° C. for 1 h. The reaction mixture was cooled to room temperature and Intermediate E—N²-((1R,4R)-4-aminocyclohexyl)-N⁴-(5-cyclopentyl-1H-pyrazol-3-yl)pyrimidine-2,4-diamine (179 mg, 0.179 mmol) was added. The reaction was heated to 95° C. for 16 h. The reaction was monitored by TLC, starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 37 1-((1R,4R)-4-((4-((5-cyclopentyl-1H-pyrazol-3-yl)amino)pyrimidin-2-yl)amino) cyclohexyl)-3-(4-(2-oxooxazolidin-3-yl)cyclohexyl)urea (30 mg, 5.5% yield) as a formic acid salt. LC purity: 95.18%; m/z: 552.4 [M+H]⁺ (Mol. formula C₂₈H₄₁N₉O₃, calcd. mol. wt. 551.70). ¹H NMR (400 MHz, CD₃OD): δ 8.40 (s, 1H), 7.75 (d, J=6.4 Hz, 1H), 6.20 (bs, 2H), 4.38-4.33 (m, 2H), 3.89-3.86 (m, 2H), 3.67-3.59 (m, 5H), 3.51-3.42 (m, 1H), 3.28-3.11 (m, 1H), 2.13-2.02 (m, 7H), 1.84-1.68 (m, 10H), 1.48-1.28 (m, 5H).

### Example 29: Synthesis of Compound 77

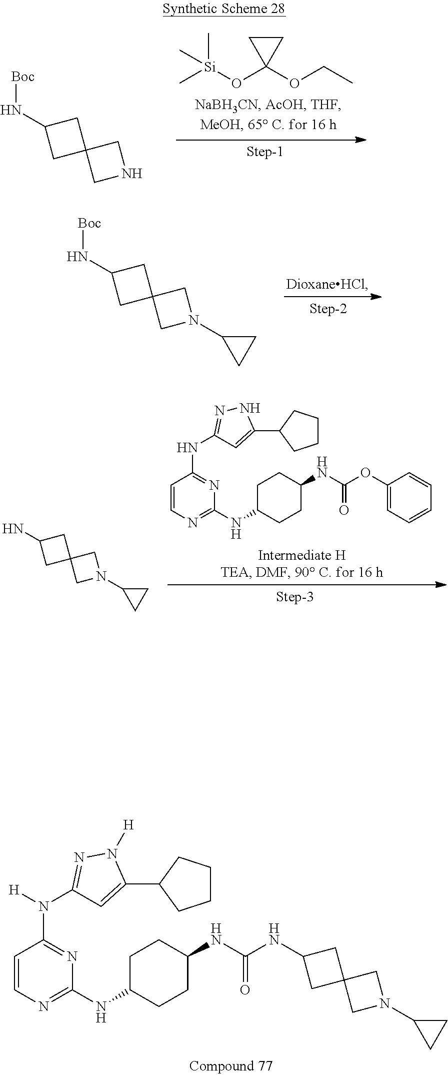

### Step-1: Synthesis of tert-butyl (2-cyclopropyl-2-azaspiro[3.3]heptan-6-yl)carbamate

To a stirred solution of tert-butyl (2-azaspiro[3.3]heptan-6-yl)carbamate (0.2 g, 0.94 mmol) in THF:MeOH 9:1 (5 mL) was added 4 Å molecular sieves (0.2 g), (1-ethoxycyclopropoxy)trimethylsilane (0.492 g, 2.8 mmol), acetic acid (0.1 mL, 0.18 mmol) and sodium cyano borohydride (0.175 g, 2.8 mmol) at room temperature. The reaction was heated at 65° C. for 16 h. The completion of the reaction was monitored by TLC. The reaction mixture was filtered and concentrated. The crude was diluted with saturated sodium bicarbonate solution and extracted with dichloromethane. The organic layer was dried over anhydrous Na₂SO₄ and concentrated under reduced pressure. The crude compound was purified by Biotage isolara using silica gel (230-400) with gradient elution of 0-90% ethyl acetate in petroleum ether to obtain tert-butyl (2-cyclopropyl-2-azaspiro[3.3]heptan-6-yl)carbamate (0.1 g, 42% yield). LC purity: 48.20%; m/z: 253.3 [M+H]⁺ (Mol. formula C₁₄H₂₄N₂O₂, calcd. mol. wt. 252.36)

### Step-2: Synthesis of 2-cyclopropyl-2-azaspiro[3.3]heptan-6-amine

To the mixture of tert-butyl (2-cyclopropyl-2-azaspiro[3.3]heptan-6-yl)carbamate (0.1 g, 0.396 mmol) in dry dichloromethane (2 mL) was added dioxane in HCl (2 mL, 4M solution). The reaction mixture was stirred at room temperature for 3 h. The progress of the reaction was monitored by TLC analysis. After completion of the reaction, the reaction mixture was concentrated to obtain 2-cyclopropyl-2-azaspiro[3.3]heptan-6-amine (82 mg, quantitative yield) which was directly taken to the next step without further purification. LC purity: 92.26%; m/z: 153.2 [M+H]⁺ (Mol. formula C₉H₁₆N₂, calcd. mol. wt. 152.24).

### Step-3: Synthesis of 1-((1R,4R)-4-((4-((5-cyclopentyl-1H-pyrazol-3-yl)amino)pyrimidin-2-yl)amino)cyclohexyl)-3-(2-cyclopropyl-2-azaspiro[3.3]heptan-6-yl)urea

To a stirred solution of Intermediate H—phenyl ((1R,4R)-4-((4-((5-cyclopentyl-1H-pyrazol-3-yl)amino)pyrimidin-2-yl)amino)cyclohexyl)carbamate (200 mg, 0.433 mmol) in anhydrous DMF (2 mL) was added triethylamine (0.179 mL, 1.3 mmol). The reaction mixture was heated to 85° C. for 1 h. The reaction mixture was cooled to room temperature and added 2-cyclopropyl-2-azaspiro[3.3]heptan-6-amine (65 mg, 0.433 mmol). The reaction was heated to 90° C. for 16 h. The reaction mixture was monitored by TLC, starting material was consumed. The reaction mixture was diluted with water and extracted with dichloromethane. The resulting organic layer was washed with brine solution then dried over anhydrous Na₂SO₄ and concentrated to get crude compound. which was purified by reverse phase prep HPLC to yield Compound 77 1-((1R,4R)-4-((4-((5-cyclopentyl-1H-pyrazol-3-yl)amino)pyrimidin-2-yl)amino)cyclohexyl)-3-(2-cyclopropyl-2-azaspiro[3.3]heptan-6-yl)urea (20 mg, 22% yield) as a TFA salt. LC purity: 98.18%; m/z: 520.4 [M+H]⁺ (Mol. formula C₂₈H₄₁N₉O, calcd. mol. wt. 519.70). ¹H NMR (400 MHz, CD₃OD): δ 7.75 (s, 1H), 6.12 (bs, 2H), 4.45-4.42 (m, 1H), 4.25 (s, 1H), 4.09-4.03 (m, 1H), 3.88-3.78 (m, 1H), 3.50 (s, 2H), 3.17-3.11 (m, 1H), 2.99-2.95 (m, 1H), 2.66-2.64 (m, 2H), 2.22-2.01 (m, 8H), 1.82-1.71 (m, 6H), 1.55-1.41 (s, 3H), 1.35-1.26 (m, 2H), 0.96-0.91 (m, 4H).

### Example 30: Synthesis of Compound 3

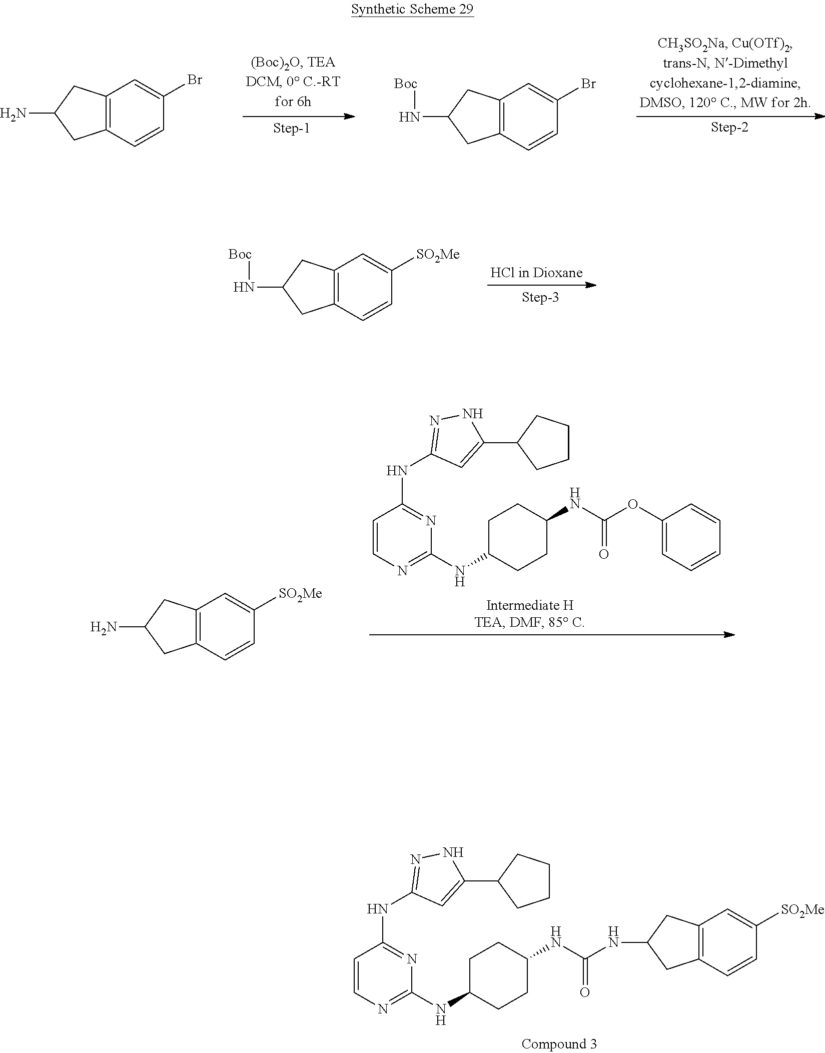

### Step-1: Synthesis of tert-butyl (5-bromo-2,3-dihydro-1H-inden-2-yl)carbamate

To a cooled 0° C. solution of 5-bromo-2,3-dihydro-1H-inden-2-amine (1 g, 4.716 mmol) in dichloromethane (20 mL) was added triethylamine (1.95 mL, 14.15 mmol) and Boc anhydride (1.5 mL, 7.075 mmol). The reaction mixture was stirred at room temperature for 16 h. After completion of the reaction (monitored by TLC), reaction mixture was diluted with water and extracted with dichloromethane. The organic layer was dried over anhydrous Na₂SO₄ and concentrated. The residue was purified by Biotage isolara using silica gel (230-400) with gradient elution of 0-60% ethyl acetate in petroleum ether to obtain tert-butyl (5-bromo-2,3-dihydro-1H-inden-2-yl)carbamate (1 g, 68% yield). LC purity: 98.97%; m/z: 258.9 [M−^{t}Bu]⁺ (Mol. formula C₁₄H₁₈BrNO₂, calcd. mol. wt. 312.21).

### Step-2: Synthesis of tert-butyl (5-(methylsulfonyl)-2,3-dihydro-1H-inden-2-yl)carbamate

In a 20 mL microwave vial a stirred solution of tert-butyl (5-bromo-2,3-dihydro-1H-inden-2-yl)carbamate (0.25 g, 0.80 mmol) in anhydrous DMSO (4 mL) was added sodium methane sulfonate (0.189 g, 1.6 mmol) and Cu(II) triflate (0.06 mg, 0.16 mmol) followed by the addition of trans-N,N′-dimethyl cyclohexane-1,2-diamine (0.01 mL, 0.08 mmol). The vial was sealed and heated to 120° C. in a microwave. After 2 h, the reaction mixture was cooled to ambient temperature, diluted with water and the organic contents were extracted with ethyl acetate. The organic layer thus obtained was dried over anhydrous Na₂SO₄, filtered and concentrated under reduced pressure. The residue thus obtained was purified by flash column chromatography on silica gel (230-400 mesh) eluting with 0-50% ethyl acetate in petroleum ether to deliver tert-butyl (5-(methylsulfonyl)-2,3-dihydro-1H-inden-2-yl)carbamate (0.08 g, 32% yield). LC purity: 99.67%; m/z: 212.2 [M−Boc]⁺ (Mol. formula C₁₅H₂₁N₉O₄S, calcd. mol. wt. 311.40).

### Step-3: Synthesis of 5-(methylsulfonyl)-2,3-dihydro-1H-inden-2-amine

To the mixture of tert-butyl (5-(methylsulfonyl)-2,3-dihydro-1H-inden-2-yl)carbamate (0.08 g, 0.257 mmol) in dry dichloromethane (2 mL) was added HCl in dioxane (2 mL, 4M solution). The reaction mixture was stirred at room temperature for 3 h. The progress of the reaction was monitored by TLC analysis. The reaction mixture was concentrated to deliver 5-(methylsulfonyl)-2,3-dihydro-1H-inden-2-amine (62 mg, quantitative yield), which was taken for next step without purification. LC purity: 56.81%; m/z: 212.0 [M+H]⁺ (Mol. formula C₁₀H₁₃NO₂S, calcd. mol. wt. 211.28).

### Step-4: Synthesis of 1-((1R,4R)-4-((4-((5-cyclopentyl-1H-pyrazol-3-yl)amino)pyrimidin-2-yl)amino)cyclohexyl)-3-(5-(methylsulfonyl)-2,3-dihydro-1H-inden-2-yl)urea

To a stirred solution of Intermediate H—phenyl ((1r,4r)-4-((4-((5-cyclopentyl-1H-pyrazol-3-yl)amino)pyrimidin-2-yl)amino)cyclohexyl)carbamate (200 mg, 0.433 mmol) in anhydrous DMF (4 mL) was added tri ethylamine (0.2 mL, 1.29 mmol). The reaction was heated to 85° C. for 1 h. The reaction mixture was cooled to room temperature and 5-(methylsulfonyl)-2,3-dihydro-1H-inden-2-amine (134 mg, 0.433 mmol) was added. The reaction mixture was heated to 95° C. for 16 h. The reaction mixture was monitored by TLC, and after complete consumption of starting material the reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 3 1-((1R,4R)-4-((4-((5-cyclopentyl-1H-pyrazol-3-yl)amino)pyrimidin-2-yl)amino) cyclohexyl)-3-(5-(methylsulfonyl)-2,3-dihydro-1H-inden-2-yl)urea (25 mg, 25% yield) as a formic acid salt. LC purity: 96.44%; m/z: 579.3 [M+H]⁺ (Mol. formula C₂₉H₃₈N₈O₃S, calcd. mol. wt. 578.74). ¹H NMR (400 MHz, CD₃OD): δ 7.79-7.68 (m, 3H), 7.46 (d, J=8 Hz, 1H), 6.52 (bs, 1H), 6.31 (bs, 1H), 4.53-4.58 (m, 1H), 3.86-3.53 (m, 1H), 3.50 (t, J=11.2 Hz, 1H), 3.34-3.29 (m, 1H), 3.11-3.09 (m, 1H), 3.07 (s, 3H), 2.89-2.83 (m, 2H), 2.09-2.02 (m, 6H), 1.79-1.68 (m, 6H), 1.49-1.44 (m, 2H), 1.31-1.22 (m, 3H).

### Example 31: Synthesis of Compound 103

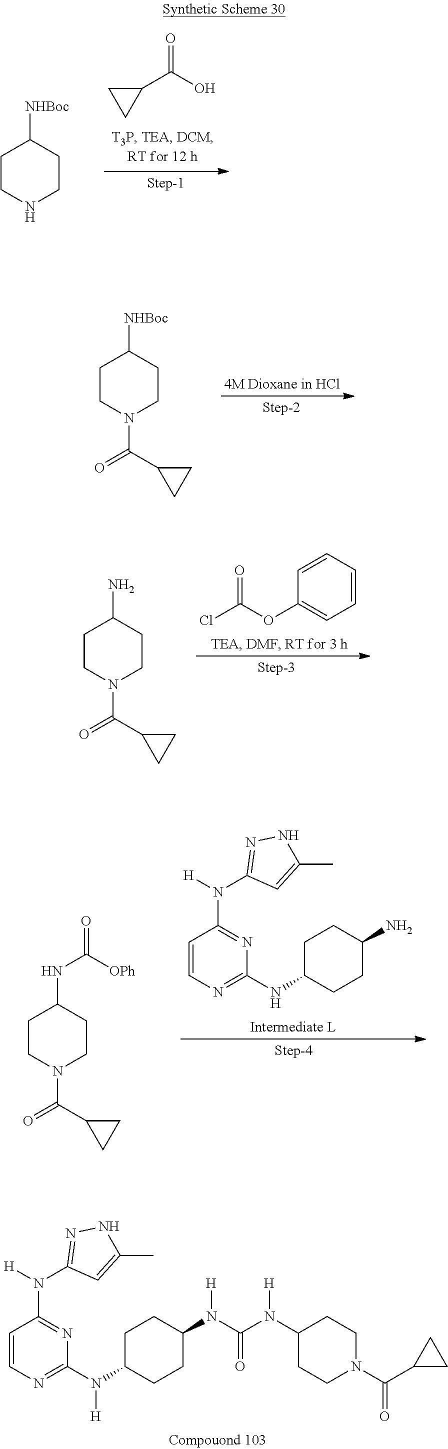

### Step-1: Synthesis of tert-butyl (1-(cyclopropanecarbonyl)piperidin-4-yl)carbamate

To a solution of tert-butyl piperidin-4-ylcarbamate (500 mg, 2.496 mmol) and cyclopropane carboxylic acid (258 mg, 2.995 mmol) in dry dichloromethane (8 mL) was added triethylamine (1.03 mL, 7.489 mmol) drop-wise at 0° C. followed by T₃P (1.2 mL, 3.744 mmol, 50% solution in ethylacetate). The reaction was warmed to room temperature and stirred for 12 h. After the completion of the reaction, the reaction mixture was diluted with dichloromethane, washed with water and brine and dried over anhydrous Na₂SO₄. The resulting solution was then concentrated to yield the crude product. The crude compound was purified by reverse phase preparative HPLC to get tert-butyl (1-(cyclopropanecarbonyl)piperidin-4-yl)carbamate (480 mg, 71.6% yield). LC purity: 85.34%; m/z: 269.3 [M+H]⁺ (Mol. formula C₁₄H₂₄N₂O₃, calcd. mol. wt. 268.36).

### Step-2: Synthesis of (4-aminopiperidin-1-yl)(cyclopropyl)methanone

To a stirred solution of tert-butyl (1-(cyclopropanecarbonyl)piperidin-4-yl)carbamate (480 mg, 1.791 mmol) in dry dichloromethane (5 mL) was added 4M HCl in dioxane drop wise (5 mL) at 0° C. The reaction mixture slowly warmed to room temperature and stirred for 3 h. The solvents were removed under reduced pressure and co-distilled with toluene to obtain crude (4-aminopiperidin-1-yl)(cyclopropyl)methanone (350 mg, quantitative yield). LC purity: 60.61%; m/z: 169.2 [M+H]⁺ (Mol. formula C₉H₁₆N₂O, calcd. mol. wt. 168.24).

### Step-3: Synthesis of phenyl (1-(cyclopropanecarbonyl)piperidin-4-yl)carbamate

To a stirred solution of (4-aminopiperidin-1-yl)(cyclopropyl)methanone (350 mg, 2.083 mmol) in dry DMF (5 ml) was added triethylamine (0.86 mL, 6.249 mmol) and phenyl chloroformate (0.39 mL, 3.124 mmol) at 0° C. The reaction was stirred at ambient temperature for 3 h. The reaction was monitored by TLC, after complete conversion of starting material the reaction mixture was diluted with water, extracted with dichloromethane. The resulting organic layer was washed with brine solution then dried over anhydrous Na₂SO₄ and concentrated to obtain crude compound. The crude thus obtained was washed with pet ether and concentrated to yield phenyl (1-(cyclopropanecarbonyl)piperidin-4-yl)carbamate (150 mg, 25% yield). LC purity: 90.94%; m/z: 289.3 [M+H]⁺ (Mol. formula C₁₆H₂₀N₂O₃, calcd. mol. wt. 288.35).

### Step-4: Synthesis of 1-(1-(cyclopropanecarbonyl)piperidin-4-yl)-3-((1R,4R)-4-((4-((5-methyl-1H-pyrazol-3-yl)amino)pyrimidin-2-yl)amino)cyclohexyl)urea

To a stirred solution of phenyl (1-(cyclopropanecarbonyl)piperidin-4-yl)carbamate (150 mg, 0.5208 mmol) in dry DMF (5 mL) was added triethylamine (0.21 mL, 1.562 mmol). The reaction mixture was heated to 65° C. for 1 h. The reaction mixture was cooled to room temperature and Intermediate L—N²-((1R,4R)-4-aminocyclohexyl)-N⁴-(5-methyl-1H-pyrazol-3-yl)pyrimidine-2,4-diamine (150 mg, 0.5208 mmol) was added. The reaction mixture was heated to 90° C. for 16 h. The reaction mixture was diluted with water and extracted with dichloromethane. The resulting organic layer was washed with brine solution then dried over anhydrous Na₂SO₄ and concentrated to obtain the crude compound. The crude compound was purified by reverse phase preparative HPLC to yield Compound 103 1-(1-(cyclopropanecarbonyl)piperidin-4-yl)-3-((1R,4R)-4-((4-((5-methyl-1H-pyrazol-3-yl)amino)pyrimidin-2-yl)amino)cyclohexyl)urea (12 mg, 5% yield) as a formic acid salt. LC purity: 95.91%; m/z: 482.0 [M+H]⁺ (Mol. formula C₂₄H₃₅N₉O₂, calcd. mol. wt. 481.61). ¹H NMR (400 MHz, CD₃OD): δ 7.76 (s, 1H), 6.25 (bs, 2H), 4.37-4.24 (m, 2H), 3.81-3.75 (m, 2H), 3.56-3.50 (m, 1H), 2.92-2.87 (m, 1H), 2.30 (s, 3H), 2.14-1.88 (m, 7H), 1.49-1.29 (m, 7H), 0.92-0.79 (m, 4H).

### Example 32: Synthesis of Compound 5

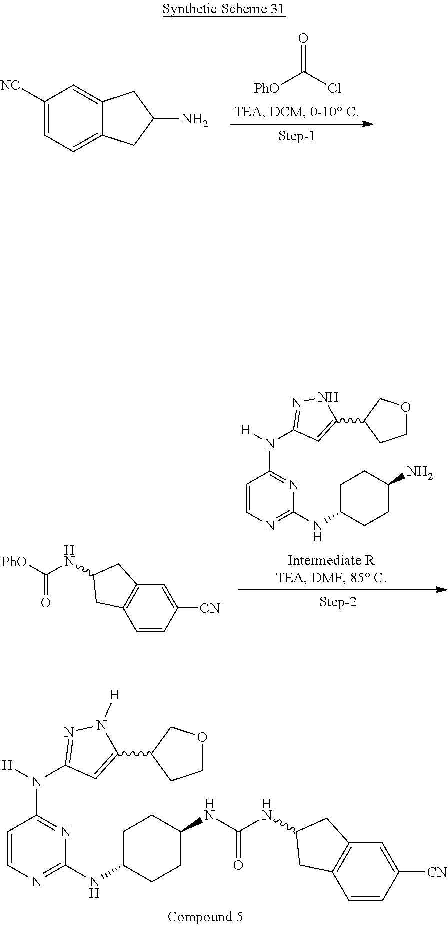 [SMILES: N#Cc1ccc2c(c1)CC(N)C2 ; O=C(Cl)Oc1ccccc1 ; N[C@H]1CC[C@H](Nc2nccc(Nc3cc(C4CCOC4)[nH]n3)n2)CC1 ; N#Cc1ccc2c(c1)CC(NC(=O)Oc1ccccc1)C2 ; N#Cc1ccc2c(c1)CC(NC(=O)N[C@H]1CC[C@H](Nc3nccc(Nc4cc(C5CCOC5)[nH]n4)n3)CC1)C2]

### Step-1: Synthesis of phenyl(5-cyano-2,3-dihydro-1H-indene-2yl)carbamate

To a solution of 2-amino-2,3-dihydro-1H-indene-5-carbonitrile (0.200 g, 1.03 mmol) in dichloromethane (5 mL) was added triethylamine (0.35 mL, 2.5 mmol) dropwise at 0° C., followed by addition of phenyl chloroformate (0.13 mL, 1.03 mmol). The reaction was stirred at 0° C. to 10° C. for 2 h. The progress of the reaction was monitored by TLC and after complete consumption of staring material, the reaction mixture was diluted with dichloromethane and washed with water. The organic layer separated was dried over anhydrous Na₂SO₄, filtered and concentrated under reduced pressure. The solid obtained was triturated with pet ether to deliver phenyl(5-cyano-2,3-dihydro-1H-indene-2yl)carbamate (220 mg, 76.9% yield) as a white solid, which was taken to next step without further purification. LC purity: 91%; m/z: 279 [M+H]⁺ (Mol. C₁₇H₁₄N₂O₂ calcd, mol. wt. 278.31).

### Step-2: Synthesis of 1-(5-cyano-2,3-dihydro-1H-indene-2-yl)-3-((1R,4R)-4-((4-((5-(tetrahydrofuran-3yl)-1H-pyrazol-3-yl)amino)pyrimidine-2-yl)amino)cyclohexyl)urea

To a solution of phenyl (5-cyano-2,3-dihydro-1H-indene-2yl)carbamate (0.162 g, 0.58 mmol) in N,N-dimethyl formamide (5 mL) was added triethylamine (0.24 mL, 1.74 mmol) and the solution was heated at 65° C. for 1 h. After 1 h, the reaction mixture was cooled to room temperature followed by addition of Intermediate R—N²-((1R,4R)-4-aminocyclohexyl)-N⁴-(5-(tetrahydrofuran-3-yl)-1H-pyrazol-3-yl)pyrimidine-2,4-diamine (0.200 g, 0.58 mmol) and the reaction was heated at 85° C. for 16 h. The reaction mixture was cooled to room temperature was added water and extracted with dichloromethane. The organic layer was dried over anhydrous Na₂SO₄, filtered and concentrated under reduced pressure. The residue was purified by reverse phase prep HPLC to yield Compound 5—1-(5-cyano-2,3-dihydro-1H-indene-2-yl)-3-((1R,4R)-4-((4-((5-(tetrahydrofuran-3-yl)-1H-pyrazol-3-yl)amino)pyrimidine-2-yl)amino)cyclohexyl)urea (50 mg, 16.2% yield) as a TFA salt. LC purity: 98.62%; m/z: 528.3 [M+H]⁺ (Mol. C₂₈H₃₃N₉O₂ calcd. mol. wt. 527.63). ¹H NMR (400 MHz, CD₃OD): δ 7.51 (d, J=8 Hz, 1H), 7.59 (s, 1H), 7.53 (d, J=8 Hz, 1H), 7.41 (d, J=7.6 Hz, 1H), 6.63-6.28 (m, 2H), 4.53-4.50 (m, 2H), 4.12-4.08 (m, 2H), 4.01-3.90 (m, 3H), 3.58-3.50 (m, 2H), 2.86-2.84 (m, 2H), 2.48-2.41 (m, 1H), 2.11-2.04 (m, 5H), 1.52-1.46 (m, 2H), 1.38-1.30 (m, 3H).

### Example 33: Synthesis of Compound 7

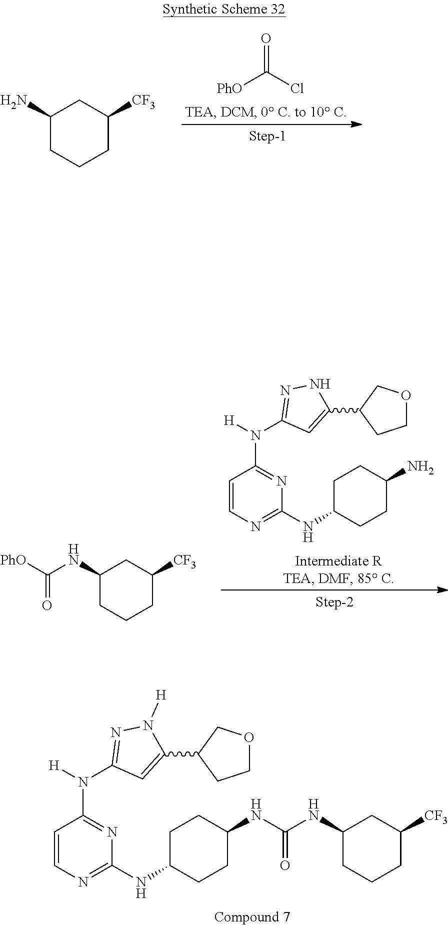 [SMILES: O=C(Cl)Oc1ccccc1 ; N[C@@H]1CCC[C@H](C(F)(F)F)C1 ; N[C@H]1CC[C@H](Nc2nccc(Nc3cc(C4CCOC4)[nH]n3)n2)CC1 ; O=C(N[C@@H]1CCC[C@H](C(F)(F)F)C1)Oc1ccccc1 ; O=C(N[C@@H]1CCC[C@H](C(F)(F)F)C1)N[C@H]1CC[C@H](Nc2nccc(Nc3cc(C4CCOC4)[nH]n3)n2)CC1]

### Step-1: Synthesis of phenyl((1R,3S)-3-(trifluoromethyl)cyclohexyl)carbamate

To a solution of (1R,3S)-3-(trifluromethyl)cyclohexane-1-amine (0.200 g, 0.98 mmol) in dichloromethane (5 mL) was added triethylamine (0.34 mL, 2.45 mmol) dropwise at 0° C., followed by addition of phenyl chloroformate (0.12 mL, 0.98 mmol). The reaction mixture was stirred at 0° C. to 10° C. for 2 h. The reaction was monitored by TLC. After complete consumption of the staring material, the reaction mixture was diluted with dichloromethane and washed with water. The organic layer separated was dried over anhydrous Na₂SO₄, filtered and concentrated under reduced pressure. The solid obtained was triturated with petroleum ether to get phenyl((1R,3S)-3-(trifluoromethyl)cyclohexyl)carbamate (250 mg, 87.1% yield) as a white solid, which was taken to next step without further purification. LC purity: 97.9%; m/z: 288.2 [M+H]⁺ (Mol. C₁₄H₁₆F₃NO₂ calcd, mol. wt. 287.11).

### Step-2: Synthesis of 1-((1R,4R)-4-((4-((5-(tetrahydrofuran-3-yl)1H-pyrazol-3-yl)amino) pyrimidine-2-yl)amino)cyclohexyl)-3-(trifluromethyl)cyclohexanyl)urea

To a solution of phenyl((1R,3S)-3-(trifluoromethyl)cyclohexyl)carbamate (0.167 g, 0.58 mmol) in N,N-dimethyl formamide (5 mL) was added triethylamine (0.243 mL, 1.74 mmol) and heated at 65° C. for 1 h. After 1 h, the reaction mixture was cooled to room temperature followed by addition of N²-((1R,4R)-4-aminocyclohexyl)-N⁴-(5-(tetrahydrofuran-3-yl)-1H-pyrazol-3-yl)pyrimidine-2,4-diamine (0.200 g, 0.58 mmol) and the reaction was heated at 85° C. for 16 h. The reaction mixture was cooled to room temperature was diluted with water and extracted with dichloromethane. The organic layer was dried over anhydrous Na₂SO₄, filtered and concentrated under reduced pressure. The residue was purified by reverse phase prep HPLC to yield Compound 7 1-((1R,4R)-4-((4-((5-(tetrahydrofuran-3-yl)1H-pyrazol-3-yl)amino)pyrimidin-2-yl)amino)cyclohexyl)-3-(trifluromethyl)cyclohexanyl)urea (15 mg, 5% yield) as a TFA salt. LC purity: 99.49%; m/z: 537.2 [M+H]⁺ (Mol. C₂₅H₃₅F₃N₈O₂ calcd. mol. wt. 536.60). ¹H NMR (400 MHz, CD₃OD): δ 7.70 (d, J=7.28 Hz, 1H), 6.65 (s, 1H), 6.30 (d, J=7.3 Hz, 1H), 4.13-4.02 (m, 2H), 3.95-3.86 (m, 2H), 3.56-3.50 (m, 3H), 2.45-2.38 (m, 1H), 2.35-2.28 (m, 1H), 2.16-2.07 (m, 6H), 1.92-1.89 (m, 3H), 1.56-1.46 (m, 2H), 1.42-1.30 (m, 4H), 1.24-1.10 (m, 3H).

### Example 34: Synthesis of Compound 74

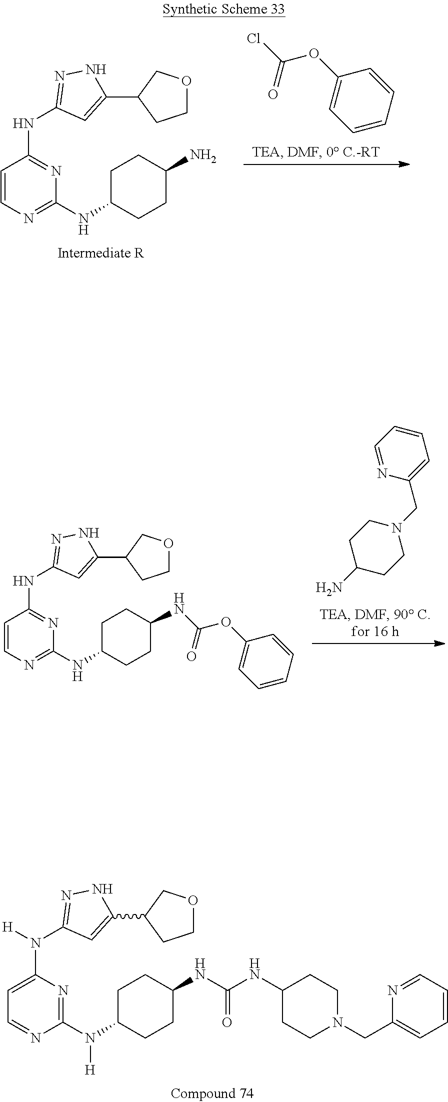 [SMILES: N[C@H]1CC[C@H](Nc2nccc(Nc3cc(C4CCOC4)[nH]n3)n2)CC1 ; O=C(Cl)Oc1ccccc1 ; O=C(N[C@H]1CC[C@H](Nc2nccc(Nc3cc(C4CCOC4)[nH]n3)n2)CC1)Oc1ccccc1 ; NC1CCN(Cc2ccccn2)CC1 ; O=C(NC1CCN(Cc2ccccn2)CC1)N[C@H]1CC[C@H](Nc2nccc(Nc3cc(C4CCOC4)[nH]n3)n2)CC1]

### Step-1: Synthesis of phenyl ((1R,4R)-4-((4-((5-(tetrahydrofuran-3-yl)-1H-pyrazol-3-yl)amino) pyrimidin-2-yl)amino)cyclohexyl)carbamate (IS_18000911_40)

To a solution of Intermediate R—N²-((1R,4R)-4-aminocyclohexyl)-N⁴-(5-(tetrahydrofuran-3-yl)-1H-pyrazol-3-yl)pyrimidine-2,4-diamine (0.200 g, 0.58 mmol) in N,N-dimethyl formamide (5 mL) was added triethylamine (0.8 mL, 1.16 mmol) dropwise at 0° C., followed by addition of phenyl chloroformate (0.09 mL, 0.69 mmol). The reaction mixture was stirred at 0° C. to 10° C. for 2 h. The reaction was monitored by TLC and after complete consumption of staring material, the reaction mixture was diluted with dichloromethane and washed with water. The organic layer separated was dried over anhydrous Na₂SO₄, filtered and concentrated under reduced pressure. The solid obtained was triturated with pet ether to obtain phenyl ((1R,4R)-4-((4-((5-(tetrahydrofuran-3-yl)-1H-pyrazol-3-yl)amino)pyrimidin-2-yl)amino)cyclohexyl)carbamate (220 mg, 81.7% yield) as a white solid. This material was taken to next step without further purification. LC purity: 54.5%; m/z: 463.3 [M]⁺ (Mol. C₂₄H₂₉N₇O₃ calcd. mol. wt. 463.54).

### Step-2: Synthesis of 1-(1-(pyridin-2-ylmethyl)piperdine-4-yl)-3((1R,4R)-4-((4-((5-(tetrahydrofuran-3-yl)-1H-pyrazol-3-yl)amino)pyrimidin-2yl)amino)cyclohexyl)urea

To a solution of phenyl ((1R,4R)-4-((4-((5-(tetrahydrofuran-3-yl)-1H-pyrazol-3-yl)amino)pyrimidin-2-yl)amino)cyclohexyl)carbamate (0.220 g, 0.47 mmol) in N,N-dimethyl formamide (3 mL) was added triethylamine (0.19 mL, 1.41 mmol), and the reaction mixture was heated at 65° C. for 1 h. After 1 h, the reaction mixture was allowed to cool to room temperature, followed by addition of 1-(pyridine-2-ylmethyl)piperdine-4-amine (0.090 g, 0.47 mmol) and the reaction was heated at 85° C. for 16 h. The reaction mixture was cooled to room temperature, was diluted with water and extracted with dichloromethane. The organic layer was dried over anhydrous Na₂SO₄, filtered and concentrated under reduced pressure. The residue was purified by reverse phase prep HPLC to yield Compound 74—1-(1-(pyridin-2-ylmethyl)piperidin-4-yl)-3-((1R,4R)-4-((4-((5-(tetrahydrofuran-3-yl)-1H-pyrazol-3-yl)amino)pyrimidin-2-yl)amino)cyclohexyl)urea (25 mg, 9.3% yield) as a TFA salt. LC purity: 96.7%; m/z: 562.3 [M+H]⁺ (Mol. C₂₉H₄₀N₁₀O₂ calcd. mol. wt. 560.71). ¹H NMR (400 MHz, CD₃OD): δ 8.7 (d, J=4.6 Hz, 1H), 7.94-7.92 (m, 1H), 7.7 (d, J=7.2 Hz, 1H), 7.49-7.46 (m, 2H), 6.65 (s, 1H), 6.30 (d, J=6.6 Hz, 1H), 4.49 (s, 2H), 4.12-4.02 (m, 2H), 3.95-3.89 (m, 4H), 3.58-3.55 (m, 4H), 3.28-3.25 (m, 1H), 2.45-2.40 (m, 1H), 2.18-2.04 (m, 7H), 1.84-1.81 (m, 2H), 1.53-1.36 (m, 4H).

### Example 35: Synthesis of Compound 52

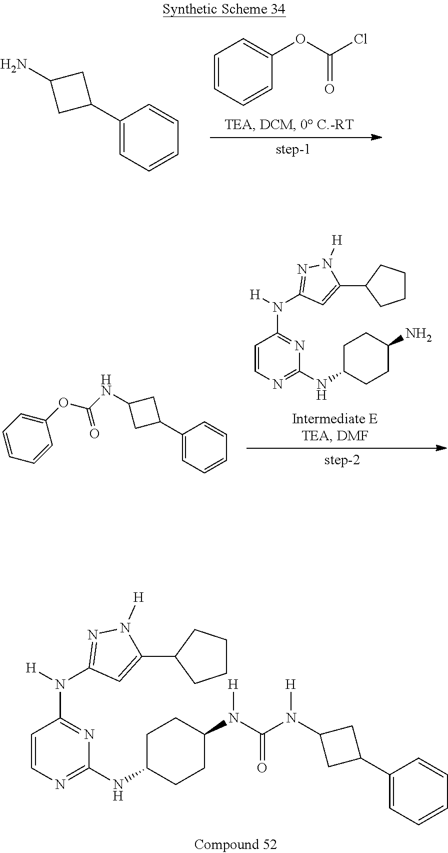 [SMILES: NC1CC(c2ccccc2)C1 ; O=C(Cl)Oc1ccccc1 ; N[C@H]1CC[C@H](Nc2nccc(Nc3cc(C4CCCC4)[nH]n3)n2)CC1 ; O=C(NC1CC(c2ccccc2)C1)Oc1ccccc1 ; O=C(NC1CC(c2ccccc2)C1)N[C@H]1CC[C@H](Nc2nccc(Nc3cc(C4CCCC4)[nH]n3)n2)CC1]

### Step-1: Synthesis of phenyl (3-phenylcyclobutyl)carbamate

To an ice cooled solution of 3-phenylcyclobutan-1-amine (200 mg, 1.36 mmol) in dry DMF (4 mL) was added triethylamine (0.56 mL, 4.08 mmol) and phenyl chloroformate (0.2 mL, 1.63 mmol). The reaction was stirred at room temperature for 3 h. The progress of the reaction was monitored by TLC analysis. After completion of the reaction, the reaction mixture was quenched with water and extracted with ethyl acetate. The combined organic layers were washed with brine solution then dried over anhydrous Na₂SO₄ and concentrated under reduced pressure to obtain phenyl (3-phenylcyclobutyl)carbamate (320 mg, crude) as a light yellow solid. The crude material was taken to the next step without any purification. LC purity: 18.1%; m/z: 268.1 [M+H]⁺ (Mol. formula C₁₇H₁₇NO₂, calcd. mol. wt. 267.33).

### Step-2: Synthesis of 1-((1R,4R)-4-((4-((5-cyclopentyl-1H-pyrazol-3-yl)amino)pyrimidin-2-yl)amino)cyclohexyl)-3-(3-phenylcyclobutyl)urea

To a stirred solution of phenyl (3-phenylcyclobutyl)carbamate (320 mg, 1.19 mmol) in dry DMF (3 mL) was added triethylamine (0.49 mL, 3.59 mmol). The reaction mixture was heated to 65° C. for 1 h. The reaction mixture was cooled to room temperature and added Intermediate E—N²-((1R,4R)-4-aminocyclohexyl)-N⁴-(5-cyclopentyl-1H-pyrazol-3-yl)pyrimidine-2,4-diamine (326 mg, 0.958 mmol). The reaction was heated to 90° C. for 18 h. The reaction mixture was monitored by TLC, starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 52—1-((1R,4R)-4-((4-((5-cyclopentyl-1H-pyrazol-3-yl)amino)pyrimidin-2-yl)amino)cyclohexyl)-3-(3-phenylcyclobutyl)urea (35 mg, 6% yield) as a TFA salt. LC purity: 96.4%; m/z: 515.3 [M+H]⁺ (Mol. formula C₂₉H₃₈N₈O, calcd. mol. wt. 514.68). ¹H NMR (400 MHz, CD₃OD): δ 7.67 (d, J=7.2 Hz, 1H), 7.29-7.26 (m, 4H), 7.17-7.15 (m, 1H), 6.29-6.27 (m, 2H), 4.31-4.28 (m, 1H), 3.91-3.85 (m, 1H), 3.54-3.51 (m, 2H), 3.30-3.13 (m, 1H), 2.52-2.46 (m, 2H), 2.38-2.31 (m, 2H), 2.12-1.98 (m, 6H), 1.84-1.67 (m, 6H), 1.52-1.29 (m, 4H).

### Example 36: Synthesis of Compound 73

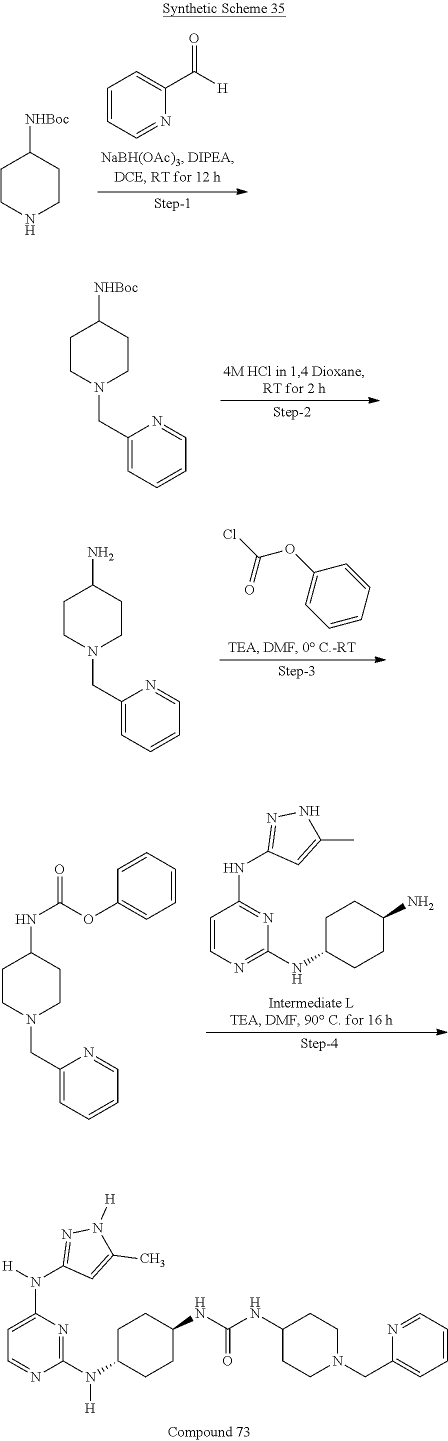

### Step-1: Synthesis of tert-butyl (1-(pyridin-2-ylmethyl)piperidin-4-yl)carbamate

To a solution of 4-(boc-amino)piperidine (2.0 g, 9.986 mmol) in 1,2 dichloroethane (60 mL) under nitrogen was added 2-pyridinecarboxaldehyde (0.96 mL, 9.986 mmol) and DIPEA (1.76 mL, 9.986 mmol) followed by sodium triacetoxyborohydride (2.54 g, 11.983 mmol). The reaction mixture was stirred at room temperature for 12 h. The solvent was removed in vacuo and the residue dissolved in dichloromethane. The organic layer was washed with sodium bicarbonate solution, washed with brine, separated, dried over anhydrous Na₂SO₄, filtered and concentrated to give a yellow solid. This was dissolved in dry methanol and to this mixture added a solution of 2M HCl in diethyl ether (12 mL). The reaction was stirred at room temperature for overnight. The resulting precipitate was filtered off, washed with diethyl ether and dried in vacuo to yield the tert-butyl (1-picolinoylpiperidin-4-yl)carbamate as a green solid (1.1 g, 37% yield). LC purity: 67.3%; m/z: 292.2 [M+H]⁺ (Mol. formula C₁₆H₂₅N₃O₂, calcd. mol. wt. 291.40).

### Step-2: Synthesis of 1-(pyridin-2-ylmethyl)piperidin-4-amine

To a stirred solution of tert-butyl (1-(pyridin-2-ylmethyl)piperidin-4-yl)carbamate (1.1 g, 3.43 mmol) in anhydrous dichloromethane (20 mL). The reaction mixture was cooled to 0° C. and added HCl in dioxane (10 mL, 4M solution). The reaction mixture was allowed to stir at room temperature for 2 h. The progress of the reaction mixture was monitored by TLC, after complete consumption of starting material, the resulting mixture was concentrated to give 1-(pyridin-2-ylmethyl)piperidin-4-amine as a hydrochloride salt (1.1 g, quantitative yield). LC purity: 99.6%; m/z: 192.3 [M+H]⁺ (Mol. formula C₁₁H₁₇N₃, calcd. mol. wt. 191.28).

### Step-3: Synthesis of phenyl (1-(pyridin-2-ylmethyl)piperidin-4-yl)carbamate

To a solution of 1-(pyridin-2-ylmethyl)piperidin-4-amine (1.1 g, 5.76 mmol) in dry DMF (15 mL) at 0° C. was added triethylamine (3.4 mL, 28.8 mmol) and phenyl chloroformate (0.86 mL, 6.91 mmol). The reaction mixture was stirred at room temperature for 2 h. The progress of the reaction was monitored by TLC analysis. After completion of the reaction, the reaction mixture was quenched with water and extracted with ethyl acetate. The combined organic layers were washed with brine solution then dried over anhydrous Na₂SO₄ and concentrated under reduced pressure to obtain phenyl (1-(pyridin-2-ylmethyl)piperidin-4-yl)carbamate (600 mg, 33% yield) as a brown liquid. LC purity: 98.99%; m/z: 312.2 [M+H]⁺ (Mol. formula C₁₈H₂₁N₃O₂, calcd. mol. wt. 311.39).

### Step-4: Synthesis of 1-((1R,4R)-4-((4-((5-methyl-1H-pyrazol-3-yl)amino)pyrimidin-2-yl)amino)cyclohexyl)-3-(1-(pyridin-2-ylmethyl)piperidin-4-yl)urea

To a stirred solution of phenyl (1-(pyridin-2-ylmethyl)piperidin-4-yl)carbamate (600 mg, 1.926 mmol) in anhydrous DMF (6 mL) was added tri ethylamine (0.8 mL, 5.780 mmol). The reaction mixture was heated to 140° C. for 2 h. The reaction mixture was cooled to room temperature and added Intermediate L—N2-((1R,4R)-4-aminocyclohexyl)-N4-(5-methyl-1H-pyrazol-3-yl)pyrimidine-2,4-diamine (553 mg, 1.926 mmol). The reaction was heated to 90° C. for 16 h. The reaction mixture was monitored by TLC, starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 36—1-((1R,4R)-4-((4-((5-methyl-1H-pyrazol-3-yl)amino)pyrimidin-2-yl)amino)cyclohexyl)-3-(1-(pyridin-2-ylmethyl) piperidin-4-yl)urea (25 mg, 20% yield) as a TFA salt. LC purity: 96.44%; m/z: 505.3 [M+H]⁺ (Mol. formula C₂₆H₃₆N₁₀O, calcd. mol. wt. 504.64). ¹H NMR (400 MHz, CD₃OD): δ 8.47 (s, 1H), 7.81-7.77 (m, 2H), 7.51 (d, J=7.6 Hz, 1H), 7.30-7.27 (m, 1H), 6.29-6.13 (m, 2H), 3.71-3.65 (m, 3H), 3.57-3.51 (m, 2H), 2.89-2.80 (m, 2H), 2.28-2.23 (m, 6H), 2.11-2.08 (m, 2H), 2.01-1.98 (m, 2H), 1.88-1.86 (m, 2H), 1.53-1.43 (m, 2H), 1.39-1.26 (m, 3H).

### Example 37: Synthesis of Compound 16

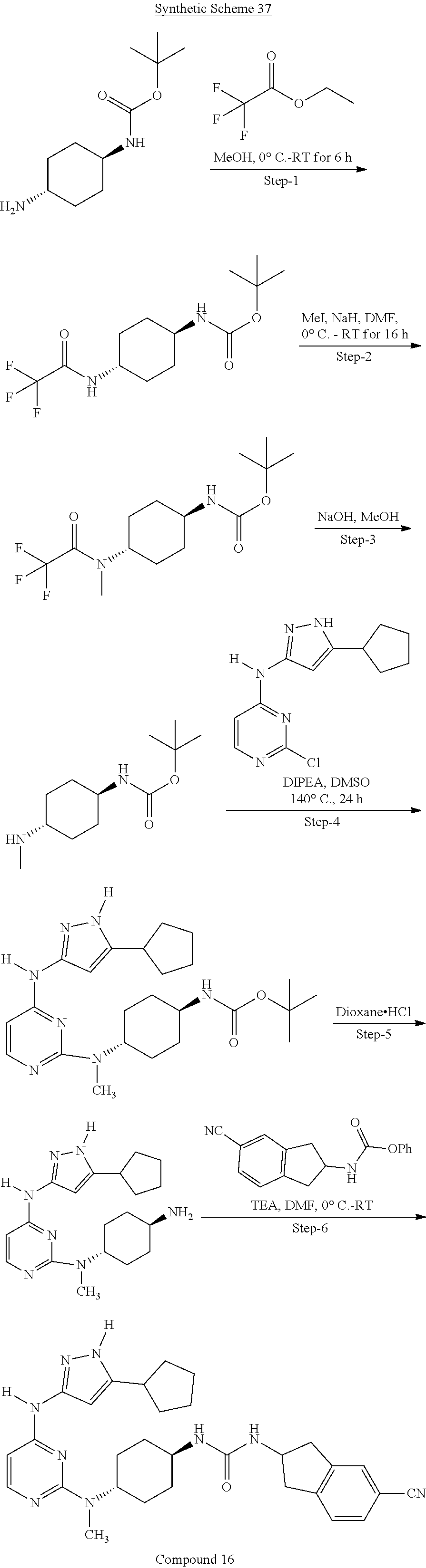

### Step-1: Synthesis of tert-butyl ((1R,4R)-4-(2,2,2-trifluoroacetamido)cyclohexyl)carbamate

To a solution of tert-butyl ((1R,4R)-4-aminocyclohexyl)carbamate (1 g, 4.67 mmol) in methanol (10 mL) was cooled to 0° C. and added ethyl trifluoro acetate (1.71 mL, 23.1 mmol). The reaction was stirred at ambient temperature for 3 h. Completion of the reaction was monitored by TLC, after completion of the reaction, the mixture was filtered off and washed with cold methanol to yield tert-butyl ((1R,4R)-4-(2,2,2-trifluoroacetamido)cyclohexyl)carbamate (1.1 g, 70% yield). LC purity: 99.7%; m/z: 309.0 [M−H]⁺ (Mol. formula C₂₂H₃₄N₈O₂, calcd. mol. wt. 310.15). ¹H NMR (300 MHz, DMSO-d₆): δ 9.25 (d, J=6 Hz, 1H), 6.75 (d, J=7.8 Hz, 1H), 3.54 (d, J=7.2 Hz, 1H), 3.17 (d, J=5.4 Hz, 1H), 1.77-1.75 (m, 4H), 1.36 (s, 9H), 1.30-1.18 (m, 4H).

### Step-2: Synthesis of tert-butyl ((1R,4R)-4-(2,2,2-trifluoro-N-methylacetamido) cyclohexyl)carbamate

To a suspension of tert-butyl ((1R,4R)-4-(2,2,2-trifluoroacetamido)cyclohexyl)carbamate (1.0 g, 3.21 mmol) in dry DMF (10 mL) was added sodium hydride (0.16 g, 6.81 mmol) in portionwise at 0° C. The slightly cloudy reaction mixture was stirred at room temperature for 20 minutes. Then the reaction mixture was cooled to 0° C. and added methyl iodide (0.24 mL, 3.81 mmol) dropwise. The reaction was stirred at room temperature for 16 h. After completion of the reaction (monitored by TLC), the reaction mixture was poured in to an ice cold water and neutralized with citric acid. The precipitate formed was filtered off, washed with water and dried under vacuum to get tert-butyl ((1R,4R)-4-(2,2,2-trifluoro-N-methylacetamido)cyclohexyl)carbamate (0.85 g, 85% yield). LC purity: 99.3%; m/z: 225.2 [M−Boc]⁺, 269.1 [M−^{t}Bu]⁺ (Mol. formula C₁₄H₂₃N₂O₃, calcd. mol. wt. 324.34). ¹H NMR (300 MHz, DMSO-d₆): Not clean.

### Step-3: Synthesis of tert-butyl ((1R,4R)-4-(methylamino)cyclohexyl)carbamate

A Suspension of tert-butyl ((1R,4R)-4-(2,2,2-trifluoro-N-methylacetamido)cyclohexyl) carbamate (600 mg, 1.849 mmol) in methanol (6 mL) was heated at 50° C. with stirring until clear solution formed. Then 0.6 mL 2N NaOH solution was added to it with constant stirring. A slightly cloudy solution obtained, which was stirred for further 2-3 h at ambient temperature. The reaction mixture was concentrated, the residue was added to 2N citric acid solution and extracted with 10% methanol in dichloromethane. Then it is made alkaline with 2N NaOH solution and again extracted with 10% methanol in dichloromethane. The combined organic layers dried over anhydrous Na₂SO₄ and concentrated to get tert-butyl ((1R,4R)-4-(methylamino)cyclohexyl)carbamate (600 mg, quantitative) as a white solid. LC purity: 95%; m/z: 229.2 [M+H]⁺ (Mol. formula C₁₂H₂₄N₂O₂, calcd. mol. wt. 228.34). ¹H NMR (400 MHz, DMSO-d₆): δ 6.69 (d, J=7.6 Hz, 1H), 3.24-3.14 (m, 1H), 2.25 (s, 3H), 2.20-2.17 (m, 1H), 1.86-1.81 (m, 2H), 1.75-1.72 (m 2H), 1.39 (s, 9H), 1.24-1.15 (m, 2H), 1.12-1.02 (m, 3H).

### Step-4: Synthesis of tert-butyl ((1R,4R)-4-((4-((5-cyclopentyl-1H-pyrazol-3-yl)amino)pyrimidin-2-yl)(methyl)amino)cyclohexyl)carbamate

To a stirred solution of 2-chloro-N-(5-cyclopentyl-1H-pyrazol-3-yl)pyrimidine-4-amine (600 mg, 2.281 mmol) in dry DMSO (6.0 mL) in a sealed tube was added DIPEA (0.78 mL, 4.562 mmol). The reaction mixture was cooled to 0° C. and added tert-butyl ((1R,4R)-4-(methylamino) cyclohexyl)carbamate (0.52 g, 2.281 mmol). The reaction was heated to 140° C. for 16 h. The reaction mixture was quenched with ice cold water and extracted with ethyl acetate. The resulting organic layer was washed with brine solution then dried over anhydrous Na₂SO₄ and concentrated to obtain crude compound. The obtained crude product was purified by using flash column chromatography 230-400 silica mesh and methanol in DCM as a eluent to yield tert-butyl ((1R,4R)-4-((4-((5-cyclopentyl-1H-pyrazol-3-yl)amino)pyrimidin-2-yl)(methyl) amino)cyclohexyl)carbamate (300 mg, 30%). LC purity: 72.98%; m/z: 456.3 [M+H]⁺ (Mol. formula C₂₄H₃₇N₇O₂, calcd. mol. wt. 455.61).

### Step-5: Synthesis of N2-((1R,4R)-4-aminocyclohexyl)-N4-(5-cyclopentyl-1H-pyrazol-3-yl)-N2-methylpyrimidine-2,4-diamine

To a stirred solution of tert-butyl ((1R,4R)-4-((4-((5-cyclopentyl-1H-pyrazol-3-yl)amino)pyrimidin-2-yl)(methyl)amino)cyclohexyl)carbamate (0.3 g, 0.659 mmol) in dry dichloromethane (6 mL). The reaction mixture was cooled to 0° C. and added HCl in dioxane (3 mL, 4M solution). The reaction was allowed to stir at room temperature for 2 h. The progress of the reaction was monitored by TLC, after complete consumption of starting material, the resulting mixture was concentrated to N²-((1R,4R)-4-aminocyclohexyl)-N⁴-(5-cyclopentyl-1H-pyrazol-3-yl)-N2-methylpyrimidine-2,4-diamine as a hydrochloride salt (300 mg, quantitative yield). LC purity: 82.6%; m/z: 356.2 [M+H]⁺ (Mol. formula C₁₉H₂₉N₇, calcd. mol. wt. 355.49).

### Step-6: Synthesis of 1-(5-cyano-2,3-dihydro-1H-inden-2-yl)-3-((1R,4R)-4-((4-((5-cyclopentyl-1H-pyrazol-3-yl)amino)pyrimidin-2-yl)(methyl)amino)cyclohexyl)urea

To a stirred solution of phenyl (5-cyano-2,3-dihydro-1H-inden-2-yl)carbamate (281 mg, 1.014 mmol) in dry DMF (6 mL) was added triethylamine (0.58 mL, 4.225 mmol). The reaction mixture was heated to 80° C. for 1 h. The reaction mixture was cooled to room temperature and N2-((1R,4R)-4-aminocyclohexyl)-N4-(5-cyclopentyl-1H-pyrazol-3-yl)-N2-methylpyrimidine-2,4-diamine (300 mg, 0.845 mmol) was added. The reaction was heated to 85° C. for 16 h. The reaction mixture was monitored by TLC, and starting material was consumed. The reaction mixture was diluted with water and extracted with dichloromethane. The resulting organic layer was washed with brine solution then dried over anhydrous Na₂SO₄ and concentrated to obtain the crude compound. The crude compound was purified by reverse phase prep HPLC to yield Compound 16—1-(5-cyano-2,3-dihydro-1H-inden-2-yl)-3-((1R,4R)-4-((4-((5-cyclopentyl-1H-pyrazol-3-yl)amino)pyrimidin-2-yl)(methyl)amino)cyclohexyl)urea (35 mg, 8%) as a TFA salt. LC purity: 95.08%; m/z: 540.2 [M+H]⁺ (Mol. formula C₃₀H₃₇N₉O, calcd. mol. wt. 539.69). ¹H NMR (400 MHz, CD₃OD): δ 7.72 (d, J=6.8 Hz, 1H), 7.60 (s, 1H), 7.54 (d, J=7.6 Hz, 1H), 7.42 (d, J=8 Hz, 1H), 6.48 (s, 1H), 6.35 (d, J=6.8 Hz, 1H), 4.56-4.50 (m, 1H), 3.54-3.39 (m, 1H), 3.15-2.91 (m, 5H), 2.89-2.83 (m, 2H), 2.18-2.12 (m, 4H), 1.83-1.70 (m, 12H), 1.39-1.30 (m, 3H).

### Chiral Separation of 1-(5-cyano-2,3-dihydro-1H-inden-2-yl)-3-((1R,4R)-4-((4-((5-cyclopentyl-1H-pyrazol-3-yl)amino)pyrimidin-2-yl)(methyl)amino)cyclohexyl)urea

The individual stereoisomers were separated by chiral SFC using the Column: Chiral Pak OX-H Flowrate: 5 ml/min, Co-Solvent: 50%, Co-Solvent: Methanol, Injected Volume: 15 μl, Outlet Pressure: 100 bar, Temperature: 35° C. The first eluted (RT=7.72) fractions were collected and concentrated under reduced pressure and the second eluted fractions were collected and concentrated under reduced pressure (RT=11.35).

### First Eluting Isomer

LC Purity: 99.2%; m/z: 540.2 [M+H]⁺ (Mol. formula C₃₀H₃₇N₉O, calcd. mol. wt. 539.69). ¹H NMR (400 MHz, CD₃OD): δ 7.84 (s, 1H), 7.58 (s, 1H), 7.53 (d, J=8 Hz, 1H), 7.41 (d, J=8 Hz, 1H), 6.55-6.12 (m, 2H), 4.54-4.51 (m, 2H), 3.52-3.46 (m, 1H), 3.32-3.31 (m, 1H), 3.29-3.06 (m, 2H), 2.99 (s, 3H), 2.88-2.82 (m, 2H), 2.08-2.05 (m, 4H), 1.94-1.69 (m, 10H), 1.40-1.30 (m, 2H).

### Second Eluting Isomer

LC Purity: 97.6%; m/z: 540.2 [M+H]⁺ (Mol. formula C₃₀H₃₇N₉O, calcd. mol. wt. 539.69). ¹H NMR (400 MHz, CD₃OD): δ 7.84 (s, 1H), 7.58 (s, 1H), 7.53 (d, J=8 Hz, 1H), 7.41 (d, J=8 Hz, 1H), 6.55-6.12 (m, 2H), 4.54-4.51 (m, 2H), 3.52-3.46 (m, 1H), 3.32-3.31 (m, 1H), 3.29-3.06 (m, 2H), 2.99 (s, 3H), 2.88-2.82 (m, 2H), 2.08-2.05 (m, 4H), 1.94-1.65 (m, 10H), 1.37-1.29 (m, 2H).

### Example 38: Synthesis of Compound 8

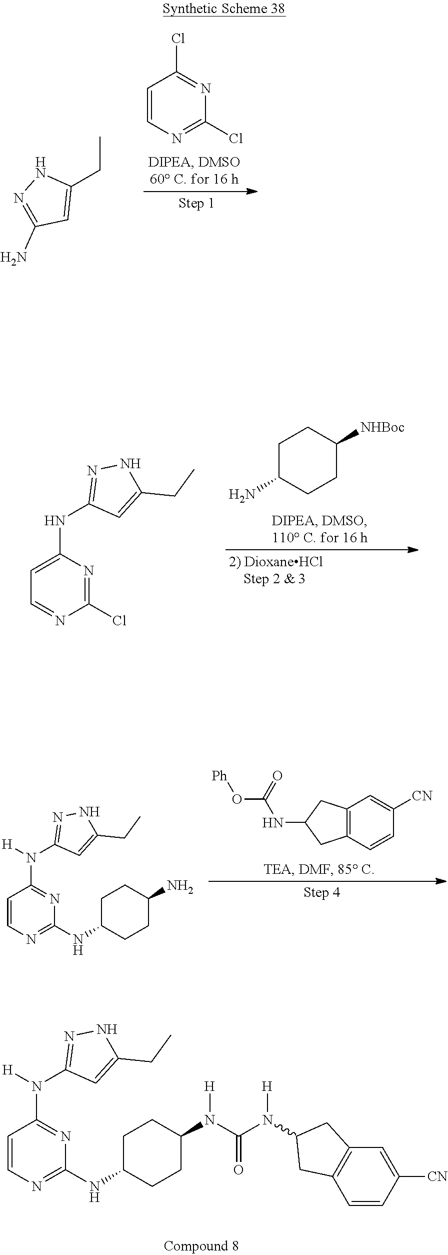

### Step-1: Synthesis of 2-chloro-N-(5-ethyl-1H-pyrazol-3-yl)pyrimidin-4-amine

A mixture of 2,4-dichloropyrimidine (1.6 g, 10.8 mmol), 5-ethyl-1H-pyrazol-3-amine (1.0 g, 9.00 mmol) and DIPEA (2.3 ml, 13.5 mmol) in DMSO (10 mL) were stirred at 60° C. for 16 h. The progress of the reaction was monitored by TLC, after complete consumption of starting material, the reaction mixture was cooled to room temperature and water was added. The solid precipitated was filtered, washed with pet ether and dried under vacuum to yield 2-chloro-N-(5-ethyl-1H-pyrazol-3-yl)pyrimidine-4-amine (1.4 g, 70% yield). LC purity: 91.7%; m/z: 224.2 [M+H]⁺ (Mol. formula C₉H₁₀ClN₅, calcd. mol. wt. 223.66). ¹H NMR (300 MHz, DMSO-d₆): δ 12.16 (s, 1H), 10.30 (s, 1H), 8.15 (s, 1H), 7.24 (s, 1H), 6.16 (s, 1H), 2.60-2.50 (m, 2H), 1.21-1.16 (m, 3H).

### Step-2: Synthesis of tert-butyl ((1R,4R)-4-((4-((5-ethyl-1H-pyrazol-3-yl)amino)pyrimidin-2-yl)amino)cyclohexyl)carbamate

A mixture of 2-chloro-N-(5-ethyl-1H-pyrazol-3-yl)pyrimidine-4-amine (1.0 g, 4.48 mmol) tert-butyl ((1r,4r)-4-aminocyclohexyl)carbamate (1.14 g, 5.36 mmol) and DIPEA (1.14 mL, 6.71 mmol) in DMSO (10 mL) were stirred at 110° C. for 16 h. The reaction mixture was monitored by LCMS. The reaction mixture was cooled to room temperature and extracted with dichloromethane. The organic layer was washed with water and brine and dried over anhydrous Na₂SO₄ and concentrated to obtain the crude compound. The crude compound was purified by Biotage iolera using (60-120 silica gel) with gradient elution of 0-10% ethyl acetate in pet ether to yield tert-butyl ((1R,4R)-4-((4-((5-ethyl-1H-pyrazol-3-yl)amino)pyrimidin-2-yl)amino)cyclohexyl)carbamate (0.6 g, 33% yield). LC purity: 89.98%; m/z: 402.3 [M+H]⁺ (Mol. formula C₂₀H₃₁N₇O₂, calcd. mol. wt. 401.52). ¹H NMR (300 MHz, DMSO-d₆): δ 11.87 (s, 1H), 9.31 (s, 1H), 7.77 (s, 1H), 6.76 (s, 1H), 6.42-6.15 (m, 2H), 3.61-3.55 (m, 1H), 3.33-3.20 (m, 1H), 2.56-2.50 (m, 2H), 1.92-1.80 (m, 4H), 1.38 (s, 9H), 1.25-1.17 (m, 3H), 1.08-0.93 (m, 3H).

### Step-3: Synthesis of N²-((1R,4R)-4-aminocyclohexyl)-N⁴-(5-ethyl-1H-pyrazol-3-yl)pyrimidine-2,4-diamine

To a stirred solution of tert-butyl ((1r,4r)-4-((4-((5-ethyl-1H-pyrazol-3-yl)amino)pyrimidin-2-yl)amino)cyclohexyl)carbamate (0.3 g, 0.725 mmol) in dry dichloromethane (6 mL). The reaction mixture was cooled to 0° C. and added HCl in dioxane (3 mL, 4M solution). The reaction was allowed to stir at room temperature for 2 h. The resulting mixture was concentrated to N²-((1R,4R)-4-aminocyclohexyl)-N⁴-(5-ethyl-1H-pyrazol-3-yl)pyrimidine-2,4-diamine as (0.3 g, quantitative yield) a HCl salt. LC purity: 93.5%; m/z: 302.2 [M+H]⁺ (Mol. formula C₁₅H₂₃N₇, calcd. mol. wt. 301.40). ¹H NMR (300 MHz, DMSO-d₆): δ 12.21 (s, 1H), 11.22 (s, 1H), 8.56 (s, 1H), 8.23-7.80 (m, 4H), 6.43-6.30 (m, 2H), 3.56 (s, 3H), 3.03-2.88 (m, 1H), 2.03-1.71 (m, 4H), 1.59-1.43 (m, 4H), 1.25-1.12 (m, 3H).

### Step-6: Synthesis of 1-(5-cyano-2,3-dihydro-1H-inden-2-yl)-3-((1R,4R)-4-((4-((5-ethyl-1H-pyrazol-3-yl)amino)pyrimidin-2-yl)amino)cyclohexyl)urea

To a stirred solution of phenyl (5-cyano-2,3-dihydro-1H-inden-2-yl)carbamate (0.221 g, 0.798 mmol) in dry DMF (4.0 mL) was added triethylamine (0.3 mL, 1.99 mmol). The reaction mixture was heated to 65° C. for 1 h. The reaction mixture was cooled to room temperature and added N²-((1R,4R)-4-aminocyclohexyl)-N⁴-(5-ethyl-1H-pyrazol-3-yl)pyrimidine-2,4-diamine (0.2 g, 0.665 mmol). The reaction was heated to 85° C. for 16 h. The progress of the reaction was monitored by TLC and after complete conversion of starting material, the reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 8—1-(5-cyano-2,3-dihydro-1H-inden-2-yl)-3-((1R,4R)-4-((4-((5-ethyl-1H-pyrazol-3-yl)amino)pyrimidin-2-yl)amino)cyclohexyl)urea (225 mg, 9%) as a TFA salt. LC purity: 99.01%; m/z: 486.3 [M+H]⁺ (Mol. formula C₂₆H₃₁N₉O, calcd. mol. wt. 485.60). ¹H NMR (400 MHz, CD₃OD): δ 7.69 (s, 1H), 7.56 (s, 1H), 7.51 (d, J=8.0 Hz, 1H), 7.39 (d, J=7.6 Hz, 1H), 6.60 (s, 1H), 6.33 (s, 1H), 4.59-4.49 (m, 1H), 3.82-3.85 (m, 1H), 3.57-3.49 (m, 1H), 3.29-3.27 (m, 2H), 2.90-2.82 (m, 2H), 2.73-2.67 (m, 2H), 2.14-2.04 (m, 4H), 1.55-1.46 (m, 2H), 1.34-1.26 (m, 5H).

### Example 39: Synthesis of Compound 21

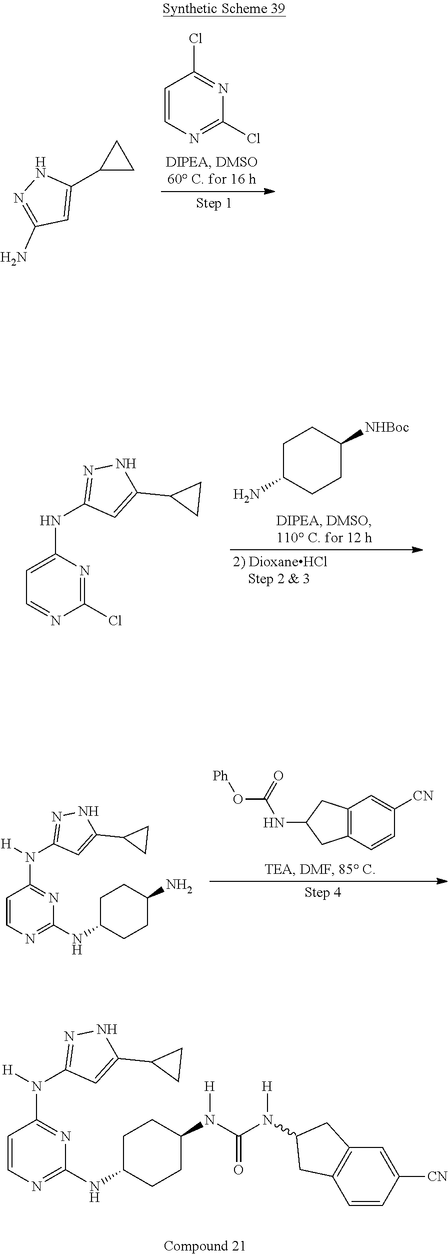

### Step-1: Synthesis of 2-chloro-N-(5-cyclopropyl-1H-pyrazol-3-yl)pyrimidin-4-amine

A mixture of 2,4-dichloropyrimidine (1.45 g, 9.76 mmol), 5-cyclopropyl-1H-pyrazol-3-amine (1.0 g, 8.13 mmol), and DIPEA (2.12 mL, 12.2 mmol) in DMSO (10 mL) were stirred at 60° C. for 16 h. The progress of the reaction was monitored by TLC after complete consumption of starting material, the reaction mixture was cooled to room temperature, water was added, and solid product was precipitated. The solid was filtered, washed with pet ether and dried under vacuum to yield 2-chloro-N-(5-cyclopropyl-1H-pyrazol-3-yl)pyrimidin-4-amine (1.2 g, 63% yield). LC purity: 96.69%; m/z: 210.2 [M+H]⁺ (Mol. formula C₁₀H₁₀ClN₅, calcd. mol. wt. 235.68). ¹H NMR (300 MHz, DMSO-d₆): δ 12.18 (s, 1H), 10.26 (s, 1H), 8.14 (d, J=5.7 Hz, 1H), 7.20 (s, 1H), 6.01 (s, 1H), 1.92-1.86 (m, 1H), 0.96-0.89 (m, 2H), 0.71-0.69 (m, 2H).

### Step-2: Synthesis of tert-butyl ((1R,4R)-4-((4-((5-cyclopropyl-1H-pyrazol-3-yl)amino)pyrimidin-2-yl)amino)cyclohexyl)carbamate

A mixture of 2-chloro-N-(5-cyclopropyl-1H-pyrazol-3-yl)pyrimidine-4-amine (0.9, 3.82 mmol), tert-butyl ((1r,4r)-4-aminocyclohexyl)carbamate (0.98 g, 4.59 mmol) and DIPEA (0.98 mL, 5.73 mmol) in DMSO (10 mL) were stirred at 110° C. for 16 h. The reaction mixture was monitored by LCMS, The reaction mixture was cooled to room temperature was added water and extracted with dichloromethane. The organic layer was dried over anhydrous Na₂SO₄ and concentrated to get residue. The residue was purified by silica gel column chromatography with gradient elution of 0-10% ethyl acetate in pet ether to yield tert-butyl ((1R,4R)-4-((4-((5-cyclopropyl-1H-pyrazol-3-yl)amino)pyrimidin-2-yl)amino)cyclohexyl)carbamate (0.54 g, 35% yield). LC purity: 87.4%; m/z: 414.2 [M+H]⁺ (Mol. formula C₂₁H₃₁N₇O₂, calcd. mol. wt. 413.53). ¹H NMR (300 MHz, DMSO-d₆): δ 9.26 (s, 1H), 7.75 (s, 1H), 6.75 (s, 1H), 6.41-6.12 (m, 3H), 3.59-3.45 (m, 1H), 3.17 (m, 1H), 1.92-1.80 (m, 5H), 1.38-1.25 (m, 14H), 0.91-0.90 (m, 2H), 0.67-0.58 (m, 2H).

### Step-3: Synthesis of N²-((1R,4R)-4-aminocyclohexyl)-N⁴-(5-cyclopropyl-1H-pyrazol-3-yl)pyrimidine-2,4-diamine

To a stirred solution of tert-butyl ((1R,4R)-4-((4-((5-cyclopropyl-1H-pyrazol-3-yl)amino)pyrimidin-2-yl)amino)cyclohexyl)carbamate (0.3 g, 0.725 mmol) in dichloromethane (6 mL) was cooled to 0° C. and added HCl in dioxane (3 mL, 4M solution). The reaction was allowed to stir at room temperature for 2 h. The progress of the reaction was monitored by TLC, and after complete consumption of starting material, the resulting mixture was concentrated to N²-((1R,4R)-4-aminocyclohexyl)-N⁴-(5-cyclopropyl-1H-pyrazol-3-yl)pyrimidine-2,4-diamine (300 mg, quantitative yield) as a HCl salt. LC purity: 89.2%; m/z: 314.2 [M+H]⁺ (Mol. formula C₁₆H₂₃N₇, calcd. mol. wt. 313.41).

### Step-4: Synthesis of 1-(5-cyano-2,3-dihydro-1H-inden-2-yl)-3-((1R,4R)-4-((4-((5-cyclopropyl-1H-pyrazol-3-yl)amino)pyrimidin-2-yl)amino)cyclohexyl)urea

To a stirred solution of phenyl (5-cyano-2,3-dihydro-1H-inden-2-yl)carbamate (0.21 g, 0.767 mmol) in dry DMF (4.0 mL) was added triethylamine (0.27 mL, 1.92 mmol). The reaction mixture was heated to 65° C. for 1 h. The reaction mixture was cooled to room temperature and N²-((1r,4r)-4-aminocyclohexyl)-N⁴-(5-cyclopropyl-1H-pyrazol-3-yl)pyrimidine-2,4-diamine (0.2 g, 1.92 mmol) was added. The reaction was heated to 85° C. for 16 h. The progress of the reaction was monitored by TLC, and after complete consumption of starting material, the reaction mixture was diluted with water and extracted with dichloromethane. The resulting organic layer was washed with brine solution then dried over anhydrous Na₂SO₄ and concentrated to obtain the crude compound. The crude compound was purified by reverse phase prep HPLC to yield Compound 21—1-(5-cyano-2,3-dihydro-1H-inden-2-yl)-3-((1R,4R)-4-((4-((5-cyclopropyl-1H-pyrazol-3-yl)amino)pyrimidin-2-yl)amino)cyclohexyl)urea (35 mg, 12%) as a TFA salt. LC purity: 98.98%; m/z: 498.3 [M+H]⁺ (Mol. formula C₂₇H₃₁N₉O, calcd. mol. wt. 497.61). ¹H NMR (400 MHz, CD₃OD): δ 7.68 (d, J=6 Hz, 1H), 7.56 (s, 1H), 7.51 (d, J=7.6 Hz, 1H), 7.39 (d, J=7.6 Hz, 1H), 6.37 (s, 1H), 6.29 (s, 1H), 4.57-4.49 (m, 1H), 3.89-3.82 (m, 1H), 3.57-3.51 (m, 1H), 3.29-3.27 (m, 2H), 2.90-2.82 (m, 2H), 2.13-2.05 (m, 4H), 1.96-1.90 (m, 1H), 1.55-1.46 (m, 2H), 1.35-1.27 (m, 2H), 1.04-1.00 (m, 2H), 0.99-0.75 (m, 2H).

### Example 40: Synthesis of Compound 81

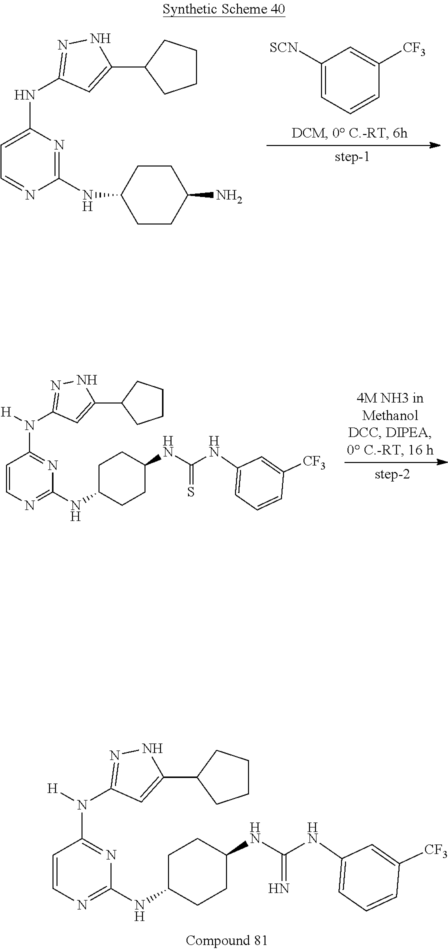

### Step-1: Synthesis of 1-((1R,4R)-4-((4-((5-cyclopentyl-1H-pyrazol-3-yl)amino)pyrimidin-2-yl)amino)cyclohexyl)-3-(3-(trifluoromethyl)phenyl)thiourea

To a stirred solution of 2-((4-aminophenyl)thio)-N-(5-cyclopentyl-1H-pyrazol-3-yl)pyrimidin-4-amine (0.15 g, 0.42 mmol) in dichloromethane (2 mL) and added 3-trifluoromethyl phenyl isothiocynate (0.08 g, 0.42 mmol) at 0° C. The resultant reaction was stirred at room temperature for 16 h. The completion of reaction was monitored by TLC and then the reaction mixture was allowed to cool to room temperature and then filtered off the resultant white precipitate through sintered funnel and the solid was washed with dichloromethane several times to afford crude 1-(4-((4-((5-cyclopentyl-1H-pyrazol-3-yl)amino)pyrimidin-2-yl)thio)phenyl)-3-(3-(trifluoromethyl)phenyl)urea (0.06 g, 26%) as white solid, which was taken to the next step without further purification. LC purity: 33.1%; m/z: 545.3 [M+H]⁺ (Mol. formula C₂₆H₃₁F₃N₈S, calcd. mol. wt. 544.65).

### Step-2: Synthesis of 1-((1R,4R)-4-((4-((5-cyclopentyl-1H-pyrazol-3-yl)amino)pyrimidin-2-yl)amino)cyclohexyl)-3-(3-(trifluoromethyl)phenyl)guanidine

To a cooled 0° C. solution of 1-((1R,4R)-4-((4-((5-cyclopentyl-1H-pyrazol-3-yl)amino)pyrimidin-2-yl)amino)cyclohexyl)-3-(3-(trifluoromethyl)phenyl)thiourea (0.5 g, 0.42 mmol) was added 4 M ammonia in methanol (10 mL). The resulting reaction mixture was stirred at 0° C. for 30 min. Then DIPEA (0.5 mL, w/v) was added followed by the addition of DCC (0.5 g, w/w). The reaction mixture was stirred at ambient temperature for 16 h. The progress of the reaction was monitored by LCMS, after complete consumption of the starting material, the reaction mixture was concentrated. The crude was added water and extracted 10% methanol in dichloromethane. The organic layer was dried over anhydrous Na₂SO₄ and concentrated to obtain the residue. The residue was purified by reverse phase preparative HPLC to yield 1-((1R,4R)-4-((4-((5-cyclopentyl-1H-pyrazol-3-yl)amino)pyrimidin-2-yl)amino)cyclohexyl)-3-(3-(trifluoromethyl)phenyl)guanidine (25 mg, 5%) as a formic acid salt. LC purity: 99.75%; m/z: 526.3 [M+H]⁻ (Mol. formula C₂₆H₃₂F₃N₉, calcd. mol. wt. 527.6). ¹¹H NMR (400 MHz, CD₃OD): δ 7.77 (d, J=6.4 Hz, 1H), 7.68-7.62 (m, 2H), 7.58-7.53 (m, 2H), 6.28 (d, J=6.4 Hz, 1H), 6.16 (s, 1H), 3.82-7.75 (m, 1H), 3.63-3.58 (m, 1H), 3.12-3.08 (m, 1H), 2.22-2.06 (m, 6H), 1.83-1.76 (m, 2H), 1.76-1.66 (m, 4H), 1.64-1.50 (m, 4H).

### Example 41: Synthesis of Compound 87

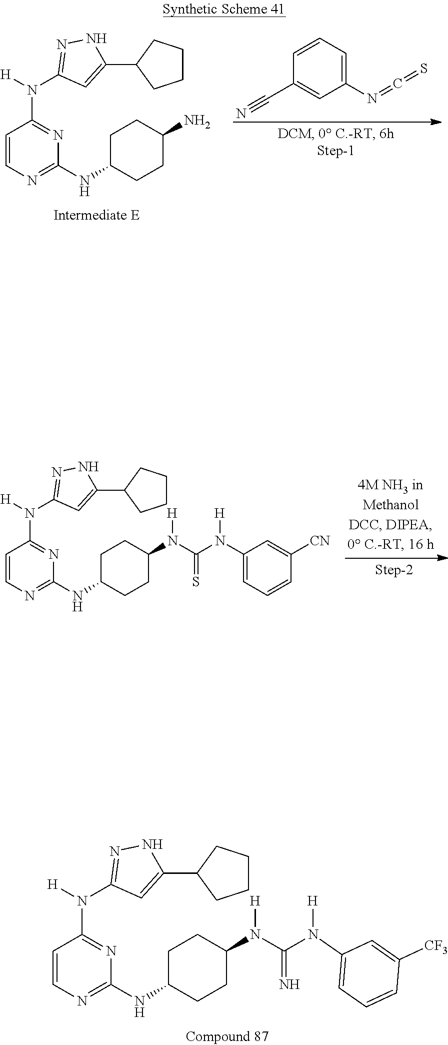 [SMILES: N[C@H]1CC[C@H](Nc2nccc(Nc3cc(C4CCCC4)[nH]n3)n2)CC1 ; N#Cc1cccc(N=C=S)c1 ; N#Cc1cccc(NC(=S)N[C@H]2CC[C@H](Nc3nccc(Nc4cc(C5CCCC5)[nH]n4)n3)CC2)c1 ; N=C(Nc1cccc(C(F)(F)F)c1)N[C@H]1CC[C@H](Nc2nccc(Nc3cc(C4CCCC4)[nH]n3)n2)CC1]

### Step-1: Synthesis of 1-(3-cyanophenyl)-3-((1R,4R)-4-((4-((5-cyclopentyl-1H-pyrazol-3-yl)amino)pyrimidin-2-yl)amino)cyclohexyl)thiourea

To a stirred solution of Intermediate E—N²-((1R,4R)-4-aminocyclohexyl)-N⁴-(5-cyclopentyl-1H-pyrazol-3-yl)pyrimidine-2,4-diamine (0.5 g, 1.46 mmol) in dichloromethane (10 mL) was added 3-isothiocyanatobenzonitrile (0.235 g, 1.46 mmol) at 0° C. The resultant reaction mixture was stirred at room temperature for 16 h. The completion of reaction was monitored by TLC. After consumption of the starting material, the reaction mixture was allowed to warm to room temperature, then acidified with concentrated HCl and then the resultant white precipitate was recovered using a sintered funnel and the solid was washed with dichloromethane several times to deliver 1-(3-cyanophenyl)-3-((1r,4r)-4-((4-((5-cyclopentyl-1H-pyrazol-3-yl)amino)pyrimidin-2-yl)amino)cyclohexyl)thiourea (0.5 g, crude) as white solid. LC purity: 83.04%; m/z: 502.3 [M+H]⁺ (Mol. formula C₂₆H₃₁N₉S, calcd. mol. wt. 501.66).

### Step-2: Synthesis of 1-(3-cyanophenyl)-3-((1R,4R)-4-((4-((5-cyclopentyl-1H-pyrazol-3-yl)amino)pyrimidin-2-yl)amino)cyclohexyl)guanidine

To a cooled 0° C. solution of 1-(3-cyanophenyl)-3-((1R,4R)-4-((4-((5-cyclopentyl-1H-pyrazol-3-yl)amino)pyrimidin-2-yl)amino)cyclohexyl)thiourea (0.5 g, 0.42 mmol) was added 4 M ammonia in methanol (10 mL). The resulting reaction mixture was stirred at 0° C. for 30 min. Then DIPEA (0.5 mL, w/v) was added followed by the addition of DCC (0.5 g, w/w). The reaction was stirred at ambient temperature for 16 h. The progress of the reaction was monitored by LCMS. After complete consumption of the starting material, the reaction mixture was concentrated. The crude was added water and extracted 10% methanol in dichloromethane. The organic layer was dried over anhydrous Na₂SO₄ and concentrated to get the residue. The residue was purified by reverse phase prep HPLC to yield Compound 87 1-(3-cyanophenyl)-3-((1R,4R)-4-((4-((5-cyclopentyl-1H-pyrazol-3-yl)amino)pyrimidin-2-yl)amino)cyclohexyl)guanidine (40 mg, 10%) as a TFA salt. LC purity: 99.54%; m/z: 483.3 [M−H]⁺ (Mol. formula C₂₆H₃₂N₁₀, calcd. mol. wt. 484.61). ¹H NMR (400 MHz, CD₃OD): δ 7.76 (s, 1H), 7.67-7.57 (m, 4H), 6.31 (s, 1H), 6.20 (s, 1H), 3.82-3.75 (m, 1H), 3.59-3.57 (m, 1H), 3.13-3.09 (m, 1H), 2.21-2.07 (m, 6H), 1.81-1.64 (m, 6H), 1.60-1.45 (m, 4H).

### Example 42: Synthesis of Compound 104

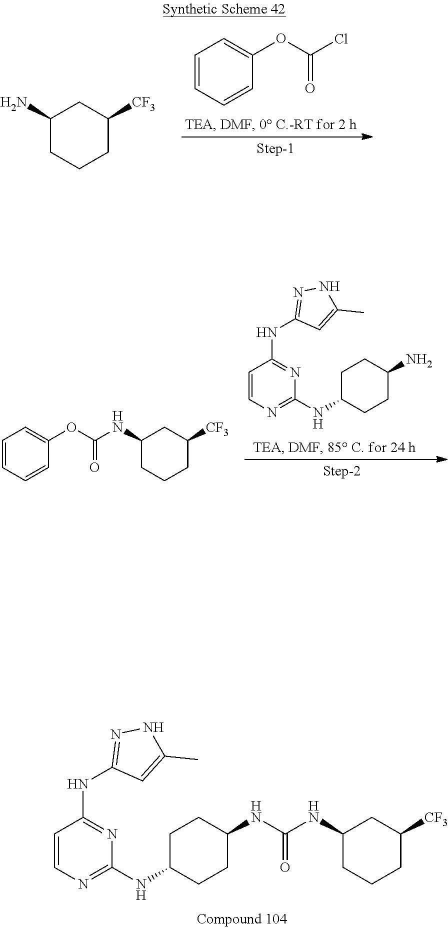 [SMILES: N[C@@H]1CCC[C@H](C(F)(F)F)C1 ; O=C(Cl)Oc1ccccc1 ; Cc1cc(Nc2ccnc(N[C@H]3CC[C@H](N)CC3)n2)n[nH]1 ; O=C(N[C@@H]1CCC[C@H](C(F)(F)F)C1)Oc1ccccc1 ; Cc1cc(Nc2ccnc(N[C@H]3CC[C@H](NC(=O)N[C@@H]4CCC[C@H](C(F)(F)F)C4)CC3)n2)n[nH]1]

### Step-1: Synthesis of phenyl ((1R,3S)-3-(trifluoromethyl)cyclohexyl)carbamate

To a solution of (1R,3S)-3-(trifluoromethyl)cyclohexan-1-amine (200 mg, 0.985 mmol) in dry DMF (5 mL) at 0° C. was added triethylamine (0.41 mL, 2.95 mmol) and phenyl chloroformate (0.15 mL, 1.18 mmol). The reaction mixture was stirred at room temperature for 2 h. The progress of the reaction was monitored by TLC analysis. After completion of the reaction, the reaction mixture was quenched with water and extracted with DCM. The combined organic layers were washed with brine solution then dried over anhydrous Na₂SO₄ and concentrated under reduced pressure to obtain phenyl ((1R,3S)-3-(trifluoromethyl)cyclohexyl)carbamate (220 mg, crude) as a light yellow solid. Crude material taken for the next step without purification. LC purity: 48.3%; m/z: 288.3 [M+H]⁺ (Mol. formula C₁₄N₁₆F₃NO₂, calcd. mol. wt. 287.28).

### Step-2: Synthesis of 1-((1R,4R)-4-((4-((5-methyl-1H-pyrazol-3-yl)amino)pyrimidin-2-yl)amino)cyclohexyl)-3-((1R,3S)-3-(trifluoromethyl)cyclohexyl)urea

To a stirred solution of phenyl ((1R,3S)-3-(trifluoromethyl)cyclohexyl)carbamate (220 mg, 0.766 mmol) in dry DMF (5 mL) was added triethylamine (0.32 mL, 2.298 mmol). The reaction mixture was heated to 65° C. for 1 h. The reaction mixture was cooled to room temperature and added N2-((1R,4R)-4-aminocyclohexyl)-N4-(5-methyl-1H-pyrazol-3-yl)pyrimidine-2,4-diamine (238 mg, 0.766 mmol). The reaction mixture was heated to 85° C. for 24 h. The reaction mixture was monitored by TLC, starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield Compound 104—1-((1R,4R)-4-((4-((5-methyl-1H-pyrazol-3-yl)amino)pyrimidin-2-yl)amino)cyclohexyl)-3-((1R,3S)-3-(trifluoromethyl)cyclohexyl)urea (30 mg, 20% yield) as a formic acid salt. LC purity: 98.72%; m/z: 481.8 [M+H]⁺ (Mol. formula C₂₂H₃₁F₃N₈O, calcd. Mol. wt. 480.54). ¹H NMR (400 MHz, CD₃OD): δ 7.75 (d, J=6 Hz, 1H), 6.22 (d, J=6.0 Hz, 1H), 6.17 (s, 1H), 3.77-3.71 (m, 1H), 3.55-3.48 (m, 2H), 2.28 (s, 3H), 2.25-2.22 (m, 1H), 2.20-2.12 (m, 3H), 2.09-2.01 (m, 2H), 1.95-1.87 (m, 3H), 1.47-1.33 (m, 6H), 1.29-1.16 (m, 3H).

### Example 43: Synthesis of Compound 105

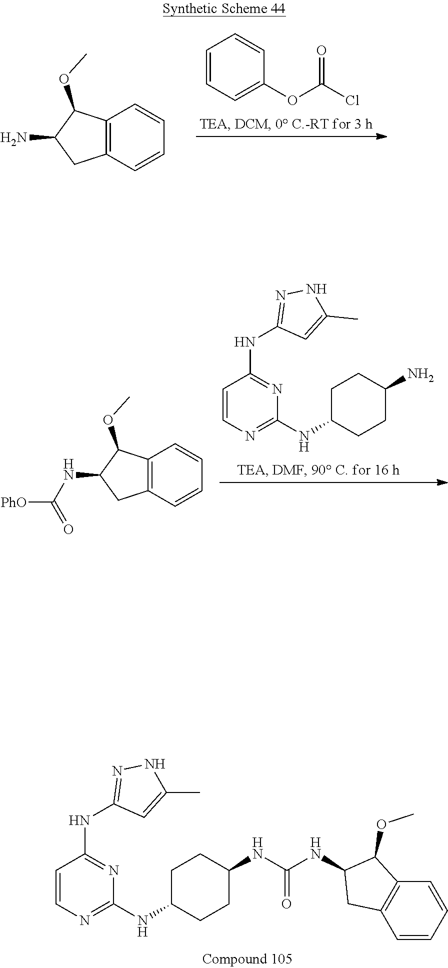

### Step-1: Synthesis of phenyl((1S,2R)-1-methoxy-2,3-dihydro-1H-inden-2-yl)carbamate

To a stirred solution of (1S,2R)-1-methoxy-2,3-dihydro-1H-inden-2-amine (250 mg, 1.533 mmol) in dry DCM (5 mL) was added triethylamine (0.3 mL, 2.300 mmol). The reaction mixture was cooled to 0° C. and added Phenyl chloroformate (280 mg, 1.840 mmol). The reaction was stirred at room temperature for 3 h. The reaction was monitored by TLC, and after consumption of starting material the reaction mixture was diluted with water and extracted with dichloromethane. The resulting organic layer was washed with brine solution then dried over anhydrous Na₂SO₄ and concentrated to obtain the crude compound. The crude compound was washed with pet ether and concentrated to yield phenyl((1S,2R)-1-methoxy-2,3-dihydro-1H-inden-2-yl)carbamate (200 mg, 46% yield). LC purity: 93.20%; m/z: 282.3 [M−H]⁺ (Mol. formula C₁₇H₁₇NO₃, calcd. mol. wt. 283.33).

### Step-2: Synthesis of 1-((1S,2R)-1-methoxy-2,3-dihydro-1H-inden-2-yl)-3-((1R,4R)-4-((4-((5methyl-1H-pyrazol-3-yl)amino)pyrimidin-2-yl)amino)cyclohexylurea

To a stirred solution of phenyl((1S,2R)-1-methoxy-2,3-dihydro-1H-inden-2-yl)carbamate (200 mg, 0.711 mmol) in dry DMF (2 mL) was added triethylamine (0.3 mL, 2.135 mmol). The reaction mixture was heated to 65° C. for 1 h. The reaction mixture was cooled to room temperature and added N2-((1R,4R)-4-aminocyclohexyl)-N4-(5methyl-1H-pyrazol-3-yl)pyrimidine-2,4-diamine (200 mg, 0.711 mmol). The reaction was heated to 90° C. for 16 h. The reaction was monitored by TLC after complete conversion of starting material, the reaction mixture was diluted with water and extracted with dichloromethane. The resulting organic layer was washed with brine solution then dried over anhydrous Na₂SO₄ and concentrated to obtain the crude compound. The crude compound was purified by reverse phase preparative HPLC to yield Compound 106—1-((1S,2R)-1-methoxy-2,3-dihydro-1H-inden-2-yl)-3-((1R,4R)-4-((4-((5-methyl-1H-pyrazol-3-yl)amino)pyrimidin-2-yl)amino)cyclohexyl)urea (30 mg, 35% yield) as a TFA salt. LC purity: 97.70%; m/z: 477.3 [M+H]⁺ (Mol. formula C₂₅H₃₂N₈O₂, calcd. mol. wt. 476.59). ¹H NMR (400 MHz, CD₃OD): δ 7.68 (s, 1H), 7.23-7.21 (m, 4H), 6.94 (s, 1H), 6.11 (d, J=138.80 Hz, 1H), 4.47-4.45 (m, 2H), 3.90-3.89 (m, 1H), 3.41 (s, 1H), 3.32-3.31 (m, 3H), 3.12-3.10 (m, 1H), 2.84-2.83 (m, 1H), 2.34 (s, 3H), 2.07 (t, J=12.40 Hz, 4H), 1.52 (d, J=10.80 Hz, 2H), 1.33 (t, J=9.60 Hz, 2H).

### Example 44: Synthesis of 1-((1R,4S)-4-((4-((5-cyclopentyl-1H-pyrazol-3-yl)amino)pyrimidin-2-yl)(methyl)amino) cyclohexyl)-3-((1S,3R)-3-(trifluoromethyl)cyclohexyl)urea (Compound 121)

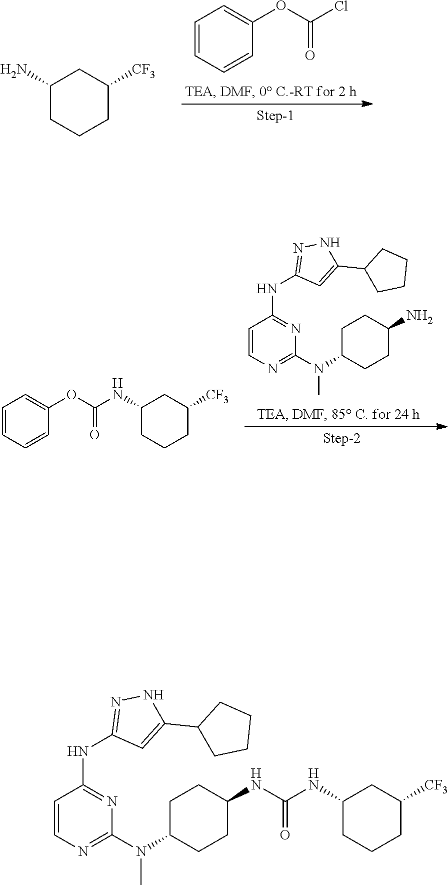 [SMILES: N[C@H]1CCC[C@@H](C(F)(F)F)C1 ; O=C(Cl)Oc1ccccc1 ; O=C(N[C@H]1CCC[C@@H](C(F)(F)F)C1)Oc1ccccc1 ; CN(c1nccc(Nc2cc(C3CCCC3)[nH]n2)n1)[C@H]1CC[C@H](N)CC1 ; CN(c1nccc(Nc2cc(C3CCCC3)[nH]n2)n1)[C@H]1CC[C@H](NC(=O)N[C@H]2CCC[C@@H](C(F)(F)F)C2)CC1]

### Step-1: Synthesis of phenyl ((1S,3R)-3-(trifluoromethyl)cyclohexyl)carbamate

To a solution of Racemic cis-(1S,3R)-3-(trifluoromethyl)cyclohexan-1-amine (500 mg, 2.99 mmol) in dry DCM (10 mL) at 0° C. was added triethylamine (1.03 mL, 7.48 mmol) and phenyl chloroformate (0.35 mL, 2.99 mmol). The reaction mixture was stirred at room temperature for 2 h. The progress of the reaction was monitored by TLC analysis. After completion of the reaction, the reaction mixture was quenched with water and extracted with DCM. The combined organic layers were washed with brine solution then dried over anhydrous Na₂SO₄ and concentrated under reduced pressure to get phenyl ((1S,3R)-3-(trifluoromethyl)cyclohexyl)carbamate (650 mg, 76%) as a white solid. LC purity: 99%; m/z: 288.3 [M+H]⁺ (Mol. formula C₁₄H₁₆F₃NO₂, calcd. mol. wt. 287.28).

### Step-2: Synthesis of 1-((1R,4S)-4-((4-((5-cyclopentyl-1H-pyrazol-3-yl)amino)pyrimidin-2-yl)(methyl)amino)cyclohexyl)-3-((1S,3R)-3-(trifluoromethyl)cyclohexyl)urea

To a stirred solution of phenyl ((1S,3R)-3-(trifluoromethyl)cyclohexyl)carbamate (404 mg, 1.406 mmol) in dry DMF (10 mL) was added triethylamine (0.58 mL, 4.218 mmol). The reaction mixture was heated to 80° C. for 2 h. Then the reaction was cooled to room temperature and added N2-((1R,4R)-4-aminocyclohexyl)-N4-(5-cyclopentyl-1H-pyrazol-3-yl)-N2-methylpyrimidine-2,4-diamine (500 mg, 1.406 mmol). The reaction mixture was heated to 85° C. for 24 h. The progress of reaction mixture was monitored by TLC, starting material was consumed. The reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield 1-((1R,4R)-4-((4-((5-methyl-1H-pyrazol-3-yl)amino)pyrimidin-2-yl)amino)cyclohexyl)-3-((1S,3R)-3-(trifluoromethyl)cyclohexyl)urea (140 mg, 18% yield) as a TFA salt. LC purity: 99.7%; m/z: 549.3 [M+H]⁺ (Mol. formula C₂₇H₃₉F₃N₈O, calcd. mol. wt. 548.66). ¹H NMR (400 MHz, CD₃OD): δ 7.72 (d, J=6.8 Hz, 1H), 6.49 (s, 1H), 6.35 (d, J=6.8 Hz, 1H), 3.56-3.50 (m, 2H), 3.33-3.08 (m, 4H), 2.28-2.26 (m, 1H), 2.16-2.14 (m, 5H), 1.95-1.73 (m, 14H), 1.46-1.30 (m, 3H), 1.24-1.06 (m, 3H).

### Chiral Separation of 1-((1R,4S)-4-((4-((5-cyclopentyl-1H-pyrazol-3-yl)amino)pyrimidin-2-yl)(methyl)amino)cyclohexyl)-3-((1S,3R)-3-(trifluoromethyl)cyclohexyl)urea

0.5% Isopropyl Amine in Methanol % Co-Solvent: Methanol, Injected Volume: 15 μl, Outlet Pressure: 100 bar, Temperature: 35° C. The first eluting (RT=4.91) fractions were collected and concentrated under reduced pressure and the second eluting (RT=5.91) fractions were collected and concentrated under reduced pressure.

### First Eluting Isomer

LC Purity: 99.8%; m/z: 549.3 [M+H]⁺ (Mol. formula C₂₇H₃₉F₃N₈O, calcd. mol. wt. 548.66). ¹H NMR (400 MHz, CD₃OD): δ 7.72 (s, J=6.56 Hz, 1H), 6.49 (s, 1H), 6.35 (d, J=6.92 Hz, 1H), 4.89 (s, 1H), 3.57-3.50 (m, 2H), 3.33-3.08 (m, 4H), 2.29-2.25 (m, 2H), 2.17-2.14 (m, 5H), 1.95-1.73 (m, 12H), 1.40-1.20 (m, 3H), 1.17-1.07 (m, 3H).

### Second Eluting Isomer

LC Purity: 99.4%; m/z: 549.3 [M+H]⁺ (Mol. formula C₂₇H₃₉F₃N₈O, calcd. mol. wt. 548.66). ¹H NMR (400 MHz, CD₃OD): δ 7.72 (s, J=6.56 Hz, 1H), 6.49 (s, 1H), 6.35 (d, J=6.92 Hz, 1H), 4.89 (s, 1H), 3.57-3.50 (m, 2H), 3.33-3.08 (m, 4H), 2.29-2.25 (m, 2H), 2.17-2.14 (m, 5H), 1.95-1.73 (m, 12H), 1.40-1.20 (m, 3H), 1.17-1.07 (m, 3H).

### Example 45: Synthesis of 1-(5-cyano-2,3-dihydro-1H-inden-2-yl)-3-((1R,4R)-4-((4-((5-cyclopropyl-1H-pyrazol-3-yl)amino)pyrimidin-2-yl)(methyl)amino)cyclohexyl)urea (Compound 142)

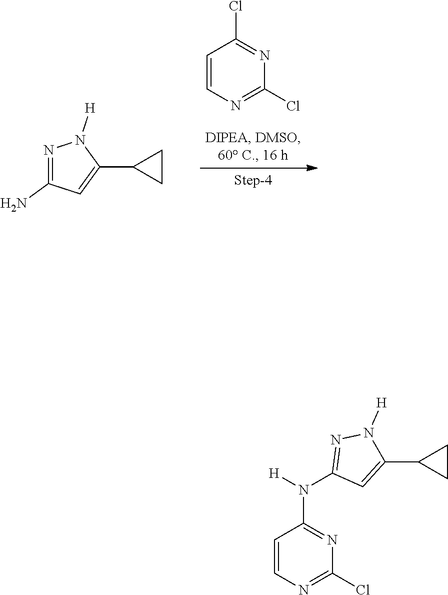 [SMILES: Clc1ccnc(Cl)n1 ; Nc1cc(C2CC2)[nH]n1 ; Clc1nccc(Nc2cc(C3CC3)[nH]n2)n1]

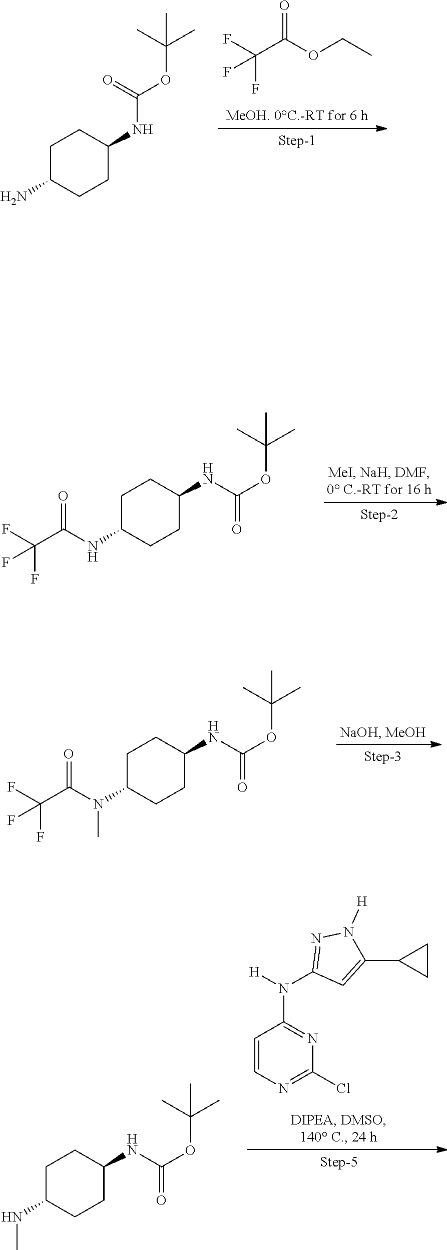

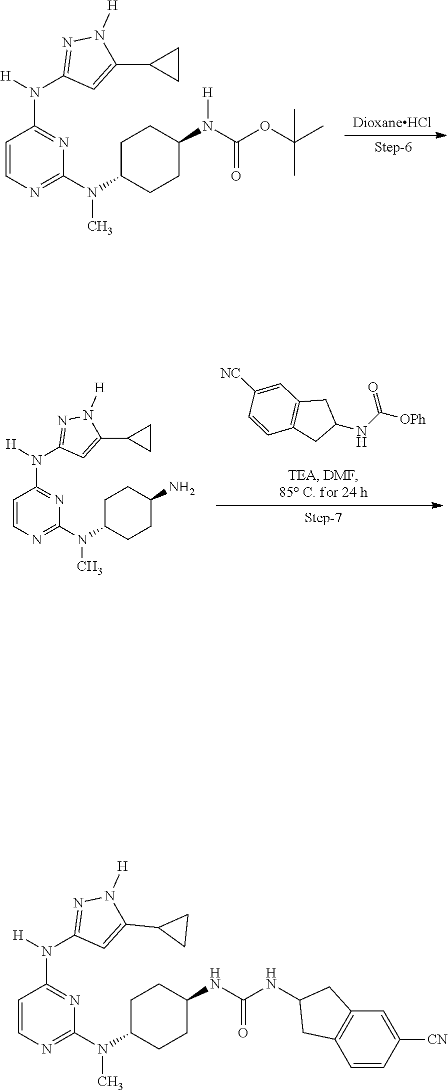 [SMILES: CN(c1nccc(Nc2cc(C3CC3)[nH]n2)n1)[C@H]1CC[C@H](NC(=O)OC(C)(C)C)CC1 ; CN(c1nccc(Nc2cc(C3CC3)[nH]n2)n1)[C@H]1CC[C@H](N)CC1 ; N#Cc1ccc2c(c1)CC(NC(=O)Oc1ccccc1)C2 ; CN(c1nccc(Nc2cc(C3CC3)[nH]n2)n1)[C@H]1CC[C@H](NC(=O)NC2Cc3ccc(C#N)cc3C2)CC1]

### Step-1: Synthesis of tert-butyl ((1R,4R)-4-(2,2,2-trifluoroacetamido)cyclohexyl)carbamate

To a solution of tert-butyl ((1R,4R)-4-aminocyclohexyl)carbamate (60 g, 279 mmol) in methanol (600 mL) at 0° C. was added ethyl trifluoro acetate (40.11 mL, 336 mmol). After complete addition the reaction was stirred at ambient temperature for 6 h. The reaction was monitored by TLC, after complete consumption of starting material the reaction mass was filtered and the solid was washed with cold methanol to yield tert-butyl ((1R,4R)-4-(2,2,2-trifluoroacetamido)cyclohexyl)carbamate (60 g, 69%) as a white solid. LC purity: 99.8%; m/z: 309.2 [M−H]⁻ (Mol. formula C₁₃H₂₁F₃N₂O₃, calcd. mol. wt. 310.32).

### Step-2: Synthesis of tert-butyl ((1R,4R)-4-(2,2,2-trifluoro-N-methylacetamido) cyclohexyl)carbamate

To a solution of tert-butyl ((1R,4R)-4-(2,2,2-trifluoroacetamido)cyclohexyl)carbamate (30 g, 96.67 mmol) in anhydrous DMF (300 mL) was cooled to 0° C. and added sodium hydride (4.9 g, 203.2 mmol, 60% in mineral oil) portion wise. The reaction mixture was stirred at 0° C. for 1 h followed by addition of methyl Iodide (7.2 mL, 116.0 mmol). The reaction mass was stirred at ambient temperature for 16 h. The reaction was monitored by LCMS, after complete consumption of the starting material, the reaction mixture was diluted with ice cold water and neutralized by using 2N citric acid solution. The resultant white precipitate was filtered off through sintered funnel and washed well with water, dried under vacuum to get tert-butyl ((1R,4R)-4-(2,2,2-trifluoro-N-methylacetamido)cyclohexyl)carbamate (29 g, 92%). LC purity: 92.6%; m/z: 225.2 [M−Boc]⁺ (Mol. formula C₁₄H₂₃F₃N₂O₃, calcd. mol. wt. 324.34).

### Step-3: Synthesis of tert-butyl ((1R,4R)-4-(methylamino)cyclohexyl)carbamate

Suspension of tert-butyl ((1R,4R)-4-(2,2,2-trifluoro-N-methylacetamido)cyclohexyl) carbamate (40 g, 0.123 mol) in methanol (400 mL) was heated at 50° C. with stirring until clear solution formation. Then 2N NaOH solution (80 mL) was added to it with constant stirring. Resultant slight cloudy solution obtained was stirred for further for 3 h at ambient temperature. The reaction was monitored by LCMS, after complete consumption of the starting material, the reaction was concentrated under reduced pressure to get residue. The residue was added 2N citric acid solution and extracted with 10% methanol in dichloromethane. Then aqueous layer was made alkaline with 2 N NaOH solution and again extracted with DCM:MeOH (9:1). The combined organic layer was dried over anhydrous sodium sulphate and concentrated to get tert-butyl ((1R,4R)-4-(methylamino)cyclohexyl)carbamate (23 g, 81%) as a white solid. LC purity: 96%; m/z: 229.4 [M+H]⁺ (Mol. formula C₁₂H₂₄N₂O₂, calcd. mol. wt. 228.34)

### Step-4: Synthesis of 2-chloro-N-(5-cyclopropyl-1H-pyrazol-3-yl)pyrimidin-4-amine

To a stirred solution of 5-cyclopropyl-1H-pyrazol-3-amine (1.0 g, 8.13 mmol) in DMSO (10 mL) was added DIPEA (2.12 mL, 12.2 mmol) and 2, 4-dichloropyrimidine (1.45 g, 9.76 mmol). The reaction was heated to 60° C. for 16 h. The progress of the reaction was monitored by TLC after complete consumption of starting material, the reaction mixture was quenched with ice water and the solid was filtered, washed with dichloromethane and dried under vacuum to get 2-chloro-N-(5-cyclopropyl-1H-pyrazol-3-yl)pyrimidin-4-amine (1.2 g, 63% yield) as white solid. LC purity: 87%; m/z: 236.1 [M+H]⁺ (Mol. formula C₁₀H₁₀ClN₅, calcd. mol. wt. 235.68). ¹H NMR (400 MHz, DMSO-d6): δ 12.10 (s, 1H), 10.26 (s, 2H), 8.15 (d, 1H), 7.20 (s, 1H), 6.01 (s, 1H), 1.92-1.86 (m, 1H), 0.96-0.89 (m, 2H), 0.70 (d, J=8.0 Hz, 2H).

### Step-5: Synthesis of tert-butyl ((1R,4R)-4-((4-((5-cyclopropyl-1H-pyrazol-3-yl)amino) pyrimidin-2-yl)(methyl)amino)cyclohexyl)carbamate

To a solution of 2-chloro-N-(5-cyclopropyl-1H-pyrazol-3-yl)pyrimidin-4-amine (0.5 g, 2.131 mmol) in DMSO (5 mL) was added DIPEA (0.74 mL, 4.252 mmol) and tert-butyl ((1R,4R)-4-(methylamino)cyclohexyl)carbamate (0.58 g, 2.550 mmol). The reaction mixture was heated to 140° C. for 24 h. After complete consumption of the starting material (monitored by TLC), the reaction mixture was cooled to room temperature and water was added. Thus, obtained white solid was filtered through sintered funnel washed with water and pet ether then dried under vacuum. The crude was purified by Biotage-Isolera using silica gel (230-400 mesh) with a gradient elution of 0-85% ethyl acetate in pet ether to obtain tert-butyl ((1R,4R)-4-((4-((5-cyclopropyl-1H-pyrazol-3-yl)amino)pyrimidin-2-yl)(methyl)amino)cyclohexyl)carbamate (0.3 g, 32%) as a white solid. LC purity: 76%; m/z: 428.4 [M+H]⁺ (Mol. formula C₂₂H₃₃N₇O₂, calcd. mol. wt. 427.55).

### Step-6: Synthesis of N2-((1R,4R)-4-aminocyclohexyl)-N4-(5-cyclopropyl-1H-pyrazol-3-yl)-N2-methylpyrimidine-2,4-diamine

To a stirred solution of tert-butyl ((1R,4R)-4-((4-((5-cyclopropyl-1H-pyrazol-3-yl)amino) pyrimidin-2-yl)(methyl)amino)cyclohexyl)carbamate (0.35 g, 0.818 mmol) in dry DCM (7 mL) was cooled to 0° C. and added HCl in Dioxane (3.5 mL, 4M solution). The reaction was allowed to stir at room temperature for 3 h. The progress of the reaction was monitored by TLC after complete consumption of starting material, the resulting mixture was concentrated and triturated with pet ether and concentrated under high vacuum to yield N2-((1R,4R)-4-aminocyclohexyl)-N4-(5-cyclopropyl-1H-pyrazol-3-yl)-N2-methylpyrimidine-2,4-diamine (0.35 g, quantitative yield) as a HCl salt. LC purity: 54%; m/z: 328.3 [M+H]⁺ (Mol. formula C₁₇H₂₅N₇, calcd. mol. wt. 327.44).

### Step-7: Synthesis of 1-(5-cyano-2,3-dihydro-1H-inden-2-yl)-3-((1R,4R)-4-((4-((5-cyclopropyl-1H-pyrazol-3-yl)amino)pyrimidin-2-yl)(methyl)amino)cyclohexyl)urea

To a solution of phenyl (5-cyano-2,3-dihydro-1H-inden-2-yl)carbamate (169 mg, 0.612 mmol) in DMF (4.0 mL) was added triethylamine (0.25 mL, 1.832 mmol) and resultant reaction was gradually heated to 85° C. for 2 h. The reaction was cooled to room temperature and added N2-((1R,4R)-4-aminocyclohexyl)-N4-(5-cyclopropyl-1H-pyrazol-3-yl)-N2-methylpyrimidine-2,4-diamine (200 mg, 0.612 mmol). The reaction mixture was heated to 85° C. for 24 h. The progress of the reaction was monitored by TLC after complete consumption of starting material, the reaction mixture was concentrated then added water and extracted with 10% methanol in dichloromethane. The combined organic layers were washed with brine, dried over anhydrous Na₂SO₄ and concentrated. The crude compound was purified by reverse phase prep HPLC to yield 1-(5-cyano-2,3-dihydro-1H-inden-2-yl)-3-((1R,4R)-4-((4-((5-cyclopropyl-1H-pyrazol-3-yl)amino)pyrimidin-2-yl)(methyl)amino)cyclohexyl)urea (30 mg, 10% yield) as a TFA salt. LC purity: 97.6%; m/z: 512.2 [M+H]⁺ (Mol. formula C₂₈H₃₃N₉O, calcd. mol. wt. 511.63). ¹H NMR (400 MHz, CD₃OD): δ 7.78 (s, 1H), 7.72 (d, J=6.36 Hz, 1H), 7.54 (d, J=7.64 Hz, 1H), 7.42 (d, J=7.76 Hz, 1H), 6.36-6.33 (m, 2H), 4.54-4.51 (m, 2H), 3.52 (d, J=10.36 Hz, 1H), 3.33-3.28 (m, 2H), 3.07 (s, 3H), 2.91-2.83 (m, 2H), 2.13 (d, J=6.92 Hz, 2H), 1.95 (d, J=4.88 Hz, 1H), 1.84 (s, 4H), 1.40-1.33 (m, 2H), 1.06-1.03 (m, 2H), 0.81-0.65 (m, 2H).

### Example 46: Synthesis of 3-(5-cyano-2,3-dihydro-1H-inden-2-yl)-1-((1R,4R)-4-((4-((5-cyclopentyl-1H-pyrazol-3-yl)amino)pyrimidin-2-yl)(methyl)amino)cyclohexyl)-1-methylurea (Compound 143)

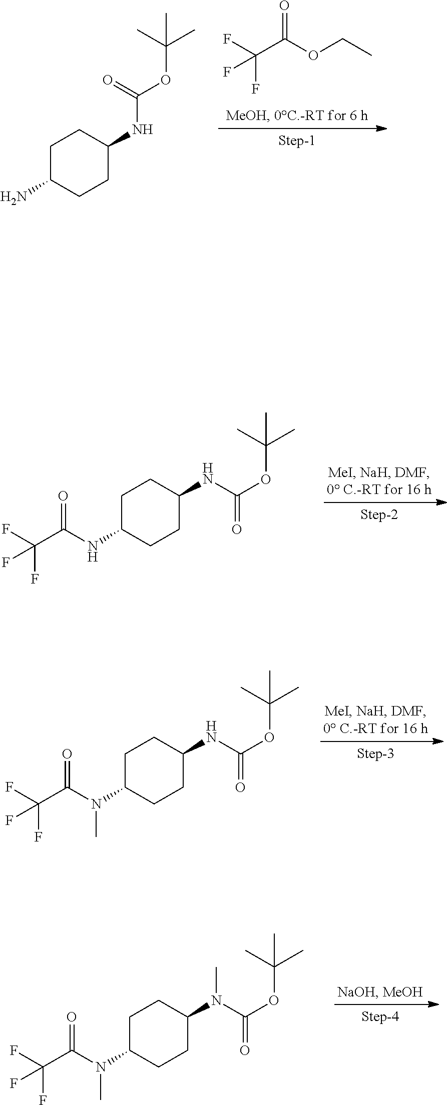

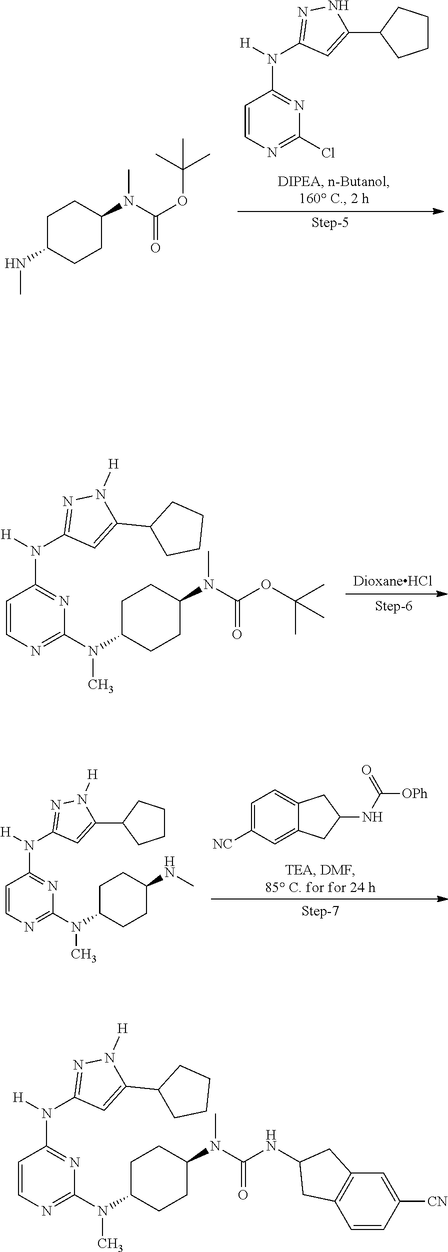

### Step-1: Synthesis of tert-butyl ((1R,4R)-4-(2,2,2-trifluoroacetamido)cyclohexyl) carbamate

To a solution of tert-butyl ((1R,4R)-4-aminocyclohexyl)carbamate (60 g, 279 mmol) in methanol (600 mL) at 0° C. was added ethyl trifluoro acetate (40.11 mL, 336 mmol). After complete addition the reaction mass was stirred at ambient temperature 6 h. The reaction was monitored by TLC, after complete consumption starting material the reaction mass was filtered and the solid obtained was washed with cold methanol to yield tert-butyl ((1R,4R)-4-(2,2,2-trifluoroacetamido)cyclohexyl)carbamate (60 g, 69%) as a white solid. LC purity: 99.8%; m/z: 309.2 [M−H]⁻ (Mol. formula C₁₃H₂₁F₃N₂O₃, calcd. mol. wt. 310.32).

### Step-2: Synthesis of tert-butyl ((1R,4R)-4-(2,2,2-trifluoro-N-methylacetamido) cyclohexyl)carbamate

To a solution of tert-butyl ((1R,4R)-4-(2,2,2-trifluoroacetamido)cyclohexyl)carbamate (30 g, 96.67 mmol) in anhydrous DMF (300 mL) was cooled to 0° C. and sodium hydride (4.9 g, 203.2 mmol, 60% in mineral oil) was added portion wise. The reaction mixture was stirred at 0° C. for 1 h followed by addition of methyl Iodide (7.2 mL, 116.0 mmol). The reaction mass was stirred at ambient temperature for 16 h. The reaction was monitored by LCMS, after majority of the starting material consumed, the reaction mixture was poured on ice cold water and neutralized by using 2N citric acid solution. The resultant white precipitate was filtered off through sintered funnel washed well with water and dried under vacuum to get tert-butyl ((1R,4R)-4-(2,2,2-trifluoro-N-methylacetamido)cyclohexyl)carbamate (29 g, 92%). LC purity: 92.6%; m/z: 225.2 [M−Boc]⁺ (Mol. formula C₁₄H₂₃F₃N₂O₃, calcd. mol. wt. 324.34).

### Step-3: Synthesis tert-butyl methyl ((1R,4R)-4-(2,2,2-trifluoro-N-methylacetamido)cyclohexyl)carbamate

To a solution of tert-butyl ((1R,4R)-4-(2,2,2-trifluoroacetamido)cyclohexyl)carbamate (2.0 g, 6.19 mmol) in anhydrous DMF (20 mL) was cooled to 0° C. and sodium hydride (0.74 g, 30.87 mmol) was added portion wise. The reaction mass was stirred at 0° C. for 30 min, followed by addition of methyl Iodide (0.77 mL, 12.34 mmol). The reaction was stirred at ambient temperature for 16 h. The reaction was monitored by LCMS, after complete consumption of the starting material, the reaction mixture was poured on ice cold water and neutralized with 2N citric acid solution. The resultant white precipitate was filtered off through sintered funnel, washed well with water and dried under vacuum to get tert-butyl methyl((1R,4R)-4-(2,2,2-trifluoro-N-methylacetamido)cyclohexyl)carbamate (1.6 g, 76.9%). LC purity: 85%; m/z: 239.2 [M−Boc]⁺ (Mol. formula C₁₅H₂₅F₃N₂O₃, calcd. mol. wt. 338.37).

### Step-4: Synthesis of tert-butyl methyl((1R,4R)-4-(methylamino)cyclohexyl)carbamate

A suspension of tert-butyl methyl((1R,4R)-4-(2,2,2-trifluoro-N-methylacetamido)cyclohexyl)carbamate (1.6 g, 6.61 mmol) in methanol (16 mL) was heated at 50° C. with stirring until clear solution formation. Then 2N NaOH solution (8 mL) was added to it with constant stirring till we get slightly cloudy precipitate which was further stirred at room temperature for 3 h. The reaction was monitored by LCMS, after complete consumption of the starting material, the reaction mixture was concentrated under reduced pressure to get residue. The residue thus obtained was dissolved in 2N citric acid solution and extracted with 10% methanol in dichloromethane. The aqueous layer separated was made alkaline with 2 N NaOH solution and again extracted with DCM:MeOH (9:1). The combined organic layer was dried over anhydrous sodium sulphate and concentrated to get tert-butyl methyl((1R,4R)-4-(methylamino)cyclohexyl)carbamate (750 mg, crude) as a white solid. LC purity: 57%; m/z: No ionisation (Mol. formula C₁₃H₂₆N₂O₂, calcd. mol. wt. 242.36).

### Step-5: Synthesis of tert-butyl ((1R,4R)-4-((4-((5-cyclopentyl-1H-pyrazol-3-yl)amino)pyrimidin-2-yl)(methyl)amino)cyclohexyl)(methyl)carbamate

In a 20 mL microwave vial a mixture of 2-chloro-N-(5-cyclopentyl-1H-pyrazol-3-yl)pyrimidin-4-amine (300 mg, 1.138 mmol), tert-butyl methyl((1R,4R)-4-(methylamino) cyclohexyl)carbamate (0.28 g, 1.138 mmol) in n-Butanol (6 mL) was added DIPEA (0.4 mL, 2.276 mmol). The reaction mixture was heated at 160° C. in a microwave for 2 h. The progress of the reaction was monitored by TLC after complete consumption of starting material, the reaction mixture was cooled to room temperature and concentrated to remove n-butanol. The obtained residue was purified by using column chromatography using silica gel (230-400 mesh) with a gradient elution of 0-100% ethyl acetate in pet ether to get tert-butyl ((1R,4R)-4-((4-((5-cyclopentyl-1H-pyrazol-3-yl)amino)pyrimidin-2-yl)(methyl) amino)cyclohexyl)(methyl)carbamate (300 mg, 56%). LC purity: 83%; m/z: 470.3 [M+H]⁺ (Mol. formula C₂₅H₃₉N₇O₂, calcd. mol. wt. 469.63).

### Step-6: Synthesis of N4-(5-cyclopentyl-1H-pyrazol-3-yl)-N2-methyl-N2-((1R,4R)-4-(methylamino)cyclohexyl)pyrimidine-2,4-diamine

To a solution of tert-butyl ((1R,4R)-4-((4-((5-cyclopentyl-1H-pyrazol-3-yl)amino) pyrimidin-2-yl)(methyl)amino)cyclohexyl)(methyl)carbamate (300 mg, 0.64 mmol) in DCM (6.0 mL) was cooled to 0° C. 4 M Hydrochloric acid in 1,4 dioxane (3.0 mL) was added. The reaction mass was allowed to stir at room temperature for 3 h. The reaction was monitored by TLC, after consumption of starting material, the reaction mixture was concentrated to obtain N4-(5-cyclopropyl-1H-pyrazol-3-yl)-N2-methyl-N2-((1R,4R)-4-(methylamino)cyclohexyl) pyrimidine-2,4-diamine (300 mg, quantitative yield) as a HCl salt. LC purity: 85%; m/z: 370.1 [M+H]⁺ (Mol. formula C₁₈H₂₇N₇, calcd. mol. wt. 369.52)

### Step-7: Synthesis of 3-(5-cyano-2,3-dihydro-1H-inden-2-yl)-1-((1R,4R)-4-((4-((5-cyclopentyl-1H-pyrazol-3-yl)amino)pyrimidin-2-yl)(methyl)amino)cyclohexyl)-1-methylurea

To a solution of phenyl (5-cyano-2,3-dihydro-1H-inden-2-yl)carbamate (151 mg, 0.542 mmol) in DMF (14 mL) at 0° C. and was added triethylamine (0.23 mL, 1.63 mmol). The reaction mass was stirred at 0° C. for 1 h. Then N4-(5-cyclopentyl-1H-pyrazol-3-yl)-N2-methyl-N2-((1R,4R)-4-(methylamino)cyclohexyl)pyrimidine-2,4-diamine (200 mg, 0.542 mmol) was added portion wise and the reaction mixture was heated to 85° C. for 24 h. The progress of the reaction was monitored by TLC after complete consumption of starting material, the reaction mixture was concentrated, diluted with water and extracted using 10% methanol in dichloromethane. The combined organic layers were washed with brine, dried over anhydrous Na₂SO₄ and concentrated. The crude compound was purified by reverse phase prep HPLC to yield 3-(5-cyano-2,3-dihydro-1H-inden-2-yl)-1-((1R,4R)-4-((4-((5-cyclopentyl-1H-pyrazol-3-yl)amino) pyrimidin-2-yl)(methyl)amino)cyclohexyl)-1-methylurea (25 mg, 8% yield) as a TFA salt. LC purity: 98.9%; m/z: 554.2 [M+H]⁺ (Mol. formula C₃₁H₃₉N₉O, calcd. mol. wt. 553.72). ¹H NMR (400 MHz, CD₃OD): δ 7.78 (s, 1H), 7.57 (s, 1H), 7.53 (d, J=7.68 Hz, 1H), 7.40 (d, J=7.76 Hz, 1H), 6.37 (m, 2H), 4.63-4.59 (m, 2H), 4.12 (s, 1H), 3.37-3.35 (m, 1H), 3.06-2.94 (m, 6H), 2.94 (s, 3H), 2.14 (d, J=5.6 Hz, 2H), 1.88-1.67 (m, 15H).

### Example 47: Synthesis of 1-(5-cyano-2,3-dihydro-1H-inden-2-yl)-3-((1R,4R)-4-((4-((5-cyclobutyl-1H-pyrazol-3-yl)amino)pyrimidin-2-yl)(methyl)amino)cyclohexyl)urea (Compound 144)

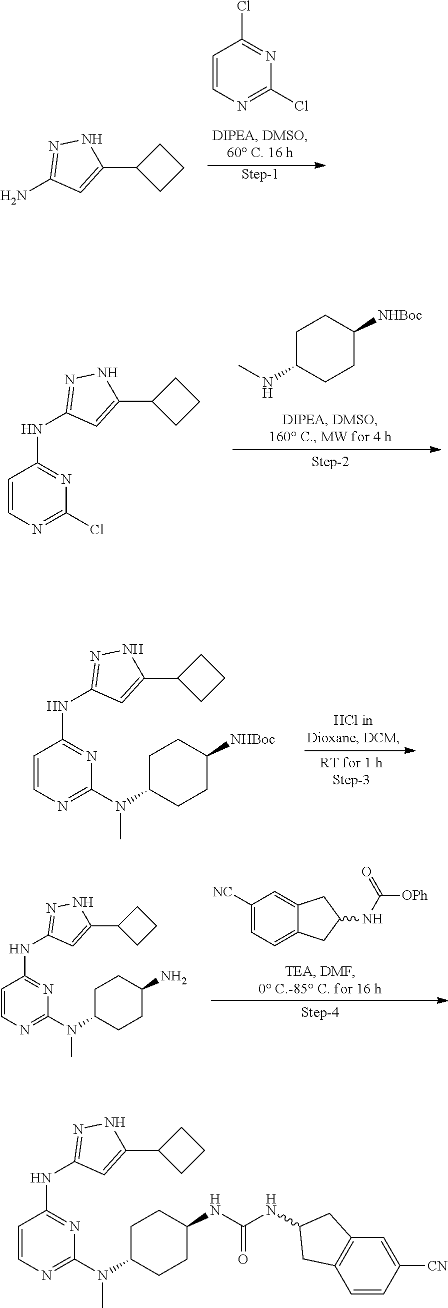

### Step-1: Synthesis of 2-chloro-N-(5-cyclobutyl-1H-pyrazol-3-yl)pyrimidin-4-amine

To a stirred solution of 5-cyclobutyl-1H-pyrazol-3-amine (3 g, 21.89 mmol) in DMSO (15 mL) was added 2,4-dichloropyrimidine (3.88 g, 26.27 mmol) and DIPEA (5.72 mL, 32.84 mmol). The reaction mixture was heated to 60° C. for 16 h. After completion of the reaction (monitored by TLC), the reaction mixture was poured into ice cold water. The precipitate formed was filtered off, washed with water and pet ether then dried under vacuum to get 2-chloro-N-(5-cyclobutyl-1H-pyrazol-3-yl)pyrimidin-4-amine (3.2 g, 58.69%). LC purity: 87.6%; m/z: 250.0 [M+H]⁺ (Mol. formula C₁₁H₁₂ClN₅, calcd. mol. wt. 249.70).

### Step-2: Synthesis of tert-butyl ((1R,4R)-4-((4-((5-cyclobutyl-1H-pyrazol-3-yl)amino) pyrimidin-2-yl)(methyl)amino)cyclohexyl)carbamate

In a 20 mL microwave vial a mixture of tert-butyl ((1R,4R)-4-(methylamino)cyclohexyl)carbamate (3.2 g, 14.035 mmol), 2-chloro-N-(5-cyclobutyl-1H-pyrazol-3-yl)pyrimidin-4-amine (2.79 g, 11.22 mmol) in n-butanol (30 mL) was added DIPEA (7.33 mL, 42.105 mmol). The reaction heated to 160° C. for 4 h in a microwave. The reaction mixture was cooled to room temperature and concentrated to remove n-butanol. The residue thus obtained was purified by Biotage Isolera using silica gel (230-400 mesh) column chromatography with gradient elution of 0-10% methanol in dichloromethane to get tert-butyl ((1R,4R)-4-((4-((5-cyclobutyl-1H-pyrazol-3-yl)amino)pyrimidin-2-yl)(methyl)amino) cyclohexyl)carbamate (1.15 g, 18.58%). LC purity: 93.4%; m/z: 442.2 [M+H]⁺ (Mol. formula C₂₃H₃₅N₇O₂ calcd. mol. wt. 441.58).

### Step-3: Synthesis of N2-((1R,4R)-4-aminocyclohexyl)-N4-(5-cyclobutyl-1H-pyrazol-3-yl)-N2-methylpyrimidine-2,4-diamine

To a stirred solution of tert-butyl ((1R,4R)-4-((4-((5-cyclobutyl-1H-pyrazol-3-yl)amino) pyrimidin-2-yl)(methyl)amino)cyclohexyl)carbamate (1.15 g, 2.607 mmol) in dichloromethane (12 mL) was cooled to 0° C. and HCl in dioxane (10 mL, 4M solution) was added. The reaction mass was allowed to stir at room temperature for 1 h. After complete consumption of the starting material (monitored by TLC), reaction mixture was concentrated to get N2-((1R,4R)-4-aminocyclohexyl)-N4-(5-cyclobutyl-1H-pyrazol-3-yl)-N2-methylpyrimidine-2,4-diamine (1.1 g, quantitative yield). LC purity: 98.2%; m/z: 342.3 [M+H]⁺ (Mol. formula C₁₈H₂₇N₇ calcd. mol. wt. 341.46).

### Step-4: Synthesis of 1-(5-cyano-2,3-dihydro-1H-inden-2-yl)-3-((1R,4R)-4-((4-((5-cyclobutyl-1H-pyrazol-3-yl)amino)pyrimidin-2-yl)(methyl)amino)cyclohexyl)urea

To a stirred solution of phenyl (5-cyano-2,3-dihydro-1H-inden-2-yl)carbamate (896.7 mg, 3.225 mmol) in dry DMF (10 mL) was added triethylamine (1.35 mL, 9.677 mmol). The reaction mixture was stirred at 0° C. to room temperature for 1 h. The reaction was cooled to room temperature N2-((1R,4R)-4-aminocyclohexyl)-N4-(5-cyclobutyl-1H-pyrazol-3-yl)-N2-methylpyrimidine-2,4-diamine (1.1 g, 3.225 mmol) was added. The reaction mass was heated to 85° C. for 16 h. After complete conversion of the starting material (monitored by TLC), the reaction mixture was diluted with water and extracted with dichloromethane. The organic layer separated was washed with water, brine solution then dried over anhydrous Na₂SO₄ and concentrated to obtain crude compound. The crude thus obtained was purified by reverse phase preparative HPLC to get 1-(5-cyano-2,3-dihydro-1H-inden-2-yl)-3-((1R,4R)-4-((4-((5-cyclobutyl-1H-pyrazol-3-yl)amino)pyrimidin-2-yl)(methyl)amino)cyclohexyl)urea (530 mg, 31.3%) as a TFA salt. LC purity: 99.86%; m/z: 526.1 [M+H]⁺ (Mol. formula C₂₉H₃₅N₉O, calcd. mol. wt. 525.66). ¹H NMR (400 MHz, CD₃OD): δ 7.74 (d, J=6.4 Hz, 1H), 7.59 (s, 1H), 7.54 (d, J=8 Hz, 1H), 7.42 (d, J=7.6 Hz, 1H), 6.54 (s, 1H), 6.35 (s, 1H), 4.54-4.51 (m, 2H), 3.61-3.53 (m, 2H), 3.08 (s, 3H), 2.90-2.83 (m, 2H), 2.13-2.05 (m, 2H), 2.44-2.40 (m, 2H), 2.26-2.21 (m, 2H), 2.14-2.07 (m, 3H), 1.96-1.85 (m, 5H), 1.45-1.35 (m, 2H).

### Example 48: Synthesis of 1-(5-cyano-2,3-dihydro-1H-inden-2-yl)-3-((1R,4R)-4-((4-((5-(3,3-difluorocyclobutyl)-1H-pyrazol-3-yl)amino)pyrimidin-2-yl)(methyl)amino)cyclohexyl)urea (Compound 145)

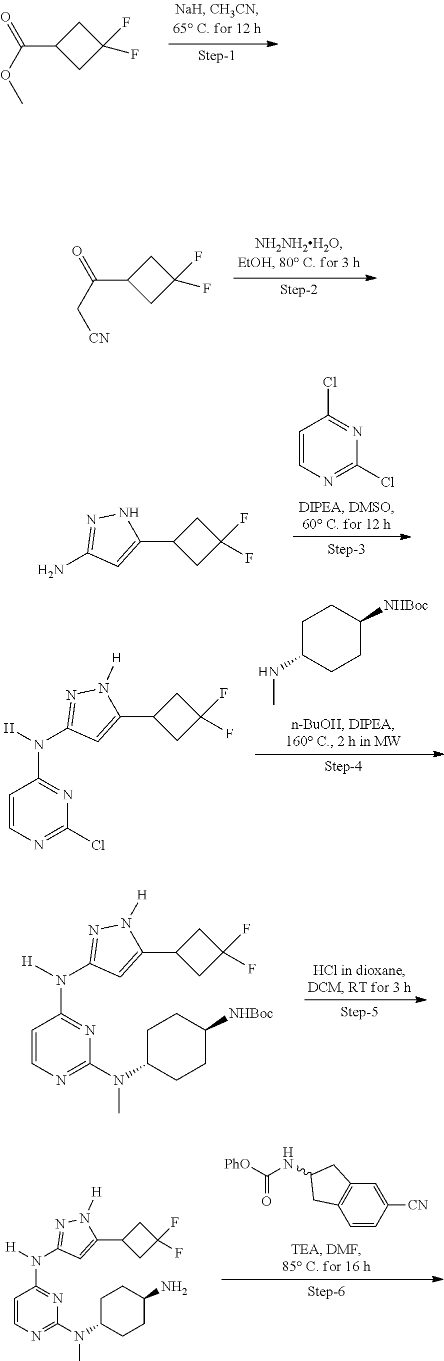

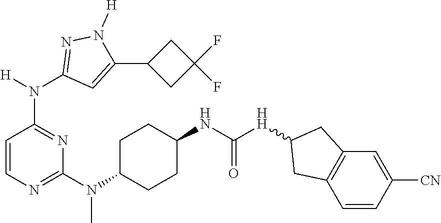

### Step 1: Synthesis of 3-(3,3-difluorocyclobutyl)-3-oxopropanenitrile

To a slurry of sodium hydride (1.06 g, 26.6 mmol) in THE (600 mL) at 65° C. was added a mixture of methyl 3,3-difluorocyclobutane-1-carboxylate (2 g, 13.3 mmol) and acetonitrile (1.4 mL, 26.6 mmol) drop wise. The reaction mixture was stirred at 65° C. for 12 h. The progress of the reaction was monitored by TLC after complete consumption of starting material, the reaction mass was poured into ice and extracted with diethyl ether. The aqueous layer was acidified to a pH=4-5 using 1.5 N HCl and then extracted with diethyl ether. The organic layer separated was dried over anhydrous Na₂SO₄, filtered and the solvent was concentrated to get 3-(3,3-difluorocyclobutyl)-3-oxopropanenitrile (1.7 g, 80.1% yield). LC purity: Not ionized (Mol. formula C₇H₇F₂NO calcd. mol. wt 159.14).

### Step 2: Synthesis of 5-(3,3-diflurocyclobutyl)-1H-pyrazol-3-amine

To a stirred solution of 3-(3,3-difluorocyclobutyl)-3-oxopropanenitrile (1.7 g, 10.69 mmol) in ethanol (20 mL) was added hydrazine hydrate (1.069 mL, 21.38 mmol). The reaction mixture was heated to 80° C. for 3 h. The progress of the reaction was monitored by TLC after complete consumption of starting material, the reaction mixture was concentrated under reduced pressure and the crude was washed with diethyl ether to get 5-(3,3-diflurocyclobutyl)-1H-pyrazol-3-amine (1.7 g, 92.3%). LC purity: 87.15%; m/z: 174.1 [M+H]⁺ (Mol. formula C₇H₉F₂N₃ calcd. mol. wt. 173.17).

### Step 3: Synthesis of 2-chloro-N-(5-(3,3-difluorocyclobutyl)-1H-pyrazol-3-yl)pyrimidin-4-amine

To a stirred solution of 5-(3,3-difluorocyclobutyl)-1H-pyrazol-3-amine (1.7 g, 9.82 mmol) in DMSO (10 mL) was added DIPEA (2.56 mL, 14.73 mmol) and 2, 4-dichloropyrimidine (1.74 g, 11.78 mmol). The reaction was heated to 60° C. for 12 h. The progress of the reaction was monitored by TLC after complete consumption of starting material, the reaction was diluted with ethyl acetate and water wash was given, the organic layer was dried over anhydrous sodium sulphate, filtered and concentrated under reduced pressure. The crude was purified by Biotage-Isolera using silica gel (230-400 mesh) with a gradient elution of 0-60% ethyl acetate in pet ether to yield 2-chloro-N-(5-(3,3-difluorocyclobutyl)-1H-pyrazol-3-yl)pyrimidin-4-amine (900 mg, 32.14%). LC purity: 93.4%; m/z: 286, 288 [M+H]⁺ (Mol. formula C₁₁H₁₀ClF₂N₅ calcd. mol. wt. 285.68).

### Step-4: Synthesis of tert-butyl((1R,4R)-4-((4-((5-(3,3-difluorocyclobutyl-1H-pyrazol-3-yl) amino)pyrimidin-2-yl)(methyl)amino)cyclohexyl)carbamate

In a 20 mL microwave vial a mixture of 2-chloro-N-(5-(3,3-difluorocyclobutyl-1H-pyrazol-3-yl)pyrimidin-4-amine (0.900 g, 3.15 mmol) in n-Butanol (10 mL) was added DIPEA (1.65 mL, 9.45 mmol) and tert-butyl((1R,4R)-4-(methyl amino)cyclohexyl)carbamate (1.44 g, 6.31 mmol). The reaction mixture was subjected to microwave heating at 160° C. for 2 h. After complete consumption of starting material (monitored by TLC), the reaction mixture was concentrated under reduced pressure to remove n-butanol. The crude was purified by Biotage-Isolera using silica gel (230-400 mesh) with gradient elution of 0-80% pet ether-ethyl acetate to yield tert-butyl((1R,4R)-4-((4-((5-(3,3-difluorocyclobutyl-1H-pyrazol-3-yl)amino) pyrimidin-2-yl) (methyl)amino)cyclohexyl) carbamate (450 mg, 30%) LC purity: 59.4%; m/z: 478.3 [M+H]⁺ (Mol. formula C₂₃H₃₃F₂N₇O₂ calcd. mol. wt. 477.56).

### Step-5: Synthesis of N2-((1R,4R)-4-aminocyclohexyl)-N4-(5-(3,3-difluorocyclobutyl)-1H-pyrazol-3-yl)-N2-methylpyrimidine-2,4-diamine

To a stirred solution of tert-butyl ((1R,4R)-4-((4-((5-(3,3-difluorocyclobutyl)-1H-pyrazol-3-yl)amino)pyrimidin-2-yl)(methyl)amino)cyclohexyl)carbamate (0.450 g, 0.94 mmol) in dry DCM (4 mL) was cooled to 0° C. and added HCl in Dioxane (5 mL, 4M solution). The reaction mass was allowed to stir at room temperature for 3 h. The progress of the reaction was monitored by TLC. After complete consumption of starting material, the reaction mixture was concentrated and triturated with pet ether and concentrated under high vaccum to yield N2-((1R,4R)-4-aminocyclohexyl)-N4-(5-(3,3-difluorocyclobutyl)-1H-pyrazol-3-yl)-N2-methylpyrimidine-2,4-diamine as a HCl salt (380 mg, quantitative yield). LC purity: 95.79%; m/z: 378.3 [M+H]⁺ (Mol. formula C₁₈H₂₅F₂N₇ calcd. mol. wt. 377.74).

### Step-6: Synthesis of 1-(5-cyano-2,3-dihydro-1H-inden-2-yl)-3-((1R,4R)-4-((4-((5-(3,3-difluorocyclobutyl)-1H-pyrazol-3-yl)amino)pyrimidin-2-yl)(methyl)amino)cyclohexyl)urea

To a solution of phenyl(5-cyano-2,3-dihydro-1H-indene-2yl)carbamate (0.147 g, 0.53 mmol) in dry DMF (6 mL) was added triethylamine (0.22 mL, 1.59 mmol) and heated at 65° C. for 1 h. After 1 h, the reaction mixture was cooled to room temperature followed by addition of N2-((1R,4R)-4-aminocyclohexyl)-N4-(5-(3,3-difluorocyclobutyl)-1H-pyrazol-3-yl)-N2-methylpyrimidine-2,4-diamine (0.200 g, 0.53 mmol) and the reaction was heated at 85° C. for 16 h. The reaction mixture was cooled to room temperature, diluted with water and extracted using dichloromethane. The organic layer was dried over anhydrous Na₂SO₄, filtered and concentrated under reduced pressure. The residue was purified by reverse phase preparatory HPLC to yield 1-(5-cyano-2,3-dihydro-1H-inden-2-yl)-3-((1R,4R)-4-((4-((5-(3,3-difluorocyclobutyl)-1H-pyrazol-3-yl)amino)pyrimidin-2-yl)(methyl)amino)cyclohexyl)urea (70 mg, 23.5%) as TFA salt. LC purity: 98.3%; m/z: 562.5 [M+H]⁺ (Mol. formula C₂₉H₃₃F₂N₉O calcd. mol. wt. 561.64). ¹H NMR (400 MHz, CD₃OD): δ 7.74 (d, J=7.2 Hz, 1H), 7.59 (s, 1H), 7.54 (d, J=7.6 Hz, 1H), 7.41 (d, J=7.6 Hz, 1H), 6.60 (s, 1H), 6.35 (d, J=7.2 Hz, 1H), 4.54-4.51 (m, 1H), 3.52-3.50 (m, 2H), 3.09 (s, 3H), 3.05-3.03 (m, 2H) 2.91-2.87 (m, 2H), 2.85-2.79 (m, 2H), 2.11 (m, 2H), 1.92-1.84 (m, 4H).

### Example 49: Synthesis of 1-((1R,4R)-4-((4-((5-cyclopentyl-1H-pyrazol-3-yl)amino)pyrimidin-2-yl)(methyl)amino) cyclohexyl)-3-(3-(trifluoromethyl)phenyl)urea (Compound 146)

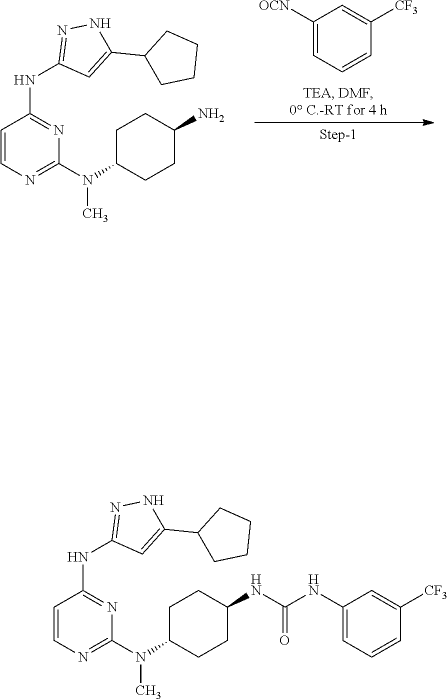

### Step-1: Synthesis of 1-((1R,4R)-4-((4-((5-cyclopentyl-1H-pyrazol-3-yl)amino)pyrimidin-2-yl)(methyl) amino)cyclohexyl)-3-(3-(trifluoromethyl)phenyl)urea

To a stirred solution of N2-((1R,4R)-4-aminocyclohexyl)-N4-(5-cyclopentyl-1H-pyrazol-3-yl)-N2-methylpyrimidine-2,4-diamine (200 mg, 0.563 mmol) in dry DCM (3 mL) at 0° C. triethylamine (0.08 mL, 0.563 mmol) and 1-isocyanato-3-(trifluoromethyl)benzene (0.06 mg, 0.450 mmol) were added drop wise. The reaction mass was stirred at room temperature for 4 h (The reaction mixture was monitored by TLC). After completion of the reaction, reaction mixture was diluted with dichloromethane washed with water, brine, dried over anhydrous Na₂SO₄ and concentrated to yield the residue. The residue was purified by reverse phase preparatory HPLC to yield 1-((1R,4R)-4-((4-((5-cyclopentyl-1H-pyrazol-3-yl)amino)pyrimidin-2-yl)(methyl) amino)cyclohexyl)-3-(3-(trifluoromethyl)phenyl)urea (30 mg, 10%) as a free salt. LC purity: 99.47%; m/z: 543.3 [M+H]⁺ (Mol. formula C₂₇H₃₃F₃N₈O, calcd. mol. wt. 542.61). ¹H VTNMR (400 MHz, CD₃OD): δ 7.88 (d, J=5.6 Hz, 1H), 7.82 (s, 1H), 7.53 (d, J=8.4 Hz, 1H), 7.44-7.40 (m, 1H), 7.24 (d, J=7.6 Hz, 1H), 6.32 (s, 1H), 6.15 (s, 1H), 3.70-3.57 (m, 2H), 3.13-3.09 (m, 1H), 3.02 (s, 3H), 2.16-2.13 (m, 4H), 1.82-1.77 (m, 10H), 1.54-1.48 (m, 2H).

### Example 50: Synthesis of 1-((1R,4R)-4-((4-((5-cyclopropyl-1H-pyrazol-3-yl)amino)pyrimidin-2-yl)(methyl)amino) cyclohexyl)-3-(3-(trifluoromethyl)phenyl)urea (Compound 147)

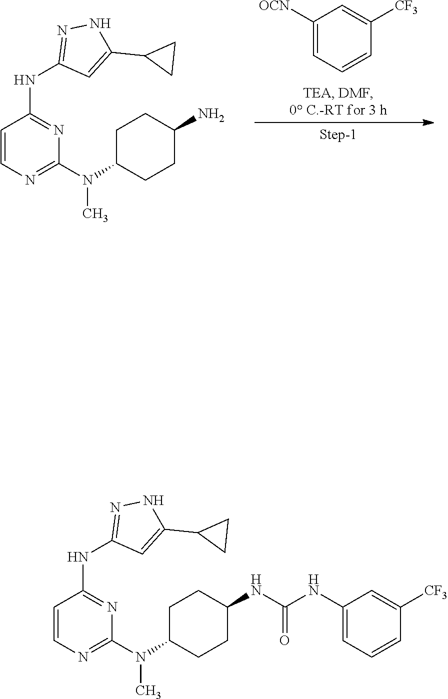

### Step-1: Synthesis of 1-((1R,4R)-4-((4-((5-cyclopropyl-1H-pyrazol-3-yl)amino)pyrimidin-2-yl)(methyl)amino)cyclohexyl)-3-(3-(trifluoromethyl)phenyl)urea

A mixture of N²-((1R,4R)-4-aminocyclohexyl)-N⁴-(5-cyclopropyl-1H-pyrazol-3-yl)-N²-methylpyrimidine-2,4-diamine (200 mg, 0.611 mmol) in DCM (1 mL) at 0° C., triethyl amine (0.08 mL, 0.611 mmol) was added. The resultant mixture was stirred for 10 min at room temperature. Then 1-isocyanato-3-(trifluoromethyl)benzene (91.25 mg, 0.488 mmol) was added and stirred for 3 h at room temperature. The progress of the reaction was monitored by TLC after complete consumption of starting material, water was added and extracted with DCM. The organic layer separated was dried over anhydrous sodium sulphate and concentrated to get crude. The crude compound was purified by reverse phase preparative HPLC to afford pure 1-((1R,4R)-4-((4-((5-cyclopropyl-1H-pyrazol-3-yl)amino)pyrimidin-2-yl)(methyl)amino)cyclohexyl)-3-(3-(trifluoromethyl)phenyl)urea (30 mg, 9% yield). LC purity: 99.59%; m/z: 515.24 [M+H]⁺ (Mol. formula C₂₅H₂₉F₃N₈O, calcd. mol. wt. 514.56). ¹H NMR (400 MHz, CD₃OD): δ 7.88 (d, J=8.0 Hz 1H), 7.82 (s, 1H), 7.53 (d, J=8.0 Hz, 1H), 7.42 (t, J=8.0 Hz, 1H), 7.24 (d, J=8.0 Hz, 1H), 6.12 (d, J=8.0 Hz, 2H), 3.62-3.58 (m, 1H), 3.02-3.00 (m, 4H), 2.16-2.13 (m, 2H), 1.94-1.90 (m, 1H), 1.82-1.76 (m, 4H), 1.48-1.44 (m, 2H), 1.00-0.96 (m, 2H), 0.77-0.73 (m, 2H).

### Example 51: Synthesis of 1-(5-cyano-2,3-dihydro-1H-inden-2-yl)-3-((1R,4R)-4-(methyl(4-((5-(spiro[2.3]hexan-5-yl)-1H-pyrazol-3-yl)amino)pyrimidin-2-yl)amino)cyclohexyl)urea (Compound 148)

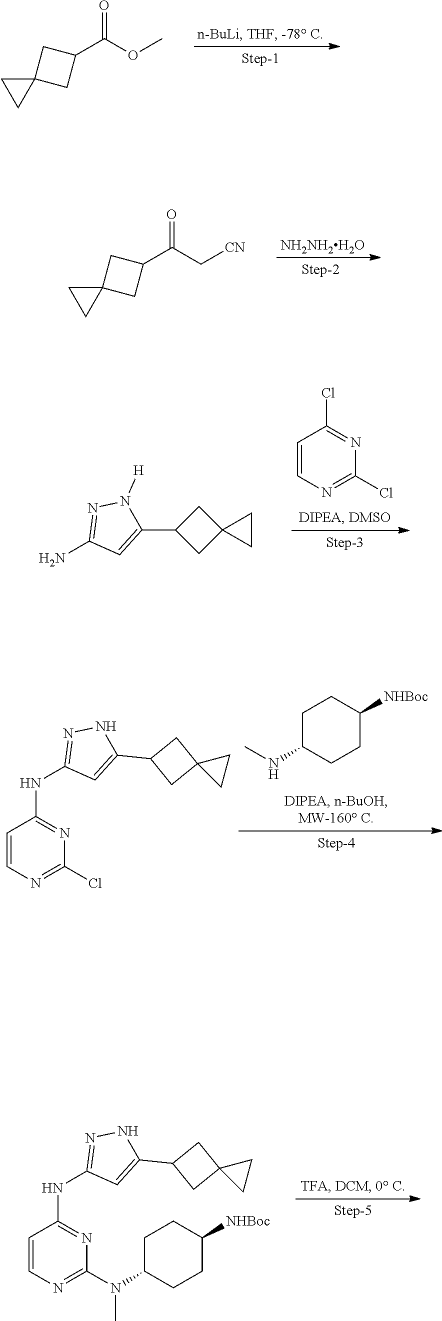

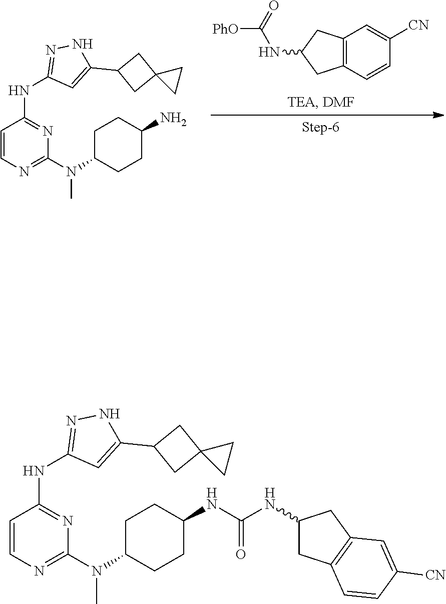 [SMILES: CN(c1nccc(Nc2cc(C3CC4(CC4)C3)[nH]n2)n1)[C@H]1CC[C@H](N)CC1 ; N#Cc1ccc2c(c1)CC(NC(=O)Oc1ccccc1)C2 ; CN(c1nccc(Nc2cc(C3CC4(CC4)C3)[nH]n2)n1)[C@H]1CC[C@H](NC(=O)NC2Cc3ccc(C#N)cc3C2)CC1]

### Step-1: Synthesis of 3-oxo-3-(spiro[2.3]hexan-5-yl)propanenitrile

To a solution of acetonitrile (0.43 mL, 7.133 mmol) in dry THF (5 mL) was cooled to −78° C. and n-BuLi (3.5 mL, 8.917 mmol, 2.5 M in hexane) was added dropwise. The reaction mass was stirred at −78° C. for 30 min. Then methyl spiro[2.3]hexane-5-carboxylate (500 mg, 3.566 mmol) in THF (2 mL) was added dropwise. The reaction mixture was stirred at −40° C. for 2 h. After completion of the reaction (monitored by TLC), the reaction was quenched with saturated ammonium chloride solution and extracted with ethyl acetate. The organic layer was washed with water, dried over anhydrous sodium sulphate, filtered and concentrated under reduced pressure to get 3-oxo-3-(spiro[2.3]hexan-5-yl)propanenitrile (550 mg, crude). LC purity: Not ionized (Mol. formula C₉H₁₁NO, calcd. mol. wt 149.19).

### Step-2: Synthesis of 5-(Spiro [2.3] hexan-5-yl)-1H-pyrazol-3-amine

To a stirred solution of 3-oxo-3-(Spiro [2.3] hexan-5-yl)propanenitrile (550 mg, 3.686 mmol) in ethanol (10 mL) was added hydrazine hydrate (0.4 mL, 7.373 mmol). The reaction mixture was heated to 80° C. for 3 h. The progress of the reaction was monitored by TLC. After complete consumption of starting material, the reaction was concentrated under reduced pressure to get residue. The residue was washed with diethyl ether to get 5-(Spiro[2.3]hexan-5-yl)-1H-pyrazol-3-amine (500 mg, 83.3% yield). LC purity: 84.13%; m/z: 164.1 [M+H]⁺ (Mol. formula C₉H₁₃N₃, calcd. mol. wt. 163.22).

### Step-3: Synthesis of 2-chloro-N-(5-(spiro[2.3]hexan-5-yl)-1H-pyrazol-3-yl)pyrimidin-4-amine

To a stirred solution of 5-(Spiro [2.3]hexan-5-yl)-1H-pyrazol-3-amine (650 mg, 3.982 mmol) in DMSO (1 mL) was added DIPEA (1 mL, 5.973 mmol) and 2, 4-dichloropyrimidine (712 mg, 4.778 mmol). The reaction mixture was heated to 60° C. for 16 h. After complete consumption of the starting material (monitored by TLC), the reaction was quenched with ice water and extracted with dichloromethane. The organic layer was dried over anhydrous sodium sulphate, filtered and concentrated under reduced pressure to get residue. The residue was purified by using Biotage Isolera (230-400 silica gel) with gradient elution of 0-60% ethyl acetate in pet ether to get 2-chloro-N-(5-(spiro[2.3]hexan-5-yl)-1H-pyrazol-3-yl)pyrimidin-4-amine (480 mg, 44% yield). LC purity: 83.45%; m/z: 276.0 [M+H]⁺ (Mol. formula C₁₃H₁₄ClN₅, calcd. mol. wt. 275.74).

### Step-4: Synthesis of yield tert-butyl(1R,4R)-4-(methyl(4-((5-(spiro[2.3]hexan-5-yl)-1H-pyrazol-3-yl)amino)pyrimidin-2-yl)amino)cyclohexyl)carbamate

In a 20 mL microwave vial a mixture of tert-butyl ((1R,4R)-4-(methylamino)cyclohexyl)carbamate (992 mg, 4.352 mmol), 2-chloro-N-(5-(spiro[2.3]hexan-5-yl)-1H-pyrazol-3-yl)pyrimidin-4-amine (480 mg, 1.741 mmol) in n-Butanol (8 mL) was added DIPEA (0.91 mL, 5.223 mmol). The reaction was subjected to microwave at 160° C. for 4 h. The reaction was cooled to room temperature and concentrated to remove n-butanol. The obtained residue was purified by using Biotage Isolera (230-400 silica gel) with gradient elution of 0-80% ethyl acetate in pet ether to yield tert-butyl(1R,4R)-4-(methyl(4-((5-(spiro[2.3]hexan-5-yl)-1H-pyrazol-3-yl)amino)pyrimidin-2-yl)amino)cyclohexyl)carbamate (200 mg, 24.5% yield). LC purity: 87.90%; m/z: 468.4 [M+H]⁺ (Mol. formula C₂₅H₃₇N₇O₂, calcd. mol. wt. 467.62).

### Step-5: Synthesis of N2-((1R,4R)-4-aminocyclohexyl)-N2-methyl-N4-(5-(spiro[2.3]hexan-5-yl)-1H-pyrazol-3-yl)pyrimidine-2,4-diamine

To a stirred solution of tert-butyl ((1R,4R)-4-(methyl(4-((5-(spiro[2.3]hexan-5-yl)-1H-pyrazol-3-yl)amino)pyrimidin-2-yl)amino)cyclohexyl)carbamate (200 mg, 0.427 mmol) in dry DCM (2 mL) was cooled to 0° C. and TFA (2 mL) was added. The reaction mass was stirred at room temperature for 3 h. After complete consumption of the starting material (monitored by TLC), the reaction was concentrated to get residue. The residue was triturated with pet ether and concentrated under reduced pressure to yield N2-((1R,4R)-4-aminocyclohexyl)-N2-methyl-N4-(5-(spiro[2.3]hexan-5-yl)-1H-pyrazol-3-yl)pyrimidine-2,4-diamine (200 mg, quantitative yield) as TFA Salt. LC purity: 94.61%; m/z: 368.1 [M+H]⁺ (Mol. formula C₂₀H₂₉N₇, calcd. mol. wt. 367.50).

### Step-6: Synthesis of 1-(5-cyano-2,3-dihydro-1H-inden-2-yl)-3-((1R,4R)-4-(methyl(4-((5-(spiro[2.3]hexan-5-yl)-1H-pyrazol-3-yl)amino)pyrimidin-2-yl)amino)cyclohexyl)urea

To a stirred solution of phenyl (5-cyano-2,3-dihydro-1H-inden-2-yl)carbamate (151 mg, 0.544 mmol) in dry DMF (2 mL) was cooled 0° C. and added triethylamine (0.07 mL, 0.544 mmol). The reaction was stirred at 0° C. for 1 h. In another vial N2-((1R,4R)-4-aminocyclohexyl)-N2-methyl-N4-(5-(spiro[2.3]hexan-5-yl)-1H-pyrazol-3-yl)pyrimidine-2,4-diamine (200 mg, 0.544 mmol) in dry DMF (1 mL) was added triethylamine (0.15 mL, 1.088 mmol) and stirred for 15 mins. The resulting solution was added to the above reaction at RT. The reaction was heated to 85° C. for 16 h. After completion of the starting material (monitored by TLC), the reaction was diluted with water and extracted with dichloromethane. The resulting organic layer was washed with brine solution then dried over anhydrous Na₂SO₄ and concentrated to get crude. The crude was purified by reverse phase preparative HPLC to get 1-(5-cyano-2,3-dihydro-1H-inden-2-yl)-3-((1R,4R)-4-(methyl(4-((5-(spiro[2.3]hexan-5-yl)-1H-pyrazol-3-yl)amino)pyrimidin-2-yl)amino)cyclohexyl)urea (60 mg, 20% yield) as TFA Salt. LC purity: 99.94%; m/z: 552.2 [M+H]⁺ (Mol. formula C₃₁H₃₇N₉O, calcd. mol. wt. 551.70). ¹H NMR (400 MHz, CD₃OD): δ 7.72 (d, J=7.20 Hz, 1H), 7.57 (s, 1H), 7.52 (d, J=7.60 Hz, 1H), 7.40 (d, J=7.60 Hz, 1H), 6.55-6.32 (m, 2H), 4.58-4.51 (m, 1H), 3.56-3.49 (m, 1H), 3.36-3.34 (m, 1H), 3.33-3.32 (m, 1H), 3.30-3.28 (m, 2H), 3.09 (s, 3H), 2.91-2.87 (m, 2H), 2.47-2.44 (m, 4H), 2.12 (d, J=10 Hz, 2H), 1.86-1.82 (m, 4H), 1.40-1.35 (m, 2H), 0.57-0.53 (m, 2H), 0.47-0.43 (m, 2H).

### Example 52: Synthesis of 1-(5-cyano-2,3-dihydro-1H-inden-2-yl)-3-((1R,4R)-4-((4-((5-(3,3-difluorocyclopentyl)-1H-pyrazol-3-yl)amino)pyrimidin-2-yl)(methyl)amino)cyclohexyl)urea (Compound 149)

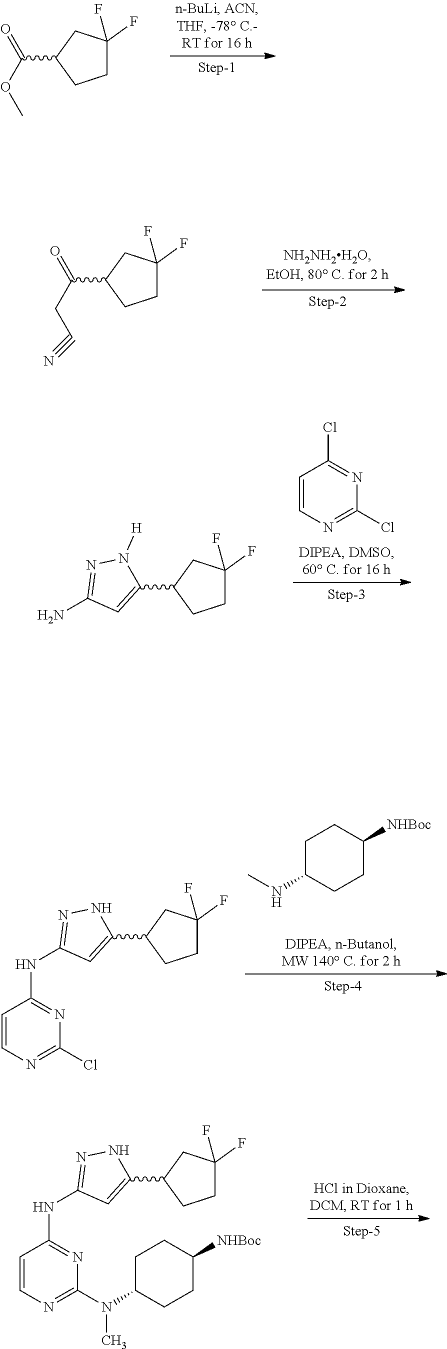

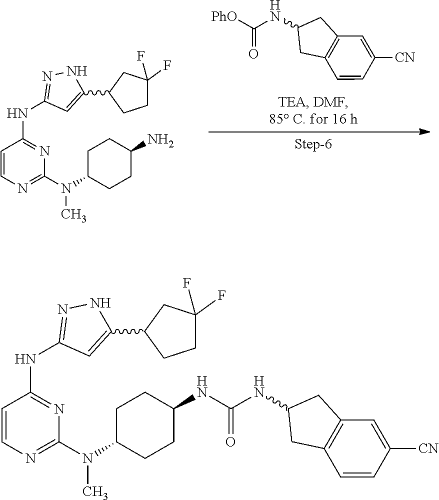 [SMILES: CN(c1nccc(Nc2cc(C3CCC(F)(F)C3)[nH]n2)n1)[C@H]1CC[C@H](N)CC1 ; N#Cc1ccc2c(c1)CC(NC(=O)Oc1ccccc1)C2 ; CN(c1nccc(Nc2cc(C3CCC(F)(F)C3)[nH]n2)n1)[C@H]1CC[C@H](NC(=O)NC2Cc3ccc(C#N)cc3C2)CC1]

### Step-1: Synthesis of 3-(3,3-difluorocyclopentyl)-3-oxopropanenitrile

To a solution of Acetonitrile (1.59 mL, 30.48 mmol) in dry tetrahydrofuran (50 mL) at −78° C. n-BuLi (9.14 mL, 22.86 mmol, 2.5M solution in THF) was added dropwise. The reaction mixture was stirred for 30 min to 1 h at −50° C. The reaction mixture was again cooled to −78° C. and a solution of methyl 3, 3-difluorocyclopentane-1-carboxylate (2.5 g, 15.24 mmol) in dry tetrahydrofuran (10 mL) was added dropwise and was stirred for 1 h at −78° C. The reaction mixture was allowed to attain RT and stirred for 16 h. After completion of the reaction, reaction mixture was quenched with saturated ammonium chloride solution and extracted with ethyl acetate. The combined organic layer was washed with water, brine, dried over anhydrous Na₂SO₄ and concentrated to get crude compound 3-(3,3-difluorocyclopentyl)-3-oxopropanenitrile (2.7 g, quantitative yield). Which was directly used for next step. LC purity: 86.73%; m/z: 172.1 [M−H]⁺ (Mol. formula C₈H₉F₂NO, calcd. mol. wt. 173.16).

### Step-2: Synthesis of 5-(3,3-difluorocyclopentyl)-1H-pyrazol-3-amine

To a solution of 3-(3,3-difluorocyclopentyl)-3-oxopropanenitrile (2.7 g, 15.60 mmol) in EtOH (30 mL) was added Hydrazine hydrate (1.57 mL, 31.21 mmol). The reaction was stirred at 80° C. for 2 h. After completion of the reaction, the reaction mixture was concentrated and washed with 10% diethyl ether in pet ether to remove hydrazine hydrate. Reaction mixture was concentrated to get crude compound 5-(3,3-difluorocyclopentyl)-1H-pyrazol-3-amine (3 g, quantitative yield) which was taken for next step without further purification. LC purity: 87.62%; m/z: 188.2 [M+H]⁺ (Mol. formula C₈H₁₁F₂N₃, calcd. mol. wt. 187.19).

### Step-3: Synthesis of 2-chloro-N-(5-(3,3-difluorocyclopentyl)-1H-pyrazol-3-yl)pyrimidin-4-amine

A mixture of 2,4-dichloropyrimidine (2.2 g, 14.86 mmol), 5-(3,3-difluorocyclopentyl)-1H-pyrazol-3-amine (3.05 g, 16.35 mmol) and DIPEA (7.98 mL, 44.59 mmol) in DMSO (20 mL) were stirred at 60° C. for 16 h. The reaction mixture was monitored by TLC. After completion of starting material, the reaction mixture was cooled to room temperature, water was added and solid was precipitated. The solid was filtered, washed with pet ether and dried under vacuum to yield 2-chloro-N-(5-(3,3-difluorocyclopentyl)-1H-pyrazol-3-yl)pyrimidin-4-amine (1.8 g, 49.54% yield). LC purity: 84.33%; m/z: 300.0 [M+H]⁺ (Mol. formula C₁₂H₁₂ClF₂N₅, calcd. mol. wt. 299.71).

### Step-4: Synthesis of tert-butyl ((1R,4R)-4-((4-((5-(3,3-difluorocyclopentyl)-1H-pyrazol-3-yl)amino)pyrimidin-2-yl)(methyl)amino)cyclohexyl)carbamate

In a 20 mL microwave vial a solution of 2-chloro-N-(5-(3,3-difluorocyclopentyl)-1H-pyrazol-3-yl)pyrimidin-4-amine (0.5 g, 1.67 mmol) in n-BuOH (10 mL) was added DIPEA (0.9 mL, 5.01 mmol) and tert-butyl ((1R,4R)-4-(methylamino)cyclohexyl)carbamate (0.457 g, 2.0 mmol). The reaction mixture was subjected to microwave at 140° C. for 2 h. After completion of the starting material (monitored by TLC), the reaction was cooled to room temperature concentrated to remove n-BuOH to provide crude compound. The crude compound was purified by Biotage Isolera using silica gel (230-400) with gradient elution of 50-100% ethyl acetate in pet ether to get tert-butyl ((1R,4R)-4-((4-((5-(3,3-difluorocyclopentyl)-1H-pyrazol-3-yl)amino)pyrimidin-2-yl)(methyl)amino)cyclohexyl)carbamate (350 mg, 42.6% yield). LC purity: 84.98%; m/z: 492.2 [M+H]⁺ (Mol. formula C₂₄H₃₅F₂N₇O₂, calcd. mol. wt. 491.59).

### Step-5: Synthesis of N2-((1R,4R)-4-aminocyclohexyl)-N4-(5-(3,3-difluorocyclopentyl)-1H-pyrazol-3-yl)-N2-methylpyrimidine-2,4-diamine

To a solution tert-butyl ((1R,4R)-4-((4-((5-(3,3-difluorocyclopentyl)-1H-pyrazol-3-yl)amino)pyrimidin-2-yl)(methyl)amino)cyclohexyl)carbamate (0.35 g, 0.712 mmol) in dry DCM (10 mL) was added HCl in Dioxane (5 mL, 4M solution). The reaction mass was allowed to stir at room temperature for 1 h. The progress of the reaction was monitored by TLC. After complete consumption of starting material, the resulting mixture was concentrated and triturated with pet ether and concentrated under high vacuum to yield N2-((1R,4R)-4-aminocyclohexyl)-N4-(5-(3,3-difluorocyclopentyl)-1H-pyrazol-3-yl)-N2-methylpyrimidine-2,4-diamine (200 mg, quantitative yield). LC purity: 81.39%; m/z: 392.2 [M+H]⁺ (Mol. formula C₁₉H₂₇F₂N₇, calcd. mol. wt. 391.47).

### Step-6: Synthesis of 1-(5-cyano-2,3-dihydro-1H-inden-2-yl)-3-((1R,4R)-4-((4-((5-(3,3-difluorocyclopentyl)-1H-pyrazol-3-yl)amino)pyrimidin-2-yl)(methyl)amino)cyclohexyl)urea

To a stirred solution of phenyl (5-cyano-2,3-dihydro-1H-inden-2-yl)carbamate (142 mg, 0.511 mmol) in dry DMF (4 mL) was added triethylamine (0.21 mL, 1.53 mmol). The reaction mixture was stirred at 65° C. for 1 h and added N2-((1R,4R)-4-aminocyclohexyl)-N4-(5-(3,3-difluorocyclopentyl)-1H-pyrazol-3-yl)-N2-methylpyrimidine-2,4-diamine (200 mg, 0.511 mmol) at RT. The reaction was stirred at 85° C. for 16 h. The reaction mixture was monitored by TLC. After completion of the reaction, the reaction mixture was diluted with water and extracted with dichloromethane. The resulting organic layer was washed with brine solution then dried over anhydrous Na₂SO₄ and concentrated to obtain crude compound which was then purified by reverse phase preparative HPLC to obtain 1-(5-cyano-2,3-dihydro-1H-inden-2-yl)-3-((1R,4R)-4-((4-((5-(3,3-difluorocyclopentyl)-1H-pyrazol-3-yl)amino)pyrimidin-2-yl)(methyl)amino)cyclohexyl)urea (100 mg, 34.01% yield) as TFA salt. LC purity: 95.30%; m/z: 576.0 [M+H]⁺ (Mol. formula C₃₀H₃₅F₂N₉O, calcd. mol. wt. 575.67). ¹H NMR (400 MHz, CD₃OD): δ 7.73 (d, J=7.2 Hz, 1H), 7.56 (s, 1H), 7.52 (d, J=7.6 Hz, 1H), 7.39 (d, J=7.6 Hz, 1H), 6.55-6.30 (m, 2H), 4.58-4.51 (m, 2H), 3.51-3.47 (m, 2H), 3.35-3.28 (m, 2H), 3.09 (s, 3H), 2.91-2.83 (m, 2H), 2.54-2.64 (m, 1H), 2.33-2.11 (m, 6H), 1.98-1.95 (m, 1H), 1.86-1.82 (m, 4H), 1.36-1.32 (m, 2H).

### Example 53: Synthesis of 1-((1R,4R)-4-((4-((5-cyclopropyl-1H-pyrazol-3-yl)amino)pyrimidin-2-yl)(methyl)amino) cyclohexyl)-3-(2,3-dihydro-1H-inden-2-yl)urea (Compound 150)

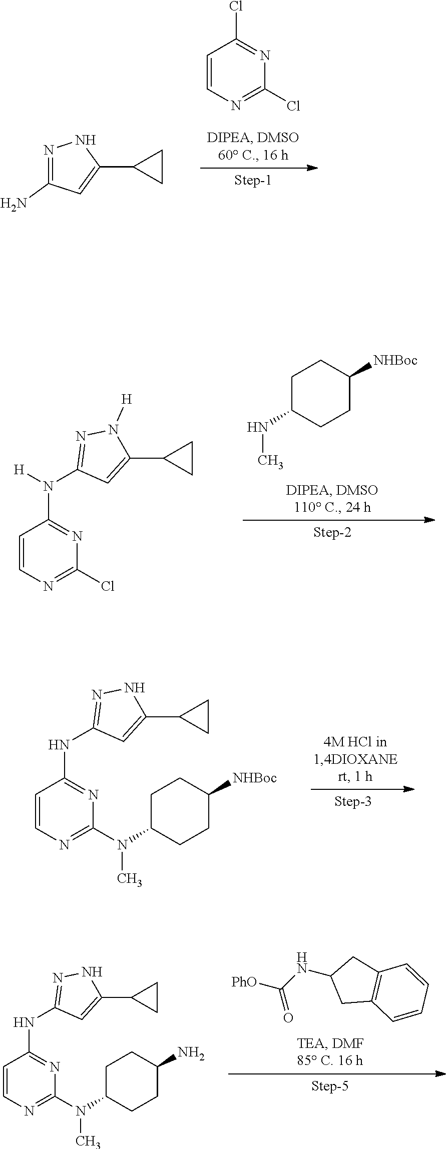

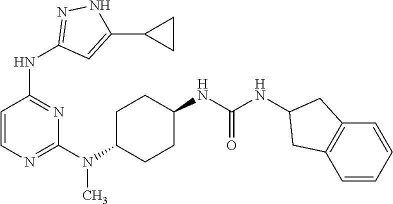 [SMILES: CN(c1nccc(Nc2cc(C3CC3)[nH]n2)n1)[C@H]1CC[C@H](NC(=O)NC2Cc3ccccc3C2)CC1]

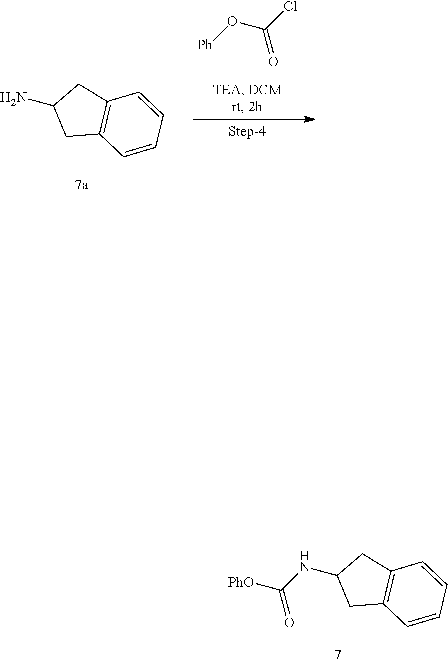 [SMILES: O=C(Cl)Oc1ccccc1 ; NC1Cc2ccccc2C1 ; O=C(NC1Cc2ccccc2C1)Oc1ccccc1]

### Step-1: Synthesis of 2-chloro-N-(5-cyclopropyl-1H-pyrazol-3-yl)pyrimidin-4-amine

A mixture of 2,4-dichloropyrimidine (14.43 g, 97.56 mmol), 5-cyclopropyl-1H-pyrazol-3-amine (10 g, 81.3 mmol) and DIPEA (21.25 mL, 121.95 mmol) in DMSO (100 mL) was heated at 60° C. for 16 h. After complete consumption of the starting material (monitored by TLC), the reaction was cooled to room temperature and diluted with water. The solid precipitated was filtered, washed with pet ether and dried under vaccum to get 2-chloro-N-(5-cyclopropyl-1H-pyrazol-3-yl)pyrimidin-4-amine (17.0 g, 89% yield). LC purity: 72.2%; m/z: 236.07 [M+H]f (Mol. formula C₁₀H₁₀ClN₅, calcd. mol. wt. 235.68).

### Step-2: Synthesis of tert-butyl ((1R,4R)-4-((4-((5-cyclopropyl-1H-pyrazol-3-yl)amino) pyrimidin-2-yl)(methyl)amino)cyclohexyl)carbamate

A mixture of 2-chloro-N-(5-cyclopropyl-1H-pyrazol-3-yl)pyrimidin-4-amine (2 g, 8.510 mmol), tert-butyl ((1R,4R)-4-(methylamino)cyclohexyl)carbamate (2.3 g, 10.21 mmol) and DIPEA (2.96 mL, 17.02 mmol) in DMSO (20 mL) was heated in a sealed tube at 110° C. for 24 h. After complete consumption of starting material (monitored by TLC), the reaction mixture was cooled to room temperature and diluted with water. The solid precipitated was filtered, washed with pet ether and dried under vacuum to get tert-butyl ((1R,4R)-4-((4-((5-cyclopropyl-1H-pyrazol-3-yl)amino)pyrimidin-2-yl)(methyl)amino)cyclohexyl)carbamate (2 g, 55% yield). LC purity: 84.3%; m/z: 428.27 [M+H]f (Mol. formula C₂₂H₃₃N₇O₂, calcd. mol. wt. 427.55).

### Step-3: Synthesis of N²-((1R,4R)-4-aminocyclohexyl)-N⁴-(5-cyclopropyl-1H-pyrazol-3-yl)-N²-methylpyrimidine-2,4-diamine

A mixture of tert-butyl ((1R,4R)-4-((4-((5-cyclopropyl-1H-pyrazol-3-yl)amino)pyrimidin-2-yl)(methyl)amino)cyclohexyl)carbamate (2.0 g, 4.683 mmol) in DCM (20 mL) was cooled to 0° C. and 4M HC in 1,4 dioxane (15 mL) was added. The resulting mixture was stirred at room temperature for 1 h. The progress of the reaction was monitored by TLC after complete consumption of starting material, reaction mixture was concentrated under vaccum to yield N²-((1R,4R)-4-aminocyclohexyl)-N⁴-(5-cyclopropyl-1H-pyrazol-3-yl)-N²-methylpyrimidine-2,4-diamine (1.8 g, quantitative yield). LC purity: 73.1%; m/z: 328.22 [M+H]⁺ (Mol. formula C₁₇H₂₅N₇, calcd. mol. wt. 327.44).

### Step-4: Synthesis of phenyl (2,3-dihydro-1H-inden-2-yl) carbamate

To a mixture of 2,3-dihydro-1H-inden-2-amine (100 mg, 0.751 mmol) in DCM (2 mL), was added TEA (0.15 mL, 1.12 mmol) at 0° C., phenyl carbonochloridate (117.15 mg, 0.751 mmol) at same temperature and stirred at room temperature for 2 h. The progress of the reaction was monitored by TLC. After complete consumption of starting material, water was added and extracted with DCM. The organic layer separated was dried over anhydrous sodium sulphate and concentrated. The solid was washed with pet ether and dried under vaccum to yield phenyl (2,3-dihydro-1H-inden-2-yl) carbamate (150 mg, 78% yield). LC purity: 76.00%; m/z: 254.11 [M+H]⁺ (Mol. formula C₁₆H₁₅NO₂, calcd. mol. Wt. 253.30).

### Step-5: Synthesis of 1-((1R,4R)-4-((4-((5-cyclopropyl-1H-pyrazol-3-yl)amino)pyrimidin-2-yl)(methyl)amino)cyclohexyl)-3-(2,3-dihydro-1H-inden-2-yl)urea

To a stirred solution of phenyl (2,3-dihydro-1H-inden-2-yl) carbamate (154.58 mg, 0.611 mmol) in dry DMF was added trimethylamine (0.08 mL, 0.611 mmol). The reaction mixture was heated to 85° C. for 1 h. The reaction mixture was cooled to room temperature and added N2-((1R,4R)-4-aminocyclohexyl)-N4-(5-cyclopropyl-1H-pyrazol-3-yl)-N2-methylpyrimidine-2,4-diamine (200 mg, 0.611 mmol) in TEA (0.08 mL, 0.611 mmol). The reaction mixture was heated to 85° C. and stirred for 16 h. After complete consumption of starting material (monitored by TLC), the reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield 1-((1R,4R)-4-((4-((5-cyclopropyl-1H-pyrazol-3-yl)amino)pyrimidin-2-yl)(methyl)amino)cyclohexyl)-3-(2,3-dihydro-1H-inden-2-yl)urea as TFA salt (30 mg, 10% yield). LC purity: 99.95%; m/z: 487.29 [M+H]⁺ (Mol. formula C₂₇H₃₄N₈O, calcd. mol. wt. 486.62). ¹H NMR (400 MHz, CD₃OD): δ 7.71 (d, 1H), 7.20-7.17 (m, 2H), 7.14-7.11 (m, 2H), 6.36 (s, 1H), 6.33 (d, J=8 Hz, 2H), 4.48-4.45 (m, 2H), 3.53-3.49 (m, 1H), 3.26-3.21 (m, 2H), 3.06 (s, 3H), 2.79-2.74 (m, 2H), 2.14-2.11 (m, 2H), 1.94-1.82 (m, 5H), 1.39-1.32 (m, 2H), 1.05-1.00 (m, 2H), 0.77-0.73 (m, 2H).

### Example 54: Synthesis of 1-((1R,4R)-4-((4-((5-cyclopropyl-1H-pyrazol-3-yl)amino)pyrimidin-2-yl) methyl) amino)cyclohexyl)-3-(5,6-difluoro-2,3-dihydro-1H-inden-2-yl)urea (Compound 151)

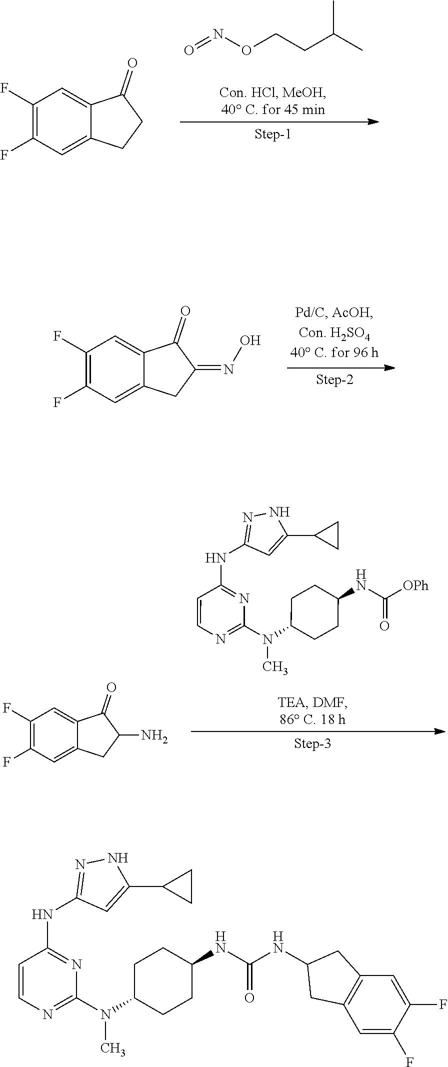

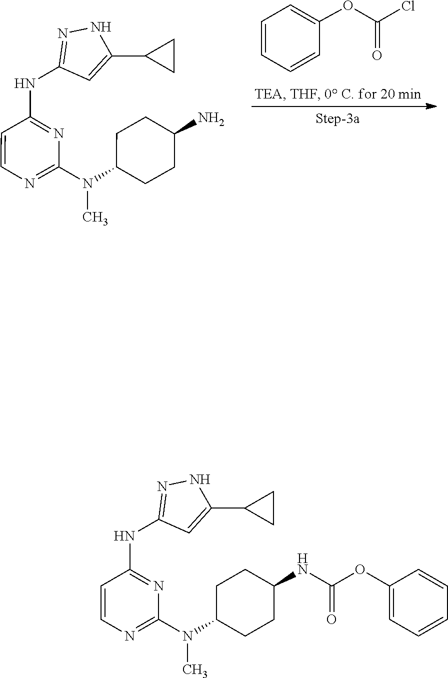 [SMILES: CN(c1nccc(Nc2cc(C3CC3)[nH]n2)n1)[C@H]1CC[C@H](N)CC1 ; O=C(Cl)Oc1ccccc1 ; CN(c1nccc(Nc2cc(C3CC3)[nH]n2)n1)[C@H]1CC[C@H](NC(=O)Oc2ccccc2)CC1]

### Step-1: Synthesis of (Z)-5,6-difluoro-2-(hydroxyimino)-2,3-dihydro-1H-inden-1-one

To a methanol solution (30 mL) containing 5,6-difluoro-2,3-dihydro-1H-inden-1-one (4.60 g, 27.4 mmol) at 40° C., was added isoamyl nitrite (4.17 g, 35.6 mmol) followed by concentrated HCl (2.7 mL). Upon heating for 45 min the solution was allowed to cool to room temperature and water was added. The resulting precipitate was collected via vacuum filtration. The solid was washed thoroughly with water to yield (Z)-5,6-difluoro-2-(hydroxyimino)-2,3-dihydro-1H-inden-1-one (3.97 g, 20.15 mmol, 73.6% yield) as a light orange solid. LC purity: 85.99%; m/z: 198.1 [M+H]⁺ (Mol. formula C₉H₅F₂NO₂, calcd. mol. wt. 197.14).

### Step-2: Synthesis of 5,6-Difluoro-2,3-dihydro-1H-inden-2-amine

A solution of (Z)-5,6-difluoro-2-(hydroxyimino)-2,3-dihydro-1H-inden-1-one (1 g, 5.07 mmol) in acetic acid (15 mL) was added 1 mL of concentrated H₂SO₄ followed by 10% Pd/C (500 mg). The reaction was hydrogenated under 10 Kg pressure for 96 h in a tiny clave at 60° C. The reaction was filtered through a celite bed and washed thoroughly with methanol. The solvent was then removed in vacuo to give crude compound which was purified by reverse phase preparative HPLC to give 5,6-Difluoro-2,3-dihydro-1H-inden-2-amine (80 mg, 9.33% yield). LC purity: 99.84%; m/z: 170.1 [M+H]⁺ (Mol. formula C₉H₉F₂N, calcd. mol. wt. 169.17).

### Step-3a: Synthesis of phenyl ((1R,4R)-4-((4-((5-cyclopropyl-1H-pyrazol-3-yl)amino)pyrimidin-2-yl)(methyl)amino)cyclohexyl)carbamate

To a stirred solution of N2-((1R,4R)-4-aminocyclohexyl)-N4-(5-cyclopropyl-1H-pyrazol-3-yl)-N2-methylpyrimidine-2,4-diamine (1 g, 3.058 mmol) in anhydrous THF (80 mL) was added tri ethylamine (3.86 mL, 27.522 mmol). The reaction mixture was stirred at 0° C. for 1 h and then added phenyl carbonochloridate (0.39 mL, 3.058 mmol) in THF (20 mL). The reaction mixture was stirred at 0° C. for 20 minutes. After completion of the reaction (monitored by TLC), the reaction mixture was diluted with water and extracted with ethyl acetate. The resulting organic layer was washed with brine solution then dried over anhydrous Na₂SO₄ and concentrated to obtain crude compound which was then purified by Biotage Isolera using 230-400 silica gel eluted with 0-100% ethyl acetate in pet ether to yield phenyl ((1R,4R)-4-((4-((5-cyclopropyl-1H-pyrazol-3-yl)amino)pyrimidin-2-yl)(methyl)amino)cyclohexyl)carbamate (200 mg, 14.64%). LC purity: 93.55%; m/z: 448.2 [M+H]⁺ (Mol. formula C₂₄H₂₉N₇O₂, calcd. mol. wt. 447.54).

### Step-3: Synthesis of 1-((1R,4R)-4-((4-((5-cyclopropyl-1H-pyrazol-3-yl)amino)pyrimidin-2-yl)(methyl)amino)cyclohexyl)-3-(5,6-difluoro-2,3-dihydro-1H-inden-2-yl)urea

To a stirred solution of phenyl ((1R,4R)-4-((4-((5-cyclopropyl-1H-pyrazol-3-yl)amino) pyrimidin-2-yl)(methyl)amino)cyclohexyl)carbamate (200 mg, 0.447 mmol) in dry DMF (4 mL) was added tri ethylamine (0.19 mL, 1.34 mmol). The reaction mixture was stirred at 65° C. for 1 h and added 5,6-difluoro-2,3-dihydro-1H-inden-2-amine (75.6 mg, 0.447 mmol) at RT. The reaction was stirred at 85° C. for 16 h. The reaction mixture was monitored by TLC. After completion of the reaction, the reaction mixture was diluted with water and extracted with dichloromethane. The resulting organic layer was washed with brine solution then dried over anhydrous Na₂SO₄ and concentrated to obtain crude compound which was then purified by reverse phase preparative HPLC to obtain 1-((1R,4R)-4-((4-((5-cyclopropyl-1H-pyrazol-3-yl)amino)pyrimidin-2-yl)(methyl)amino)cyclohexyl)-3-(5,6-difluoro-2,3-dihydro-1H-inden-2-yl)urea (20 mg, 8.58% yield) as TFA salt. LC purity: 99.74%; m/z: 523.9 [M+H]⁺ (Mol. formula C₂₇H₃₂F₂N₈O, calcd. mol. wt. 522.60). ¹H VTNMR (400 MHz, CD₃OD): δ 7.74 (d, J=6.0 Hz, 1H), 6.96 (t, J=9.2 Hz, 2H), 6.03-5.97 (m, 2H), 3.48-3.36 (m, 1H), 3.13-3.08 (m, 2H), 2.89 (s, 3H), 2.66-2.61 (m, 2H), 1.97-1.94 (m, 2H), 1.80-1.77 (m, 1H), 1.67-1.61 (m, 4H), 1.27-1.19 (m, 4H), 0.88-0.83 (m, 2H), 0.63-0.59 (m, 2H).

### Example 55: Synthesis of 1-(5-cyano-2,3-dihydro-1H-inden-2-yl)-3-((1R,4R)-4-((4-((5-cyclopropyl-1H-pyrazol-3-yl)amino)pyrimidin-2-yl)(2-methoxyethyl)amino)cyclohexyl)urea (Compound 152)

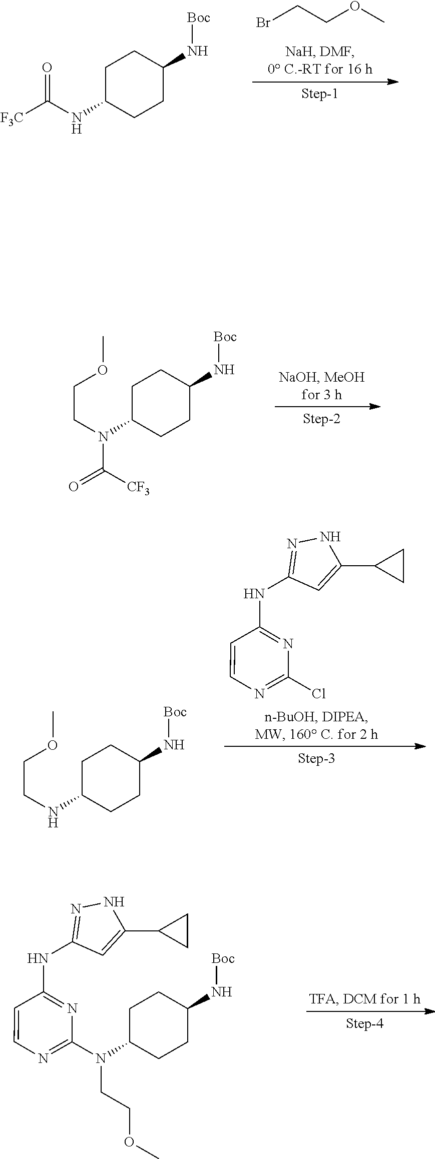

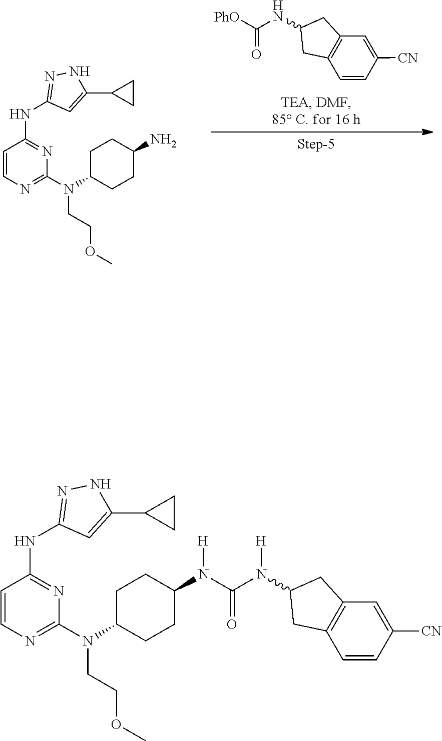 [SMILES: COCCN(c1nccc(Nc2cc(C3CC3)[nH]n2)n1)[C@H]1CC[C@H](N)CC1 ; N#Cc1ccc2c(c1)CC(NC(=O)Oc1ccccc1)C2 ; COCCN(c1nccc(Nc2cc(C3CC3)[nH]n2)n1)[C@H]1CC[C@H](NC(=O)NC2Cc3ccc(C#N)cc3C2)CC1]

### Step-1: Synthesis of tert-butyl ((1R,4R)-4-(2,2,2-trifluoro-N-(2-methoxyethyl)acetamido) cyclohexyl)carbamate

To a stirred solution of tert-butyl ((1R,4R)-4-(2,2,2-trifluoroacetamido)cyclohexyl)carbamate (3 g, 9.677 mmol) in dry DMF (30 mL) at 0° C. sodium hydride (1.935 g, 48.387 mmol) was added portion wise. The reaction mixture was stirred at room temperature for 30 minutes then added 1-bromo-2-methoxyethane (1.372 mL, 14.516 mmol) dropwise at 0° C. The reaction mixture was allowed to stir for 16 h. After completion of the reaction the reaction (monitored by LCMS), the reaction mixture was quenched with ice, neutralized with 2N citric acid and extracted with dichloromethane. The organic layer was dried over anhydrous sodium sulphate, filtered and concentrated under reduced pressure to get tert-butyl ((1R,4R)-4-(2,2,2-trifluoro-N-(2-methoxyethyl)acetamido)cyclohexyl)carbamate (3 g, 84.26%). LC purity: 90.43%; m/z: 313.2 [M−(t−Bu)]⁺ (Mol. formula C₁₆H₂₇F₃N₂O₄ calcd. mol. wt. 368.40).

### Step-2: Synthesis of tert-butyl ((1R,4R)-4-((2-methoxyethyl)amino)cyclohexyl) carbamate

A suspension of tert-butyl ((1R,4R)-4-(2,2,2-trifluoro-N-(2-methoxyethyl)acetamido) cyclohexyl)carbamate (3 g, 8.152 mmol) in methanol (30 mL) was heated to 50° C. with stirring for a few minutes until a clear solution was formed. Then 2N sodium hydroxide solution was added with stirring till slightly cloudy solution was formed. The resulting mixture was stirred for 3 at room temperature. After completion of the reaction (monitored by LCMS), the reaction mixture was concentrated, the residue was taken up in 2N citric acid solution and extracted with dichloromethane/methanol (9:1). The organic layer was dried over anhydrous sodium sulphate, filtered and concentrated under reduced pressure to get tert-butyl ((1R,4R)-4-((2-methoxyethyl)amino)cyclohexyl)carbamate (1.8 g, 81.44%). LC purity: 94.02%; m/z: 217.3 [M−(t−Bu)]⁺ (Mol. formula C₁₄H₂₈N₂O₃ calcd. mol. wt. 272.39).

### Step-3: Synthesis of tert-butyl ((1R,4R)-4-((4-((5-cyclopropyl-1H-pyrazol-3-yl)amino) pyrimidin-2-yl)(2-methoxyethyl)amino)cyclohexyl)carbamate

In a 20 mL microwave vial a mixture of tert-butyl ((1R,4R)-4-((2-methoxyethyl)amino) cyclohexyl)carbamate (1.8 g, 6.617 mmol), 2-chloro-N-(5-cyclopropyl-1H-pyrazol-3-yl) pyrimidin-4-amine (770 mg, 3.308 mmol) in n-butanol (18 mL) was added DIPEA (1.73 mL, 9.926 mmol). The reaction mixture was heated in a microwave at 160° C. for 2 h. The reaction mixture was cooled to room temperature and concentrated to remove n-butanol. The obtained residue was purified by Biotage Isolera using silica gel (230-400 mesh) with gradient elution of 0-10% methanol in dichloromethane to yield tert-butyl ((1R,4R)-4-((4-((5-cyclopropyl-1H-pyrazol-3-yl)amino)pyrimidin-2-yl)(2-methoxyethyl)amino)cyclohexyl) carbamate (635 mg, 20.37%). LC purity: 40.43%; m/z: 472.2 [M+H]⁺ (Mol. formula C₂₄H₃₇N₇O₃ calcd. mol. wt. 471.6).

### Step-4: Synthesis of N2-((1R,4R)-4-aminocyclohexyl)-N4-(5-cyclopropyl-1H-pyrazol-3-yl)-N2-(2-methoxyethyl) pyrimidine-2,4-diamine

To a stirred solution of tert-butyl ((1R,4R)-4-((4-((5-cyclopropyl-1H-pyrazol-3-yl)amino)pyrimidin-2-yl)(2-methoxyethyl)amino)cyclohexyl)carbamate (600 mg, 1.273 mmol) in DCM (7 mL) added trifluoroacetic acid (6 mL) dropwise at 0° C. and allowed to stir at the same temperature for 1 h. After completion of the reaction (monitored by TLC), the reaction mixture was concentrated to get N2-((1R,4R)-4-aminocyclohexyl)-N4-(5-cyclopropyl-1H-pyrazol-3-yl)-N2-(2-methoxyethyl)pyrimidine-2,4-diamine (450 mg, quantitative yield). LC purity: 57.92%; m/z: 372.1 [M+H]⁺ (Mol. formula C₁₉H₂₉N₇O calcd. mol. wt. 371.4).

### Step-5: Synthesis of 1-(5-cyano-2,3-dihydro-1H-inden-2-yl)-3-((1R,4R)-4-((4-((5-cyclopropyl-1H-pyrazol-3-yl)amino)pyrimidin-2-yl)(2-methoxyethyl)amino)cyclohexyl)urea

To a stirred solution of phenyl (5-cyano-2,3-dihydro-1H-inden-2-yl)carbamate (187 mg, 0.673 mmol) in dry DMF (2.5 mL) triethylamine (0.28 mL, 2.021 mmol) was added and stirred at 0° C. to room temperature for 1 h. To this N2-((1R,4R)-4-aminocyclohexyl)-N4-(5-cyclopropyl-1H-pyrazol-3-yl)-N2-(2-methoxyethyl)pyrimidine-2,4-diamine (250 mg, 0.673 mmol) was added and heated to 85° C. for 16 h. After complete conversion of the starting material (monitored by TLC), the reaction mixture was diluted with water and extracted with dichloromethane. The resulting organic layer was washed with brine solution then dried over anhydrous Na₂SO₄ and concentrated to obtain crude compound. The crude compound was purified by reverse phase preparative HPLC to yield 1-(5-cyano-2,3-dihydro-1H-inden-2-yl)-3-((1R,4R)-4-((4-((5-cyclopropyl-1H-pyrazol-3-yl)amino)pyrimidin-2-yl)(2-methoxyethyl) amino)cyclohexyl)urea (40 mg, 10.72%) as a TFA salt. LC purity: 97.28%; m/z: 556.1 [M+H]⁺ (Mol. formula C₃₀H₃₇N₉O₂, calcd. mol. wt. 555.69). ¹H NMR (400 MHz, CD₃OD): δ 7.69 (d, J=7.2 Hz, 1H), 7.62 (s, 1H), 7.55 (d, J=7.6 Hz, 1H), 7.44 (d, J=8.0 Hz, 1H), 6.41 (s, 1H), 6.29 (d, J=6.0 Hz, 1H), 4.53-4.50 (m, 1H), 4.09 (t, J=8 Hz, 1H), 3.90-3.86 (m, 1H), 3.47-3.46 (m, 2H), 3.36-3.29 (m, 3H), 3.14 (s, 3H), 2.94-2.87 (m, 2H), 2.18 (d, J=6 Hz, 2H), 1.97 (s, 1H), 1.83 (s, 2H), 1.68-1.52 (m, 4H), 1.30 (s, 1H), 1.04 (s, 2H), 0.77 (d, J=3.2 Hz, 2H).

### Example 56: Synthesis of 1-(5-cyano-2,3-dihydro-1H-inden-2-yl)-3-((1R,4R)-4-((4-((5-cyclopentyl-1H-pyrazol-3-yl)amino)pyrimidin-2-yl)(methyl)amino)cyclohexyl)-1-methylurea (Compound 153)

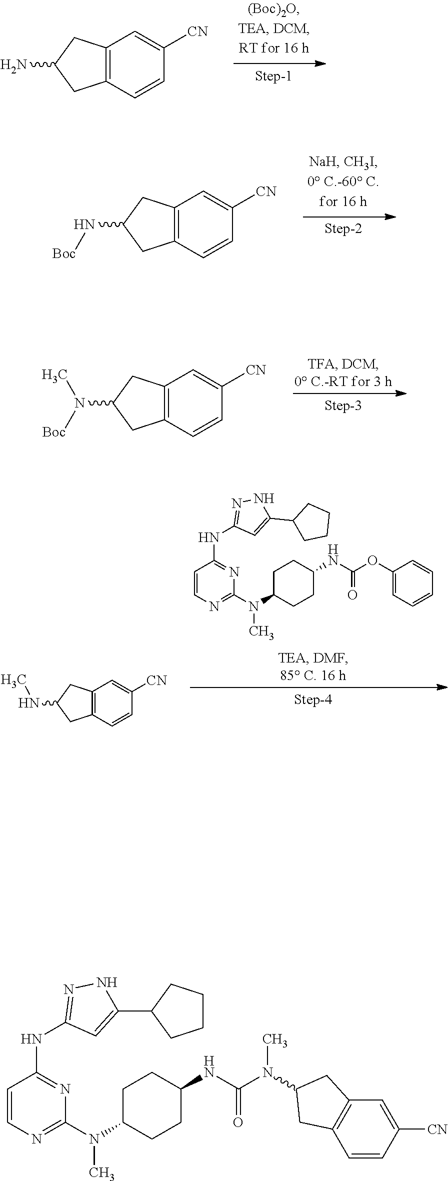 [SMILES: N#Cc1ccc2c(c1)CC(N)C2 ; CC(C)(C)OC(=O)NC1Cc2ccc(C#N)cc2C1 ; CN(C(=O)OC(C)(C)C)C1Cc2ccc(C#N)cc2C1 ; CN(c1nccc(Nc2cc(C3CCCC3)[nH]n2)n1)[C@H]1CC[C@H](NC(=O)Oc2ccccc2)CC1 ; CNC1Cc2ccc(C#N)cc2C1 ; CN(C(=O)N[C@H]1CC[C@H](N(C)c2nccc(Nc3cc(C4CCCC4)[nH]n3)n2)CC1)C1Cc2ccc(C#N)cc2C1]

### Step-1: Synthesis of tert-butyl (5-cyano-2,3-dihydro-1H-inden-2-yl)carbamate

To a solution of 2-amino-2,3-dihydro-1H-indene-5-carbonitrile (100 mg, 0.632 mmol) in DCM (0.2 mL) was added triethylamine (0.26 mL, 1.896 mmol) at 0° C. followed by addition of (Boc)₂O (0.16 mL, 0.758 mmol). The reaction mixture was stirred at room temperature for 16 h. After completion of the reaction (monitored by TLC), the reaction mixture was diluted with dichloromethane and washed with water. The organic layer was dried over anhydrous sodium sulphate, filtered and concentrated under reduced pressure. The solid was triturated with pet ether to obtain tert-butyl (5-cyano-2,3-dihydro-1H-inden-2-yl)carbamate (155 mg, 96.8%). LC purity: 860%; m/z: 259 [M+H]⁺ (Mol. formula C₁₅H₁₈N₂O₂, calcd. mol. wt. 258.32).

### Step-2: Synthesis of tert-butyl (5-cyano-2, 3-dihydro-1H-inden-2-yl) (methyl)carbamate

In a 100 mL sealed tube tert-butyl (5-cyano-2,3-dihydro-1H-inden-2-yl)carbamate (180 mg, 0.700 mmol) in dry DMF (2 mL) was cooled to 0° C. and sodium hydride (84 mg, 3.501 mmol) was added portion wise under N₂ atmosphere. The reaction mixture was stirred for 20 mins at ambient temperature. Then methyl iodide (0.052 mL, 0.840 mmol) was added dropwise at 0° C. then the reaction was heated to 60° C. for 16 h. The completion of the reaction was monitored by TLC. After completion of the reaction, the reaction was quenched with ice water and extracted with ethyl acetate. The resulting organic layer was washed with brine solution then dried over anhydrous Na₂SO₄ and concentrated to obtain tert-butyl (5-cyano-2, 3-dihydro-1H-inden-2-yl)(methyl)carbamate (150 mg, 79.3% yield). LC purity: 68.03%; m/z: 173.1 [M−Boc]⁺ (Mol. formula C₁₆H₂₀N₂O₂, calcd. mol. wt. 272.35)

### Step-3: Synthesis of 2-(methylamino)-2,3-dihydro-1H-indene-5-carbonitrile

To a stirred solution of tert-butyl (5-cyano-2,3-dihydro-1H-inden-2-yl)(methyl)carbamate (150 mg, 0.550 mmol) in dry DCM (2 mL) was cooled to 0° C. and TFA (1.5 mL) was added. The reaction mixture was stirred at room temperature for 3 h. The progress of the reaction was monitored by TLC after complete consumption of the starting material, the reaction was concentrated to get 2-(methylamino)-2,3-dihydro-1H-indene-5-carbonitrile (150 mg, quantitative yield) as TFA Salt. LC purity: 86.29%; m/z: 173.2 [M+H]⁺ (Mol. formula C₁₁H₁₂N₂, calcd. mol. wt. 172.23).

### Step-4: Synthesis of 1-(5-cyano-2,3-dihydro-1H-inden-2-yl)-3-((1R,4R)-4-((4-((5-cyclopentyl-1H-pyrazol-3-yl)amino)pyrimidin-2-yl)(methyl)amino)cyclohexyl)-1-methylurea

To a stirred solution phenyl ((1R,4R)-4-((4-((5-cyclopentyl-1H-pyrazol-3-yl)amino)pyrimidin-2-yl)(methyl)amino)cyclohexyl)carbamate (414 mg, 0.871 mmol) in dry DMF (5 mL) was added triethylamine (0.12 mL, 0.871 mmol). The reaction was stirred at 0° C. for 1 h. In another vial 2-(methylamino)-2,3-dihydro-1H-indene-5-carbonitrile (150 mg, 0.871 mmol) in dry DMF (2 mL) was added triethylamine (0.23 mL, 1.744 mmol) and stirred for 15 min. The resulting solution was added to the above reaction at RT. The reaction was heated to 85° C. for 16 h. After completion of the starting material (monitored by TLC), reaction was diluted with water and extracted with dichloromethane. The resulting organic layer was washed with brine solution then dried over anhydrous Na₂SO₄ and concentrated to get crude compound. which was then purified by reverse phase preparative HPLC to obtain 1-(5-cyano-2,3-dihydro-1H-inden-2-yl)-3-((1R,4R)-4-((4-((5-cyclopentyl-1H-pyrazol-3-yl)amino)pyrimidin-2-yl)(methyl)amino) cyclohexyl)-1-methylurea (40 mg, 8.3% yield). LC purity: 96.40%; m/z: 554.2 [M+H]⁺ (Mol. formula C₃₁H₃₉N₉O, calcd. mol. wt. 553.72). ¹H NMR (400 MHz, CD₃OD): δ 7.81 (dd, J=12, 5.6 Hz, 1H), 7.57 (s, 1H), 7.52 (d, J=8 Hz, 1H), 7.40 (d, J=8 Hz, 1H), 6.30-6.25 (m, 2H), 5.21-5.17 (m, 1H), 4.53 (s, 1H), 3.68-3.60 (m, 1H), 3.32-3.21 (m, 2H), 3.19-3.03 (m, 6H), 2.75 (s, 3H), 2.12-2.09 (m, 4H), 1.82-1.67 (m, 10H), 1.51-1.46 (m, 2H).

### Example 57: Degradation of MycN Protein

### Cell Culture

In a T150 flask was plated either 2×10⁶ of HL-60 (human leukemia cell line) or SKNBE2 (neuroblastoma cell line) cells in a total of 30 mL containing Roswell Park Memorial Institute (RPMI) 1640 Medium (Thermo Fisher, Cat. #11875-085), 10% Fetal Bovine Serum (FBS, Thermo Fisher: Cat. #10437-028), 1% Penicillin/streptomycin (Thermo Fisher: Cat. #10378016), and 1% Amophotericin B (Thermo Fisher, Cat. #15290026). The cells were split every 72 hours by reseeding the HL-60 or SKNBE2 cells, and the passage number was noted. It is noteworthy that cell cultures that were above 40 passages were not used, and most experiments were done with cells of less than 30 passages.

### Western Blotting

In a 6-well cell culture plate, a total of 3 mL of either 1×10⁶ cells/mL of HL-60 or SKNBE2 cells were seeded, resulting in a total of 3×10⁶ cells per well. To each well containing 3 mL of either HL-60 or SKNBE2 cell line was added 6 uL of MycN modulating compound (1 mM) and the resulting plate was shaken from left to right, and not swirled. After 6 hours, the cells were placed in a 15 mL falcon tube and spun at 500 Gs at 4° C. in a swinging bucket centrifuge. The medium was then carefully removed without disturbing the pellet. The pellet was then washed with 3 mLs of chilled phosphate buffered saline (PBS) and subjected to the spin cycle. PBS was then removed and the pellet was lysed in 200 uL of radioimmunoprecipitation (RIPA) lysis buffer (Thermo Fisher: Cat. #899000) that is supplemented with protease and phosphatase inhibitors (Thermo Fisher: Cat. #A32959). The cell lysate was then subjected to spinning in a centrifuge for 10 minutes at 13000G at 4° C. The supernatant was then carefully transferred to a fresh eppendorf tube without disturbing the pellet (˜180 uL). The protein concentration of the cell lysate was then determined by using a bicinchoninic acid (BCA assay) according to manufacturer's protocol (Thermo Fisher: Cat. #23227).

### Gel Running and Transfer

Cell lysate, approximately 25 ug-30 ug, was loaded per well in a 4-20% polyacrylamide gel (Biorad. Cat. #5671094). After running the dye front off of the gel, the gel was transferred to a nitrocellulose membrane (Biorad: Cat. #1704159) using the transblot turbo system (Biorad: Cat. #1704150) according to manufacturer's protocol. After transferring for 30 minutes, the membrane was blocked with 5% BSA for 1 hour at room temperature. The BSA was then washed off and the primary antibody of choice (1:500) was added, and the membrane was incubated with the primary antibody at 4° C. for overnight. The next morning, the primary antibody was removed and the membrane was washed with 1×-TBST for 10 minutes and repeated three more times. Following the last wash, a secondary antibody (Molecular Devices. Cat. #R8209 or R8208) was added at 1:5000 dilution and incubated for 1 hour at room temperature. Following the incubation with the secondary antibody, the membrane was washed with 1×-TBST for 10 minutes and repeated three more times. Following the last wash, the membrane was washed with de-ionized water twice and dried for at least two hours. Once the membrane is completely dry, the Molecular Devices Spectra Max western system was used to observe the bands. The western image was saved and the band density was measured with ImageJ software.

### Example 45: Percent Degradation of MycN Protein by Various Compounds

TABLE 1
Percent Degradation of MycN/MycC Protein

| Cmpd | % Degradation | % Degradation |
|---|---|---|
| No. | of MycN | of MycC |
| 2 | ** | * |
| 3 | ** | *** |
| 4 | ** | **** |
| 5 | ** | * |
| 6 | * | ** |
| 7 | ** | * |
| 8 | * | ** |
| 11 | **** | **** |
| (Racemic) | | |
| 11 | ** | **** |
| (Fraction 1) | | |
| 11 | *** | **** |
| (Fraction 2) | | |
| 12 | * | * |
| 16 | **** | **** |
| (Racemic) | | |
| 16 | *** | **** |
| (Fraction 1) | | |
| 16 | *** | **** |
| (Fraction 2) | | |
| 20 | **** | **** |
| 21 | ** | * |
| 24 | * | ** |
| 25 | * | * |
| 26 | ** | * |
| 27 | * | * |
| 32 | * | * |
| 34 | * | *** |
| 35 | **** | ** |
| 37 | * | * |
| 38 | ** | *** |
| 39 | *** | ** |
| 40 | *** | *** |
| 43 | ** | ** |
| 44 | * | ** |
| 46 | ** | * |
| 47 | * | * |
| 48 | * | ** |
| 49 | * | * |
| 52 | **** | **** |
| 53 | * | * |
| 56 | * | ** |
| 57 | * | *** |
| 60 | * | *** |
| 61 | * | |
| 63 | *** | ** |
| 67 | * | ** |
| 68 | ** | |
| 70 | * | ** |
| 72 | * | ** |
| 73 | * | * |
| 74 | ** | * |
| 75 | * | ** |
| 77 | * | ** |
| 79 | * | ** |
| 81 | * | **** |
| 82 | ** | |
| 83 | * | * |
| 84 | * | * |
| 87 | ** | ** |
| 96 | ** | ** |
| 97 | ** | |
| 98 | * | |
| 99 | * | |
| 100 | * | |
| 101 | * | **** |
| 102 | * | * |
| 103 | * | * |
| 104 | ** | * |
| 105 | * | * |
| 106 | ** | |
| 107 | * | *** |
| 112 | * | * |
| 115 | * | * |
| 116 | * | * |
| 119 | * | * |
| 120 | * | * |
| 121 | *** | *** |
| (Racemic) | | |
| 121 | *** | *** |
| (Fraction 1) | | |
| 121 | *** | ** |
| (Fraction 2) | | |
| 142 | **** | **** |
| 143 | *** | *** |
| 144 | *** | **** |
| 145 | **** | *** |
| 146 | *** | |
| 147 | **** | |
| 148 | **** | *** |
| 149 | *** | * |
| 150 | **** | **** |
| 151 | *** | |
| 152 | **** | **** |
| 153 | ** | *** |

Key: ****: degradation 80-100%
***: degradation 50-79%
**: degradation 20-49%
*: degradation <20%

The practice of the present disclosure will employ, unless otherwise indicated, conventional methods of organic chemistry, protein chemistry, biochemistry, recombinant DNA techniques and pharmacology, within the skill of the art. While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

What is claimed is:

1. A compound of Formula (I): 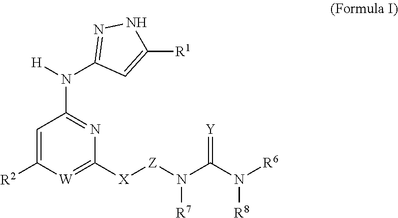
or a pharmaceutically acceptable salt, stereoisomer and/or N-oxide thereof, wherein:
W is selected from the group consisting of N, C—H, and C—F;
X is selected from the group consisting of N—R^{A}, O, S, CH₂, C(CH₃)₂, CF₂ and C(CH₂)₂;
Y is selected from the group consisting of O and N—R^{B};
Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, and —C(O)—O—C₁₋₄alkyl;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —O-methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-heteroaryl, —C(O)-heteroaryl, and phenoxy; wherein R⁶ may be optionally substituted by one, two or three substituents each independently selected from the group consisting of R^{P};
R⁷ is selected from the group consisting of H and C₁-C₆ alkyl; wherein C₁-C₆ alkyl may be optionally substituted by one, two or three substituents each independently selected from the group consisting of halogen, hydroxyl, cyano, oxo and C₁₋₆alkoxy (optionally substituted by one, two or three substituents each selected from halo, cyano, hydroxyl, and C₁₋₃alkoxy);
R⁸ is selected from the group consisting of H and C₁-C₆-alkyl; wherein C₁-C₆ alkyl may be optionally substituted by one, two or three substituents each independently selected from the group consisting of halogen, hydroxyl, cyano, oxo and C₁₋₆alkoxy (optionally substituted by one, two or three substituents each selected from halo, cyano, hydroxyl, and C₁₋₃alkoxy);
wherein at least one of R⁷ or R⁸ must be H;
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{B} is selected from the group consisting of H, C₁-C₄ alkyl, —C(O)—C₁₋₄ alkyl, S(O)_{w}—C₁₋₄alkyl, (wherein w is 0, 1 or 2) and cyano; wherein C₁-C₄ alkyl may be optionally substituted by one, two or three flouro substituents;
R^{P} is selected from the group consisting of halo, cyano, C₁₋₆alkyl, C₂₋₆alkenyl, C₂₋₆ alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆ alkenyl, C₂₋₆alkynyl and C₃₋₆cycloalkyl may each be optionally substituted by one, two or three substituents each selected from halo, cyano, hydroxyl, heteroaryl, and NR′R′; and
R′ for each occurrence is independently selected from the group consisting of H, methyl, ethyl, heterocyclyl (optionally substituted by C₁₋₃alkyl or halo), phenyl, and C₃₋₆ cycloalkyl, or two R's together with the nitrogen to which they are attached form a heterocyclyl which may optionally be substituted by methyl, halo, cyano, oxo, or hydroxyl.

2. The compound according to claim 1, wherein W is N, and having the Formula Ia: 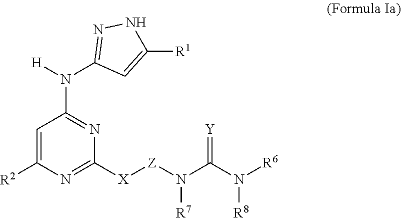
or a pharmaceutically acceptable salt, stereoisomer and/or N-oxide thereof.

3. The compound according to claim 1 or 2, wherein Y is O, and having the Formula Ib: 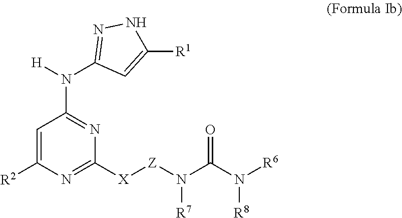
or a pharmaceutically acceptable salt, stereoisomer and/or N-oxide thereof.

4. The compound according to claim 1 or 2, wherein Y is N—R^{B}, and having the Formula Ic: 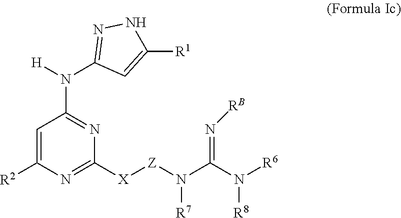
or a pharmaceutically acceptable salt, stereoisomer and/or N-oxide thereof, wherein:
R^{B} is selected from the group consisting of H, C₁-C₄ alkyl, —SO₂—C₁-C₄-alkyl, C(O)C₁-C₄-alkyl, CN, and CH₂CF₃.

5. The compound of any one of claims 1-4, wherein R¹ is a 5-6 membered heterocyclyl or C₃₋₆cycloalkyl.

6. The compound of any one of claims 1-5, wherein R¹ is selected from the group consisting of: 2-tetrahydrofuranyl, 3-tetrahydrofuranyl, 2-oxetanyl, cyclohexyl, cyclopropyl, cyclobutyl and cyclopentyl.

7. The compound of any one of claims 1-4, wherein R¹ is selected from the group consisting of methyl and ethyl.

8. The compound according to any one of claims 1-7, wherein Z is selected from the group consisting of cyclohexyl, cyclopentyl and cyclobutyl.

9. The compound of any one of claims 1-7, wherein Z is a C₅-C₉ bridged cycloalkyl.

10. The compound of any one of claims 1-7, wherein Z is a spiro C₅-C₁₀ bicycloalkyl.

11. The compound of any one of claims 1-7, wherein Z is a fused bicycloalkyl.

12. The compound of any one of claims 1-6, wherein Z is selected from the group consisting 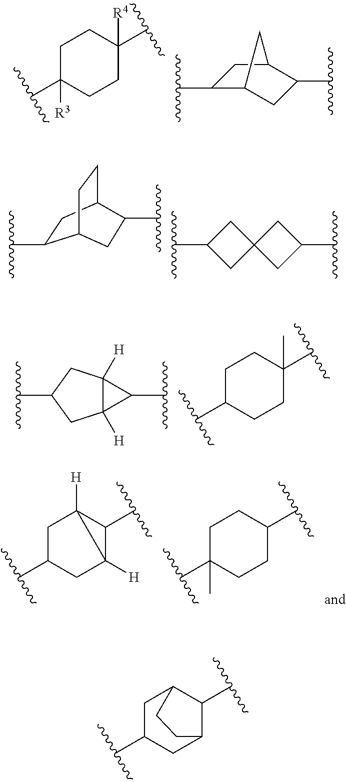
or a pharmaceutically acceptable salt, stereoisomer and/or N-oxide thereof, wherein:
R³ is selected from the group consisting of H, C₁-C₄-alkyl, CO₂H and —C(O)—O—C₁₋₄alkyl;
R⁴ is H or C₁-C₄-alkyl; or
R³ and R⁴ together form —CH₂— or —CH₂CH₂—.

13. The compound of claim 12, wherein Z is selected from the group consisting of:

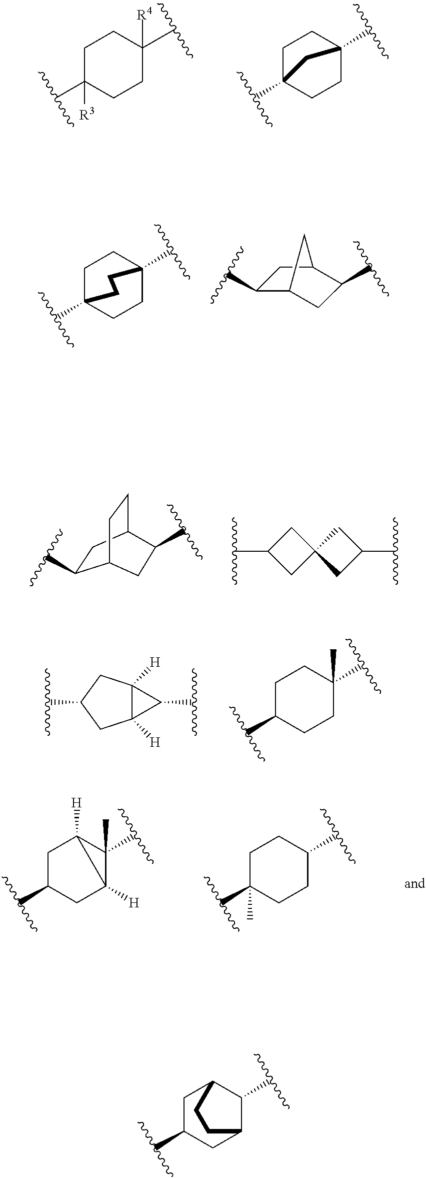

14. The compound of claim 13, represented by Formula II: 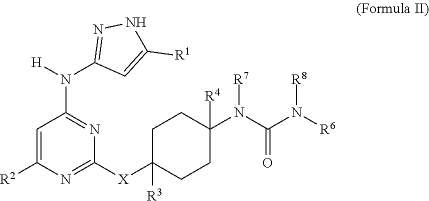
or a pharmaceutically acceptable salt, stereoisomer and/or N-oxide thereof, wherein:
R³ is selected from the group consisting of H, C₁-C₄alkyl, CO₂H and —C(O)—O—C₁₋₄ alkyl; and
R⁴ is selected from H or C₁-C₄alkyl.

15. The compound of claim 13, represented by Formula IIa: 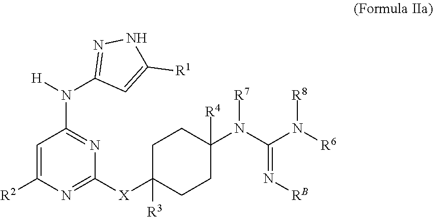
or a pharmaceutically acceptable salt, stereoisomer and/or N-oxide thereof, wherein:
R³ is selected from the group consisting of H, C₁-C₄-alkyl, CO₂H and —C(O)—O—C₁₋₄ alkyl; and
R⁴ is selected from the group consisting of H and C₁-C₄-alkyl;
R^{B} is selected from the group consisting of H, C₁-C₄ alkyl, —SO₂—C₁-C₄-alkyl, C(O)C₁-C₄-alkyl, CN, and CH₂CF₃.

16. The compound of any one of claims 1-15, wherein R⁶ is selected from the group consisting of a 8-10 membered bicyclic cycloalkyl and a 8-10 membered bicyclic heterocyclyl.

17. The compound of any one of claims 1-15, wherein R⁶ is selected from the group consisting of a monocyclic or bridged C₃₋₆cycloalkyl, a monocyclic or bridged heterocyclyl, a bicyclic or fused heterocyclyl, and a heteroaryl.

18. The compound of any one of claims 1-15, wherein R⁶ is selected from the group consisting of: indanyl, cyclohexyl, cyclobutyl, and cyclopentyl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms), C₁₋₄alkoxy (optionally substituted by one, two or three fluorine atoms), S(O)₂—CH₃; cyclopropyl, cyclobutyl, —O-heterocyclyl, heterocyclyl and heteroaryl.

19. The compound of claim 18, wherein R⁶ is indanyl.

20. The compound of any one of claims 1-15, wherein R⁶ is selected from the group consisting of heterocyclyl, phenyl, and heteroaryl.

21. The compound of any one of claims 1-15, wherein R⁶ is represented by: 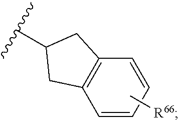
wherein R⁶⁶ is selected from the group consisting of hydrogen, cyano, heterocyclyl, heteroaryl, —C(═N)—R′R′; and S(O)₂—CH₃.

22. The compound of claim 21, wherein R⁶ is selected from the group consisting of:

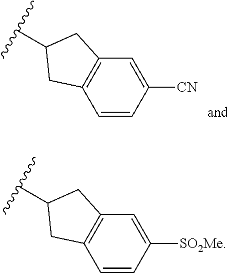 [SMILES: *C1Cc2ccc(C#N)cc2C1 ; *C1Cc2ccc(S(C)(=O)=O)cc2C1]

23. The compound of any one of claims 1-15, wherein R⁶ is represented by: 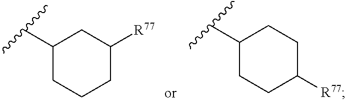
wherein R⁷⁷ is selected from the group of hydrogen, C₁₋₄alkyl (optionally substituted by one, two or three fluorine atoms), C₁₋₄alkoxy (optionally substituted by methoxy or by one, two or three fluorine atoms), heterocyclyl, and S(O)₂—C₁₋₄alkyl.

24. The compound of claim 23, wherein R⁷⁷ is selected from the group consisting of —CF₃, —OCH₃, —OCHF₂, —SO₂CH₃, and —OCH₂CH₂OCH₃.

25. The compound of claim 23 or 24, wherein R⁶ is selected from the group consisting of:

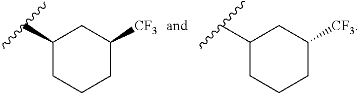 [SMILES: *[C@@H]1CCC[C@H](C(F)(F)F)C1 ; *C1CCC[C@@H](C(F)(F)F)C1]

26. The compound of any one of claims 1-15, wherein R⁶ is selected from the group consisting of:

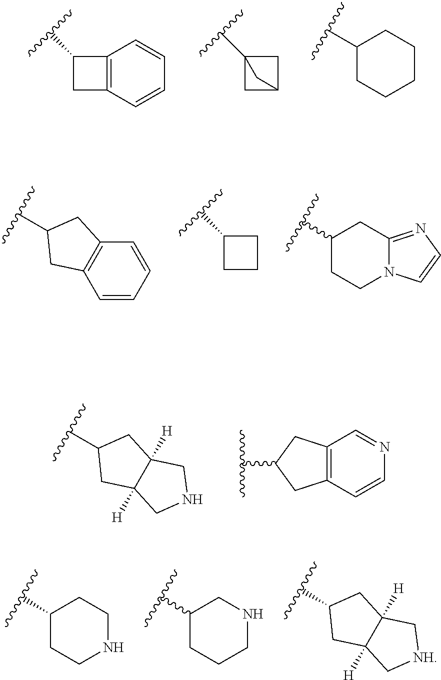

27. The compound of any one of claims 1-15, wherein R⁶ is selected from the group consisting of: 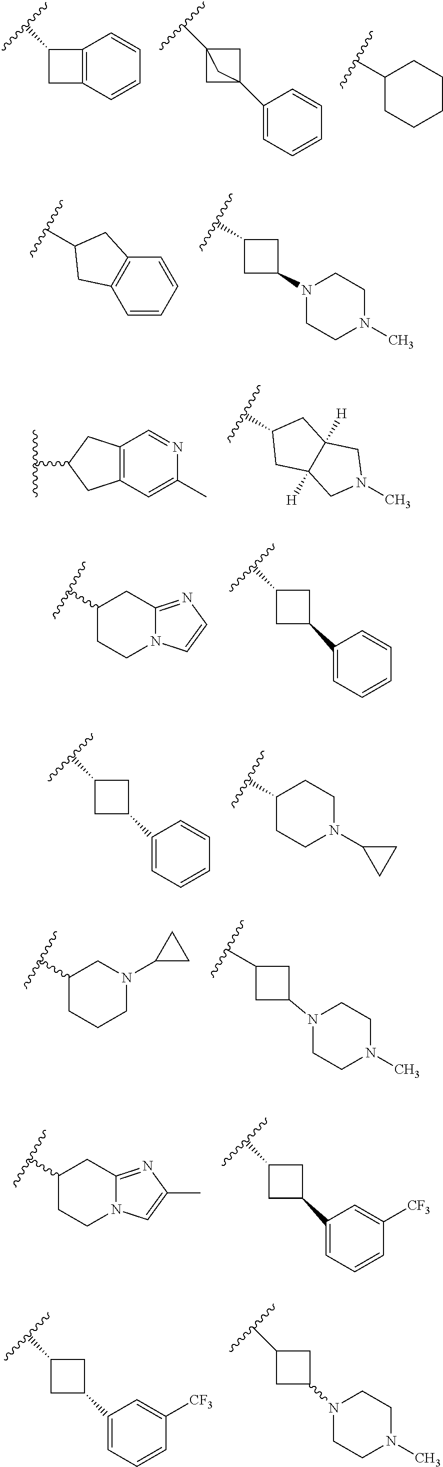 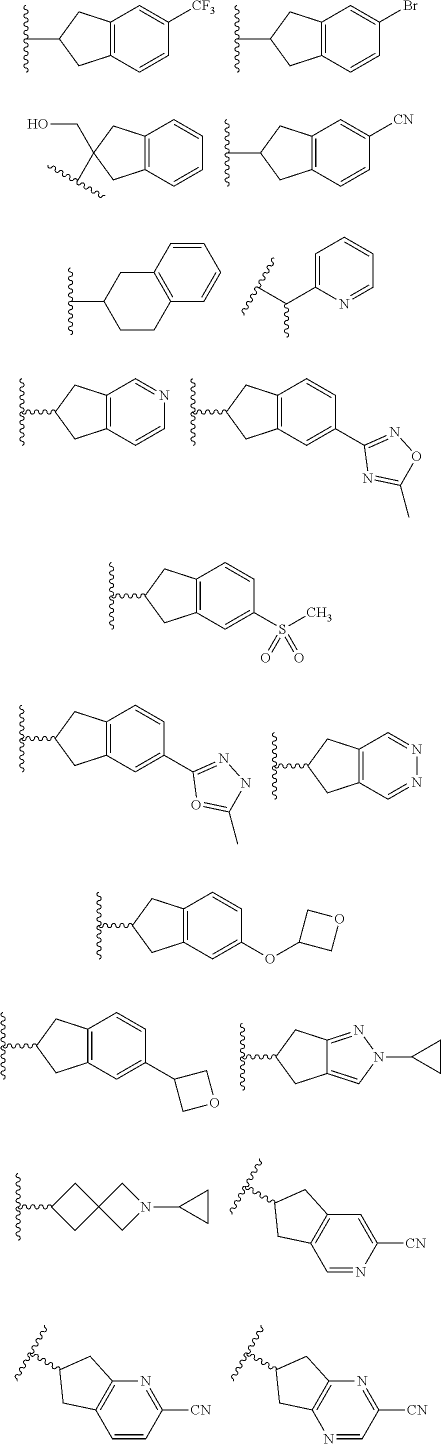 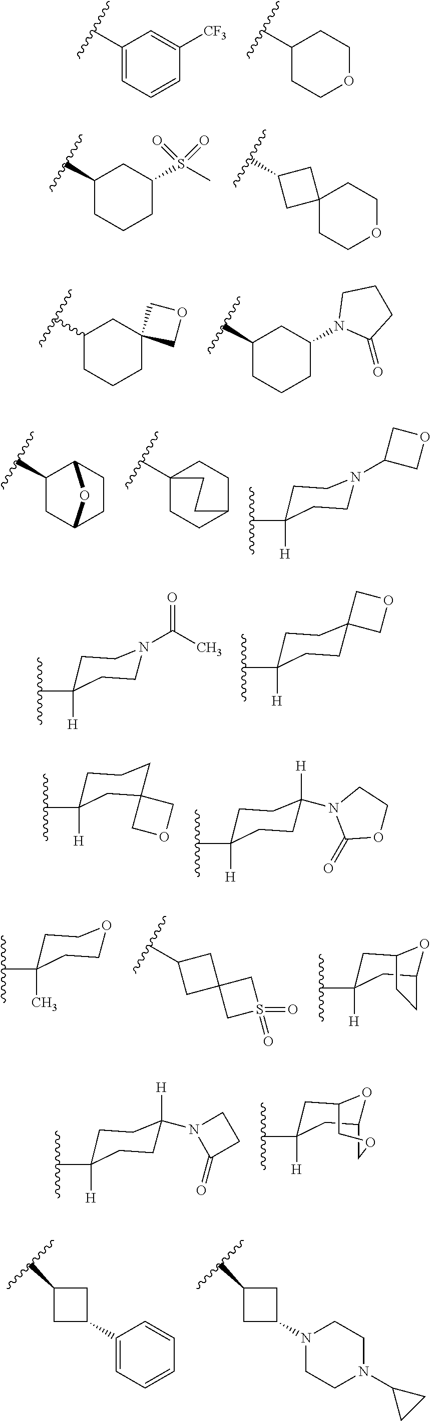 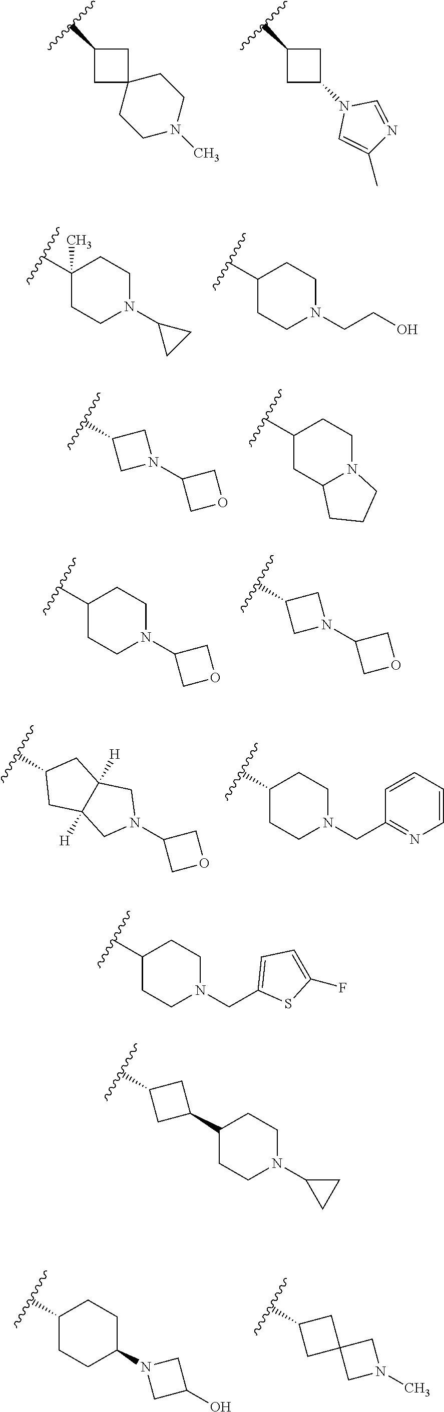 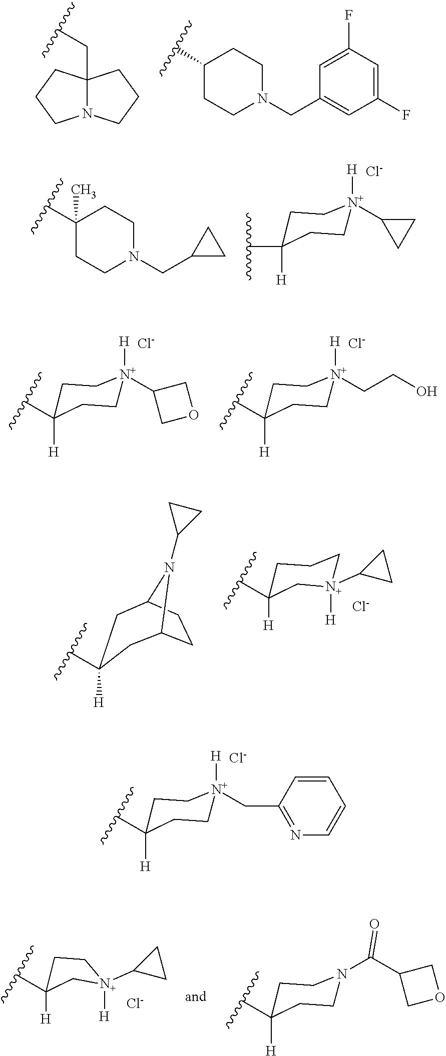
or a pharmaceutically acceptable salt, stereoisomer and/or N-oxide thereof.

28. The compound of any one of claims 1-27, wherein X is N—H.

29. The compound of any one of claims 1-27, wherein X is O.

30. The compound of any one of claims 1-29, wherein R⁷ is H and R⁸ is methyl

31. The compound of any one of claims 1-29, wherein R⁷ is methyl and R⁸ is H.

32. The compound of any one of claims 1-29 wherein R⁷ and R⁸ are each H.

33. A compound represented by Formula (III): 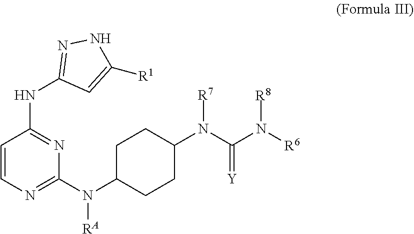
or a pharmaceutically acceptable salt, stereoisomer and/or N-oxide thereof, wherein:
R¹ is selected from the group consisting of C₃-C₆cycloalkyl, heterocyclyl, and methyl, wherein R¹ is optionally substituted by halogen;
R⁶ is selected from the group consisting of a saturated C₃-C₆ monocyclic carbocyclic ring, a saturated or partially unsaturated 8-10 membered bicyclic carbocylic ring, a monocyclic or bicyclic saturated or partially unsaturated heterocyclic ring having at least one heteroatom moiety selected from O, S(O)_{w} (wherein w is 0, 1, or 2), and NR^{C}, phenyl, phenoxy, naphthyl, a monocylic or bicyclic heteroaryl, benzyl, and —CR⁷R⁸— heteroaryl; wherein:
R⁶ is optionally substituted on an available carbon by one, two or three substituents each independently selected from the group consisting of halogen, cyano, hydroxyl, oxo, C₁-C₆-alkyl (optionally substituted by one, two or three halogens or hydroxyl), C₃-C₆-cycloalkyl (optionally substituted by one, two or three halogens or hydroxyl), C₁-C₆-alkoxy (optionally substituted by one, two or three substituents each selected from the group consisting of halogen, methyoxy, and ethyoxy), heterocyclyl (optionally substituted by one or more substituents each selected from methyl, ethyl, hydroxyl, halogen and oxo), heterocyclyloxy (optionally substituted by one or more substituents each selected from methyl, ethyl, halogen, hydroxyl and oxo), heteroaryl (optionally substituted by one or more substituents each selected from methyl, ethyl, hydroxyl, halogen and oxo), heteroaryloxy (optionally substituted by one or more substituents each selected from methyl, ethyl, halogen, hydroxyl and oxo), —NR^{a}R^{b}; —C(O)O(R^{a}), —C(O)—N(R^{b})(R^{c}), —S(O)_{w}—R^{a}, —NR^{b}—S(O)_{w}—R^{a}, and —S(O)_{w}—N(R^{b})(R^{c}) (wherein w is 0, 1, or 2); and wherein
R^{C}, if present, is selected from the group consisting of hydrogen, C₁-C₆alkyl (optionally substituted by phenyl or heteroaryl; wherein phenyl or heteroaryl is optionally substituted by halogen, hydroxyl, or methyl), cyclopropyl, C(O)O(R^{a}), C(O)R^{a}, and —S(O)_{w}—R^{a} (wherein w is 0, 1, or 2);
R^{A} is selected from the group consisting of H and methyl;
R⁷ is selected from the group consisting of H and methyl;
R⁷ is selected from the group consisting of H and methyl;
wherein at least one of R⁷ and R⁸ must be hydrogen;
R^{a} is independently selected for each occurrence from the group consisting of hydrogen, C₁-C₆-alkyl, C₃-C₆-cycloalkyl, heterocyclyl, phenyl and heteroaryl; wherein C₁-C₆alkyl, C₃-C₆cycloalkyl, heterocyclyl, phenyl or heteroaryl may optionally be substituted by one or more substituents each independently selected from the group consisting of halogen, cyano, oxo, and hydroxyl;
R^{b} and R^{c} are each independently selected for each occurrence from the group consisting of hydrogen, C₁-C₆alkyl, C₃-C₆cycloalkyl, phenyl, benzyl, and heteroaryl; or
R^{b} and R^{c} may form, together with the nitrogen to which they are attached, a 4-6 membered heterocyclyl which may have an additional heteroatom and may be optionally substituted with oxo, C₁-C₃alkyl, or cyclopropyl;
R⁷ and R⁸ are each independently selected from the group consisting of hydrogen, halogen, and C₁-C₃alkyl (optionally substituted by one, two or three halogens), or R⁷ and R⁸ taken together form an oxo;
Y is selected from O and N—R^{B}; and
R^{B} is selected from the group consisting of H, C₁-C₄alkyl, —S(O)_{w}—C₁-C₄alkyl (where w is 0, 1, or 2), —C(O)C₁-C₄alkyl, and CN; wherein C₁-C₄alkyl is optionally substituted by one, two or three halogens.

34. The compound of claim 33, wherein R⁶ is a partially unsaturated bicyclic carbocycle.

35. The compound of claim 33 or 34, wherein R⁶ is represented by: 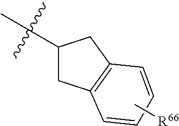
wherein R⁶⁶ is selected from the group consisting of cyano, heterocyclyl, heterocyclyloxy, C₁-C₃-alkyl (optionally substituted by halogen), —C(═N)—R^{b}R^{c}; and —S(O)₂—C₁-C₃-alkyl.

36. The compound of claim 33, wherein R⁶ is a heterocyclic ring having a heteroatom moeity NR^{C}.

37. The compound of claim 36, wherein R⁶ is represented by 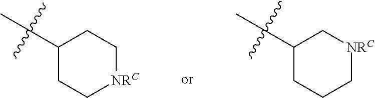
wherein R^{C} is selected from the group consisting of hydrogen, C₁-C₃-alkyl (optionally substituted by phenyl or heteroaryl; wherein phenyl or heteroaryl is optionally substituted by halogen, hydroxyl, or methyl), C₃-C₆-cycloalkyl, C(O)O(R^{a}), C(O)R^{a}, and —S(O)_{w}—R^{a}.

38. The compound of claim 37, wherein R^{C} is C₁-C₆ alkyl, C₃-C₈-cycloalkyl, C₃-C₈-heterocycloalkyl, —CH₂-heteroaryl, —C(O)—C₁-C₆-alkyl, —C(O)-heteroaryl, —C(O)—C₃-C₈-cycloalkyl, and —C(O)—C₃-C₈-heterocycloalkyl.

39. The compound of claim 33, wherein R⁶ is selected from the group consisting of: indanyl, cyclohexyl, cyclobutyl, and cyclopentyl, wherein R⁶ is optionally substituted by one or two substituents each selected from the group consisting of: cyano, halo, phenyl, —C(═N)—NR′R′, C₁₋₄alkyl (optionally substituted by one, two or three fluoros), C₁₋₄alkoxy (optionally substituted by methoxy or one, two or three fluoros), S(O)₂—CH₃; cyclopropyl, cyclobutyl, —O-heterocyclyl, and heterocyclyl.

40. The compound of claim 39, wherein R⁶ is represented by: 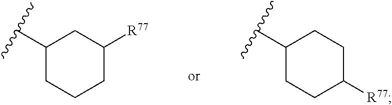
wherein R⁷⁷ is selected from the group of hydrogen, C₁₋₄alkyl (optionally substituted by one, two or three fluorine atoms), C₁₋₄alkoxy (optionally substituted by methoxy or by one, two or three fluorine atoms), heterocyclyl, and S(O)₂—C₁₋₄alkyl.

41. The compound of claim 36 or 3, wherein R⁷⁷ is selected from the group consisting of —CF₃, —OCH₃, OCHF₂, —SO₂CH₃, —OCH₂CH₂OCH₃.

42. The compound of claim 39, wherein R⁶ is selected from the group consisting of:

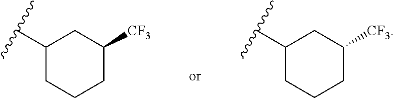 [SMILES: *C1CCC[C@H](C(F)(F)F)C1 ; *C1CCC[C@@H](C(F)(F)F)C1]

43. A compound selected from the group consisting of: 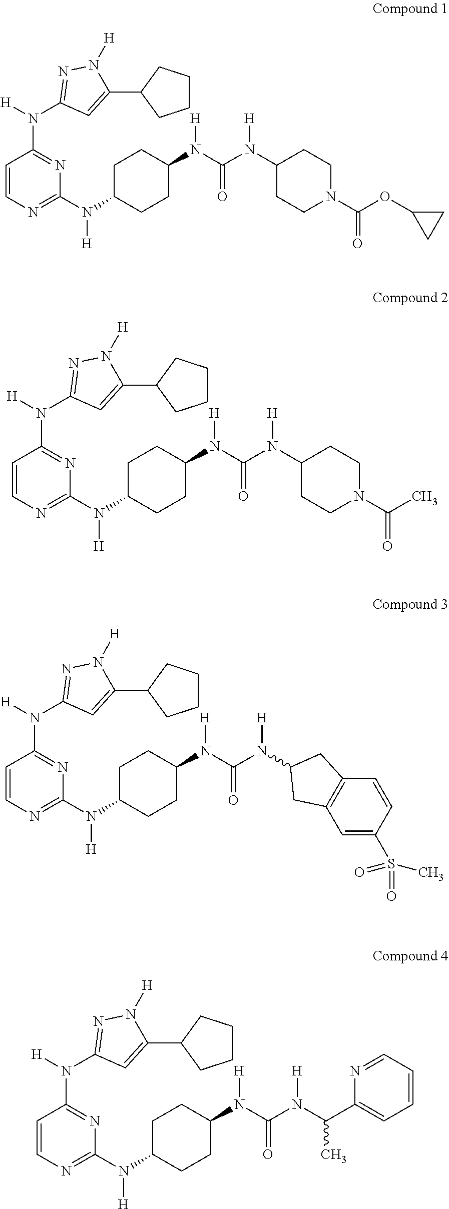 [SMILES: O=C(NC1CCN(C(=O)OC2CC2)CC1)N[C@H]1CC[C@H](Nc2nccc(Nc3cc(C4CCCC4)[nH]n3)n2)CC1 ; CC(=O)N1CCC(NC(=O)N[C@H]2CC[C@H](Nc3nccc(Nc4cc(C5CCCC5)[nH]n4)n3)CC2)CC1 ; CS(=O)(=O)c1ccc2c(c1)CC(NC(=O)N[C@H]1CC[C@H](Nc3nccc(Nc4cc(C5CCCC5)[nH]n4)n3)CC1)C2 ; CC(NC(=O)N[C@H]1CC[C@H](Nc2nccc(Nc3cc(C4CCCC4)[nH]n3)n2)CC1)c1ccccn1] 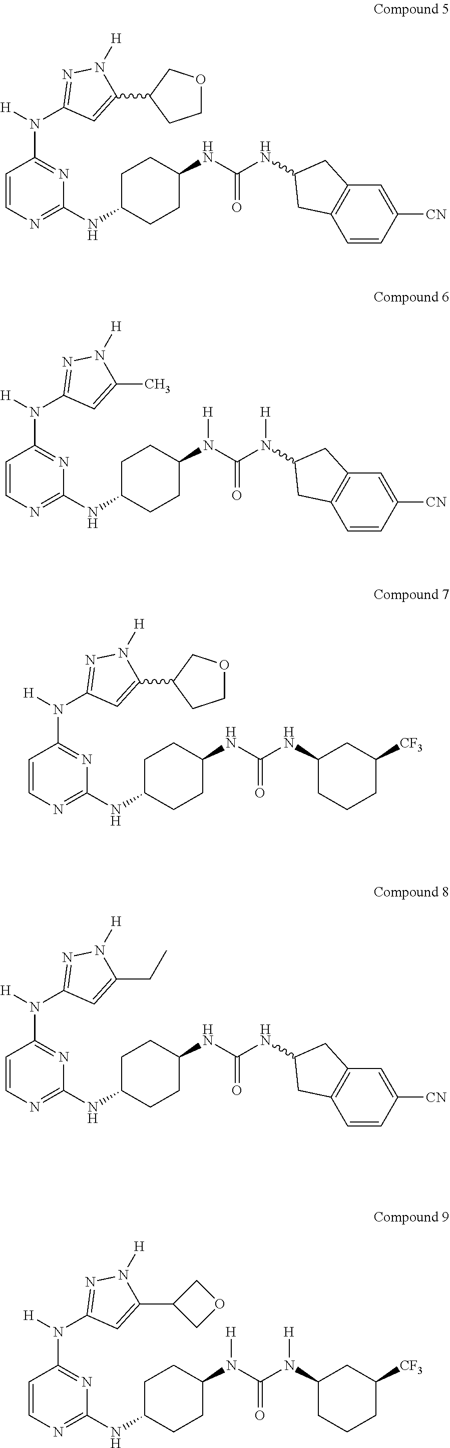 [SMILES: N#Cc1ccc2c(c1)CC(NC(=O)N[C@H]1CC[C@H](Nc3nccc(Nc4cc(C5CCOC5)[nH]n4)n3)CC1)C2 ; Cc1cc(Nc2ccnc(N[C@H]3CC[C@H](NC(=O)NC4Cc5ccc(C#N)cc5C4)CC3)n2)n[nH]1 ; O=C(N[C@@H]1CCC[C@H](C(F)(F)F)C1)N[C@H]1CC[C@H](Nc2nccc(Nc3cc(C4CCOC4)[nH]n3)n2)CC1 ; CCc1cc(Nc2ccnc(N[C@H]3CC[C@H](NC(=O)NC4Cc5ccc(C#N)cc5C4)CC3)n2)n[nH]1 ; O=C(N[C@@H]1CCC[C@H](C(F)(F)F)C1)N[C@H]1CC[C@H](Nc2nccc(Nc3cc(C4COC4)[nH]n3)n2)CC1] 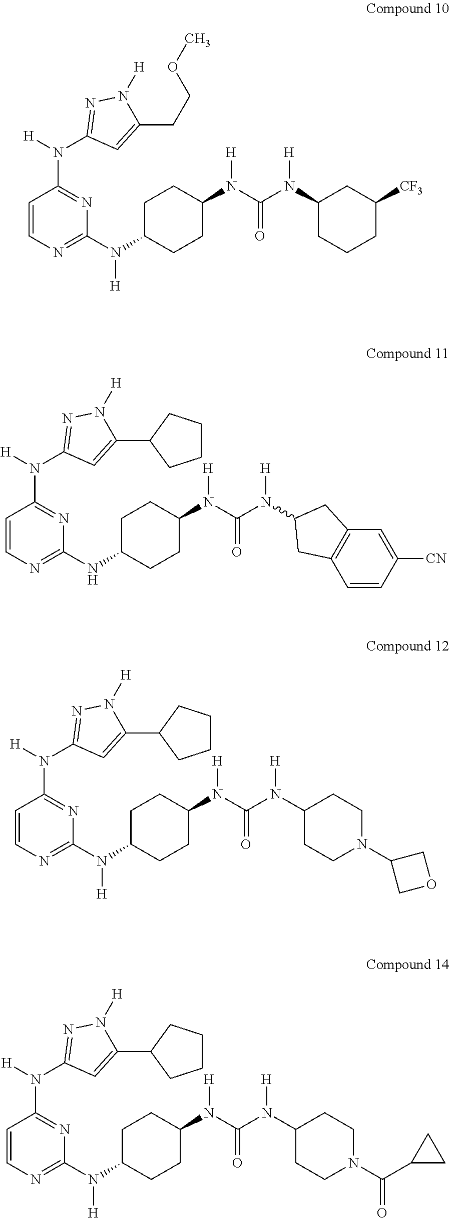 [SMILES: COCCc1cc(Nc2ccnc(N[C@H]3CC[C@H](NC(=O)N[C@@H]4CCC[C@H](C(F)(F)F)C4)CC3)n2)n[nH]1 ; N#Cc1ccc2c(c1)CC(NC(=O)N[C@H]1CC[C@H](Nc3nccc(Nc4cc(C5CCCC5)[nH]n4)n3)CC1)C2 ; O=C(NC1CCN(C2COC2)CC1)N[C@H]1CC[C@H](Nc2nccc(Nc3cc(C4CCCC4)[nH]n3)n2)CC1 ; O=C(NC1CCN(C(=O)C2CC2)CC1)N[C@H]1CC[C@H](Nc2nccc(Nc3cc(C4CCCC4)[nH]n3)n2)CC1] 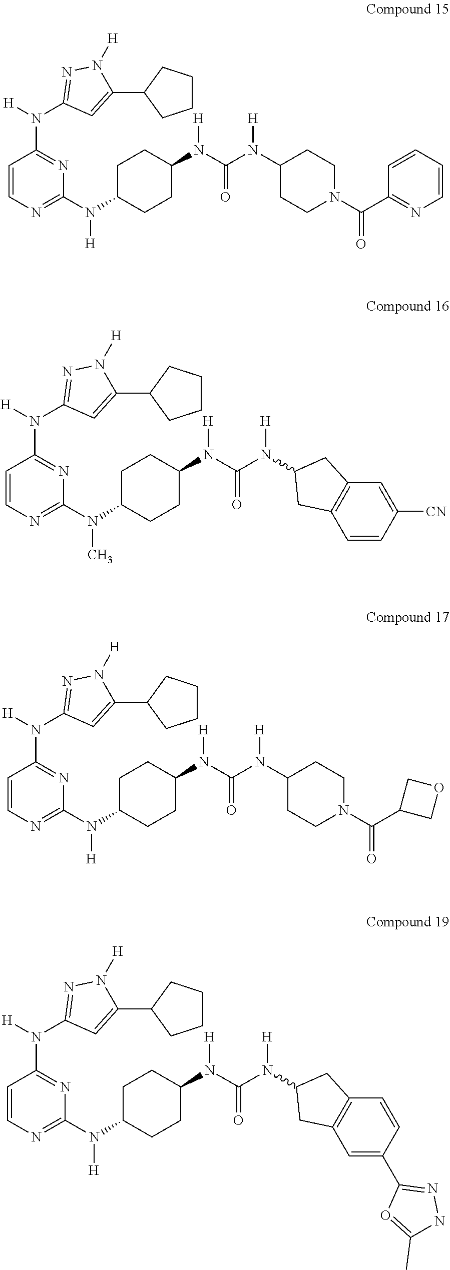 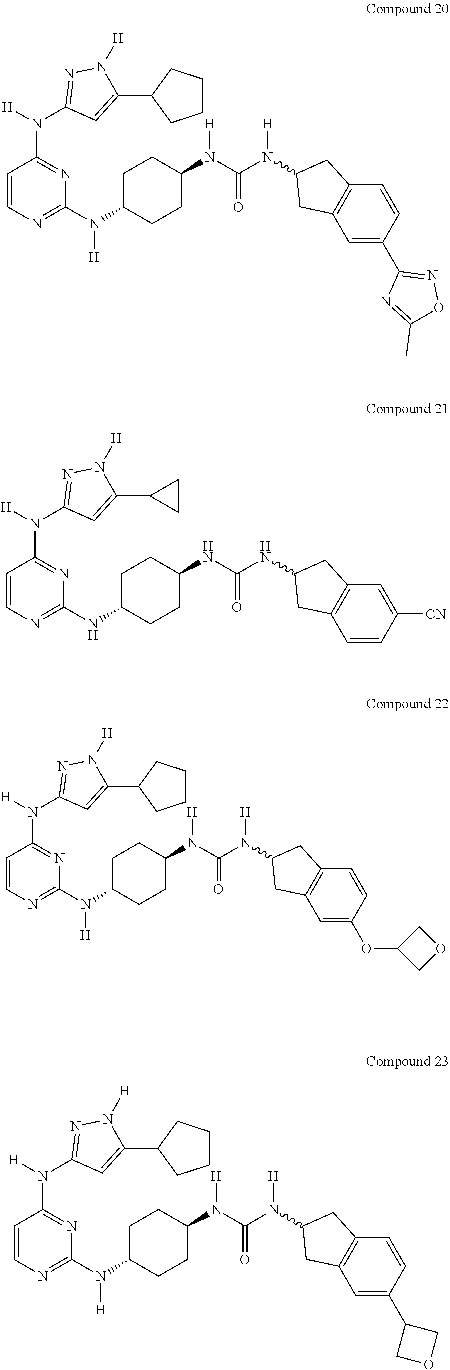 [SMILES: Cc1nc(-c2ccc3c(c2)CC(NC(=O)N[C@H]2CC[C@H](Nc4nccc(Nc5cc(C6CCCC6)[nH]n5)n4)CC2)C3)no1 ; N#Cc1ccc2c(c1)CC(NC(=O)N[C@H]1CC[C@H](Nc3nccc(Nc4cc(C5CC5)[nH]n4)n3)CC1)C2 ; O=C(NC1Cc2ccc(OC3COC3)cc2C1)N[C@H]1CC[C@H](Nc2nccc(Nc3cc(C4CCCC4)[nH]n3)n2)CC1 ; O=C(NC1Cc2ccc(C3COC3)cc2C1)N[C@H]1CC[C@H](Nc2nccc(Nc3cc(C4CCCC4)[nH]n3)n2)CC1] 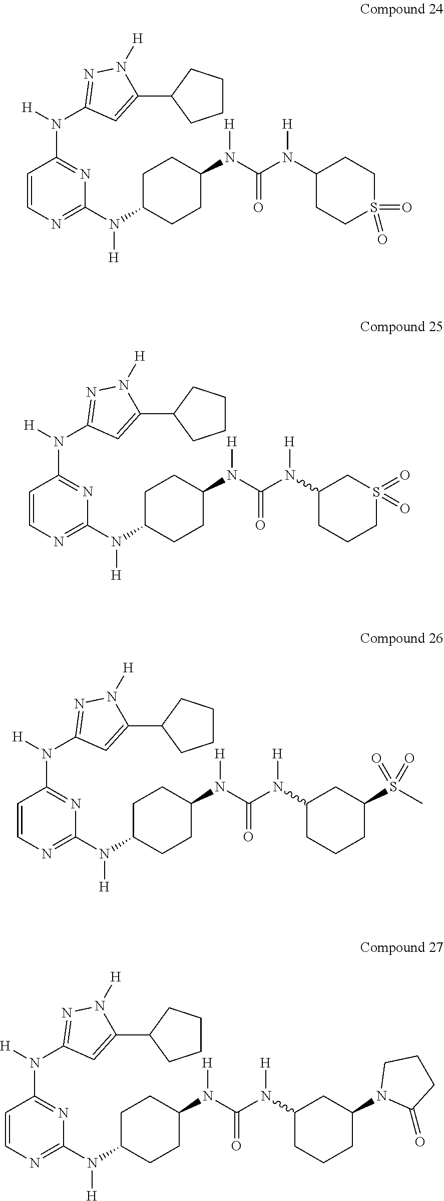 [SMILES: O=C(NC1CCS(=O)(=O)CC1)N[C@H]1CC[C@H](Nc2nccc(Nc3cc(C4CCCC4)[nH]n3)n2)CC1 ; O=C(NC1CCCS(=O)(=O)C1)N[C@H]1CC[C@H](Nc2nccc(Nc3cc(C4CCCC4)[nH]n3)n2)CC1 ; CS(=O)(=O)[C@H]1CCCC(NC(=O)N[C@H]2CC[C@H](Nc3nccc(Nc4cc(C5CCCC5)[nH]n4)n3)CC2)C1 ; O=C(NC1CCC[C@H](N2CCCC2=O)C1)N[C@H]1CC[C@H](Nc2nccc(Nc3cc(C4CCCC4)[nH]n3)n2)CC1] 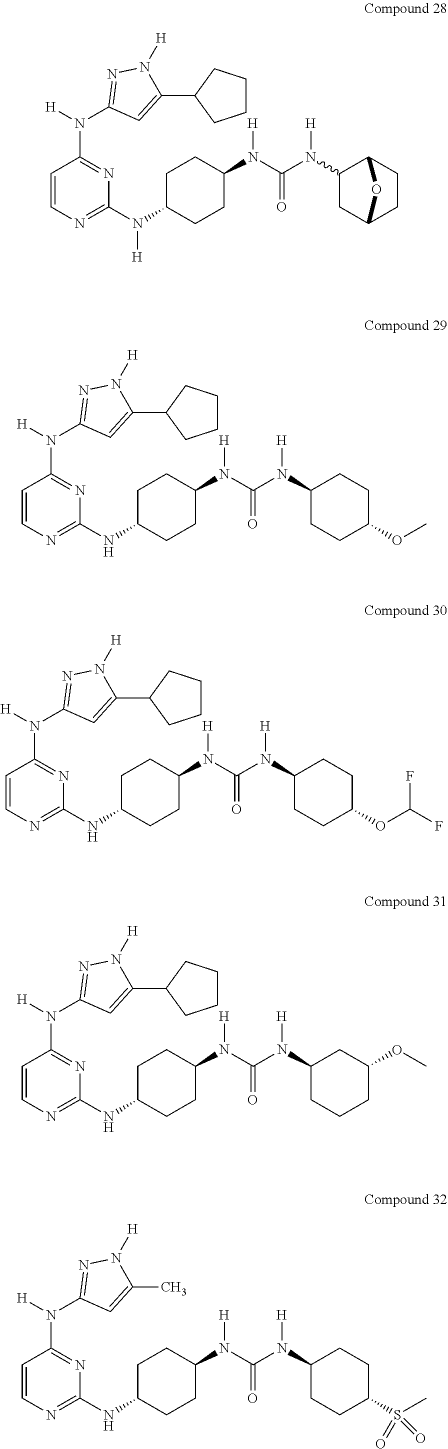 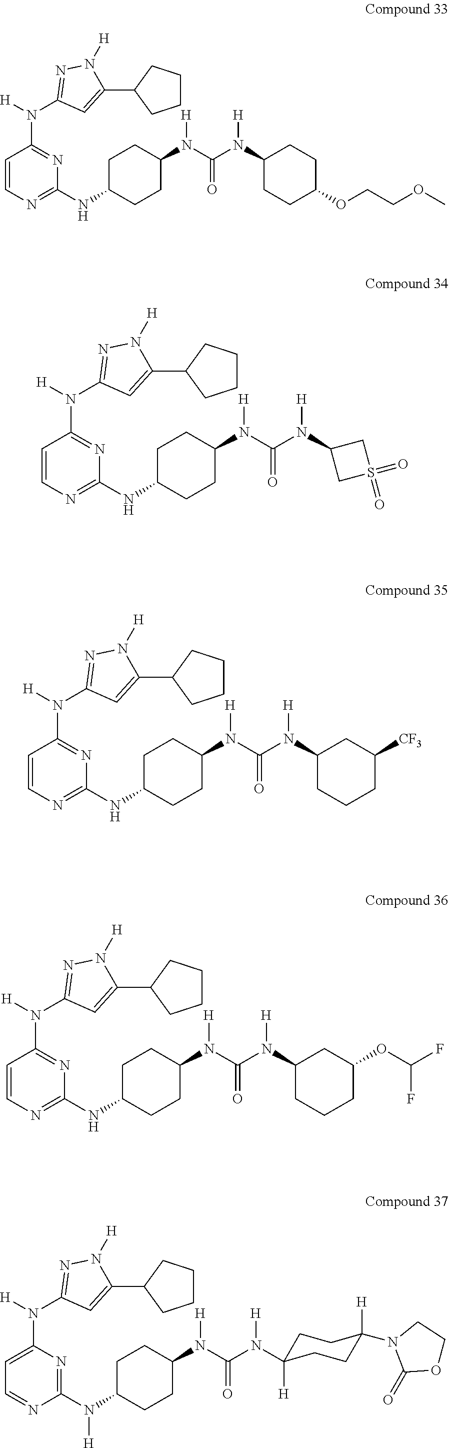 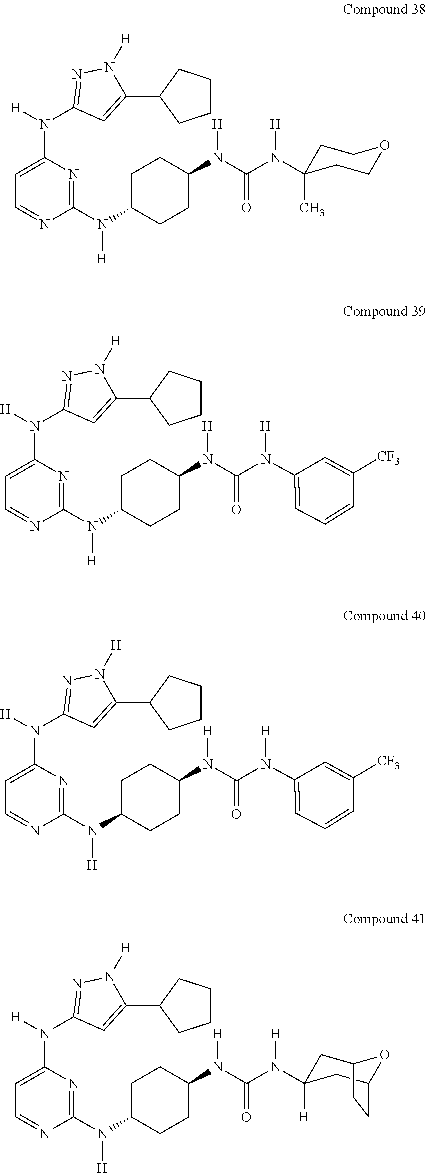 [SMILES: CC1(NC(=O)N[C@H]2CC[C@H](Nc3nccc(Nc4cc(C5CCCC5)[nH]n4)n3)CC2)CCOCC1 ; O=C(Nc1cccc(C(F)(F)F)c1)N[C@H]1CC[C@H](Nc2nccc(Nc3cc(C4CCCC4)[nH]n3)n2)CC1 ; O=C(Nc1cccc(C(F)(F)F)c1)N[C@H]1CC[C@@H](Nc2nccc(Nc3cc(C4CCCC4)[nH]n3)n2)CC1 ; O=C(NC1CC2CCC(C1)O2)N[C@H]1CC[C@H](Nc2nccc(Nc3cc(C4CCCC4)[nH]n3)n2)CC1] 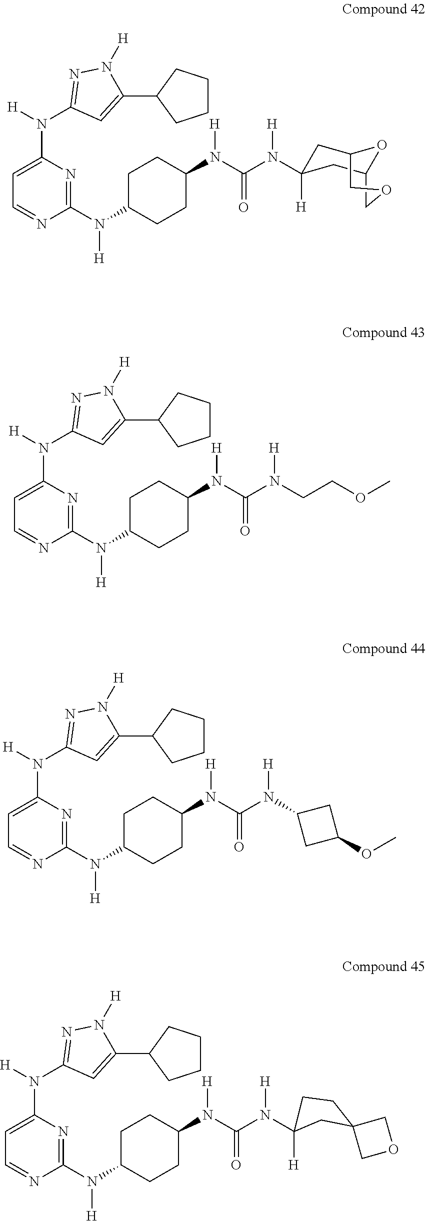 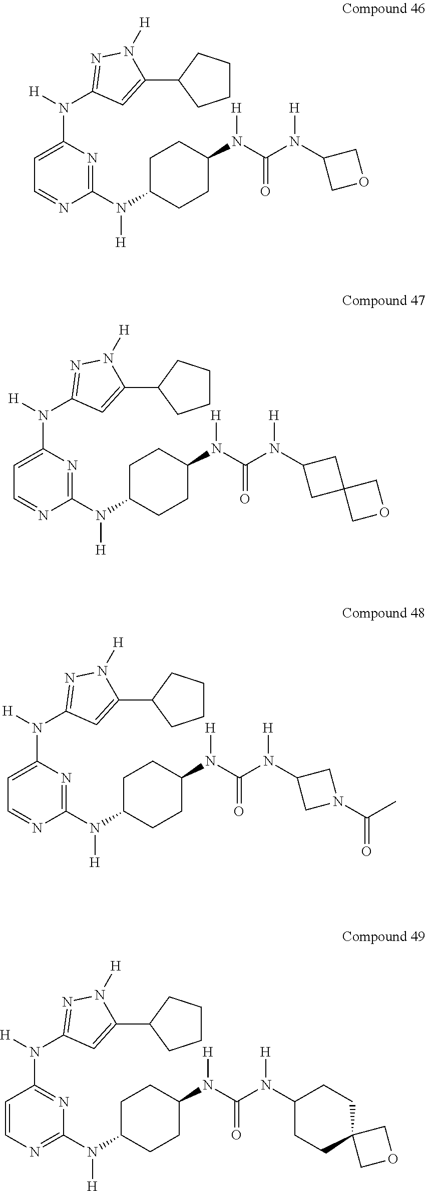 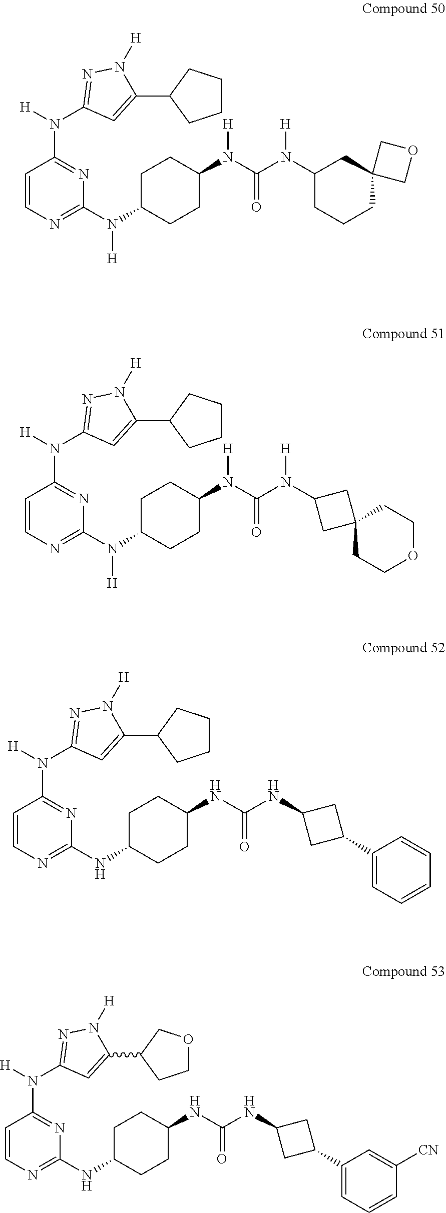 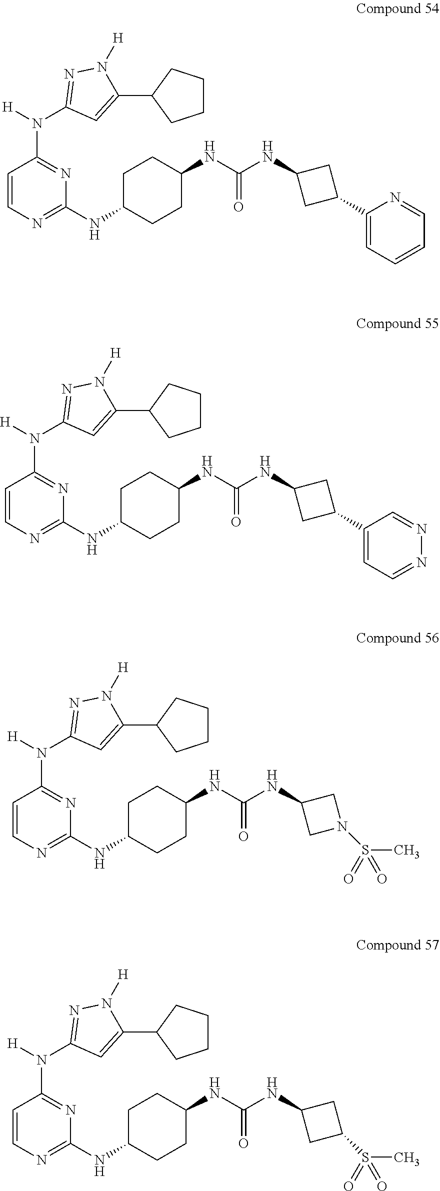 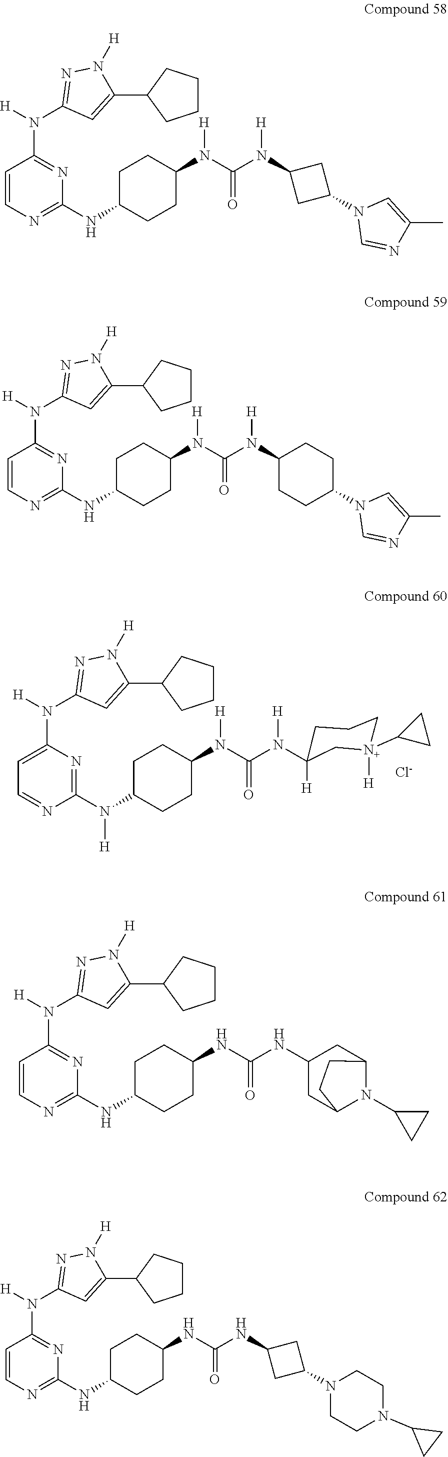 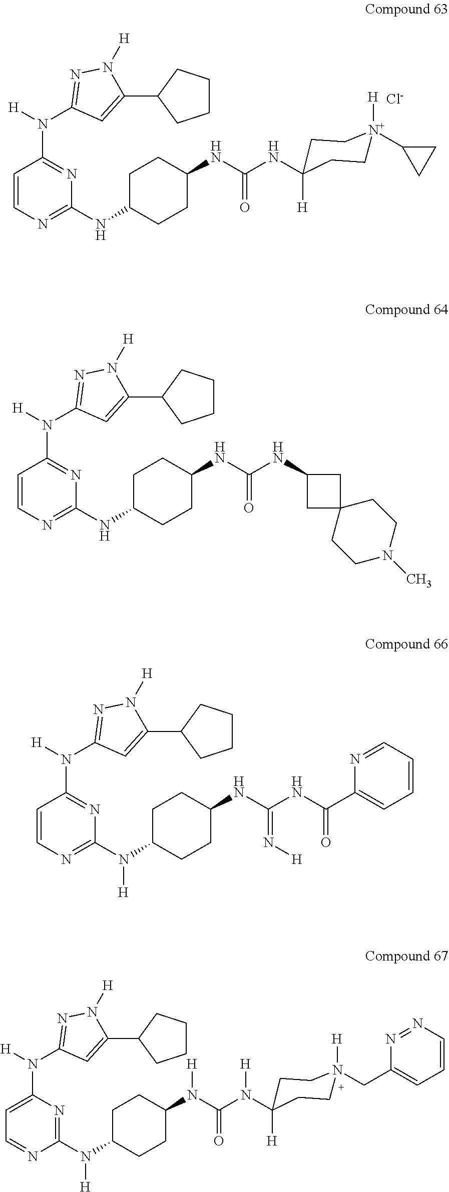 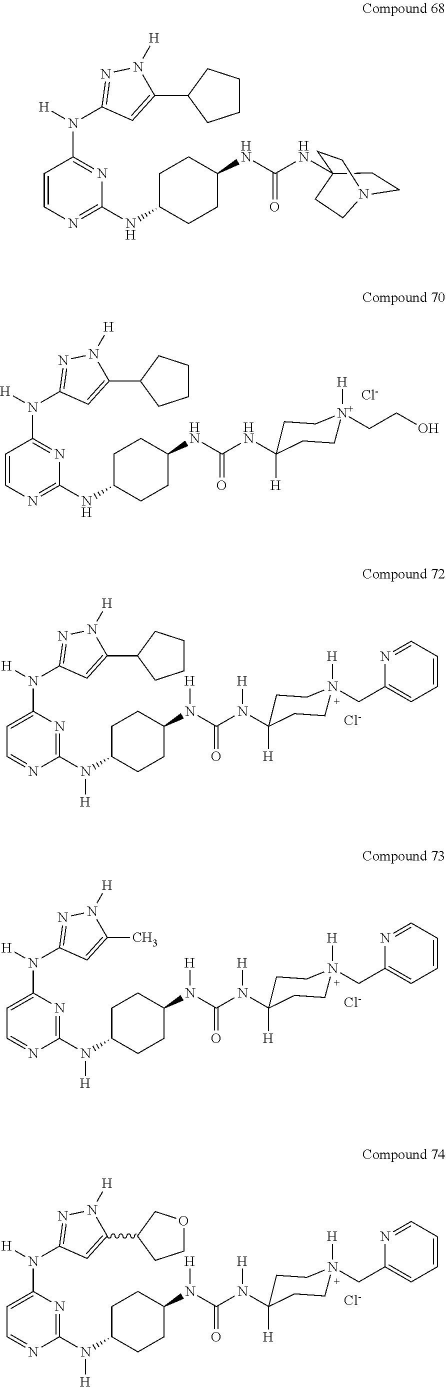 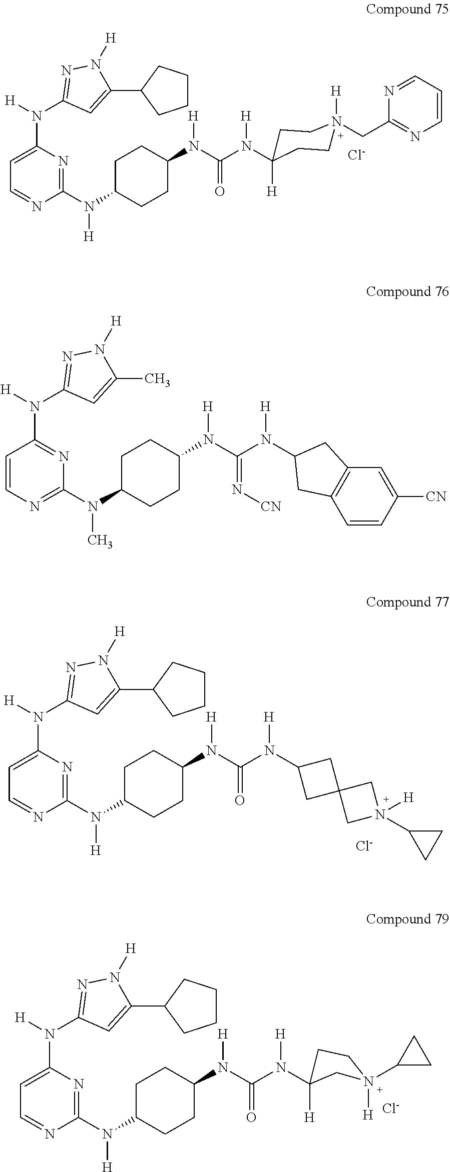 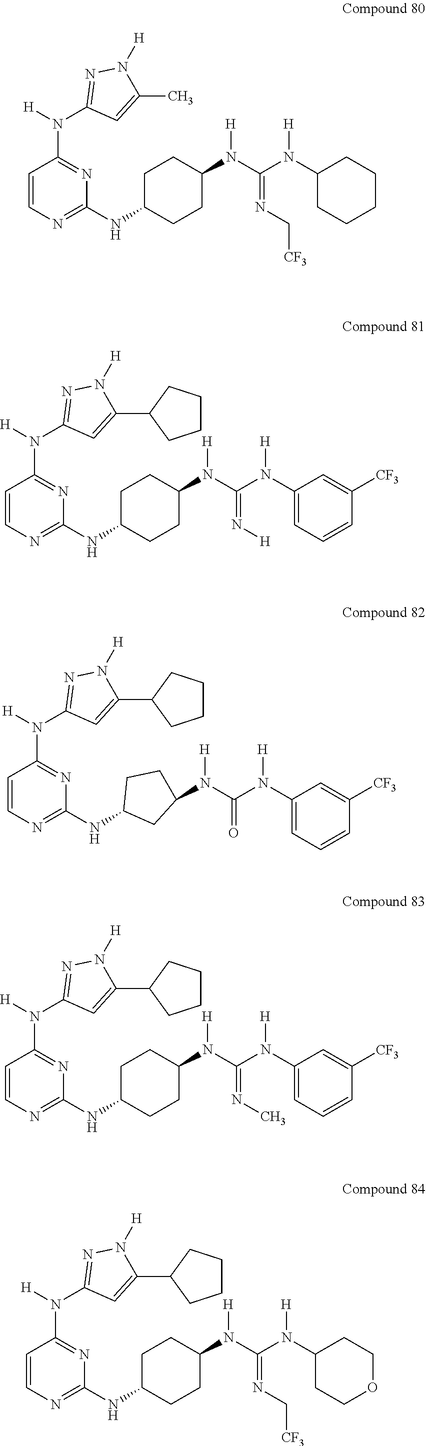 [SMILES: Cc1cc(Nc2ccnc(N[C@H]3CC[C@H](N/C(=N/CC(F)(F)F)NC4CCCCC4)CC3)n2)n[nH]1 ; [H]/N=C(\Nc1cccc(C(F)(F)F)c1)N[C@H]1CC[C@H](Nc2nccc(Nc3cc(C4CCCC4)[nH]n3)n2)CC1 ; O=C(Nc1cccc(C(F)(F)F)c1)N[C@@H]1CC[C@@H](Nc2nccc(Nc3cc(C4CCCC4)[nH]n3)n2)C1 ; C/N=C(\Nc1cccc(C(F)(F)F)c1)N[C@H]1CC[C@H](Nc2nccc(Nc3cc(C4CCCC4)[nH]n3)n2)CC1 ; FC(F)(F)C/N=C(\NC1CCOCC1)N[C@H]1CC[C@H](Nc2nccc(Nc3cc(C4CCCC4)[nH]n3)n2)CC1] 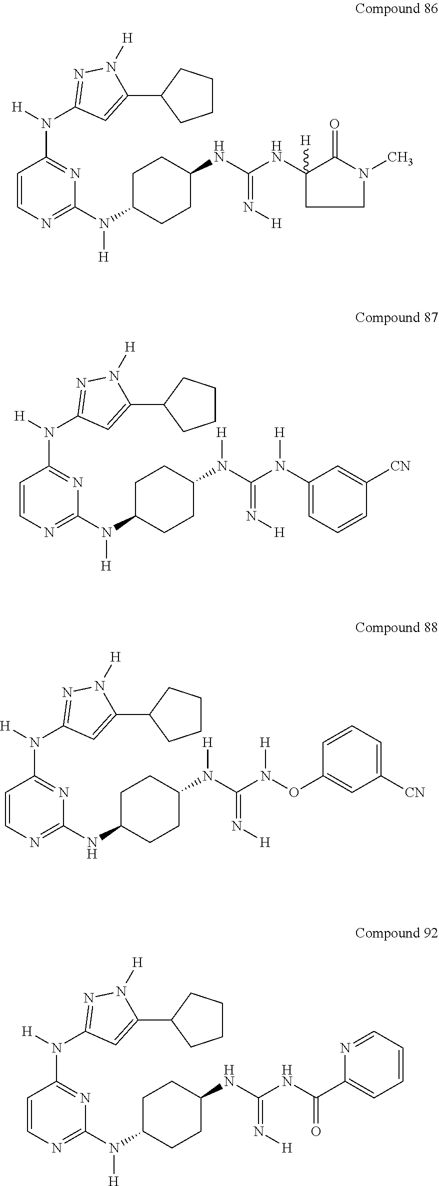 [SMILES: [H]/N=C(\NC1CCN(C)C1=O)N[C@H]1CC[C@H](Nc2nccc(Nc3cc(C4CCCC4)[nH]n3)n2)CC1 ; [H]/N=C(\Nc1cccc(C#N)c1)N[C@H]1CC[C@H](Nc2nccc(Nc3cc(C4CCCC4)[nH]n3)n2)CC1 ; [H]/N=C(\NOc1cccc(C#N)c1)N[C@H]1CC[C@H](Nc2nccc(Nc3cc(C4CCCC4)[nH]n3)n2)CC1 ; [H]/N=C(\NC(=O)c1ccccn1)N[C@H]1CC[C@H](Nc2nccc(Nc3cc(C4CCCC4)[nH]n3)n2)CC1] 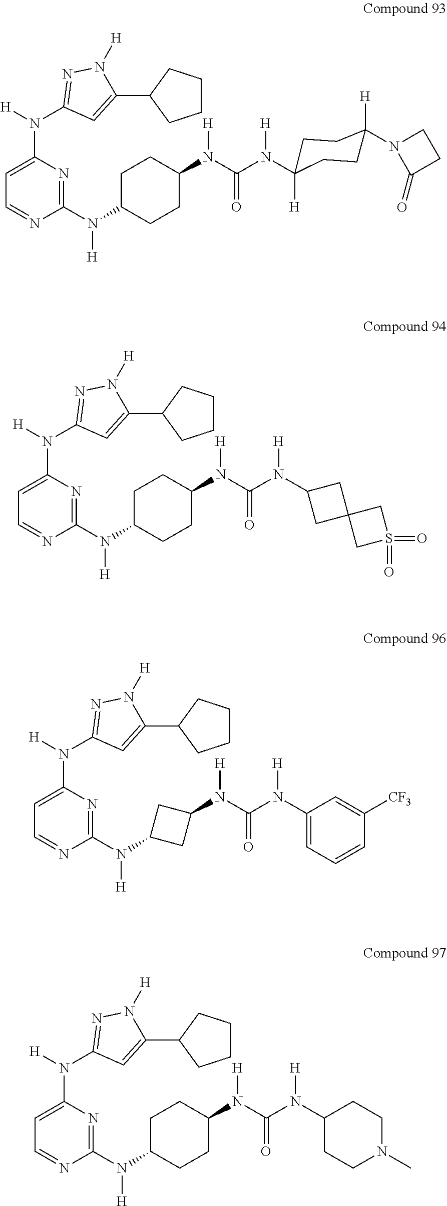 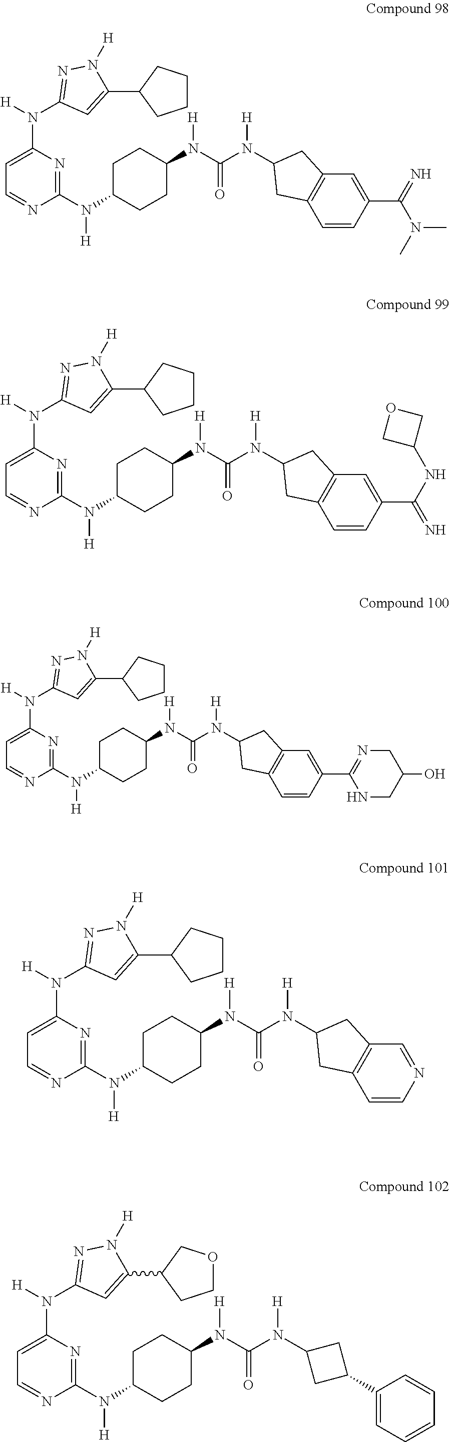 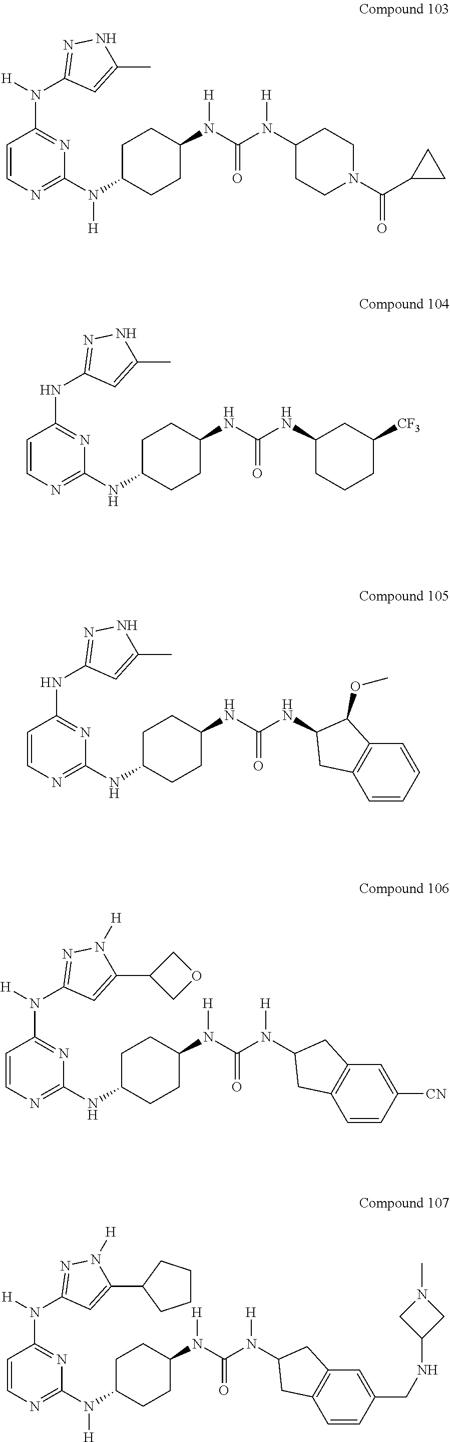 [SMILES: Cc1cc(Nc2ccnc(N[C@H]3CC[C@H](NC(=O)NC4CCN(C(=O)C5CC5)CC4)CC3)n2)n[nH]1 ; Cc1cc(Nc2ccnc(N[C@H]3CC[C@H](NC(=O)N[C@@H]4CCC[C@H](C(F)(F)F)C4)CC3)n2)n[nH]1 ; CO[C@H]1c2ccccc2C[C@H]1NC(=O)N[C@H]1CC[C@H](Nc2nccc(Nc3cc(C)[nH]n3)n2)CC1 ; N#Cc1ccc2c(c1)CC(NC(=O)N[C@H]1CC[C@H](Nc3nccc(Nc4cc(C5COC5)[nH]n4)n3)CC1)C2 ; CN1CC(NCc2ccc3c(c2)CC(NC(=O)N[C@H]2CC[C@H](Nc4nccc(Nc5cc(C6CCCC6)[nH]n5)n4)CC2)C3)C1] 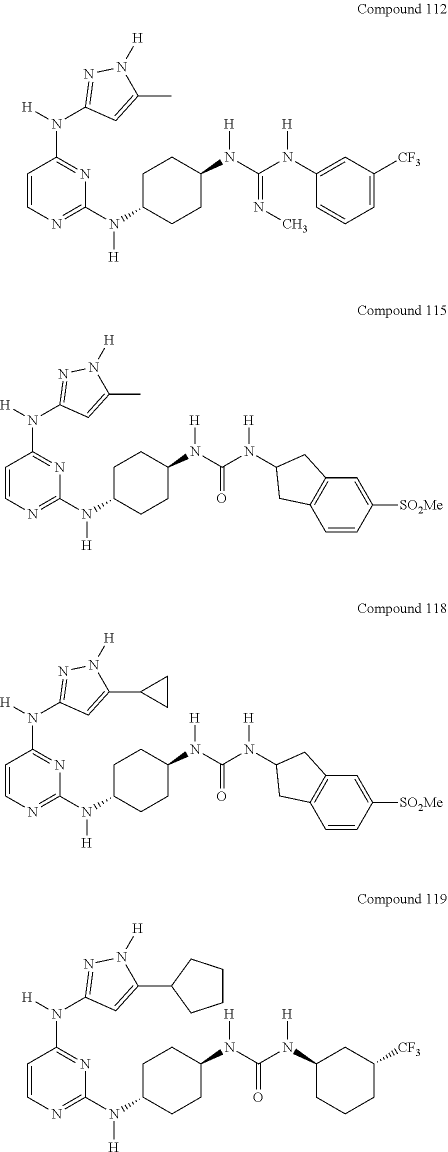 [SMILES: C/N=C(\Nc1cccc(C(F)(F)F)c1)N[C@H]1CC[C@H](Nc2nccc(Nc3cc(C)[nH]n3)n2)CC1 ; Cc1cc(Nc2ccnc(N[C@H]3CC[C@H](NC(=O)NC4Cc5ccc(S(C)(=O)=O)cc5C4)CC3)n2)n[nH]1 ; CS(=O)(=O)c1ccc2c(c1)CC(NC(=O)N[C@H]1CC[C@H](Nc3nccc(Nc4cc(C5CC5)[nH]n4)n3)CC1)C2 ; O=C(N[C@@H]1CCC[C@@H](C(F)(F)F)C1)N[C@H]1CC[C@H](Nc2nccc(Nc3cc(C4CCCC4)[nH]n3)n2)CC1] 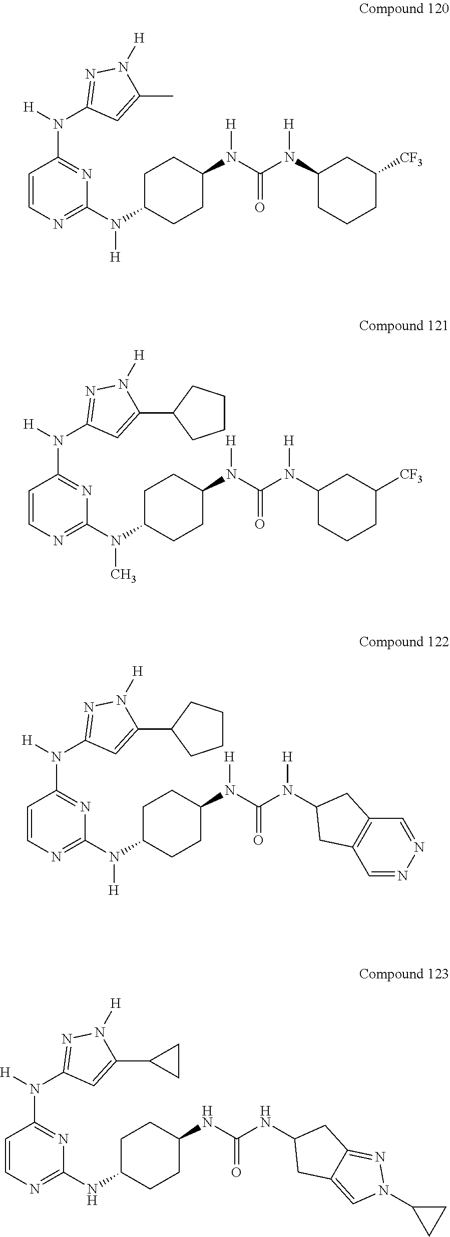 [SMILES: Cc1cc(Nc2ccnc(N[C@H]3CC[C@H](NC(=O)N[C@@H]4CCC[C@@H](C(F)(F)F)C4)CC3)n2)n[nH]1 ; CN(c1nccc(Nc2cc(C3CCCC3)[nH]n2)n1)[C@H]1CC[C@H](NC(=O)NC2CCCC(C(F)(F)F)C2)CC1 ; O=C(NC1Cc2cnncc2C1)N[C@H]1CC[C@H](Nc2nccc(Nc3cc(C4CCCC4)[nH]n3)n2)CC1 ; O=C(NC1Cc2cn(C3CC3)nc2C1)N[C@H]1CC[C@H](Nc2nccc(Nc3cc(C4CC4)[nH]n3)n2)CC1] 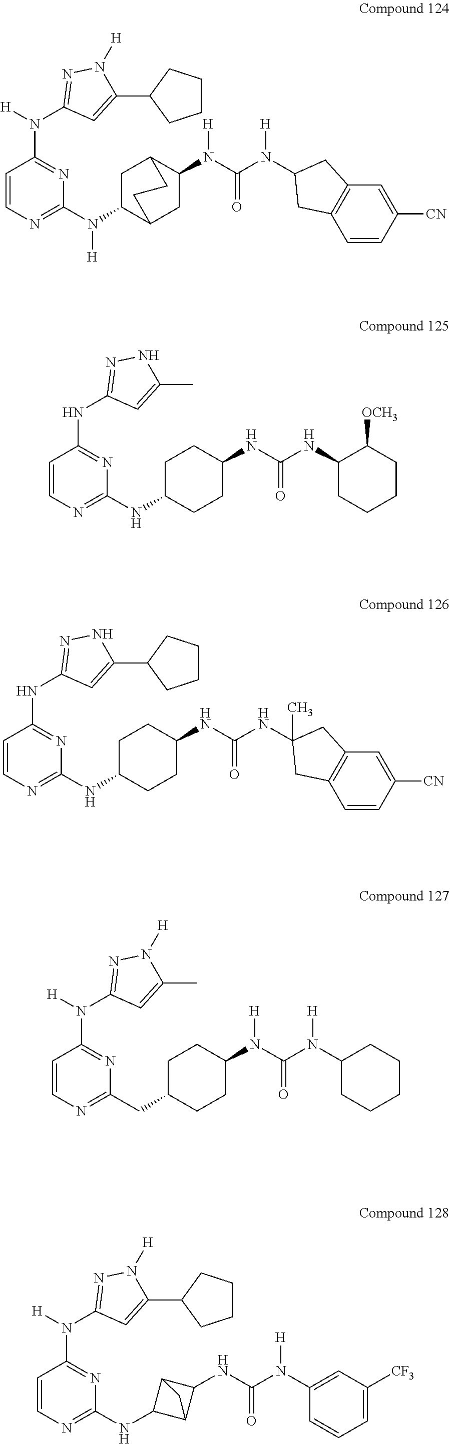 [SMILES: N#Cc1ccc2c(c1)CC(NC(=O)N[C@H]1CC3CCC1C[C@H]3Nc1nccc(Nc3cc(C4CCCC4)[nH]n3)n1)C2 ; CO[C@H]1CCCC[C@H]1NC(=O)N[C@H]1CC[C@H](Nc2nccc(Nc3cc(C)[nH]n3)n2)CC1 ; CC1(NC(=O)N[C@H]2CC[C@H](Nc3nccc(Nc4cc(C5CCCC5)[nH]n4)n3)CC2)Cc2ccc(C#N)cc2C1 ; Cc1cc(Nc2ccnc(C[C@H]3CC[C@H](NC(=O)NC4CCCCC4)CC3)n2)n[nH]1 ; O=C(Nc1cccc(C(F)(F)F)c1)NC1C2CC1C2Nc1nccc(Nc2cc(C3CCCC3)[nH]n2)n1] 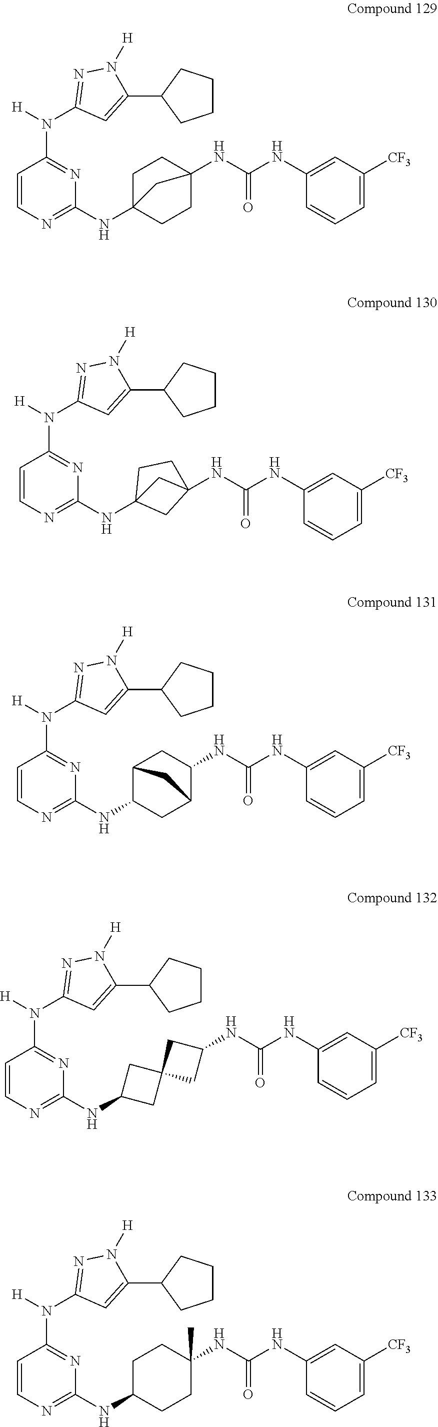 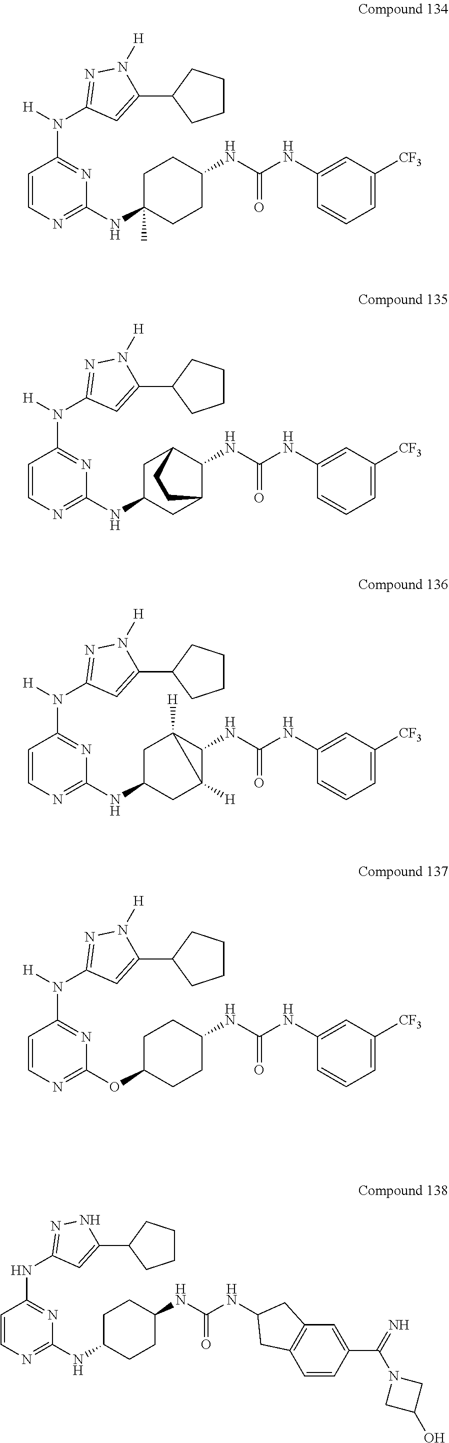 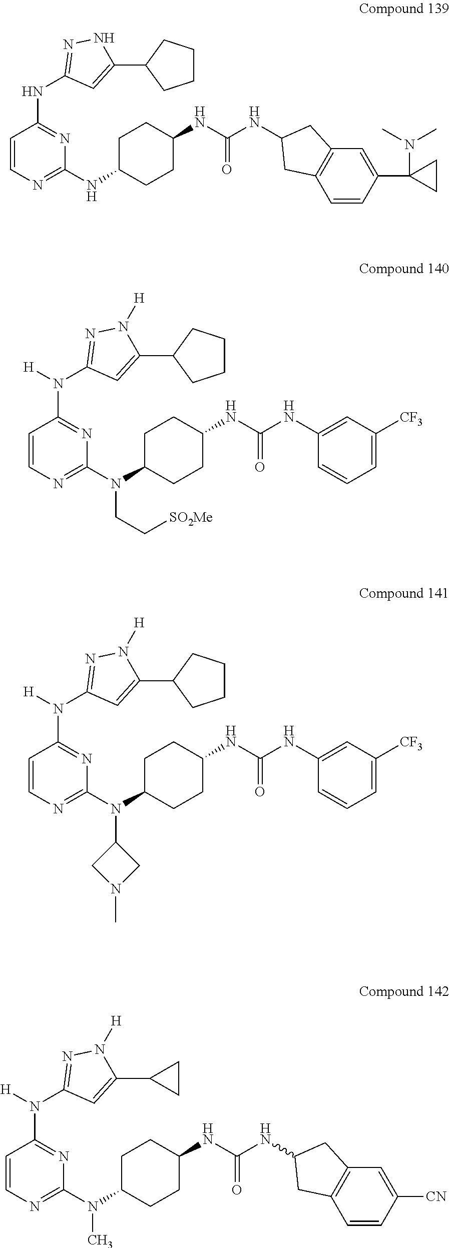 [SMILES: CN(C)C1(c2ccc3c(c2)CC(NC(=O)N[C@H]2CC[C@H](Nc4nccc(Nc5cc(C6CCCC6)[nH]n5)n4)CC2)C3)CC1 ; CS(=O)(=O)CCN(c1nccc(Nc2cc(C3CCCC3)[nH]n2)n1)[C@H]1CC[C@H](NC(=O)Nc2cccc(C(F)(F)F)c2)CC1 ; CN1CC(N(c2nccc(Nc3cc(C4CCCC4)[nH]n3)n2)[C@H]2CC[C@H](NC(=O)Nc3cccc(C(F)(F)F)c3)CC2)C1 ; CN(c1nccc(Nc2cc(C3CC3)[nH]n2)n1)[C@H]1CC[C@H](NC(=O)NC2Cc3ccc(C#N)cc3C2)CC1] 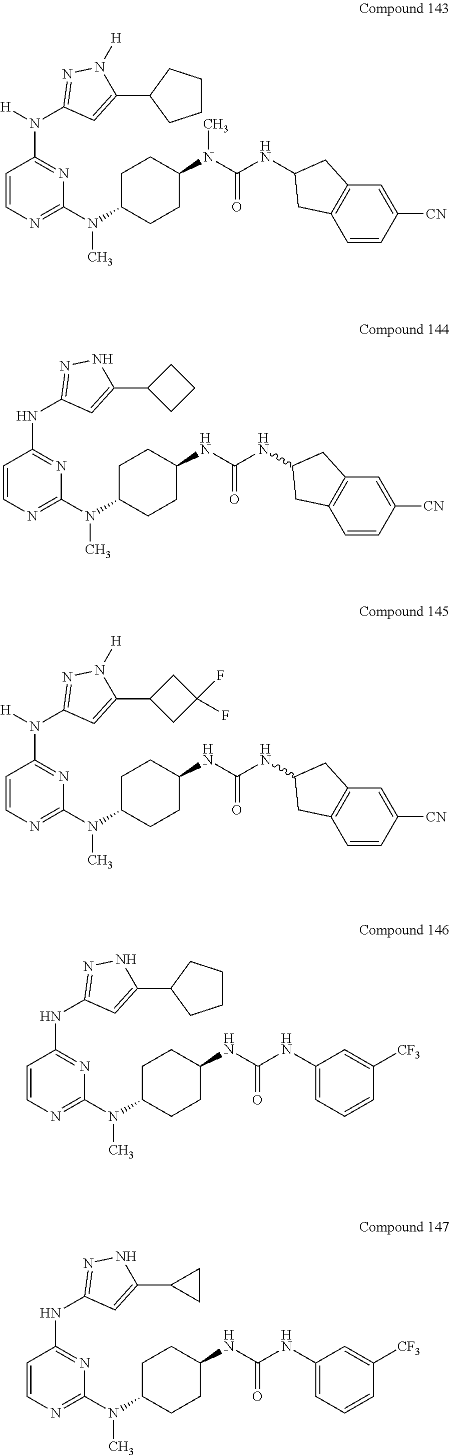 [SMILES: CN(C(=O)NC1Cc2ccc(C#N)cc2C1)[C@H]1CC[C@H](N(C)c2nccc(Nc3cc(C4CCCC4)[nH]n3)n2)CC1 ; CN(c1nccc(Nc2cc(C3CCC3)[nH]n2)n1)[C@H]1CC[C@H](NC(=O)NC2Cc3ccc(C#N)cc3C2)CC1 ; CN(c1nccc(Nc2cc(C3CC(F)(F)C3)[nH]n2)n1)[C@H]1CC[C@H](NC(=O)NC2Cc3ccc(C#N)cc3C2)CC1 ; CN(c1nccc(Nc2cc(C3CCCC3)[nH]n2)n1)[C@H]1CC[C@H](NC(=O)Nc2cccc(C(F)(F)F)c2)CC1 ; CN(c1nccc(Nc2cc(C3CC3)[nH]n2)n1)[C@H]1CC[C@H](NC(=O)Nc2cccc(C(F)(F)F)c2)CC1] 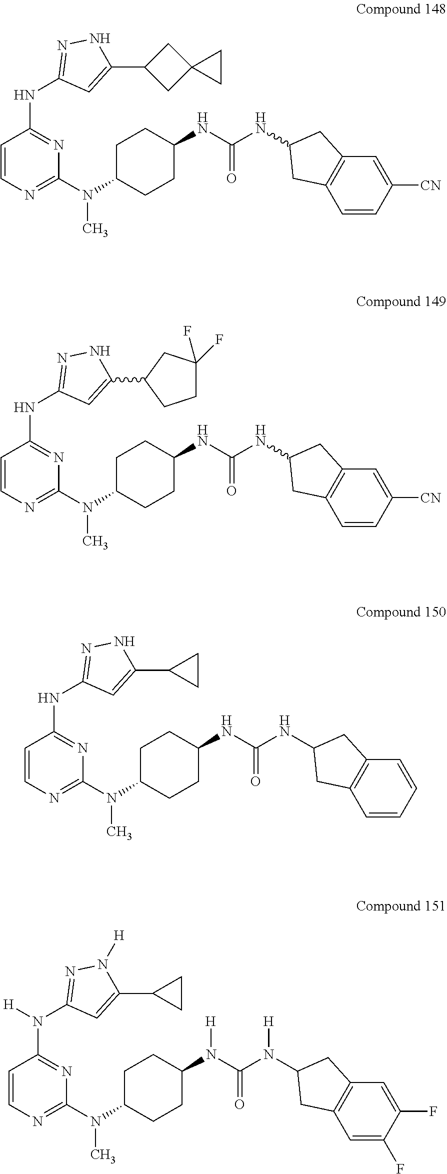 [SMILES: CN(c1nccc(Nc2cc(C3CC4(CC4)C3)[nH]n2)n1)[C@H]1CC[C@H](NC(=O)NC2Cc3ccc(C#N)cc3C2)CC1 ; CN(c1nccc(Nc2cc(C3CCC(F)(F)C3)[nH]n2)n1)[C@H]1CC[C@H](NC(=O)NC2Cc3ccc(C#N)cc3C2)CC1 ; CN(c1nccc(Nc2cc(C3CC3)[nH]n2)n1)[C@H]1CC[C@H](NC(=O)NC2Cc3ccccc3C2)CC1 ; CN(c1nccc(Nc2cc(C3CC3)[nH]n2)n1)[C@H]1CC[C@H](NC(=O)NC2Cc3cc(F)c(F)cc3C2)CC1] 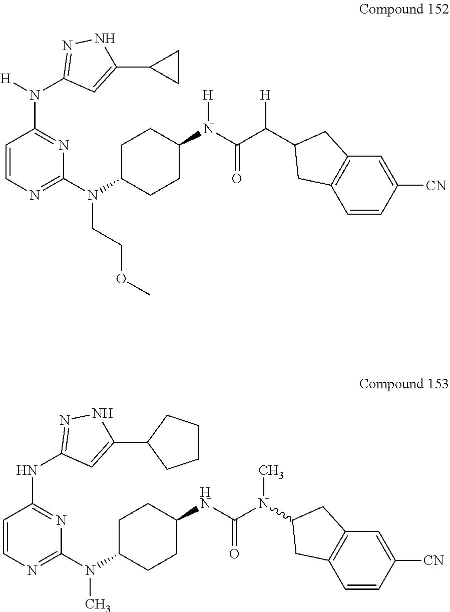 [SMILES: COCCN(c1nccc(Nc2cc(C3CC3)[nH]n2)n1)[C@H]1CC[C@H](NC(=O)CC2Cc3ccc(C#N)cc3C2)CC1 ; CN(C(=O)N[C@H]1CC[C@H](N(C)c2nccc(Nc3cc(C4CCCC4)[nH]n3)n2)CC1)C1Cc2ccc(C#N)cc2C1]
and a pharmaceutically acceptable salt, stereoisomer and/or N-oxide thereof.

44. A pharmaceutical composition comprising a compound according to any one of claims 1-43 or or a pharmaceutically acceptable salt, stereoisomer and/or N-oxide thereof, and at least one pharmaceutically acceptable carrier or diluent.

45. The pharmaceutical composition of claim 44, wherein the composition is formulated for parenteral administration.

46. The pharmaceutical composition of claim 44, wherein the composition is formulated for intravenous administration.

47. The pharmaceutical composition of claim 44, wherein the composition is formulated for subcutaneous administration.

48. A method of treating a proliferative disease, comprising:
administering to a subject with a proliferative disease a therapeutically effective amount of a compound according to any one of claims 1-43, or a pharmaceutically acceptable salt, stereoisomer and/or N-oxide thereof, or a therapeutically effective amount of the pharmaceutical composition of any one of claims 44-47.

49. The method of claim 48, wherein the proliferative disease is cancer.

50. The method of claim 49, wherein the cancer is selected from the group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

51. A method of modulating MycN in cells of a subject in need thereof, comprising:
administering to a subject in need thereof an amount of a compound according to any one of claims 1-43, or a pharmaceutically acceptable salt, stereoisomer and/or N-oxide thereof, or a pharmaceutical composition according to any one of claims 44-47, that is effective to cause MycN modulation in cells of the subject.

52. The method of any one of claims 48-51, further comprising administering to the subject a second therapy.

53. The method of claim 52, wherein the second therapy is an antineoplastic therapy.

54. The method of claim 53, wherein the antineoplastic therapy is administration of one or more agents selected from a DNA topoisomerase I or II inhibitor, a DNA damaging agent, an immunotherapeutic agent, an antimetabolite or a thymidylate synthase (TS) inhibitor, a microtubule targeted agent, ionising radiation, an inhibitor of a mitosis regulator or a mitotic checkpoint regulator, an inhibitor of a DNA damage signal transducer, and an inhibitor of a DNA damage repair enzyme.

55. The method of claim 53, wherein the antineoplastic therapy is selected from the group consisting of immunotherapy, radiation therapy, photodynamic therapy, gene-directed enzyme prodrug therapy (GDEPT), antibody-directed enzyme prodrug therapy (ADEPT), gene therapy, and controlled diets.
